(19) United States
(12) Patent Application Publication
(10) Pub. No.: US 2024/0391930 A1
(43) Pub. Date: Nov. 28, 2024

(54) SPIROCYCLIC BICYCLIC MODULATORS OF CHOLESTEROL BIOSYNTHESIS AND THEIR USE FOR PROMOTING REMYELINATION

(71) Applicants: GENENTECH, INC., SOUTH SAN FRANCISCO, CA (US); CONVELO THERAPEUTICS, INC., Cleveland, OH (US)

(72) Inventors: Marie-Gabrielle BRAUN, South San Francisco, CA (US); Georgette CASTANEDO, South San Francisco, CA (US); William VERNIER, South San Francisco, CA (US); Matthew VOLGRAF, South San Francisco, CA (US); Michael SIU, South San Francisco, CA (US); James Guy BREITENBUCHER, Valley View, OH (US)

(21) Appl. No.: 18/670,537

(22) Filed: May 21, 2024

Related U.S. Application Data
(63) Continuation of application No. PCT/US2022/080354, filed on Nov. 22, 2022.
(60) Provisional application No. 63/282,357, filed on Nov. 23, 2021.

(51) Int. Cl. C07D 495/10; A61K 31/4155; A61K 31/4196; A61K 31/438; A61K 31/4439; A61K 31/444; A61K 31/506

(52) U.S. Cl. CPC C07D 495/10; A61K 31/4155; A61K 31/4196; A61K 31/438; A61K 31/4439; A61K 31/444; A61K 31/506

(57) ABSTRACT

The subject matter described herein is directed to myelin-promoting compounds of Formula I and pharmaceutical salts thereof, methods of preparing the compounds, pharmaceutical compositions comprising the compounds, and methods of administering the compounds for the treatment of disorders, such as myelin-related disorders.

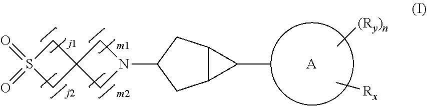

### CROSS REFERENCE TO RELATED APPLICATIONS

This application is a bypass continuation of international application no. PCT/US2022/080354 filed Nov. 22, 2022, which claims the benefit of priority to U.S. Provisional Patent Application No. 63/282,357, filed on Nov. 23, 2021, the contents of which is incorporated by reference herein in its entirety for all purposes.

### FIELD

The subject matter described herein is directed to myelin-promoting compounds of Formula I, methods of making the compounds, their pharmaceutical compositions, and their use in the treatment of myelin-related disorders.

### BACKGROUND

Myelin-related disorders are disorders that result in abnormalities of the myelin sheath (e.g., dysmyelination, demyelination and hypomyelination) in a subject's neural cells, e.g., CNS neurons including their axons. Loss or degradation of the myelin sheath in such disorders produces a slowing or cessation of nerve cell conduction. The resulting myelin related disorders are characterized by deficits in sensation, motor function, cognition, or other physiological functions. Myelin related disorders include, but are not limited to, multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy, and radiation-induced demyelination.

MS is the most common myelin-related disorder affecting several million people globally and is estimated to result in about 18,000 deaths per year. MS is a complex neurological disease characterized by deterioration of central nervous system (CNS) myelin. Myelin, composed in its majority by lipids (70% lipids, 30% protein), protects axons and makes saltatory conduction possible, which speeds axonal electric impulse. Demyelination of axons in chronic MS can result in axon degeneration and neuronal cell death. Additionally, MS destroys oligodendrocytes, the highly specialized CNS cells that generate and maintain myelin. A repair process, called remyelination, takes place in early phases of the disease, but over time, the oligodendrocytes are unable to completely rebuild and restore the myelin sheath. Repeated attacks lead to successively less effective remyelination, until a scar-like plaque is built up around the damaged axons. These scars are the origin of the symptoms.

At present, there is no cure for myelin-related disorders, and only a handful of disease-modifying therapies are available. Accordingly, there is a need for new therapeutic approaches to the treatment of myelin-related disorders, including the promotion of remyelination. The subject matter described herein addresses this unmet need.

### BRIEF SUMMARY

In certain embodiments, the subject matter described herein is directed to a compound of Formula I or a pharmaceutically acceptable salt thereof.

In certain embodiments, the subject matter described herein is directed to a pharmaceutical composition comprising a compound of Formula I or a pharmaceutically acceptable salt thereof, and a pharmaceutically acceptable excipient.

In certain embodiments, the subject matter described herein is directed to methods of treating a disorder in a subject in need thereof, wherein the disorder is a myelin-related disorder, comprising administering to the subject an effective amount of a compound of Formula I, or a pharmaceutically acceptable salt thereof; or a pharmaceutical composition comprising a compound of Formula I, or a pharmaceutically acceptable salt thereof, and a pharmaceutically acceptable excipient.

In certain embodiments, the subject matter described herein is directed to a compound of Formula I, or a pharmaceutically acceptable salt thereof, or a pharmaceutical composition comprising a compound of Formula I, or a pharmaceutically acceptable salt thereof, for use in treating a myelin-related disorder.

In certain embodiments, the subject matter described herein is directed to methods of promoting myelination in a subject in need thereof, comprising administering to the subject a therapeutically effective amount of a compound of Formula I, or a pharmaceutically acceptable salt thereof; or a pharmaceutical composition comprising a compound of Formula I, or a pharmaceutically acceptable salt thereof, and a pharmaceutically acceptable excipient.

In certain embodiments, the subject matter described herein is directed to the use of a compound of Formula I, or a pharmaceutically acceptable salt thereof, or a pharmaceutical composition comprising a compound of Formula I, or a pharmaceutically acceptable salt thereof, in the manufacture of a medicament for treating a myelin-related disorder.

In certain embodiments, the subject matter described herein is directed to methods of preparing compounds of Formula I, or a pharmaceutically acceptable salt thereof.

Other embodiments are also described.

### DETAILED DESCRIPTION

Described herein are compounds of Formula I, methods of making the compounds, their pharmaceutical compositions, and their use in the treatment of myelin-related disorders. In some embodiments, the compounds provided herein are myelin-promoting.

Without wishing to be bound by theory, the enhancement and/or inducement of the accumulation of Δ8,9-unsaturated sterol intermediates of the cholesterol biosynthesis pathway in oligodendrocyte progenitor cells (OPCs) can induce oligodendrocyte generation. Enhancement and/or inducement of the accumulation of Δ8,9-unsaturated sterol intermediates can be provided, for example, by modulating and/or inhibiting the enzymes within the OPC cholesterol biosynthesis pathway that inhibit Δ8,9-unsaturated sterol intermediate accumulation and/or for which the Δ8,9-unsaturated sterol intermediates are substrates, as well as directly and/or indirectly administering Δ8,9-unsaturated sterol intermediates to the OPCs. Enhancement and/or inducement of the accumulation of Δ8,9-unsaturated sterol intermediates may promote OPC differentiation, survival, proliferation, and/or maturation, and it is thought this might treat disease and/or disorders in subjects where myelination is beneficial to the subject.

As such, in some embodiments, an agent, such as a compound of Formula I, or a pharmaceutically acceptable salt thereof, that can enhance and/or induce accumulation of Δ8,9-unsaturated sterol intermediates of the cholesterol biosynthesis pathway in OPCs can be administered to a subject, and/or to the OPCs, at an amount effective to promote and/or induce OPC differentiation, proliferation, and/or maturation, as well as oligodendrocyte generation. In certain embodiments, the agent, for example a compound of Formula I, or a pharmaceutically acceptable salt thereof, is a compound that inhibits enzyme-mediated synthesis of one or more sterol intermediates in the cholesterol biosynthesis pathway of the OPCs, and/or promotes accumulation of Δ8,9-unsaturated sterol intermediates.

In certain embodiments, the compound of Formula I, or a pharmaceutically acceptable salt thereof, can modulate and/or inhibit one or more enzyme-mediated conversion steps of the cholesterol biosynthises pathway, such as in the pathway from lanosterol to cholesterol, for example, between lanosterol and/or lathosterol; modulating and/or inhibiting one or more of these steps in OPCs may promote and/or induce oligodendrocyte generation. For example, in some embodiments, a compound of Formula I or pharmaceutically acceptable salt thereof can inhibit CYP51, sterol 14-reductase (TM7SF2 and/or LBR), SC4MOL, NSDHL, and/or emopamil binding protein (EBP) enzyme mediated synthesis of sterol intermediates in the cholesterol biosynthesis pathway. In certain embodiments, a compound of Formula I or pharmaceutically acceptable salt thereof can inhibit CYP51, sterol 14-reductase and/or EBP. In certain embodiments, the compound of Formula I or pharmaceutically acceptable salt thereof can inhibit EBP.

For example, in certain embodiments, the compound of Formula I, or pharmaceutically acceptable salt thereof, used in the methods described herein can inhibit enzyme mediated conversion of zymostenol to lathosterol through the inhibition of emopamil binding protein (EBP) isomerase enzyme activity. Alternatively, in certain embodiments, the compound of Formula I, or pharmaceutically acceptable salt thereof, used in the methods described herein can inhibit sterol C14 reductase enzyme activity or CYP51 enzyme activity in the cholesterol biosynthesis pathway.

Emopamil Binding Protein (EBP) is an enzyme responsible for one of the final steps in the production of cholesterol. Specifically, EBP converts zymostenol to lathosterol, where other enzymes then modify lathosterol to produce cholesterol. EBP is also referred to as Δ8-Δ7-sterol isomerase, 3-beta-hydroxysteroid-Delta(8),Delta(7)-isomerase, CDPX2, CHO2, CPX, or CPXD).

Without being bound by a particular theory, it is believed that compounds of Formula I or a pharmaceutically acceptable salt thereof can inhibit EBP mediated conversion of zymostenol to lathosterol in the cholesterol biosynthesis pathway of OPCs resulting in enhancement and/or inducement of the accumulation of Δ8,9-unsaturated sterol intermediates. In some embodiments, enhancement and/or inducement of the accumulation of Δ8,9-unsaturated sterol intermediates can promote OPC differentiation, survival, proliferation and/or maturation and treat disease and/or disorders in subjects where myelination or myelinization is beneficial to the subject. This mechanism of promoting myelination is distinct from the primary action of immunomodulatory agents that are often used to treat myelin-related disorders.

The presently disclosed subject matter will now be described more fully hereinafter. However, many modifications and other embodiments of the presently disclosed subject matter set forth herein may come to mind to one skilled in the art to which the presently disclosed subject matter pertains having the benefit of the teachings presented in the descriptions herein. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. Unless otherwise defined, all technical and scientific terms used herein have the same meaning as commonly understood by one of ordinary skill in this field. All publications, patent applications, patents, and other references mentioned herein are incorporated by reference in their entirety. In the event that one or more of the incorporated literature, patents, and similar materials differs from or contradicts this application, including but not limited to defined terms, term usage, described techniques, or the like, this application controls.

### I. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through or perpendicular across the end of a line in a structure indicates a specified point of attachment of a group. Unless chemically or structurally required, no directionality or stereochemistry is indicated or implied by the order in which a chemical group is written or named.

The prefix “Cᵤ-Cᵥ” indicates that the following group has from u to v carbon atoms. For example, “C₁-C₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±50%. In certain other embodiments, the term “about” includes the indicated amount ±20%. In certain other embodiments, the term “about” includes the indicated amount ±10%. In other embodiments, the term “about” includes the indicated amount ±5%. In certain other embodiments, the term “about” includes the indicated amount ±1%. In certain other embodiments, the term “about” includes the indicated amount ±0.5% and in certain other embodiments, 0.1%. Such variations are appropriate to perform the disclosed methods or employ the disclosed compositions. Also, to the term “about x” includes description of “x”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁-C₂₀ alkyl), 1 to 12 carbon atoms (i.e., C₁-C₁₂ alkyl), 1 to 8 carbon atoms (i.e., C₁-C₈ alkyl), 1 to 6 carbon atoms (i.e., C₁-C₆ alkyl), 1 to 4 carbon atoms (i.e., C₁-C₄ alkyl), or 1 to 3 carbon atoms (i.e., C₁-C₃ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl or aralkyl, the last-mentioned group contains the atom by which the moiety is attached to the rest of the molecule.

“Alkenyl” refers to an alkyl group containing at least one carbon-carbon double bond and having from 2 to 20 carbon atoms (i.e., C₂-C₂₀ alkenyl), 2 to 8 carbon atoms (i.e., C₂-C₈ alkenyl), 2 to 6 carbon atoms (i.e., C₂-C₆ alkenyl) or 2 to 4 carbon atoms (i.e., C₂-C₄ alkenyl). Examples of alkenyl groups include, e.g., ethenyl, propenyl, butadienyl (including 1,2-butadienyl and 1,3-butadienyl).

“Alkynyl” refers to an alkyl group containing at least one carbon-carbon triple bond, unless otherwise described, may have from 2 to 20 carbon atoms (i.e., C₂-C₂₀ alkynyl), 2 to 8 carbon atoms (i.e., C₂-C₈ alkynyl), 2 to 6 carbon atoms (i.e., C₂-C₆ alkynyl) or 2 to 4 carbon atoms (i.e., C₂-C₄ alkynyl). The term “alkynyl” also includes those groups having one triple bond and one double bond.

“Alkoxy” refers to the group “alkyl-O—” (e.g., C₁-C₃ alkoxy or C₁-C₆ alkoxy). Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy.

“Alkylthio” refers to the group “alkyl-S—”.

“Acyl” refers to a group —C(O)R^{y}, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of acyl include, e.g., formyl, acetyl, cyclohexylcarbonyl, cyclohexylmethyl-carbonyl and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein, or R^{y} and R^{z} are taken together to form a heterocyclyl; which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆-C₂₀ aryl), 6 to 12 carbon ring atoms (i.e., C₆-C₁₂ aryl), or 6 to 10 carbon ring atoms (i.e., C₆-C₁₀ aryl). Examples of aryl groups include, e.g., phenyl, naphthyl, fluorenyl and anthryl. Aryl, however, does not encompass or overlap in any way with heteroaryl defined below. If one or more aryl groups are fused with a heteroaryl, the resulting ring system is heteroaryl regardless of the point of attachment. If one or more aryl groups are fused with a heterocyclyl, the resulting ring system is heterocyclyl regardless of the point of attachment.

“Arylalkyl” or “Aralkyl” refers to the group “aryl-alkyl-”, such as (C₆-C₁₀ aryl)-C₁-C₃ alkyl. A non-limiting example of arylalkyl is benzyl.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl or heteroaryl; each of which may be optionally substituted, as defined herein.

“Cycloalkyl” refers to a saturated or partially unsaturated cyclic alkyl group having a single ring or multiple rings which may include fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃-C₂₀ cycloalkyl), 3 to 12 ring carbon atoms (i.e., C₃-C₁₂ cycloalkyl), 3 to 10 ring carbon atoms (i.e., C₃-C₁₀ cycloalkyl), 3 to 8 ring carbon atoms (i.e., C₃-C₈ cycloalkyl), 3 to 7 ring carbon atoms (i.e., C₃-C₇ cycloalkyl), or 3 to 6 ring carbon atoms (i.e., C₃-C₆ cycloalkyl). Monocyclic groups include, for example, cyclopropyl, cyclobutyl, cyclopentyl, cyclohexyl, cycloheptyl and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. Further, the term cycloalkyl is intended to encompass any moiety comprising a non-aromatic alkyl ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl. As used herein, “halocycloalkyl,” such as C₃-C₇ halocycloalkyl, refers to a C₃-C₇ cycloalkyl group that is substituted with one or more halogens.

“Cycloalkylalkyl” refers to the group “cycloalkyl-alkyl-”, such as (C₃-C₆ cycloalkyl)-C₁-C₃ alkyl.

“Guanidino” refers to —NR^{y}C(═NR^{z})(NR^{y}R^{z}), wherein each R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Hydrazino” refers to —NHNH₂.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

“Imido” refers to a group —C(O)NR^{y}C(O)R^{z}, wherein R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

“Halogen” or “halo” refers to atoms occupying group VIIA of the periodic table, such as fluoro (fluorine), chloro (chlorine), bromo (bromine) or iodo (iodine).

“Haloalkyl” refers to an unbranched or branched alkyl, alkenyl, or alkynyl group as defined above, wherein one or more (e.g., 1 to 6, or 1 to 3) hydrogen atoms are replaced by a halogen. For example, halo-C₁-C₃ alkyl refers to an alkyl group of 1 to 3 carbons wherein at least one hydrogen atom is replaced by a halogen. Halo-C₁-C₆ alkyl refers to an alkyl group of 1 to 6 carbons wherein at least one hydrogen atom is replaced by a halogen. Halo-C₁-C₆ alkenyl refers to an alkyl group containing at least one carbon-carbon double bond and having from 1 to 6 carbon atoms, wherein at least one hydrogen atom is replaced by a halogen. Where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like.

“Haloalkoxy” refers to an alkoxy group as defined above, wherein one or more (e.g., 1 to 6, or 1 to 3) hydrogen atoms are replaced by a halogen. For example, halo-C₁-C₃ alkoxy refers to an alkoxy group of 1 to 3 carbons wherein at least one hydrogen atom is replaced by a halogen. Halo-C₁-C₆ alkoxy refers to an alkoxy group of 1 to 6 carbons wherein at least one hydrogen atom is replaced by a halogen Non-limiting examples of haloalkoxy are —OCH₂CF₃, —OCF₂H, and —OCF₃.

“Hydroxyalkyl” refers to an alkyl group as defined above, wherein one or more (e.g., 1 to 6, or 1 to 3) hydrogen atoms are replaced by a hydroxy group (e.g., hydroxy-C₁-C₃-alkyl, hydroxy-C₁-C₆-alkyl). The term “hydroxy-C₁-C₃ alkyl” refers to a one to three carbon alkyl chain where one or more hydrogens on any carbon is replaced by a hydroxy group, in particular, one hydrogen on one carbon of the chain is replaced by a hydroxy group. The term “hydroxy-C₁-C₆ alkyl” refers to a one to six carbon alkyl chain where one or more hydrogens on any carbon is replaced by a hydroxy group, in particular, one hydrogen on one carbon of the chain is replaced by a hydroxy group. Non-limiting examples of hydroxyalkyl include —CH₂OH, —CH₂CH₂OH, and —C(CH₃)₂CH₂OH.

“Heteroalkyl” refers to an alkyl group in which one or more of the carbon atoms (and any associated hydrogen atoms) are each independently replaced with the same or different heteroatomic group, provided the point of attachment to the remainder of the molecule is through a carbon atom. In certain embodiments, the heteroalkyl can have 1 to 3 carbon atoms (e.g., C₁-C₃ heteroalkyl) or 1 to 6 carbon atoms (e.g., C₁-C₆ heteroalkyl), and one or more (e.g., 1, 2, or 3) heteroatoms or heteroatomic groups. The term “heteroalkyl” includes unbranched or branched saturated chain having carbon and heteroatoms. By way of example, 1, 2, or 3 carbon atoms of the alkyl group in the “heteroalkyl” may be independently replaced with the same or different heteroatomic group. Heteroatomic groups include, but are not limited to, —NR^{y}—, —O—, —S—, —S(O)—, —S(O)₂—, and the like, wherein R is hydrogen, alkyl, alkenyl, alkynyl, cycloalkyl, heterocyclyl, aryl, heteroalkyl or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.) and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be optionally substituted, as defined herein). In certain embodiments, heteroalkyl can have 1 to 20 carbon atoms, 1 to 15 carbon atoms, 1 to 12 carbon atoms, 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁-C₂₀ heteroaryl), 3 to 12 ring carbon atoms (i.e., C₃-C₁₂ heteroaryl), or 3 to 8 carbon ring atoms (i.e., C₃-C₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 9-10 membered ring systems (i.e., 9-10 membered heteroaryl), 5-10 membered ring systems (i.e., 5-10 membered heteroaryl), 5-7 membered ring systems (i.e., 5-7 membered heteroaryl), 5-6 membered ring systems (i.e., 5-6 membered heteroaryl), or 4-6 membered ring systems (i.e., 4-6 membered heteroaryl), each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic group, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heteroarylalkyl” refers to the group “heteroaryl-alkyl-”, such as (5- to 10-membered monocyclic heteroaryl)-C₁-C₃ alkyl.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro. Any non-aromatic ring containing at least one heteroatom is considered a heterocyclyl, regardless of the attachment (i.e., can be bound through a carbon atom or a heteroatom). Further, the term heterocyclyl is intended to encompass a moiety comprising any non-aromatic ring containing at least one heteroatom, which ring may be fused to an aryl or heteroaryl ring, regardless of the attachment to the remainder of the molecule. The term heterocyclyl is also intended to encompass a moiety comprising a cycloalkyl ring which is fused to a heteroaryl ring, regardless of the attachment to the remainder of the molecule. Additionally, the term heterocyclyl is intended to encompass a moiety comprising a cycloalkyl ring which is fused to a heterocyclyl ring, regardless of the attachment to the remainder of the molecule. As used herein, heterocyclyl has 2 to 20 ring carbon atoms (i.e., C₂-C₂₀ heterocyclyl), 2 to 12 ring carbon atoms (i.e., C₂-C₁₂ heterocyclyl), 2 to 10 ring carbon atoms (i.e., C₂-C₁₀ heterocyclyl), 2 to 8 ring carbon atoms (i.e., C₂-C₈ heterocyclyl), 3 to 12 ring carbon atoms (i.e., C₃-C₁₂ heterocyclyl), 3 to 8 ring carbon atoms (i.e., C₃-C₈ heterocyclyl), or 3 to 6 ring carbon atoms (i.e., C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. When the heterocyclyl ring contains 4- to 6-ring atoms, it is also referred to herein as a 4- to 6-membered heterocyclyl. Also disclosed herein are 5- or 6-membered heterocyclyls, having 5 or 6 ring atoms, respectively, and 5- to 10-membered heterocyclyls, having 5 to 10 ring atoms. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, tetrahydropyranyl, thiomorpholinyl, thiamorpholinyl, 1-oxo-thiomorpholinyl and 1,1-dioxo-thiomorpholinyl. In certain embodiments, the term “heterocyclyl” can include “spiroheterocyclyl” when there are two positions for substitution on the same carbon atom, wherein at least one ring of the spiro system comprises at least one heteroatom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

“Heterocyclylalkyl” refers to the group “heterocyclyl-alkyl-.”

“Oxime” refers to the group —CR^{y}(═NOH) wherein R^{y} is hydrogen, alkyl, alkenyl, alkynyl, cycloalkyl, heterocyclyl, aryl, heteroalkyl or heteroaryl; each of which may be optionally substituted, as defined herein.

“Oxo” refers to the group (═O).

“Cyano” refers to the group (—CN).

“N-oxide” refers to the group (—N⁺—O—).

“Thiol” refers to the group (—SH).

“Sulfonyl” refers to the group —S(O)₂R^{y}, where R^{y} is hydrogen, alkyl, alkenyl, alkynyl, cycloalkyl, heterocyclyl, aryl, heteroalkyl or heteroaryl; each of which may be optionally substituted, as defined herein. A non-limiting example of a sulfonyl group is —SO₂(C₁-C₆ alkyl), which is herein referred to as alkylsulfonyl. Examples of sulfonyl are methylsulfonyl, ethylsulfonyl, phenylsulfonyl and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl or heteroaryl; each of which may be optionally substituted, as defined herein. Examples of sulfinyl are methylsulfinyl, ethylsulfinyl, phenylsulfinyl and toluenesulfinyl.

“Sulfonamido” refers to the groups —SO₂NR^{y}R^{z} and —NR^{y}SO₂R^{z}, where R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5, 1 to 4, or 1 to 3) hydrogen atoms on the designated atom or group may or may not be replaced by a moiety other than hydrogen.

The term “substituted” used herein means any of the above groups (i.e., alkyl, alkenyl, alkynyl, alkylene, alkoxy, haloalkyl, haloalkoxy, cycloalkyl, aryl, heterocyclyl, heteroaryl, and/or heteroalkyl) wherein at least one (e.g., 1 to 5, 1 to 4, or 1 to 3) hydrogen atom is replaced by a bond to a non-hydrogen moiety. Unless otherwise described, such non-hydrogen moieties may include, but are not limited to alkyl, alkenyl, alkynyl, alkoxy, alkylthio, acyl, amido, amino, amidino, aryl, aralkyl, azido, carbamoyl, carboxyl, carboxyl ester, cyano, cycloalkyl, cycloalkylalkyl, guanidino, halo, haloalkyl, haloalkoxy, hydroxyalkyl, heteroalkyl, heteroaryl, heteroarylalkyl, heterocyclyl, heterocyclylalkyl, —NHNH₂, =NNH₂, imino, imido, hydroxy, oxo, oxime, nitro, sulfonyl, sulfinyl, alkylsulfonyl, alkylsulfinyl, thiocyanate, —S(O)OH, —S(O)₂OH, sulfonamido, thiol, thioxo, N-oxide or —Si(R^{y})₃, wherein each R^{y} is independently hydrogen, alkyl, alkenyl, alkynyl, heteroalkyl, cycloalkyl, aryl, heteroaryl or heterocyclyl.

In certain embodiments, “substituted” includes any of the above alkyl, alkenyl, alkynyl, cycloalkyl, heterocyclyl, aryl or heteroaryl groups in which one or more (e.g., 1 to 5, 1 to 4, or 1 to 3) hydrogen atoms are independently replaced with deuterium, halo, cyano, nitro, azido, oxo, alkyl, alkenyl, alkynyl, haloalkyl, cycloalkyl, heterocyclyl, aryl, heteroaryl, —NR^{g}R^{h}, —NR^{g}C(═O)R^{h}, —NR^{g}C(═O)NR^{g}R^{h}, —NR^{g}C(═O)OR^{h}, —NR^{g}S(═O)₁₋₂R^{h}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂OR^{g}, —NR^{g}S(═O)₁₋₂NR^{g}R^{h}, ═NSO₂R^{g}, =NOR^{g}, —S(═O)₁₋₂NR^{g}R^{h}, —SF₅, —SCF₃ or —OCF₃. In certain embodiments, “substituted” also means any of the above groups in which one or more (e.g., 1 to 5, 1 to 4, or 1 to 3) hydrogen atoms are replaced with —C(═O)R^{g}, —C(═O)OR^{g}, —C(═O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more (e.g., 1 to 5, 1 to 4, or 1 to 3) hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or two of R^{g} and R^{h} and Rⁱ are taken together with the atoms to which they are attached to form a heterocyclyl ring optionally substituted with oxo, halo or alkyl optionally substituted with oxo, halo, amino, hydroxyl, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to four. In certain embodiments, as used herein, the phrase “one or more” refers to one to three.

Any compound or structure given herein, is intended to represent unlabeled forms as well as isotopically labeled forms (isotopologues) of the compounds. These forms of compounds may also be referred to as and include “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure include, for example, those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium. Further, in some embodiments, the corresponding deuterated analog is provided.

In many cases, the compounds of this disclosure are capable of forming acid and/or base salts by virtue of the presence of amino and/or carboxyl groups or groups similar thereto.

Provided also are a pharmaceutically acceptable salt, isotopically enriched analog, deuterated analog, isomer (such as a stereoisomer), and mixture of isomers (such as a mixture of stereoisomers), of the compounds described herein.

“Pharmaceutically acceptable” or “physiologically acceptable” refer to compounds, salts, compositions, dosage forms and other materials which are useful in preparing a pharmaceutical composition that is suitable for veterinary or human pharmaceutical use. Generally, such a material is not biologically or otherwise undesirable, e.g., the material may be incorporated into a pharmaceutical composition administered to a patient without causing any significant undesirable biological effects or interacting in a deleterious manner with any of the other components of the composition in which it is contained.

The term “pharmaceutically acceptable salt” of a given compound includes salts which are generally safe and not biologically or otherwise undesirable, and includes those which are acceptable for veterinary use as well as human pharmaceutical use “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃) or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like.

The term “hydrate” refers to the complex formed by the combining of a compound described herein and water.

A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid and ethanolamine. Solvates include hydrates.

Some of the compounds described herein may exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers. Another example of a compound that has several tautomers is 1,4-thiazine. The tautomers are 1λ⁴,4-thiazine, 2H-1,4-thiazine, and 4H-1,4-thiazine, wherein only 1λ⁴,4-thiazine is aromatic.

The compounds described herein, or their pharmaceutically acceptable salts, may include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high performance liquid chromatography (HPLC). When the compounds described herein contain olefinic double bonds or other centres of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Treatment” or “treating” is an approach for obtaining beneficial or desired results including but not limited to clinical results. Beneficial or desired results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease or condition, that is, causing the regression of clinical symptoms (e.g., ameliorating the disease state, providing partial or total remission of the disease or condition, enhancing effect of another medication, delaying the progression of the disease, increasing the quality of life, and/or prolonging survival). Also encompassed by “treatment” or “treating” is a reduction of pathological consequence of demyelination.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, or deuterated analog thereof means an amount sufficient to effect treatment when administered to a subject, to provide a therapeutic benefit such as amelioration of symptoms or slowing of disease progression. The therapeutically effective amount may vary depending on the subject, and disease or condition being treated, the weight and age of the subject, the severity of the disease or condition, and the manner of administering, which can readily be determined by one of ordinary skill in the art. The effective amount of a compound of the disclosure in such a therapeutic method is, for example, from about 0.01 mg/kg/day to about 1000 mg/kg/day, or from about 0.1 mg/kg/day to about 100 mg/kg/day.

The term “excipient” as used herein refers to an inert or inactive substance that may be used in the production of a drug or pharmaceutical composition, such as a tablet containing a compound as described herein (or pharmaceutically acceptable salt) as an active ingredient. Various substances may be embraced by the term excipient, including without limitation any substance used as a diluent, filler or extender, binder, disintegrant, humectant, coating, emulsifier or dispersing agent, compression/encapsulation aid, cream or lotion, lubricant, solution for parenteral administration, material for chewable tablets, sweetener or flavoring, suspending/gelling agent, or wet granulation agent. Binders may include, e.g., carbomers, povidone, xanthan gum, etc.; coatings may include, e.g., cellulose acetate phthalate, ethylcellulose, gellan gum, maltodextrin, enteric coatings, etc.; compression/encapsulation aids include e.g. calcium carbonate, dextrose, fructose dc (dc—“directly compressible”), honey dc, lactose (anhydrate or monohydrate; optionally in combination with aspartame, cellulose, or microcrystalline cellulose), starch de, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g. dextrose, fructose de, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose de, sorbitol, sucrose de, etc.; and wet granulation agents include, e.g., calcium carbonate, maltodextrin, microcrystalline cellulose, etc. In some cases, the term “excipient” encompasses pharmaceutically acceptable carriers.

Additional definitions may also be provided below as appropriate.

### II. Compounds

In certain embodiments, the subject matter described herein is directed to compounds of Formula I:

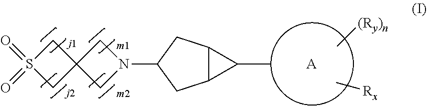

or a pharmaceutically acceptable salt thereof, wherein,
j₁ and m₁ are each independently 1, 2, or 3;
j₂ and m₂ are each independently 0, 1, 2, or 3;
   wherein the sum of j₁ and j₂ and the sum of m₁ and m₂ are each no more than 5, and the total sum of j₁, j₂, m₁, and m₂ is no more than 9; and when one of m₂ and j₂ is 0, the other is 1, 2, or 3;
Ring A is a 5-membered heteroaryl comprising one, two, or three heteroatoms independently selected from the group consisting of O, N, and S;
R_{y}, if present, in each instance is independently selected from the group consisting of halogen, C₁-C₆ alkyl, halo-C₁-C₆ alkyl, C₁-C₆ alkoxy, halo-C₁-C₆ alkoxy, C₃-C₇ cycloalkyl, C₃-C₇ halocycloalkyl, and —CN;
n is 0, 1, 2, or 3;
Rₓ is selected from the group consisting of halogen, C₁-C₁₀ alkyl, halo-C₁-C₆ alkyl, C₁-C₁₀ alkenyl, halo-C₁-C₆ alkenyl, C₁-C₆ alkoxy, halo-C₁-C₆ alkoxy, 5- to 7-membered heterocyclyl, C₃-C₇ cycloalkyl, C₆-C₁₀ aryl, 5- to 6-membered heteroaryl, and —CN;
   wherein said heterocyclyl, cycloalkyl, aryl, or heteroaryl is optionally substituted with (R_{xA})_{q};
      wherein q is 0, 1, 2, 3, 4, or 5; and
      each R_{xA} is independently selected from the group consisting of halogen, C₁-C₆ alkyl, hydroxy, C₁-C₆ alkoxy, halo-C₁-C₆ alkoxy, halo-C₁-C₆ alkyl, SO₂(C₁-C₆ alkyl), and —CN.

In certain embodiments, compounds include those of Formula I, or pharmaceutically acceptable salts thereof, where Ring A comprises one, two, or three N. In certain embodiments, compounds include those of Formula I, or pharmaceutically acceptable salts thereof, where Ring A is selected from the group consisting of pyrazolyl, triazolyl, and imidazolyl.

In certain embodiments of the compounds of Formula I, or a pharmaceutically acceptable salt thereof, the center bicyclic 3.1.0 ring is:

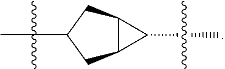

In some embodiments of Formula I, the sum of j₁ and j₂ and the sum of m₁ and m₂ are each no more than 5; and the total sum of j₁, j₂, m₁, and m₂ is no more than 7. In other embodiments, the sum of j₁ and j₂ and the sum of m₁ and m₂ are each no more than 5; and the total sum of j₁, j₂, m₁, and m₂ is no more than 6. In some embodiments, one of m₁ and m₂ is 1 and the other is 2; and j₁ and j₂ are both 1. In some other embodiments, one of m₁ and m₂ is 1 and the other is 2; and one of j₁ and j₂ is 1 and the other is 2. In some other embodiments, m₁ and m₂ are each 1; and j₁ and j₂ are each 2. In further embodiments, one of m₁ and m₂ is 1 and the other is 2; and j₂ is 0 and j₁ is 3. In some other embodiments, one of m₁ and m₂ is 3 and the other is 1; and one of j₁ and j₂ is 1 and the other is 2. In further embodiments, m₁ and m₂ are each 2; and j₂ and j₁ are each 1. In further embodiments, m₂ is 0 and m₁ is 3; and, j₂ and j₁ are each 1. Particular embodiments are shown in Table A.

TABLE A

| Spiro Ring | | | | | | | j₁ + j₂ + |
|---|---|---|---|---|---|---|---|
| Size | j₁ | j₂ | j₁ + j₂ | m₁ | m₂ | m₁ + m₂ | m₁ + m₂ |
| 4,4-fused | 1 | 1 | 2 | 1 | 1 | 2 | 4 |
| 4,5-fused | 1 | 1 | 2 | 2 | 1 | 3 | 5 |
| 4,5-fused | 1 | 1 | 2 | 1 | 2 | 3 | 5 |
| 4,6-fused | 1 | 1 | 2 | 2 | 2 | 4 | 6 |
| 5,5-fused | 2 | 1 | 3 | 2 | 1 | 3 | 6 |
| 5,6 fused | 2 | 1 | 3 | 1 | 3 | 4 | 7 |
| 5,6 fused | 2 | 1 | 3 | 3 | 1 | 4 | 7 |

In still further embodiments of a compound of Formula I, or a pharmaceutically acceptable salt thereof, m₁ and m₂ are independently 1 or 2; j₁ and j₂ are independently 1 or 2.

In certain embodiments of a compound of Formula I, or a pharmaceutically acceptable salt thereof, one of one of m₁ and m₂ is 1 and the other is 2; both of j₁ and j₂ are 1.

In certain embodiments of a compound of Formula I, or a pharmaceutically acceptable salt thereof, one of one of m₁ and m₂ is 2 and the other is 1; one of j₁ and j₂ is 2 and the other is 1.

In other embodiments of a compound of Formula I, or a pharmaceutically acceptable salt thereof, both of m₁ and m₂ are 2; both of j₁ and j₂ are 1.

In other embodiments of a compound of Formula I, or a pharmaceutically acceptable salt thereof, one of m₁ and m₂ is 3 and the other is 1; one of j₁ and j₂ is 2 and the other is 1.

In other embodiments of a compound of Formula I, or a pharmaceutically acceptable salt thereof, both of m₁ and m₂ are 1; both of j₁ and j₂ are 2.

In other embodiments of a compound of Formula I, or a pharmaceutically acceptable salt thereof, m₂ is 0 and m₁ is 3; both of j₁ and j₂ are 1.

In other embodiments of a compound of Formula I, or a pharmaceutically acceptable salt thereof, one of m₂ and m₁ is 2 and the other is 1; j₁ is 3 and j₂ is 0.

In some embodiments of the compounds of Formula I, or a pharmaceutically acceptable salt thereof, R_{y}, in each instance, is independently selected from the group consisting of C₁-C₆ alkyl, halo-C₁-C₆ alkyl, C₃-C₇ cycloalkyl, and C₃-C₇halocycloalkyl. In some embodiments, each R_{y} is independently C₁-C₆ alkyl or C₃-C₇ cycloalkyl. In certain embodiments, compounds include those where each R_{y} is independently selected from the group consisting of methyl, ethyl, propyl, butyl, and cyclobutyl. In certain embodiments, compounds include those where R_{y}, in each instance, is propyl. In certain embodiments, compounds include those where R_{y} is isopropyl. In certain embodiments, compounds include those where R_{y} is tertbutyl. In certain embodiments, compounds include those where R_{y}, in each instance, is selected from the group consisting of methyl, ethyl, propyl, butyl, cyclobutyl, trifluoromethyl, difluoromethyl, difluoroethyl, and difluoropropyl.

In certain embodiments of the compounds of Formula I, or pharmaceutically acceptable salts thereof, Rₓ is selected from the group consisting of C₁-C₆ alkyl, halo-C₁-C₆ alkyl, C₁-C₁₀ alkenyl, halo-C₁-C₆ alkenyl, halo-C₁-C₆ alkoxy, C₃-C₇ cycloalkyl, C₆-C₁₀ aryl, and 5- to 6-membered heteroaryl; wherein said cycloalkyl, aryl, or heteroaryl is substituted with (R_{xA})_{q}, wherein q is 0, 1, 2, or 3, and, if present, each R_{xA} is independently selected from the group consisting of halogen, C₁-C₆ alkyl, hydroxy, C₁-C₆ alkoxy, halo-C₁-C₆ alkoxy, and halo-C₁-C₆ alkyl. In certain embodiments, Rₓ is selected from the group consisting of C₁-C₆ alkyl, halo-C₁-C₆ alkyl, C₁-C₆ alkenyl, halo-C₁-C₆ alkenyl, halo-C₁-C₆ alkoxy, C₃-C₆ cycloalkyl, C₆ aryl, and 5- to 6-membered heteroaryl; wherein said cycloalkyl, aryl, or heteroaryl is substituted with (R_{xA})_{q}, wherein q is 0 or 1 and R_{xA} is halogen, C₁-C₆ alkyl, C₁-C₆ alkoxy, halo-C₁-C₆ alkoxy, or halo-C₁-C₆ alkyl. In some embodiments, R_{xA} is halo-C₁-C₆ alkyl or C₁-C₆ alkyl. In certain embodiments, compounds include those where Rₓ is halo-C₁-C₆ alkyl. In certain embodiments, compounds include those where Rₓ is selected from the group consisting of trifluoromethyl, difluoromethyl, fluoromethyl, trifluoroethyl, difluoroethyl, and fluoroethyl. In certain embodiments, compounds include those where Rₓ is trifluoromethyl. In certain embodiments, compounds include those where Rₓ is C₆-C₁₀ aryl or 5- to 6-membered heteroaryl substituted with (R_{xA})_{q}, wherein q is 0, 1, 2, or 3, and, if present, each R_{xA} is independently selected from the group consisting of halogen, C₁-C₆ alkyl, hydroxy, C₁-C₆ alkoxy, halo-C₁-C₆ alkoxy, and halo-C₁-C₆ alkyl. In certain embodiments, compounds include those where Rₓ is phenyl or 6-membered heteroaryl substituted with (R_{xA})_{q}. In some embodiments, q is 0 or 1 and R_{xA} is halo-C₁-C₆ alkyl or C₁-C₆ alkyl. In certain embodiments, compounds include those where Rₓ is C₃-C₇ cycloalkyl substituted with (R_{xA})_{q}, wherein q is 0, 1, 2, or 3, and, if present, each R_{xA} is independently selected from the group consisting of halogen, C₁-C₆ alkyl, hydroxy, C₁-C₆ alkoxy, halo-C₁-C₆ alkoxy, and halo-C₁-C₆ alkyl.

In yet further embodiments of the compounds of Formula I, or pharmaceutically acceptable salts thereof, n is 0, 1, or 2; each R_{y} is independently halogen, C₁-C₆ alkyl, halo-C₁-C₆ alkyl, C₃-C₇ cycloalkyl, or C₃-C₇halocycloalkyl; and Rₓ is selected from the group consisting of C₁-C₆ alkyl, halo-C₁-C₆ alkyl, C₁-C₆ alkenyl, halo-C₁-C₆ alkenyl, halo-C₁-C₆ alkoxy, C₃-C₆ cycloalkyl, C₆ aryl, and 5- to 6-membered heteroaryl; wherein said cycloalkyl, aryl, or heteroaryl is substituted with (R_{xA})_{q}, wherein q is 0 or 1 and R_{xA} is halogen, C₁-C₆ alkyl, C₁-C₆ alkoxy, halo-C₁-C₆ alkoxy, or halo-C₁-C₆ alkyl.

In certain embodiments, compounds of Formula I include those of Formula Ia:

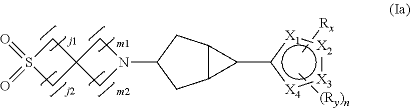

or a pharmaceutically acceptable salt thereof, wherein:
X₁, X₂, X₃, and X₄ are each individually N, NH, N substituted with Rₓ or R_{y}, C substituted with Rₓ or R_{y}, or CH, wherein one, two, or three of X₁, X₂, X₃, and X₄ are N, NH, or substituted N;
and j₁, j₂, m₁, m₂, Rₓ, R_{y}, and n are as defined for Formula (I).

It should be clear to one of skill in the art that only one Rₓ is present in compounds of Formula Ia, or pharmaceutically acceptable salts thereof; and therefore that, for example, only one of X₁, X₂, X₃, and X₄ can be C—Rₓ or N—Rₓ. Similarly, whether one or more of X₁, X₂, X₃, and X₄ is C—R_{y} or N—R_{y} depends on the value of n. When n is 0, none of X₁, X₂, X₃, and X₄ is C—R_{y} or N—R_{y}. When n is 1, one of X₁, X₂, X₃, and X₄ is C—R_{y} or N—R_{y}. When n is 2, two of X₁, X₂, X₃, and X₄ are independently C—R_{y} or N—R_{y}, wherein each R_{y} is independently selected; and so on.

In some embodiments of the compounds of Formula Ia, three of X₁, X₂, X₃, and X₄ are independently NH, N, or substituted N. In other embodiments, two of X₁, X₂, X₃, and X₄ are independently NH, N, or substituted N. In still further embodiments, one of X₁, X₂, X₃, and X₄ is NH, N, or substituted N. In certain embodiments, compounds include those of Formula Ia, or pharmaceutically acceptable salts thereof, where X₂ is C—Rₓ. In certain embodiments, compounds include those of Formula Ia, or pharmaceutically acceptable salts thereof, where X₃ is N. In certain embodiments, compounds include those where n is 1. In some embodiments of the compound of Formula Ia, or a pharmaceutically acceptable salt thereof, the sum of j₁ and j₂ and the sum of m₁ and m₂ are each no more than 5; and the total sum of j₁, j₂, m₁, and m₂ is no more than 7. In other embodiments, the sum of j₁ and j₂ and the sum of m₁ and m₂ are each no more than 5; and the total sum of j₁, j₂, m₁, and m₂ is no more than 6.

In certain embodiments of the compounds of Formula Ia, or a pharmaceutically acceptable salt thereof, the center bicyclic 3.1.0 ring is:

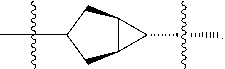

In some embodiments of Formula Ia, one of m₁ and m₂ is 1 and the other is 2; and j₁ and j₂ are both 1. In some other embodiments, one of m₁ and m₂ is 1 and the other is 2; and one of j₁ and j₂ is 1 and the other is 2. In some other embodiments, m₁ and m₂ are each 1; and j₁ and j₂ are each 2. In further embodiments, one of m₁ and m₂ is 1 and the other is 2; and j₂ is 0 and j₁ is 3. In some other embodiments, one of m₁ and m₂ is 3 and the other is 1; and one of j₁ and j₂ is 1 and the other is 2. In further embodiments, m₁ and m₂ are each 2; and j₂ and j₁ are each 1. In further embodiments, m₂ is 0 and m₁ is 3; and, j₂ and j₁ are each 1.

In still further embodiments of a compound of Formula Ia, or a pharmaceutically acceptable salt thereof, m₁ and m₂ are independently 1 or 2; j₁ and j₂ are independently 1 or 2.

In certain embodiments of a compound of Formula Ia, or a pharmaceutically acceptable salt thereof, one of one of m₁ and m₂ is 1 and the other is 2; both of j₁ and j₂ are 1.

In certain embodiments of a compound of Formula Ia, or a pharmaceutically acceptable salt thereof, one of one of m₁ and m₂ is 2 and the other is 1; one of j₁ and j₂ is 2 and the other is 1.

In other embodiments of a compound of Formula Ia, or a pharmaceutically acceptable salt thereof, both of m₁ and m₂ are 2; both of j₁ and j₂ are 1.

In other embodiments of a compound of Formula Ia, or a pharmaceutically acceptable salt thereof, one of m₁ and m₂ is 3 and the other is 1; one of j₁ and j₂ is 2 and the other is 1.

In other embodiments of a compound of Formula Ia, or a pharmaceutically acceptable salt thereof, both of m₁ and m₂ are 1; both of j₁ and j₂ are 2.

In other embodiments of a compound of Formula Ia, or a pharmaceutically acceptable salt thereof, m₂ is 0 and m₁ is 3; both of j₁ and j₂ are 1.

In other embodiments of a compound of Formula Ia, or a pharmaceutically acceptable salt thereof, one of m₂ and m₁ is 2 and the other is 1; j₁ is 3 and j₂ is 0.

In some embodiments of the compounds of Formula Ia, or a pharmaceutically acceptable salt thereof, R_{y}, in each instance, is independently selected from the group consisting of C₁-C₆ alkyl, halo-C₁-C₆ alkyl, C₃-C₇ cycloalkyl, and C₃-C₇halocycloalkyl. In some embodiments, each R_{y} is independently C₁-C₆ alkyl or C₃-C₇ cycloalkyl. In certain embodiments, compounds include those where each R_{y} is independently selected from the group consisting of methyl, ethyl, propyl, butyl, and cyclobutyl. In certain embodiments, compounds include those where R_{y}, in each instance, is propyl. In certain embodiments, compounds include those where R_{y} is isopropyl. In certain embodiments, compounds include those where R_{y} is tertbutyl. In certain embodiments, compounds include those where R_{y}, in each instance, is selected from the group consisting of methyl, ethyl, propyl, butyl, cyclobutyl, trifluoromethyl, difluoromethyl, difluoroethyl, and difluoropropyl.

In certain embodiments of the compounds of Formula Ia, or pharmaceutically acceptable salts thereof, Rₓ is selected from the group consisting of C₁-C₆ alkyl, halo-C₁-C₆ alkyl, C₁-C₁₀ alkenyl, halo-C₁-C₆ alkenyl, halo-C₁-C₆ alkoxy, C₃-C₇ cycloalkyl, C₆-C₁₀ aryl, and 5- to 6-membered heteroaryl; wherein said cycloalkyl, aryl, or heteroaryl is substituted with (R_{xA})_{q}, wherein q is 0, 1, 2, or 3, and, if present, each R_{xA} is independently selected from the group consisting of halogen, C₁-C₆ alkyl, hydroxy, C₁-C₆ alkoxy, halo-C₁-C₆ alkoxy, and halo-C₁-C₆ alkyl. In certain embodiments, Rₓ is selected from the group consisting of C₁-C₆ alkyl, halo-C₁-C₆ alkyl, C₁-C₆ alkenyl, halo-C₁-C₆ alkenyl, halo-C₁-C₆ alkoxy, C₃-C₆ cycloalkyl, C₆ aryl, and 5- to 6-membered heteroaryl; wherein said cycloalkyl, aryl, or heteroaryl is substituted with (R_{xA})_{q}, wherein q is 0 or 1 and R_{xA} is halogen, C₁-C₆ alkyl, C₁-C₆ alkoxy, halo-C₁-C₆ alkoxy, or halo-C₁-C₆ alkyl. In some embodiments, R_{xA} is halo-C₁-C₆ alkyl or C₁-C₆ alkyl. In certain embodiments, compounds include those where Rₓ is halo-C₁-C₆ alkyl. In certain embodiments, compounds include those where Rₓ is selected from the group consisting of trifluoromethyl, difluoromethyl, fluoromethyl, trifluoroethyl, difluoroethyl, and fluoroethyl. In certain embodiments, compounds include those where Rₓ is trifluoromethyl. In certain embodiments, compounds include those where Rₓ is C₆-C₁₀ aryl or 5- to 6-membered heteroaryl substituted with (R_{xA})_{q}, wherein q is 0, 1, 2, or 3, and, if present, each R_{xA} is independently selected from the group consisting of halogen, C₁-C₆ alkyl, hydroxy, C₁-C₆ alkoxy, halo-C₁-C₆ alkoxy, and halo-C₁-C₆ alkyl. In certain embodiments, compounds include those where Rₓ is phenyl or 6-membered heteroaryl substituted with (R_{xA})_{q}. In some embodiments, q is 0 or 1 and R_{xA} is halo-C₁-C₆ alkyl or C₁-C₆ alkyl.

In yet further embodiments of the compounds of Formula Ia, or pharmaceutically acceptable salts thereof, n is 0, 1, or 2; each R_{y} is independently halogen, C₁-C₆ alkyl, halo-C₁-C₆ alkyl, C₃-C₇ cycloalkyl, or C₃-C₇halocycloalkyl; and Rₓ is selected from the group consisting of C₁-C₆ alkyl, halo-C₁-C₆ alkyl, C₁-C₆ alkenyl, halo-C₁-C₆ alkenyl, halo-C₁-C₆ alkoxy, C₃-C₆ cycloalkyl, C₆ aryl, and 5- to 6-membered heteroaryl; wherein said cycloalkyl, aryl, or heteroaryl is substituted with (R_{xA})_{q}, wherein q is 0 or 1 and R_{xA} is halogen, C₁-C₆ alkyl, C₁-C₆ alkoxy, halo-C₁-C₆ alkoxy, or halo-C₁-C₆ alkyl.

In certain embodiments, the compound of Formula I is a compound of Formula Ib:

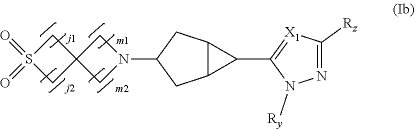

or pharmaceutically acceptable salts thereof, wherein X₁ is CH or N and R_{y} is C₁-C₆ alkyl or C₃-C₅ cycloalkyl; and j₁, j₂, m₁, m₂, and Rₓ are as defined for Formula (I).

In certain embodiments of the compounds of Formula Ib, or a pharmaceutically acceptable salt thereof, the center bicyclic 3.1.0 ring is:

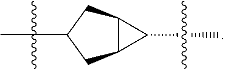

In some embodiments of Formula Ib, one of m₁ and m₂ is 1 and the other is 2; and j₁ and j₂ are both 1. In some other embodiments, one of m₁ and m₂ is 1 and the other is 2; and one of j₁ and j₂ is 1 and the other is 2. In some other embodiments, m₁ and m₂ are each 1; and j₁ and j₂ are each 2. In further embodiments, one of m₁ and m₂ is 1 and the other is 2; and j₂ is 0 and j₁ is 3. In some other embodiments, one of m₁ and m₂ is 3 and the other is 1; and one of j₁ and j₂ is 1 and the other is 2. In further embodiments, m₁ and m₂ are each 2; and j₂ and j₁ are each 1. In further embodiments, m₂ is 0 and m₁ is 3; and, j₂ and j₁ are each 1.

In still further embodiments of a compound of Formula Ib, or a pharmaceutically acceptable salt thereof, m₁ and m₂ are independently 1 or 2; j₁ and j₂ are independently 1 or 2.

In certain embodiments of a compound of Formula Ib, or a pharmaceutically acceptable salt thereof, one of one of m₁ and m₂ is 1 and the other is 2; both of j₁ and j₂ are 1.

In certain embodiments of a compound of Formula Ib, or a pharmaceutically acceptable salt thereof, one of one of m₁ and m₂ is 2 and the other is 1; one of j₁ and j₂ is 2 and the other is 1.

In other embodiments of a compound of Formula Ib, or a pharmaceutically acceptable salt thereof, both of m₁ and m₂ are 2; both of j₁ and j₂ are 1.

In other embodiments of a compound of Formula Ib, or a pharmaceutically acceptable salt thereof, one of m₁ and m₂ is 3 and the other is 1; one of j₁ and j₂ is 2 and the other is 1.

In other embodiments of a compound of Formula Ib, or a pharmaceutically acceptable salt thereof, both of m₁ and m₂ are 1; both of j₁ and j₂ are 2.

In other embodiments of a compound of Formula Ib, or a pharmaceutically acceptable salt thereof, m₂ is 0 and m₁ is 3; both of j₁ and j₂ are 1.

In other embodiments of a compound of Formula Ib, or a pharmaceutically acceptable salt thereof, one of m₂ and m₁ is 2 and the other is 1; j₁ is 3 and j₂ is 0.

In some embodiments of the compounds of Formula Ib, or a pharmaceutically acceptable salt thereof, R_{y}, in each instance, is independently selected from the group consisting of C₁-C₆ alkyl, halo-C₁-C₆ alkyl, C₃-C₇ cycloalkyl, and C₃-C₇halocycloalkyl. In some embodiments, each R_{y} is independently C₁-C₆ alkyl or C₃-C₇ cycloalkyl. In certain embodiments, compounds include those where each R_{y} is independently selected from the group consisting of methyl, ethyl, propyl, butyl, and cyclobutyl. In certain embodiments, compounds include those where R_{y}, in each instance, is propyl. In certain embodiments, compounds include those where R_{y} is isopropyl. In certain embodiments, compounds include those where R_{y} is tertbutyl. In certain embodiments, compounds include those where R_{y}, in each instance, is selected from the group consisting of methyl, ethyl, propyl, butyl, cyclobutyl, trifluoromethyl, difluoromethyl, difluoroethyl, and difluoropropyl.

In certain embodiments of the compounds of Formula Ib, or pharmaceutically acceptable salts thereof, Rₓ is selected from the group consisting of C₁-C₆ alkyl, halo-C₁-C₆ alkyl, C₁-C₁₀ alkenyl, halo-C₁-C₆ alkenyl, halo-C₁-C₆ alkoxy, C₃-C₇ cycloalkyl, C₆-C₁₀ aryl, and 5- to 6-membered heteroaryl; wherein said cycloalkyl, aryl, or heteroaryl is substituted with (R_{xA})_{q}, wherein q is 0, 1, 2, or 3, and, if present, each R_{xA} is independently selected from the group consisting of halogen, C₁-C₆ alkyl, hydroxy, C₁-C₆ alkoxy, halo-C₁-C₆ alkoxy, and halo-C₁-C₆ alkyl. In certain embodiments, Rₓ is selected from the group consisting of C₁-C₆ alkyl, halo-C₁-C₆ alkyl, C₁-C₆ alkenyl, halo-C₁-C₆ alkenyl, halo-C₁-C₆ alkoxy, C₃-C₆ cycloalkyl, C₆ aryl, and 5- to 6-membered heteroaryl; wherein said cycloalkyl, aryl, or heteroaryl is substituted with (R_{xA})_{q}, wherein q is 0 or 1 and R_{xA} is halogen, C₁-C₆ alkyl, C₁-C₆ alkoxy, halo-C₁-C₆ alkoxy, or halo-C₁-C₆ alkyl. In some embodiments, R_{xA} is halo-C₁-C₆ alkyl or C₁-C₆ alkyl. In certain embodiments, compounds include those where Rₓ is halo-C₁-C₆ alkyl. In certain embodiments, compounds include those where Rₓ is selected from the group consisting of trifluoromethyl, difluoromethyl, fluoromethyl, trifluoroethyl, difluoroethyl, and fluoroethyl. In certain embodiments, compounds include those where Rₓ is trifluoromethyl. In certain embodiments, compounds include those where Rₓ is C₆-C₁₀ aryl or 5- to 6-membered heteroaryl substituted with (R_{xA})_{q}, wherein q is 0, 1, 2, or 3, and, if present, each R_{xA} is independently selected from the group consisting of halogen, C₁-C₆ alkyl, hydroxy, C₁-C₆ alkoxy, halo-C₁-C₆ alkoxy, and halo-C₁-C₆ alkyl. In certain embodiments, compounds include those where Rₓ is phenyl or 6-membered heteroaryl substituted with (R_{xA})_{q}. In some embodiments, q is 0 or 1 and Rₓa is halo-C₁-C₆ alkyl or C₁-C₆ alkyl.

In yet further embodiments of the compounds of Formula Ib, or pharmaceutically acceptable salts thereof, n is 0, 1, or 2; each R_{y} is independently halogen, C₁-C₆ alkyl, halo-C₁-C₆ alkyl, C₃-C₇ cycloalkyl, or C₃-C₇halocycloalkyl; and Rₓ is selected from the group consisting of C₁-C₆ alkyl, halo-C₁-C₆ alkyl, C₁-C₆ alkenyl, halo-C₁-C₆ alkenyl, halo-C₁-C₆ alkoxy, C₃-C₆ cycloalkyl, C₆ aryl, and 5- to 6-membered heteroaryl; wherein said cycloalkyl, aryl, or heteroaryl is substituted with (R_{xA})_{q}, wherein q is 0 or 1 and R_{xA} is halogen, C₁-C₆ alkyl, C₁-C₆ alkoxy, halo-C₁-C₆ alkoxy, or halo-C₁-C₆ alkyl.

In yet further embodiments of the compounds of Formula Ib, or pharmaceutically acceptable salts thereof, Rₓ is a C₃-C₆ cycloalkyl, such as a cyclopentyl or cyclohexyl, each substituted with (R_{xA})_{q}, wherein q is 0, 1, 2, or 3, and, if present, each R_{xA} is independently selected from the group consisting of halogen, C₁-C₆ alkyl, hydroxy, C₁-C₆ alkoxy, halo-C₁-C₆ alkoxy, and halo-C₁-C₆ alkyl. In certain embodiments, R_{xA} is independently selected from the group consisting of methyl, ethyl, propyl, butyl, hydroxyl, fluoro, including gem di-fluoro, trifluoromethyl, difluoromethyl, difluoroethyl, and difluoropropyl. In certain embodiments, q is 1 or 2.

In certain embodiments, the compound of Formula I is a compound of Formula Ic:

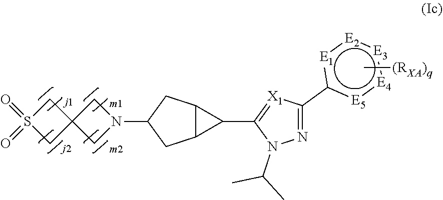

or a pharmaceutically acceptable salt thereof, wherein E₁, E₂, E₃, E₄, and E₅ are each independently N, C—R_{XA}, or CH, wherein up to three of E₁, E₂, E₃, E₄, and E₅ are N; and q is 1 or 2; and j₁, j₂, m₁, and m₂, are as defined for Formula (I).

It should be clear to one of skill in the art that whether one or two of E₁, E₂, E₃, E₄, and E₅ is C—R_{XA} depends on the value of q. When q is 1, one of E₁, E₂, E₃, E₄, and E₅ is C—R_{XA}. When q is 2, two of E₁, E₂, E₃, E₄, and E₅ are C—R_{xA}, wherein each R_{XA} is independently selected.

In certain embodiments, compounds include those of Formula Ic where E₁ is CH, E₂ is N, E₃ is C—R_{xA}, E₄ is C—R_{XA}, and E₅ is CH, or a pharmaceutically acceptable salt thereof. In certain embodiments, E₁, E₂, E₃, and E₅ are CH and E₄ is C—R_{XA}. In certain embodiments, E₁ is CH, E₂ is N, E₃ is CH, E₄ is C—R_{XA}, and E₅ is CH. In certain embodiments, E₁, E₂, E₄, and E₅ are CH, and E₃ is C—R_{XA}. In certain embodiments, E₁ is CH, E₂ is N, E₃ is C—R_{XA}, E₄ is N, and E₅ is CH. In certain embodiments, E₁, E₂, and E₃ are CH, E₄ is C—R_{XA}, and E₅ is N. In certain embodiments, E₁ is CH, E₂ is N, E₃ is C—R_{XA}, and E₄ and E₅ are CH. In certain embodiments, E₁ is CH, E₂ is CH, E₃ is N, E₄ is C—R_{XA}, and E₅ is N. In certain embodiments, E₁ is CH, E₂ is CH, E₃ is N, E₄ is C—R_{XA}, and E₅ is CH. In certain embodiments, E₁ is N, E₂ is CH, E₃ is CH, E₄ is C—R_{XA}, and E₅ is CH. In certain embodiments, compounds include those of Formula Ic where R_{XA} is halo-C₁-C₆ alkyl, or a pharmaceutically acceptable salt thereof. In certain embodiments, R_{XA} is selected from the group consisting of trifluoromethyl, difluoromethyl, fluoromethyl, trifluoroethyl, difluoroethyl, and fluoroethyl. In certain embodiments, R_{XA} is trifluoromethyl. In certain embodiments, R_{XA} is halo-C₁-C₆ alkoxy. In certain embodiments, R_{XA} is difluoromethoxy.

In certain embodiments of the compounds of Formula Ic, or a pharmaceutically acceptable salt thereof, the center bicyclic 3.1.0 ring is:

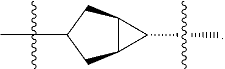

In some embodiments of Formula Ic, one of m₁ and m₂ is 1 and the other is 2; and j₁ and j₂ are both 1. In some other embodiments, one of m₁ and m₂ is 1 and the other is 2; and one of j₁ and j₂ is 1 and the other is 2. In some other embodiments, m₁ and m₂ are each 1; and j₁ and j₂ are each 2. In further embodiments, one of m₁ and m₂ is 1 and the other is 2; and j₂ is 0 and j₁ is 3. In some other embodiments, one of m₁ and m₂ is 3 and the other is 1; and one of j₁ and j₂ is 1 and the other is 2. In further embodiments, m₁ and m₂ are each 2; and j₂ and j₁ are each 1. In further embodiments, m₂ is 0 and m₁ is 3; and, j₂ and j₁ are each 1.

In still further embodiments of a compound of Formula Ic, or a pharmaceutically acceptable salt thereof, m₁ and m₂ are independently 1 or 2; j₁ and j₂ are independently 1 or 2.

In certain embodiments of a compound of Formula Ic, or a pharmaceutically acceptable salt thereof, one of one of m₁ and m₂ is 1 and the other is 2; both of j₁ and j₂ are 1.

In certain embodiments of a compound of Formula Ic, or a pharmaceutically acceptable salt thereof, one of one of m₁ and m₂ is 2 and the other is 1; one of j₁ and j₂ is 2 and the other is 1.

In other embodiments of a compound of Formula Ic, or a pharmaceutically acceptable salt thereof, both of m₁ and m₂ are 2; both of j₁ and j₂ are 1.

In other embodiments of a compound of Formula Ic, or a pharmaceutically acceptable salt thereof, one of m₁ and m₂ is 3 and the other is 1; one of j₁ and j₂ is 2 and the other is 1.

In other embodiments of a compound of Formula Ic, or a pharmaceutically acceptable salt thereof, both of m₁ and m₂ are 1; both of j₁ and j₂ are 2.

In other embodiments of a compound of Formula Ic, or a pharmaceutically acceptable salt thereof, m₂ is 0 and m₁ is 3; both of j₁ and j₂ are 1.

In other embodiments of a compound of Formula Ic, or a pharmaceutically acceptable salt thereof, one of m₂ and m₁ is 2 and the other is 1; j₁ is 3 and j₂ is 0.

In certain further embodiments of Formula Ic as described herein, or a pharmaceutically acceptable salt thereof, wherein:
E₁, E₂, E₃, E₄, and E₅ are each independently N, C when bound to R_{XA}, or CH, wherein up to three of E₁, E₂, E₃, E₄, and E₅ are N;
X₁ is N or CH;
q is 1 or 2;
each R_{xA} is independently selected from the group consisting of halogen, C₁-C₆ alkyl, hydroxy, C₁-C₆ alkoxy, halo-C₁-C₆ alkoxy, and halo-C₁-C₆ alkyl;
one of m₁ and m₂ is 2 and the other is 1, or m₁ and m₂ are each 1, or m₁ and m₂ are each 2; and
j₁ and j₂ are each 1, or one of j₁ and j₂ is 2 and the other is 1, or j₁ and j₂ are each 2.

In some such embodiments of compounds of Formula Ic, or pharmaceutically acceptable salts thereof, one of m₁ and m₂ is 2 and the other is 1. In other embodiments, m₁ and m₂ are each 1. In still further embodiments, m₁ and m₂ are each 2. In yet further embodiments, j₁ and j₂ are each 1. In other embodiments, one of j₁ and j₂ is 2 and the other is 1. In certain embodiments, j₁ and j₂ are each 2.

In certain embodiments, compounds include those of Formula Ia, Ib, or Ic, or pharmaceutically acceptable salts thereof, where X₁ is CH.

In certain embodiments of the compounds of Formula I, or a pharmaceutically acceptable salt thereof, such as compounds of Formula Ia, Ib, or Ic, or a pharmaceutically acceptable salt of any of the foregoing, compounds include those where the sum of j₁ and j₂ and the sum of m₁ and m₂ are each no more than 4, and the total sum of j₁, j₂, m₁, and m₂ is no more than 7. In certain embodiments of the compounds of Formula I, or a pharmaceutically acceptable salt thereof, such as compounds of Formula Ia, Ib, or Ic, or a pharmaceutically acceptable salt of any of the foregoing, the sum of j₁ and j₂ and the sum of m₁ and m₂ are each no more than 3, and the total sum of j₁, j₂, m₁, and m₂ is no more than 5. In certain embodiments, one of m₁ and m₂ is 1 and the other is 2. In certain embodiments, m₁ and m₂ are each 1. In certain embodiments, one of m₁ and m₂ is 3 and the other is 1. In certain embodiments, m₁ and m₂ are each 2. In certain embodiments, m₁ is 3 and m₂ is 0. In certain embodiments, one of j₁ and j₂ is 1 and the other is 2. In certain embodiments, one of j₁ and j₂ is 3 and the other is 1. In certain embodiments, j₁ and j₂ are each 1. In certain embodiments, j₁ and j₂ are each 2. In certain embodiments, j₁ is 3 and j₂ is 0.

In certain embodiments of the compounds of Formula I, or a pharmaceutically acceptable salt thereof, such as compounds of Formula Ia, Ib, or Ic, or a pharmaceutically acceptable salt of any of the foregoing, the center bicyclic 3.1.0 ring is:

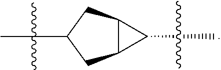

The subject matter described herein includes the following compounds in Table 1, or pharmaceutically acceptable salts thereof. Individual enantiomers and diastereomers are included in the table below by compound name, and their corresponding structures can be readily determined therefrom. In Table 1, the asterix (*) indicates an isolated isomer or isolated group of isomers, but that the stereochemistry has not been assigned. In some instances, the enantiomers or diastereomers of the present disclosure may be identified by their respective properties, for example, retention times by chiral HPLC, NMR peaks, and/or biological activities (e.g., as described further in the Examples), whereas the absolute stereo configurations of one or more chiral centers has not been assigned.

TABLE 1

| Cmpd No. | Structure | Name |
|---|---|---|
| 1 2 | 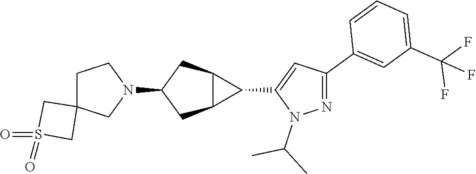 [SMILES: CC(C)n1nc(-c2cccc(C(F)(F)F)c2)cc1[C@H]1[C@@H]2C[C@H](N3CCC4(C3)CS(=O)(=O)C4)C[C@@H]21] | 6-((1R,3r,5S,6r)-6-(1-isopropyl-3-(3- (trifluoromethyl)phenyl)-1H-pyrazol-5- yl)bicyclo[3.1.0]hexan-3-yl)-2-thia-6- azaspiro[3.4]octane 2,2-dioxide 6-((1R,3s,5S,6r)-6-(1-isopropyl-3-(3- (trifluoromethyl)phenyl)-1H-pyrazol-5- yl)bicyclo[3.1.0]hexan-3-yl)-2-thia-6- azaspiro[3.4]octane 2,2-dioxide |
| | 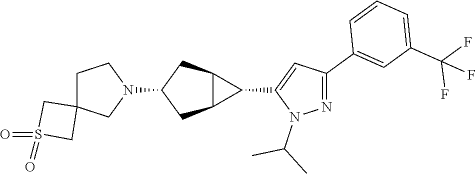 [SMILES: CC(C)n1nc(-c2cccc(C(F)(F)F)c2)cc1[C@H]1[C@@H]2C[C@@H](N3CCC4(C3)CS(=O)(=O)C4)C[C@@H]21] | |
| 3 4 | 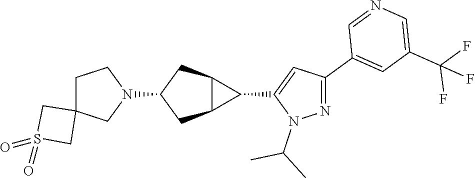 [SMILES: CC(C)n1nc(-c2cncc(C(F)(F)F)c2)cc1[C@H]1[C@@H]2C[C@@H](N3CCC4(C3)CS(=O)(=O)C4)C[C@@H]21] | 6-((1R,3s,5S,6r)-6-(1-isopropyl-3-(5- (trifluoromethyl)pyridin-3-yl)-1H-pyrazol- 5-yl)bicyclo[3.1.0]hexan-3-yl)-2-thia-6- azaspiro[3.4]octane 2,2-dioxide 6-((1R,3r,5S,6r)-6-(1-isopropyl-3-(5- (trifluoromethyl)pyridin-3-yl)-1H-pyrazol- 5-yl)bicyclo[3.1.0]hexan-3-yl)-2-thia-6- azaspiro[3.4]octane 2,2-dioxide |
| | 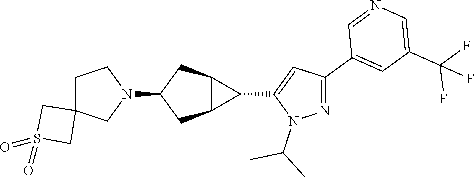 [SMILES: CC(C)n1nc(-c2cncc(C(F)(F)F)c2)cc1[C@H]1[C@@H]2C[C@H](N3CCC4(C3)CS(=O)(=O)C4)C[C@@H]21] | |
| 5 | 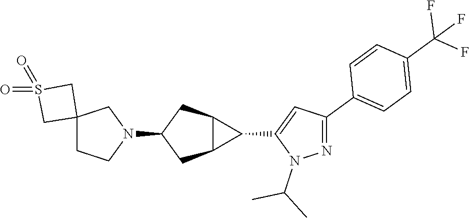 [SMILES: CC(C)n1nc(-c2ccc(C(F)(F)F)cc2)cc1[C@H]1[C@@H]2C[C@H](N3CCC4(C3)CS(=O)(=O)C4)C[C@@H]21] | 6-((1R,3r,5S,6r)-6-(1-isopropyl-3-(4- (trifluoromethyl)phenyl)-1H-pyrazol-5- yl)bicyclo[3.1.0]hexan-3-yl)-2-thia-6- azaspiro[3.4]octane 2,2-dioxide |
| 6 | 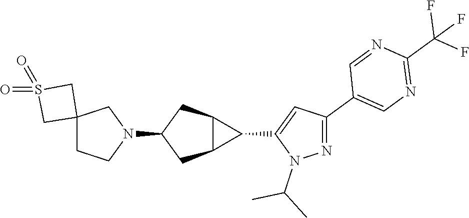 [SMILES: CC(C)n1nc(-c2cnc(C(F)(F)F)nc2)cc1[C@H]1[C@@H]2C[C@H](N3CCC4(C3)CS(=O)(=O)C4)C[C@@H]21] | 6-((1R,3r,5S,6r)-6-(1-isopropyl-3-(2- (trifluoromethyl)pyrimidin-5-yl)-1H- pyrazol-5-yl)bicyclo[3.1.0]hexan-3-yl)-2- thia-6-azaspiro[3.4]octane 2,2-dioxide |
| 7* 8* 9* 10* | 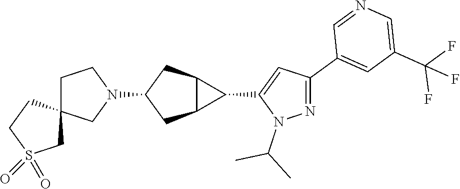 [SMILES: CC(C)n1nc(-c2cncc(C(F)(F)F)c2)cc1[C@H]1[C@@H]2C[C@@H](N3CC[C@@]4(CCS(=O)(=O)C4)C3)C[C@@H]21] | (R)-7-((1R,3s,5S,6R)-6-(1-isopropyl-3-(5- (trifluoromethyl)pyridin-3-yl)-1H-pyrazol- 5-yl)bicyclo[3.1.0]hexan-3-yl)-2-thia-7- azaspiro[4.4]nonane 2,2-dioxide (S)-7-((1R,3s,5S,6S)-6-(1-isopropyl-3-(5- (trifluoromethyl)pyridin-3-yl)-1H-pyrazol- 5-yl)bicyclo[3.1.0]hexan-3-yl)-2-thia-7- azaspiro[4.4]nonane 2,2-dioxide (R)-7-((1R,3r,5S,6R)-6-(1-isopropyl-3-(5- (trifluoromethyl)pyridin-3-yl)-1H-pyrazol- 5-yl)bicyclo[3.1.0]hexan-3-yl)-2-thia-7- azaspiro[4.4]nonane 2,2-dioxide |
| | | (S)-7-((1R,3r,5S,6S)-6-(1-isopropyl-3-(5- |
| | 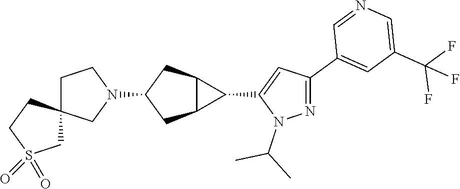 [SMILES: CC(C)n1nc(-c2cncc(C(F)(F)F)c2)cc1[C@H]1[C@@H]2C[C@@H](N3CC[C@]4(CCS(=O)(=O)C4)C3)C[C@@H]21] | (trifluoromethyl)pyridin-3-yl)-1H-pyrazol- 5-yl)bicyclo[3.1.0]hexan-3-yl)-2-thia-7- azaspiro[4.4]nonane 2,2-dioxide |
| | 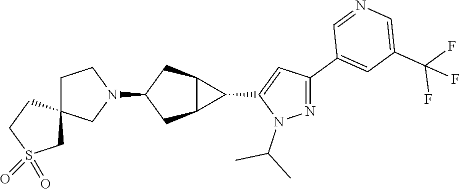 [SMILES: CC(C)n1nc(-c2cncc(C(F)(F)F)c2)cc1[C@H]1[C@@H]2C[C@H](N3CC[C@@]4(CCS(=O)(=O)C4)C3)C[C@@H]21] | |
| | 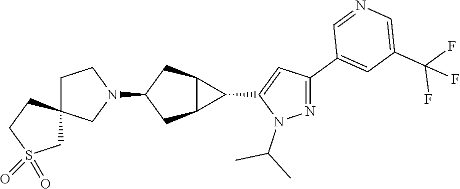 [SMILES: CC(C)n1nc(-c2cncc(C(F)(F)F)c2)cc1[C@H]1[C@@H]2C[C@H](N3CC[C@]4(CCS(=O)(=O)C4)C3)C[C@@H]21] | |
| 11 12 | 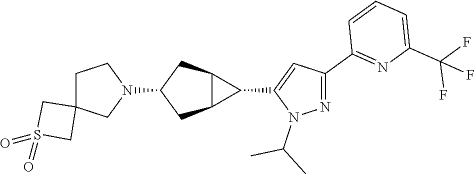 [SMILES: CC(C)n1nc(-c2cccc(C(F)(F)F)n2)cc1[C@H]1[C@@H]2C[C@@H](N3CCC4(C3)CS(=O)(=O)C4)C[C@@H]21] | 6-((1R,3s,5S,6r)-6-(1-isopropyl-3-(6- (trifluoromethyl)pyridin-2-yl)-1H-pyrazol- 5-yl)bicyclo[3.1.0]hexan-3-yl)-2-thia-6- azaspiro[3.4]octane 2,2-dioxide 6-((1R,3r,5S,6r)-6-(1-isopropyl-3-(6- (trifluoromethyl)pyridin-2-yl)-1H-pyrazol- 5-yl)bicyclo[3.1.0]hexan-3-yl)-2-thia-6- azaspiro[3.4]octane 2,2-dioxide |
| | 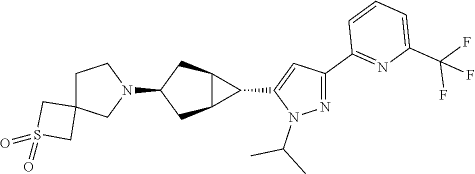 [SMILES: CC(C)n1nc(-c2cccc(C(F)(F)F)n2)cc1[C@H]1[C@@H]2C[C@H](N3CCC4(C3)CS(=O)(=O)C4)C[C@@H]21] | |
| 13 14 | 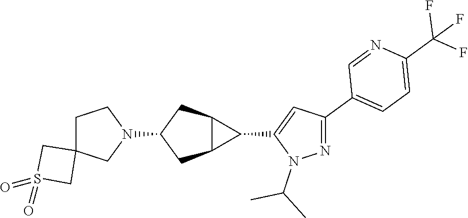 [SMILES: CC(C)n1nc(-c2ccc(C(F)(F)F)nc2)cc1[C@H]1[C@@H]2C[C@@H](N3CCC4(C3)CS(=O)(=O)C4)C[C@@H]21] | 6-((1R,3s,5S,6r)-6-(1-isopropyl-3-(6- (trifluoromethyl)pyridin-3-yl)-1H-pyrazol- 5-yl)bicyclo[3.1.0]hexan-3-yl)-2-thia-6- azaspiro[3.4 ]octane 2,2-dioxide 6-((1R,3r,5S,6r)-6-(1-isopropyl-3-(6- (trifluoromethyl)pyridin-3-yl)-1H-pyrazol- 5-yl)bicyclo[3.1.0]hexan-3-yl)-2-thia-6- azaspiro[3.4]octane 2,2-dioxide |
| | 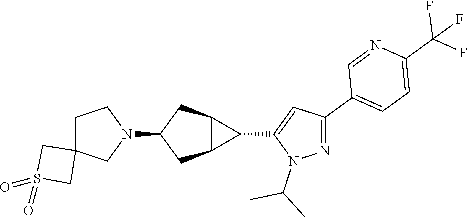 [SMILES: CC(C)n1nc(-c2ccc(C(F)(F)F)nc2)cc1[C@H]1[C@@H]2C[C@H](N3CCC4(C3)CS(=O)(=O)C4)C[C@@H]21] | |
| 15 | 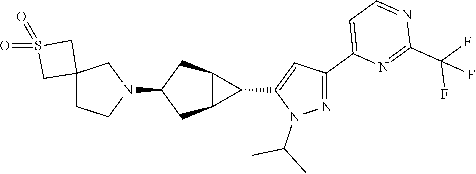 [SMILES: CC(C)n1nc(-c2ccnc(C(F)(F)F)n2)cc1[C@H]1[C@@H]2C[C@H](N3CCC4(C3)CS(=O)(=O)C4)C[C@@H]21] | 6-((1R,3r,5S,6r)-6-(1-isopropyl-3-(2- (trifluoromethyl)pyrimidin-4-yl)-1H- pyrazol-5-yl)bicyclo[3.1.0]hexan-3-yl)-2- thia-6-azaspiro[3.4]octane 2,2-dioxide |
| 16 17 | 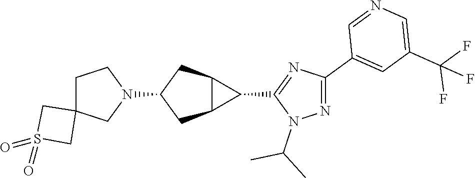 [SMILES: CC(C)n1nc(-c2cncc(C(F)(F)F)c2)nc1[C@H]1[C@@H]2C[C@@H](N3CCC4(C3)CS(=O)(=O)C4)C[C@@H]21] | 6-((1R,3s,5S,6r)-6-(1-isopropyl-3-(5- (trifluoromethyl)pyridin-3-yl)-1H-1,2,4- triazol-5-yl)bicyclo[3.1.0]hexan-3-yl)-2- thia-6-azaspiro[3.4]octane 2,2-dioxide 6-((1R,3r,5S,6r)-6-(1-isopropyl-3-(5- (trifluoromethyl)pyridin-3-yl)-1H-1,2,4- triazol-5-yl)bicyclo[3.1.0]hexan-3-yl)-2- thia-6-azaspiro[3.4]octane 2,2-dioxide |
| | 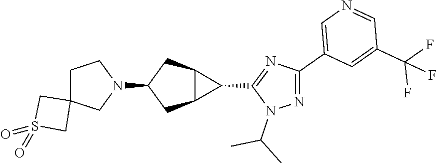 [SMILES: CC(C)n1nc(-c2cncc(C(F)(F)F)c2)nc1[C@H]1[C@@H]2C[C@H](N3CCC4(C3)CS(=O)(=O)C4)C[C@@H]21] | |
| 18 19 | 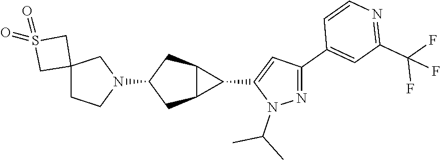 [SMILES: CC(C)n1nc(-c2ccnc(C(F)(F)F)c2)cc1[C@H]1[C@@H]2C[C@@H](N3CCC4(C3)CS(=O)(=O)C4)C[C@@H]21] | 6-((1R,3s,5S,6r)-6-(1-isopropyl-3-(2- (trifluoromethyl)pyridin-4-yl)-1H-pyrazol- 5-yl)bicyclo[3.1.0]hexan-3-yl)-2-thia-6- azaspiro[3.4]octane 2,2-dioxide 6-((1R,3r,5S,6r)-6-(1-isopropyl-3-(2- (trifluoromethyl)pyridin-4-yl)-1H-pyrazol- 5-yl)bicyclo[3.1.0]hexan-3-yl)-2-thia-6- azaspiro[3.4]octane 2,2-dioxide |
| | 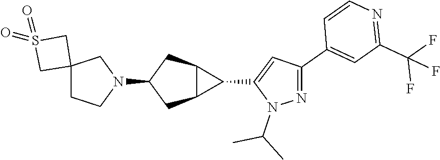 [SMILES: CC(C)n1nc(-c2ccnc(C(F)(F)F)c2)cc1[C@H]1[C@@H]2C[C@H](N3CCC4(C3)CS(=O)(=O)C4)C[C@@H]21] | |
| 20 21 | 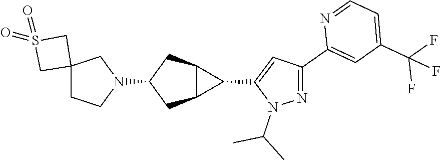 [SMILES: CC(C)n1nc(-c2cc(C(F)(F)F)ccn2)cc1[C@H]1[C@@H]2C[C@@H](N3CCC4(C3)CS(=O)(=O)C4)C[C@@H]21] | 6-((1R,3s,5S,6r)-6-(1-isopropyl-3-(4- (trifluoromethyl)pyridin-2-yl)-1H-pyrazol- 5-yl)bicyclo[3.1.0]hexan-3-yl)-2-thia-6- azaspiro[3.4]octane 2,2-dioxide 6-((1R,3r,5S,6r)-6-(1-isopropyl-3-(4- (trifluoromethyl)pyridin-2-yl)-1H-pyrazol- 5-yl)bicyclo[3.1.0]hexan-3-yl)-2-thia-6- azaspiro[3.4]octane 2,2-dioxide |
| | 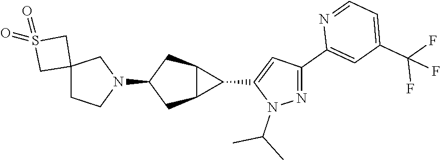 [SMILES: CC(C)n1nc(-c2cc(C(F)(F)F)ccn2)cc1[C@H]1[C@@H]2C[C@H](N3CCC4(C3)CS(=O)(=O)C4)C[C@@H]21] | |
| 22 23 | 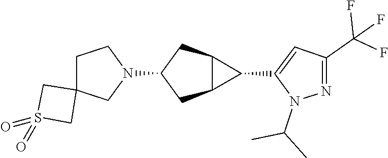 [SMILES: CC(C)n1nc(C(F)(F)F)cc1[C@H]1[C@@H]2C[C@@H](N3CCC4(C3)CS(=O)(=O)C4)C[C@@H]21] | 6-((1R,3s,5S,6r)-6-(1-isopropyl-3- (trifluoromethyl)-1H-pyrazol-5- yl)bicyclo[3.1.0]hexan-3-yl)-2-thia-6- azaspiro[3.4]octane 2,2-dioxide 6-((1R,3r,5S,6r)-6-(1-isopropyl-3- (trifluoromethyl)-1H-pyrazol-5- yl)bicyclo[3.1.0]hexan-3-yl)-2-thia-6- azaspiro[3.4]octane 2,2-dioxide |
| | 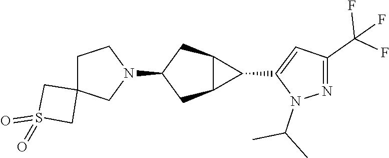 [SMILES: CC(C)n1nc(C(F)(F)F)cc1[C@H]1[C@@H]2C[C@H](N3CCC4(C3)CS(=O)(=O)C4)C[C@@H]21] | |
| 24 25 | 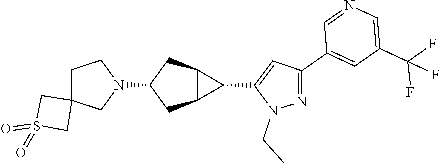 [SMILES: CCn1nc(-c2cncc(C(F)(F)F)c2)cc1[C@H]1[C@@H]2C[C@@H](N3CCC4(C3)CS(=O)(=O)C4)C[C@@H]21] | 6-((1R,3s,5S,6r)-6-(1-ethyl-3-(5- (trifluoromethyl)pyridin-3-yl)-1H-pyrazol- 5-yl)bicyclo[3.1.0]hexan-3-yl)-2-thia-6- azaspiro[3.4]octane 2,2-dioxide 6-((1R,3r,5S,6r)-6-(1-ethyl-3-(5- (trifluoromethyl)pyridin-3-yl)-1H-pyrazol- 5-yl)bicyclo[3.1.0]hexan-3-yl)-2-thia-6- azaspiro[3.4]octane 2,2-dioxide |
| | 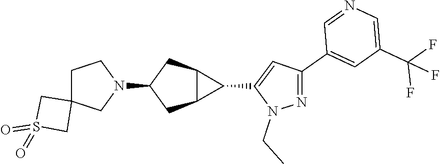 [SMILES: CCn1nc(-c2cncc(C(F)(F)F)c2)cc1[C@H]1[C@@H]2C[C@H](N3CCC4(C3)CS(=O)(=O)C4)C[C@@H]21] | |
| 26 27 | 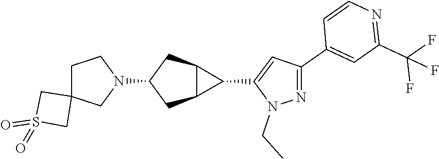 [SMILES: CCn1nc(-c2ccnc(C(F)(F)F)c2)cc1[C@H]1[C@@H]2C[C@@H](N3CCC4(C3)CS(=O)(=O)C4)C[C@@H]21] | 6-((1R,3s,5S,6r)-6-(1-ethyl-3-(2- (trifluoromethyl)pyridin-4-yl)-1H-pyrazol- 5-yl)bicyclo[3.1.0]hexan-3-yl)-2-thia-6- azaspiro[3.4]octane 2,2-dioxide 6-((1R,3r,5S,6r)-6-(1-ethyl-3-(2- (trifluoromethyl)pyridin-4-yl)-1H-pyrazol- 5-yl)bicyclo[3.1.0]hexan-3-yl)-2-thia-6- azaspiro[3.4]octane 2,2-dioxide |
| | 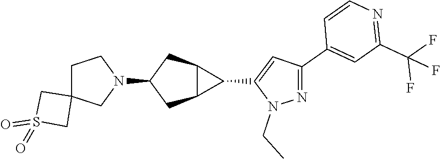 [SMILES: CCn1nc(-c2ccnc(C(F)(F)F)c2)cc1[C@H]1[C@@H]2C[C@H](N3CCC4(C3)CS(=O)(=O)C4)C[C@@H]21] | |
| 28 | 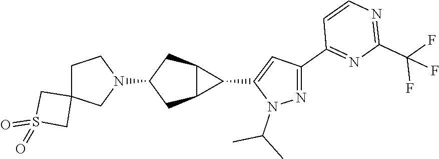 [SMILES: CC(C)n1nc(-c2ccnc(C(F)(F)F)n2)cc1[C@H]1[C@@H]2C[C@@H](N3CCC4(C3)CS(=O)(=O)C4)C[C@@H]21] | 6-((1R,3s,5S,6r)-6-(1-isopropyl-3-(2- (trifluoromethyl)pyrimidin-4-yl)-1H- pyrazol-5-yl)bicyclo[3.1.0]hexan-3-yl)-2- thia-6-azaspiro[3.4]octane 2,2-dioxide |
| 31 | 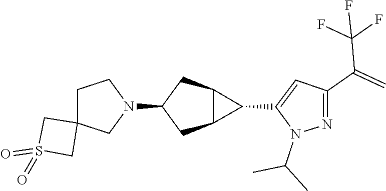 [SMILES: C=C(c1cc([C@H]2[C@@H]3C[C@H](N4CCC5(C4)CS(=O)(=O)C5)C[C@@H]32)n(C(C)C)n1)C(F)(F)F] | 6-((1R,3r,5S,6r)-6-(1-isopropyl-3-(3,3,3- trifluoroprop-1-en-2-yl)-1H-pyrazol-5- yl)bicyclo[3.1.0]hexan-3-yl)-2-thia-6- azaspiro[3.4]octane 2,2-dioxide |
| 32 | 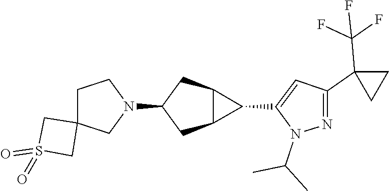 [SMILES: CC(C)n1nc(C2(C(F)(F)F)CC2)cc1[C@H]1[C@@H]2C[C@H](N3CCC4(C3)CS(=O)(=O)C4)C[C@@H]21] | 6-((1R,3r,5S,6r)-6-(1-isopropyl-3-(1- (trifluoromethyl)cyclopropyl)-1H-pyrazol- 5-yl)bicyclo[3.1.0]hexan-3-yl)-2-thia-6- azaspiro[3.4]octane 2,2-dioxide |
| 38 | 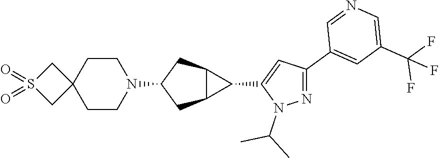 [SMILES: CC(C)n1nc(-c2cncc(C(F)(F)F)c2)cc1[C@H]1[C@@H]2C[C@@H](N3CCC4(CC3)CS(=O)(=O)C4)C[C@@H]21] | 7-((1R,3s,5S,6r)-6-(1-isopropyl-3-(5- (trifluoromethyl)pyridin-3-yl)-1H-pyrazol- 5-yl)bicyclo[3.1.0]hexan-3-yl)-2-thia-7- azaspiro[3.5]nonane 2,2-dioxide |
| 40 | 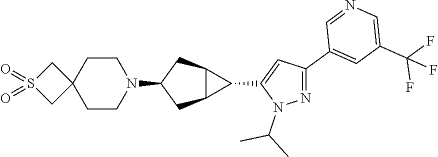 [SMILES: CC(C)n1nc(-c2cncc(C(F)(F)F)c2)cc1[C@H]1[C@@H]2C[C@H](N3CCC4(CC3)CS(=O)(=O)C4)C[C@@H]21] | 7-((1R,3r,5S,6r)-6-(1-isopropyl-3-(5- (trifluoromethyl)pyridin-3-yl)-1H-pyrazol- 5-yl)bicyclo[3.1.0]hexan-3-yl)-2-thia-7- azaspiro[3.5]nonane 2,2-dioxide |
| 43* 44* | 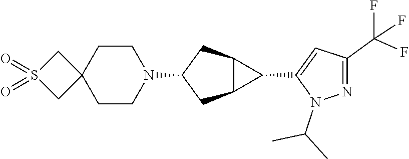 [SMILES: CC(C)n1nc(C(F)(F)F)cc1[C@H]1[C@@H]2C[C@@H](N3CCC4(CC3)CS(=O)(=O)C4)C[C@@H]21] | 7-((1R,3s,5S,6r)-6-(1-isopropyl-3- (trifluoromethyl)-1H-pyrazol-5- yl)bicyclo[3.1.0]hexan-3-yl)-2-thia-7- azaspiro[3.5]nonane 2,2-dioxide 7-((1R,3r,5S,6r)-6-(1-isopropyl-3- (trifluoromethyl)-1H-pyrazol-5- yl)bicyclo[3.1.0]hexan-3-yl)-2-thia-7- azaspiro[3.5]nonane 2,2-dioxide |
| | 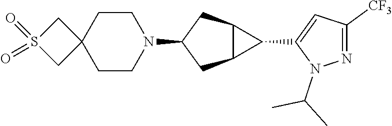 [SMILES: CC(C)n1nc(C(F)(F)F)cc1[C@H]1[C@@H]2C[C@H](N3CCC4(CC3)CS(=O)(=O)C4)C[C@@H]21] | |
| 45* 46* | 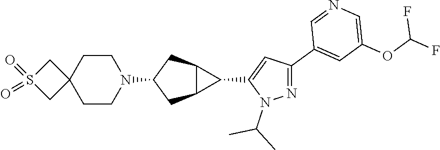 [SMILES: CC(C)n1nc(-c2cncc(OC(F)F)c2)cc1[C@H]1[C@@H]2C[C@@H](N3CCC4(CC3)CS(=O)(=O)C4)C[C@@H]21] | 7-((1R,3s,5S,6r)-6-(3-(5- (difluoromethoxy)pyridin-3-yl)-1- isopropyl-1H-pyrazol-5- yl)bicyclo[3.1.0]hexan-3-yl)-2-thia-7- azaspiro[3.5]nonane 2,2-dioxide 7-((1R,3r,5S,6r)-6-(3-(5- (difluoromethoxy)pyridin-3-yl)-1- isopropyl-1H-pyrazol-5- yl)bicyclo[3.1.0]hexan-3-yl)-2-thia-7- azaspiro[3.5]nonane 2,2-dioxide |
| | 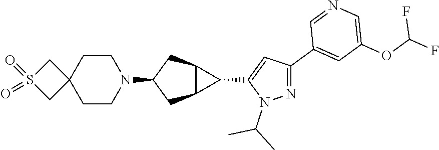 [SMILES: CC(C)n1nc(-c2cncc(OC(F)F)c2)cc1[C@H]1[C@@H]2C[C@H](N3CCC4(CC3)CS(=O)(=O)C4)C[C@@H]21] | |
| 47 | 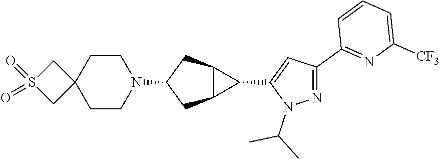 [SMILES: CC(C)n1nc(-c2cccc(C(F)(F)F)n2)cc1[C@H]1[C@@H]2C[C@@H](N3CCC4(CC3)CS(=O)(=O)C4)C[C@@H]21] | 7-((1R,3s,5S,6r)-6-(1-isopropyl-3-(6- (trifluoromethyl)pyridin-2-yl)-1H-pyrazol- 5-yl)bicyclo[3.1.0]hexan-3-yl)-2-thia-7- azaspiro[3.5]nonane 2,2-dioxide |
| 48 | 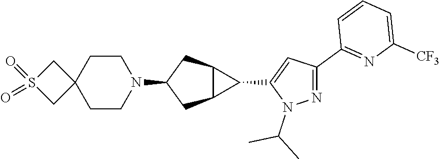 [SMILES: CC(C)n1nc(-c2cccc(C(F)(F)F)n2)cc1[C@H]1[C@@H]2C[C@H](N3CCC4(CC3)CS(=O)(=O)C4)C[C@@H]21] | 7-((1R,3r,5S,6r)-6-(1-isopropyl-3-(6- (trifluoromethyl)pyridin-2-yl)-1H-pyrazol- 5-yl)bicyclo[3.1.0]hexan-3-yl)-2-thia-7- azaspiro[3.5]nonane 2,2-dioxide |
| 49 | 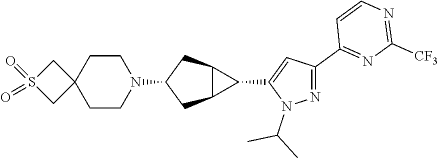 [SMILES: CC(C)n1nc(-c2ccnc(C(F)(F)F)n2)cc1[C@H]1[C@@H]2C[C@@H](N3CCC4(CC3)CS(=O)(=O)C4)C[C@@H]21] | 7-((1R,3s,5S,6r)-6-(1-isopropyl-3-(2- (trifluoromethyl)pyrimidin-4-yl)-1H- pyrazol-5-yl)bicyclo[3.1.0]hexan-3-yl)-2- thia-7-azaspiro[3.5]nonane 2,2-dioxide |
| 50 | 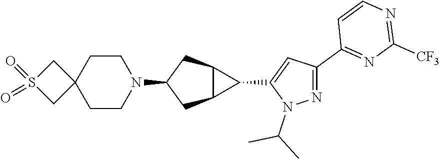 [SMILES: CC(C)n1nc(-c2ccnc(C(F)(F)F)n2)cc1[C@H]1[C@@H]2C[C@H](N3CCC4(CC3)CS(=O)(=O)C4)C[C@@H]21] | 7-((1R,3r,5S,6r)-6-(1-isopropyl-3-(2- (trifluoromethyl)pyrimidin-4-yl)-1H- pyrazol-5-yl)bicyclo[3.1.0]hexan-3-yl)-2- thia-7-azaspiro[3.5]nonane 2,2-dioxide |
| 51 | 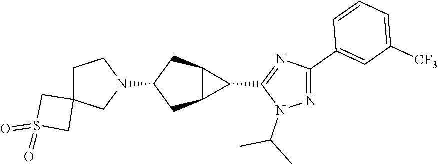 [SMILES: CC(C)n1nc(-c2cccc(C(F)(F)F)c2)nc1[C@H]1[C@@H]2C[C@@H](N3CCC4(C3)CS(=O)(=O)C4)C[C@@H]21] | 6-((1R,3s,5S,6r)-6-(1-isopropyl-3-(3- (trifluoromethyl)phenyl)-1H-1,2,4-triazol- 5-yl)bicyclo[3.1.0]hexan-3-yl)-2-thia-6- azaspiro[3.4]octane 2,2-dioxide |
| 52 | 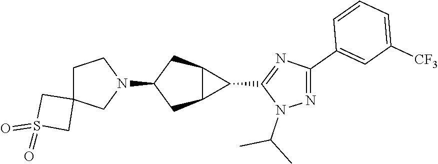 [SMILES: CC(C)n1nc(-c2cccc(C(F)(F)F)c2)nc1[C@H]1[C@@H]2C[C@H](N3CCC4(C3)CS(=O)(=O)C4)C[C@@H]21] | 6-((1R,3r,5S,6r)-6-(1-isopropyl-3-(3- (trifluoromethyl)phenyl)-1H-1,2,4-triazol- 5-yl)bicyclo[3.1.0]hexan-3-yl)-2-thia-6- azaspiro[3.4]octane 2,2-dioxide |
| 53* 54* | 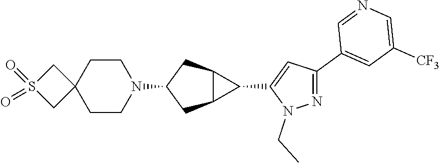 [SMILES: CCn1nc(-c2cncc(C(F)(F)F)c2)cc1[C@H]1[C@@H]2C[C@@H](N3CCC4(CC3)CS(=O)(=O)C4)C[C@@H]21] | 7-((1R,3s,5S,6r)-6-(1-ethyl-3-(5- (trifluoromethyl)pyridin-3-yl)-1H-pyrazol- 5-yl)bicyclo[3.1.0]hexan-3-yl)-2-thia-7- azaspiro[3.5]nonane 2,2-dioxide 7-((1R,3r,5S,6r)-6-(1-ethyl-3-(5- (trifluoromethyl)pyridin-3-yl)-1H-pyrazol- 5-yl)bicyclo[3.1.0]hexan-3-yl)-2-thia-7- azaspiro[3.5]nonane 2,2-dioxide |
| | 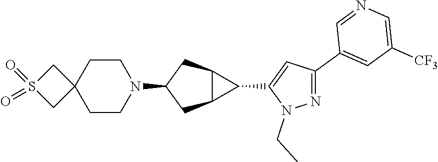 [SMILES: CCn1nc(-c2cncc(C(F)(F)F)c2)cc1[C@H]1[C@@H]2C[C@H](N3CCC4(CC3)CS(=O)(=O)C4)C[C@@H]21] | |
| 55 | 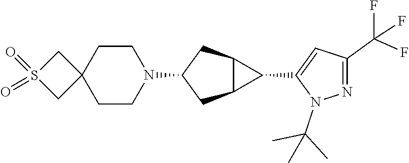 [SMILES: CC(C)(C)n1nc(C(F)(F)F)cc1[C@H]1[C@@H]2C[C@@H](N3CCC4(CC3)CS(=O)(=O)C4)C[C@@H]21] | 7-((1R,3s,5S,6r)-6-(1-(tert-butyl)-3- (trifluoromethyl)-1H-pyrazol-5- yl)bicyclo[3.1.0]hexan-3-yl)-2-thia-7- azaspiro[3.5]nonane 2,2-dioxide |
| 56 | 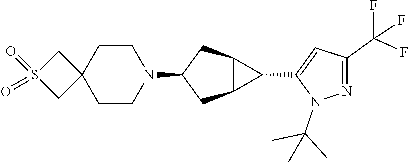 [SMILES: CC(C)(C)n1nc(C(F)(F)F)cc1[C@H]1[C@@H]2C[C@H](N3CCC4(CC3)CS(=O)(=O)C4)C[C@@H]21] | 7-((1R,3r,5S,6r)-6-(1-(tert-butyl)-3- (trifluoromethyl)-1H-pyrazol-5- yl)bicyclo[3.1.0]hexan-3-yl)-2-thia-7- azaspiro[3.5]nonane 2,2-dioxide |
| 57 | 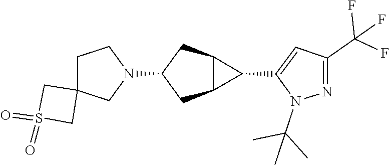 [SMILES: CC(C)(C)n1nc(C(F)(F)F)cc1[C@H]1[C@@H]2C[C@@H](N3CCC4(C3)CS(=O)(=O)C4)C[C@@H]21] | 6-((1R,3s,5S,6r)-6-(1-(tert-butyl)-3- (trifluoromethyl)-1H-pyrazol-5- yl)bicyclo[3.1.0]hexan-3-yl)-2-thia-6- azaspiro[3.4]octane 2,2-dioxide |
| 58 | 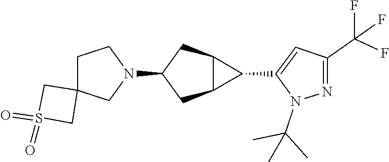 [SMILES: CC(C)(C)n1nc(C(F)(F)F)cc1[C@H]1[C@@H]2C[C@H](N3CCC4(C3)CS(=O)(=O)C4)C[C@@H]21] | 6-((1R,3r,5S,6r)-6-(1-(tert-butyl)-3- (trifluoromethyl)-1H-pyrazol-5- yl)bicyclo[3.1.0]hexan-3-yl)-2-thia-6- azaspiro[3.4]octane 2,2-dioxide |
| 62* 63* | 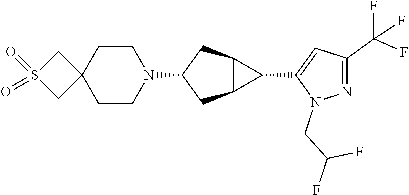 [SMILES: O=S1(=O)CC2(CCN([C@@H]3C[C@@H]4[C@H](C3)[C@H]4c3cc(C(F)(F)F)nn3CC(F)F)CC2)C1] | 7-((1R,3s,5S,6r)-6-(1-(2,2-difluoroethyl)-3- (trifluoromethyl)-1H-pyrazol-5- yl)bicyclo[3.1.0]hexan-3-yl)-2-thia-7- azaspiro[3.5]nonane 2,2-dioxide 7-((1R,3r,5S,6r)-6-(1-(2,2-difluoroethyl)-3- (trifluoromethyl)-1H-pyrazol-5- yl)bicyclo[3.1.0]hexan-3-yl)-2-thia-7- azaspiro[3.5]nonane 2,2-dioxide |
| | 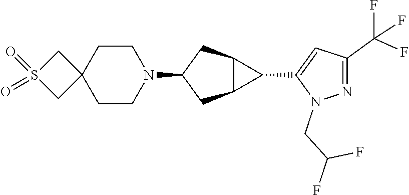 [SMILES: O=S1(=O)CC2(CCN([C@H]3C[C@@H]4[C@H](C3)[C@H]4c3cc(C(F)(F)F)nn3CC(F)F)CC2)C1] | |
| 64* 65* | 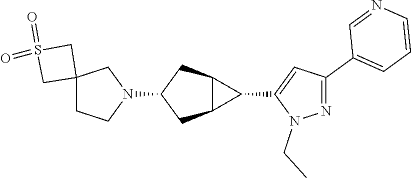 [SMILES: CCn1nc(-c2cccnc2)cc1[C@H]1[C@@H]2C[C@@H](N3CCC4(C3)CS(=O)(=O)C4)C[C@@H]21] | 6-((1R,3s,5S,6r)-6-(1-ethyl-3-(pyridin-3- yl)-1H-pyrazol-5-yl)bicyclo[3.1.0]hexan-3- yl)-2-thia-6-azaspiro[3.4]octane 2,2- dioxide 6-((1R,3r,5S,6r)-6-(1-ethyl-3-(pyridin-3- yl)-1H-pyrazol-5-yl)bicyclo[3.1.0]hexan-3- yl)-2-thia-6-azaspiro[3.4]octane 2,2- dioxide |
| | 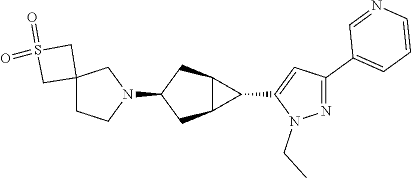 [SMILES: CCn1nc(-c2cccnc2)cc1[C@H]1[C@@H]2C[C@H](N3CCC4(C3)CS(=O)(=O)C4)C[C@@H]21] | |
| 66* 67* | 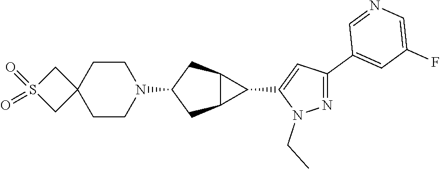 [SMILES: CCn1nc(-c2cncc(F)c2)cc1[C@H]1[C@@H]2C[C@@H](N3CCC4(CC3)CS(=O)(=O)C4)C[C@@H]21] | 7-((1R,3s,5S,6r)-6-(1-ethyl-3-(5- fluoropyridin-3-yl)-1H-pyrazol-5- yl)bicyclo[3.1.0]hexan-3-yl)-2-thia-7- azaspiro[3.5]nonane 2,2-dioxide 7-((1R,3r,5S,6r)-6-(1-ethyl-3-(5- fluoropyridin-3-yl)-1H-pyrazol-5- yl)bicyclo[3.1.0]hexan-3-yl)-2-thia-7- azaspiro[3.5]nonane 2,2-dioxide |
| | 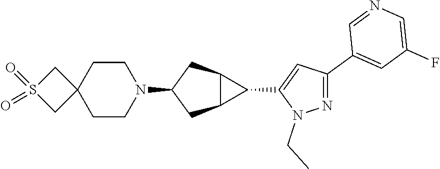 [SMILES: CCn1nc(-c2cncc(F)c2)cc1[C@H]1[C@@H]2C[C@H](N3CCC4(CC3)CS(=O)(=O)C4)C[C@@H]21] | |
| 68* 69* | 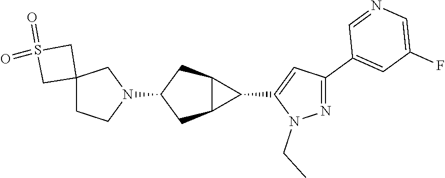 [SMILES: CCn1nc(-c2cncc(F)c2)cc1[C@H]1[C@@H]2C[C@@H](N3CCC4(C3)CS(=O)(=O)C4)C[C@@H]21] | 6-((1R,3s,5S,6r)-6-(1-ethyl-3-(5- fluoropyridin-3-yl)-1H-pyrazol-5- yl)bicyclo[3.1.0]hexan-3-yl)-2-thia-6- azaspiro[3.4]octane 2,2-dioxide 6-((1R,3r,5S,6r)-6-(1-ethyl-3-(5- fluoropyridin-3-yl)-1H-pyrazol-5- yl)bicyclo[3.1.0]hexan-3-yl)-2-thia-6- azaspiro[3.4]octane 2,2-dioxide |
| | 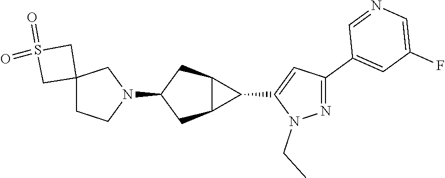 [SMILES: CCn1nc(-c2cncc(F)c2)cc1[C@H]1[C@@H]2C[C@H](N3CCC4(C3)CS(=O)(=O)C4)C[C@@H]21] | |
| 70* 71* | 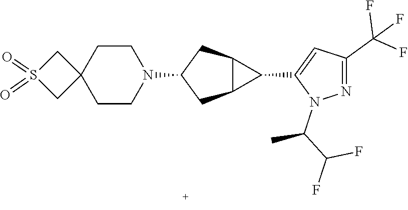 [SMILES: C[C@H](C(F)F)n1nc(C(F)(F)F)cc1[C@H]1[C@@H]2C[C@@H](N3CCC4(CC3)CS(=O)(=O)C4)C[C@@H]21] | 7-((1R,3S,5S,6r)-6-(1-((R)-1,1- difluoropropan-2-yl)-3-(trifluoromethyl)- 1H-pyrazol-5-yl)bicyclo[3.1.0]hexan-3-yl)- 2-thia-7-azaspiro[3.5]nonane 2,2-dioxide and 7-((1R,3R,5S,6r)-6-(1-((S)-1,1- difluoropropan-2-yl)-3-(trifluoromethyl)- 1H-pyrazol-5-yl)bicyclo[3.1.0]hexan-3-yl)- 2-thia-7-azaspiro[3.5]nonane 2,2-dioxide 7-((1R,3R,5S,6r)-6-(1-((R)-1,1- difluoropropan-2-yl)-3-(trifluoromethyl)- 1H-pyrazol-5-yl)bicyclo[3.1.0]hexan-3-yl)- 2-thia-7-azaspiro[3.5]nonane 2,2-dioxide and 7-((1R,3S,5S,6r)-6-(1-((S)-1,1- difluoropropan-2-yl)-3-(trifluoromethyl)- |
| | | 1H-pyrazol-5-yl)bicyclo[3.1.0]hexan-3-yl)- |
| | 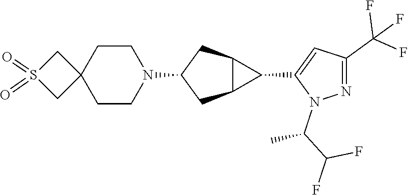 [SMILES: C[C@@H](C(F)F)n1nc(C(F)(F)F)cc1[C@H]1[C@@H]2C[C@@H](N3CCC4(CC3)CS(=O)(=O)C4)C[C@@H]21] | 2-thia-7-azaspiro[3.5]nonane 2,2-dioxide |
| | 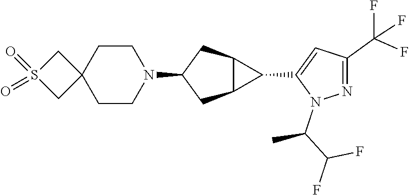 [SMILES: C[C@H](C(F)F)n1nc(C(F)(F)F)cc1[C@H]1[C@@H]2C[C@H](N3CCC4(CC3)CS(=O)(=O)C4)C[C@@H]21] | |
| | 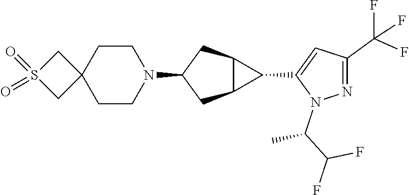 [SMILES: C[C@@H](C(F)F)n1nc(C(F)(F)F)cc1[C@H]1[C@@H]2C[C@H](N3CCC4(CC3)CS(=O)(=O)C4)C[C@@H]21] | |
| 72 | 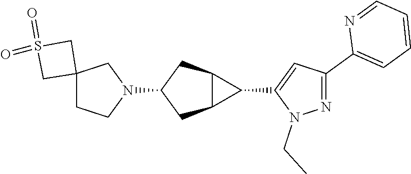 [SMILES: CCn1nc(-c2ccccn2)cc1[C@H]1[C@@H]2C[C@@H](N3CCC4(C3)CS(=O)(=O)C4)C[C@@H]21] | 6-((1R,3s,5S,6r)-6-(1-ethyl-3-(pyridin-2- yl)-1H-pyrazol-5-yl)bicyclo[3.1.0]hexan-3- yl)-2-thia-6-azaspiro[3.4]octane 2,2- dioxide |
| 73 | 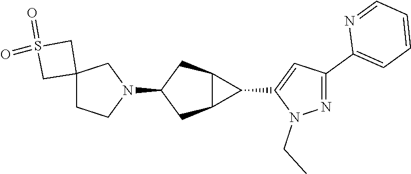 [SMILES: CCn1nc(-c2ccccn2)cc1[C@H]1[C@@H]2C[C@H](N3CCC4(C3)CS(=O)(=O)C4)C[C@@H]21] | 6-((1R,3r,5S,6r)-6-(1-ethyl-3-(pyridin-2- yl)-1H-pyrazol-5-yl)bicyclo[3.1.0]hexan-3- yl)-2-thia-6-azaspiro[3.4]octane 2,2- dioxide |
| 74 | 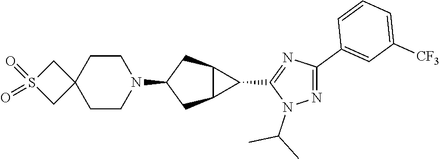 [SMILES: CC(C)n1nc(-c2cccc(C(F)(F)F)c2)nc1[C@H]1[C@@H]2C[C@H](N3CCC4(CC3)CS(=O)(=O)C4)C[C@@H]21] | 7-((1R,3r,5S,6r)-6-(1-isopropyl-3-(3- (trifluoromethyl)phenyl)-1H-1,2,4-triazol- 5-yl)bicyclo[3.1.0]hexan-3-yl)-2-thia-7- azaspiro[3.5]nonane 2,2-dioxide |
| 75 | 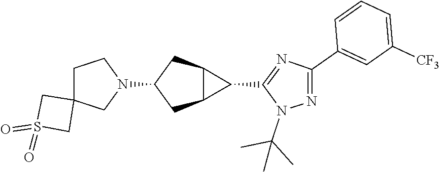 [SMILES: CC(C)(C)n1nc(-c2cccc(C(F)(F)F)c2)nc1[C@H]1[C@@H]2C[C@@H](N3CCC4(C3)CS(=O)(=O)C4)C[C@@H]21] | 6-((1R,3s,5S,6r)-6-(1-(tert-butyl)-3-(3- (trifluoromethyl)phenyl)-1H-1,2,4-triazol- 5-yl)bicyclo[3.1.0]hexan-3-yl)-2-thia-6- azaspiro[3.4]octane 2,2-dioxide |
| 76 | 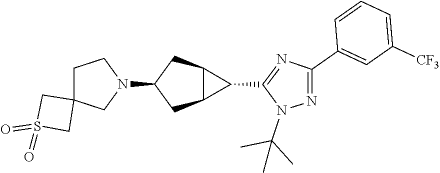 [SMILES: CC(C)(C)n1nc(-c2cccc(C(F)(F)F)c2)nc1[C@H]1[C@@H]2C[C@H](N3CCC4(C3)CS(=O)(=O)C4)C[C@@H]21] | 6-((1R,3r,5S,6r)-6-(1-(tert-butyl)-3-(3- (trifluoromethyl)phenyl)-1H-1,2,4-triazol- 5-yl)bicyclo[3.1.0]hexan-3-yl)-2-thia-6- azaspiro[3.4]octane 2,2-dioxide |
| 77* 78* | 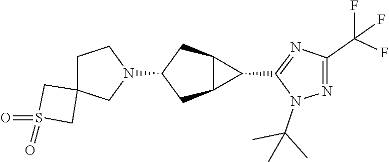 [SMILES: CC(C)(C)n1nc(C(F)(F)F)nc1[C@H]1[C@@H]2C[C@@H](N3CCC4(C3)CS(=O)(=O)C4)C[C@@H]21] | 6-((1R,3s,5S,6r)-6-(1-(tert-butyl)-3- (trifluoromethyl)-1H-1,2,4-triazol-5- yl)bicyclo[3.1.0]hexan-3-yl)-2-thia-6- azaspiro[3.4]octane 2,2-dioxide 6-((1R,3r,5S,6r)-6-(1-(tert-butyl)-3- (trifluoromethyl)-1H-1,2,4-triazol-5- yl)bicyclo[3.1.0]hexan-3-yl)-2-thia-6- azaspiro[3.4]octane 2,2-dioxide |
| | 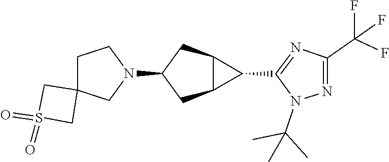 [SMILES: CC(C)(C)n1nc(C(F)(F)F)nc1[C@H]1[C@@H]2C[C@H](N3CCC4(C3)CS(=O)(=O)C4)C[C@@H]21] | |
| 79 | 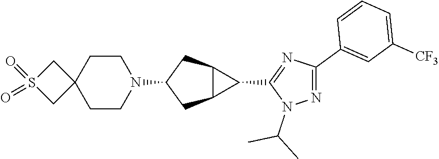 [SMILES: CC(C)n1nc(-c2cccc(C(F)(F)F)c2)nc1[C@H]1[C@@H]2C[C@@H](N3CCC4(CC3)CS(=O)(=O)C4)C[C@@H]21] | 7-((1R,3s,5S,6r)-6-(1-isopropyl-3-(3- (trifluoromethyl)phenyl)-1H-1,2,4-triazol- 5-yl)bicyclo[3.1.0]hexan-3-yl)-2-thia-7- azaspiro[3.5]nonane 2,2-dioxide |
| 80* 81* 82* 83* | 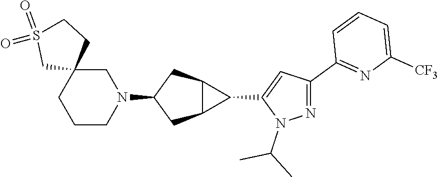 [SMILES: CC(C)n1nc(-c2cccc(C(F)(F)F)n2)cc1[C@H]1[C@@H]2C[C@H](N3CCC[C@@]4(CCS(=O)(=O)C4)C3)C[C@@H]21] | (R)-7-((1R,3r,5S,6R)-6-(1-isopropyl-3-(6- (trifluoromethyl)pyridin-2-yl)-1H-pyrazol- 5-yl)bicyclo[3.1.0]hexan-3-yl)-2-thia-7- azaspiro[4.5]decane 2,2-dioxide (R)-7-((1R,3s,5S,6R)-6-(1-isopropyl-3-(6- (trifluoromethyl)pyridin-2-yl)-1H-pyrazol- 5-yl)bicyclo[3.1.0]hexan-3-yl)-2-thia-7- azaspiro[4.5]decane 2,2-dioxide (S)-7-((1R,3s,5S,6S)-6-(1-isopropyl-3-(6- (trifluoromethyl)pyridin-2-yl)-1H-pyrazol- 5-yl)bicyclo[3.1.0]hexan-3-yl)-2-thia-7- azaspiro[4.5]decane 2,2-dioxide |
| | | (S)-7-((1R,3r,5S,6S)-6-(1-isopropyl-3-(6- |
| | 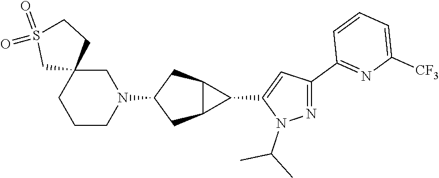 [SMILES: CC(C)n1nc(-c2cccc(C(F)(F)F)n2)cc1[C@H]1[C@@H]2C[C@@H](N3CCC[C@@]4(CCS(=O)(=O)C4)C3)C[C@@H]21] | (trifluoromethyl)pyridin-2-yl)-1H-pyrazol- 5-yl)bicyclo[3.1.0]hexan-3-yl)-2-thia-7- azaspiro[4.5]decane 2,2-dioxide |
| | 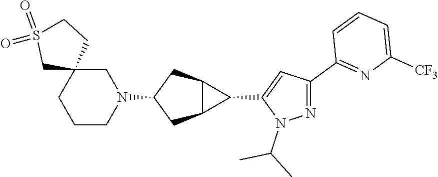 [SMILES: CC(C)n1nc(-c2cccc(C(F)(F)F)n2)cc1[C@H]1[C@@H]2C[C@@H](N3CCC[C@]4(CCS(=O)(=O)C4)C3)C[C@@H]21] | |
| | 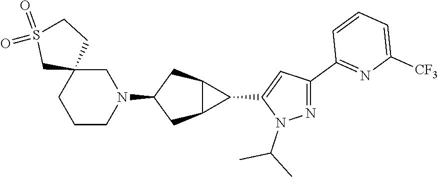 [SMILES: CC(C)n1nc(-c2cccc(C(F)(F)F)n2)cc1[C@H]1[C@@H]2C[C@H](N3CCC[C@]4(CCS(=O)(=O)C4)C3)C[C@@H]21] | |
| 86* 87* | 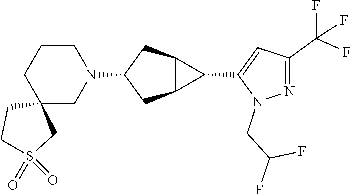 [SMILES: O=S1(=O)CC[C@@]2(CCCN([C@@H]3C[C@@H]4[C@H](C3)[C@H]4c3cc(C(F)(F)F)nn3CC(F)F)C2)C1] | (R)-7-((1R,3s,5S,6R)-6-(1-(2,2- difluoroethyl)-3-(trifluoromethyl)-1H- pyrazol-5-yl)bicyclo[3.1.0]hexan-3-yl)-2- thia-7-azaspiro[4.5]decane 2,2-dioxide (S)-7-((1R,3s,5S,6S)-6-(1-(2,2- difluoroethyl)-3-(trifluoromethyl)-1H- pyrazol-5-yl)bicyclo[3.1.0]hexan-3-yl)-2- thia-7-azaspiro[4.5]decane 2,2-dioxide |
| | 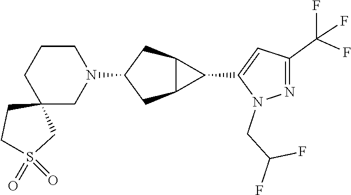 [SMILES: O=S1(=O)CC[C@]2(CCCN([C@@H]3C[C@@H]4[C@H](C3)[C@H]4c3cc(C(F)(F)F)nn3CC(F)F)C2)C1] | |
| 88* 89* | 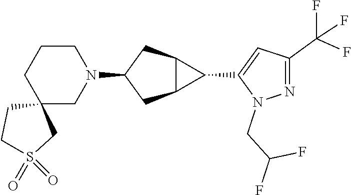 [SMILES: O=S1(=O)CC[C@@]2(CCCN([C@H]3C[C@@H]4[C@H](C3)[C@H]4c3cc(C(F)(F)F)nn3CC(F)F)C2)C1] | (R)-7-((1R,3r,5S,6R)-6-(1-(2,2- difluoroethyl)-3-(trifluoromethyl)-1H- pyrazol-5-yl)bicyclo[3.1.0]hexan-3-yl)-2- thia-7-azaspiro[4.5]decane 2,2-dioxide (S)-7-((1R,3r,5S,6S)-6-(1-(2,2- difluoroethyl)-3-(trifluoromethyl)-1H- pyrazol-5-yl)bicyclo[3.1.0]hexan-3-yl)-2- thia-7-azaspiro[4.5]decane 2,2-dioxide |
| | 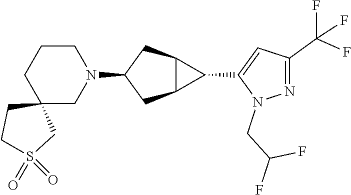 [SMILES: O=S1(=O)CC[C@]2(CCCN([C@H]3C[C@@H]4[C@H](C3)[C@H]4c3cc(C(F)(F)F)nn3CC(F)F)C2)C1] | |
| 90* 91* | 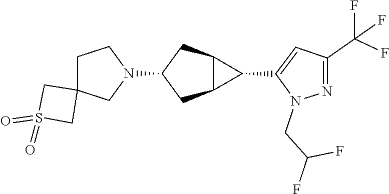 [SMILES: O=S1(=O)CC2(CCN([C@@H]3C[C@@H]4[C@H](C3)[C@H]4c3cc(C(F)(F)F)nn3CC(F)F)C2)C1] | 6-((1R,3s,5S,6r)-6-(1-(2,2-difluoroethyl)-3- (trifluoromethyl)-1H-pyrazol-5- yl)bicyclo[3.1.0]hexan-3-yl)-2-thia-6- azaspiro[3.4]octane 2,2-dioxide 6-((1R,3r,5S,6r)-6-(1-(2,2-difluoroethyl)-3- (trifluoromethyl)-1H-pyrazol-5- yl)bicyclo[3.1.0]hexan-3-yl)-2-thia-6- azaspiro[3.4]octane 2,2-dioxide |
| | 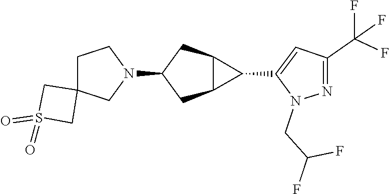 [SMILES: O=S1(=O)CC2(CCN([C@H]3C[C@@H]4[C@H](C3)[C@H]4c3cc(C(F)(F)F)nn3CC(F)F)C2)C1] | |
| 92* 93* 94* 95* | 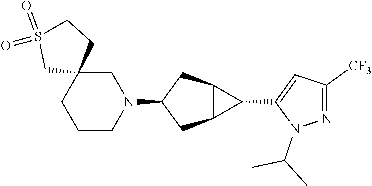 [SMILES: CC(C)n1nc(C(F)(F)F)cc1[C@H]1[C@@H]2C[C@H](N3CCC[C@@]4(CCS(=O)(=O)C4)C3)C[C@@H]21] | (R)-7-((1R,3r,5S,6R)-6-(1-isopropyl-3- (trifluoromethyl)-1H-pyrazol-5- yl)bicyclo[3.1.0]hexan-3-yl)-2-thia-7- azaspiro[4.5]decane 2,2-dioxide (R)-7-((1R,3s,5S,6R)-6-(1-isopropyl-3- (trifluoromethyl)-1H-pyrazol-5- yl)bicyclo[3.1.0]hexan-3-yl)-2-thia-7- azaspiro[4.5]decane 2,2-dioxide (S)-7-((1R,3s,5S,6S)-6-(1-isopropyl-3- (trifluoromethyl)-1H-pyrazol-5- yl)bicyclo[3.1.0]hexan-3-yl)-2-thia-7- azaspiro[4.5]decane 2,2-dioxide |
| | | (S)-7-((1R,3r,5S,6S)-6-(1-isopropyl-3- |
| | 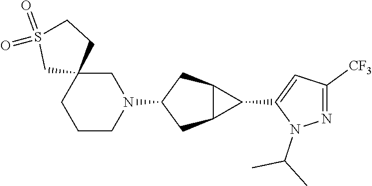 [SMILES: CC(C)n1nc(C(F)(F)F)cc1[C@H]1[C@@H]2C[C@@H](N3CCC[C@@]4(CCS(=O)(=O)C4)C3)C[C@@H]21] | (trifluoromethyl)-1H-pyrazol-5- yl)bicyclo[3.1.0]hexan-3-yl)-2-thia-7- azaspiro[4.5]decane 2,2-dioxide |
| | 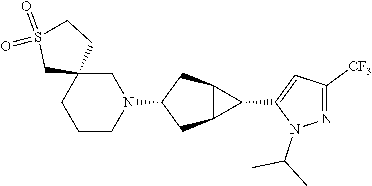 [SMILES: CC(C)n1nc(C(F)(F)F)cc1[C@H]1[C@@H]2C[C@@H](N3CCC[C@]4(CCS(=O)(=O)C4)C3)C[C@@H]21] | |
| | 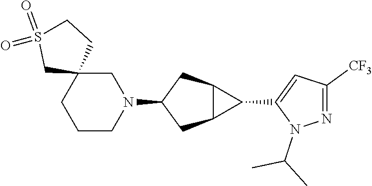 [SMILES: CC(C)n1nc(C(F)(F)F)cc1[C@H]1[C@@H]2C[C@H](N3CCC[C@]4(CCS(=O)(=O)C4)C3)C[C@@H]21] | |
| 96* | 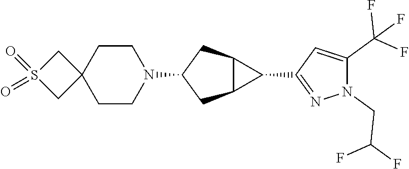 [SMILES: O=S1(=O)CC2(CCN([C@@H]3C[C@@H]4[C@H](C3)[C@H]4c3cc(C(F)(F)F)n(CC(F)F)n3)CC2)C1] | 7-((1R,3s,5S,6r)-6-(1-(2,2-difluoroethyl)-5- (trifluoromethyl)-1H-pyrazol-3- yl)bicyclo[3.1.0]hexan-3-yl)-2-thia-7- azaspiro[3.5]nonane 2,2-dioxide |
| 97 | 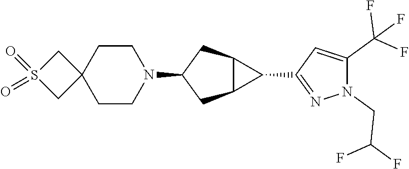 [SMILES: O=S1(=O)CC2(CCN([C@H]3C[C@@H]4[C@H](C3)[C@H]4c3cc(C(F)(F)F)n(CC(F)F)n3)CC2)C1] | 7-((1R,3r,5S,6r)-6-(1-(2,2-difluoroethyl)-5- (trifluoromethyl)-1H-pyrazol-3- yl)bicyclo[3.1.0]hexan-3-yl)-2-thia-7- azaspiro[3.5]nonane 2,2-dioxide |
| 98* 99* | 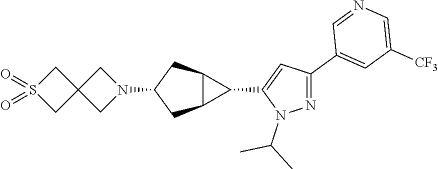 [SMILES: CC(C)n1nc(-c2cncc(C(F)(F)F)c2)cc1[C@H]1[C@@H]2C[C@@H](N3CC4(C3)CS(=O)(=O)C4)C[C@@H]21] | 6-((1R,3s,5S,6r)-6-(1-isopropyl-3-(5- (trifluoromethyl)pyridin-3-yl)-1H-pyrazol- 5-yl)bicyclo[3.1.0]hexan-3-yl)-2-thia-6- azaspiro[3.3]heptane 2,2-dioxide 6-((1R,3r,5S,6r)-6-(1-isopropyl-3-(5- (trifluoromethyl)pyridin-3-yl)-1H-pyrazol- 5-yl)bicyclo[3.1.0]hexan-3-yl)-2-thia-6- azaspiro[3.3]heptane 2,2-dioxide |
| | 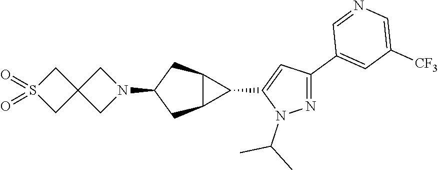 [SMILES: CC(C)n1nc(-c2cncc(C(F)(F)F)c2)cc1[C@H]1[C@@H]2C[C@H](N3CC4(C3)CS(=O)(=O)C4)C[C@@H]21] | |
| 102 103 | 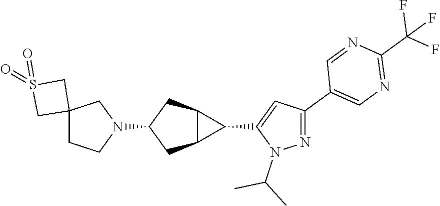 [SMILES: CC(C)n1nc(-c2cnc(C(F)(F)F)nc2)cc1[C@H]1[C@@H]2C[C@@H](N3CCC4(C3)CS(=O)(=O)C4)C[C@@H]21] | 6-((1R,3s,5S,6r)-6-(1-Isopropyl-3-(2- (trifluoromethyl)pyrimidin-5-yl)-1H- pyrazol-5-yl)bicyclo[3.1.0]hexan-3-yl)-2- thia-6-azaspiro[3.4]octane 2,2-dioxide 6-((1R,3r,5S,6r)-6-(1-Isopropyl-3-(2- (trifluoromethyl)pyrimidin-5-yl)-1H- pyrazol-5-yl)bicyclo[3.1.0]hexan-3-yl)-2- thia-6-azaspiro[3.4]octane 2,2-dioxide |
| | 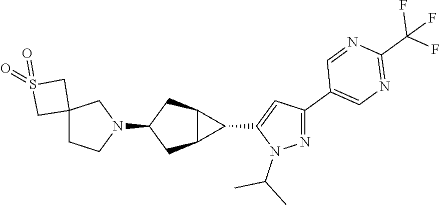 [SMILES: CC(C)n1nc(-c2cnc(C(F)(F)F)nc2)cc1[C@H]1[C@@H]2C[C@H](N3CCC4(C3)CS(=O)(=O)C4)C[C@@H]21] | |
| 104* 105* | 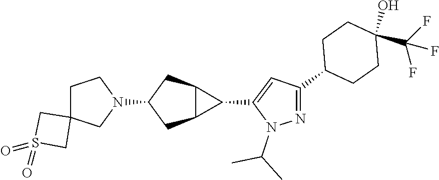 [SMILES: CC(C)n1nc([C@H]2CC[C@](O)(C(F)(F)F)CC2)cc1[C@H]1[C@@H]2C[C@@H](N3CCC4(C3)CS(=O)(=O)C4)C[C@@H]21] | 6-((1R,3S,5S,6r)-6-(3-((1s,4S)-4-Hydroxy- 4-(trifluoromethyl)cyclohexyl)-1- isopropyl-1H-pyrazol-5- yl)bicyclo[3.1.0]hexan-3-yl)-2-thia-6- azaspiro[3.4]octane 2,2-dioxide 6-((1R,3S,5S,6r)-6-(3-((1r,4R)-4-Hydroxy- 4-(trifluoromethyl)cyclohexyl)-1- isopropyl-1H-pyrazol-5- yl)bicyclo[3.1.0]hexan-3-yl)-2-thia-6- azaspiro[3.4]octane 2,2-dioxide (absolute stereochemistry assigned |
| | | arbitrarily) |
| | 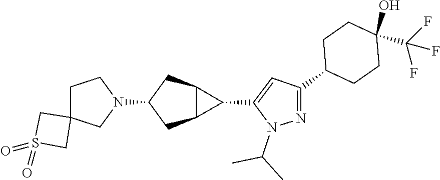 [SMILES: CC(C)n1nc([C@H]2CC[C@@](O)(C(F)(F)F)CC2)cc1[C@H]1[C@@H]2C[C@@H](N3CCC4(C3)CS(=O)(=O)C4)C[C@@H]21] | |
| 106 107 | 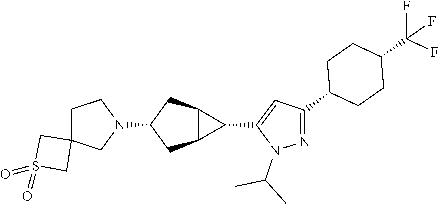 [SMILES: CC(C)n1nc([C@H]2CC[C@@H](C(F)(F)F)CC2)cc1[C@H]1[C@@H]2C[C@@H](N3CCC4(C3)CS(=O)(=O)C4)C[C@@H]21] | 6-((1R,3S,5S,6r)-6-(1-Isopropyl-3-((1s,4S)- 4-(trifluoromethyl)cyclohexyl)-1H-pyrazol- 5-yl)bicyclo[3.1.0]hexan-3-yl)-2-thia-6- azaspiro[3.4]octane 2,2-dioxide 6-((1R,3S,5S,6r)-6-(1-Isopropyl-3-((1r,4R)- 4-(trifluoromethyl)cyclohexyl)-1H-pyrazol- 5-yl)bicyclo[3.1.0]hexan-3-yl)-2-thia-6- azaspiro[3.4]octane 2,2-dioxide |
| | 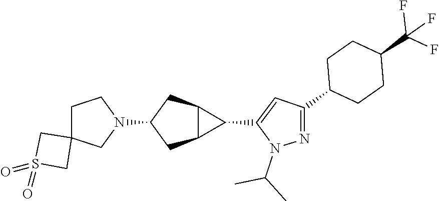 [SMILES: CC(C)n1nc([C@H]2CC[C@H](C(F)(F)F)CC2)cc1[C@H]1[C@@H]2C[C@@H](N3CCC4(C3)CS(=O)(=O)C4)C[C@@H]21] | |
| 108 | 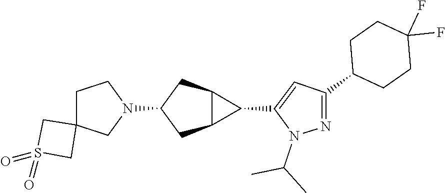 | 6-((1R,3s,5S,6r)-6-(3-(4,4- difluorocyclohexyl)-1-isopropyl-1H- pyrazol-5-yl)bicyclo[3.1.0]hexan-3-yl)-2- thia-6-azaspiro[3.4]octane 2,2-dioxide |

### III. Pharmaceutical Compositions and Modes of Administration

Compounds provided herein may be administered in the form of pharmaceutical compositions. Thus, provided herein are also pharmaceutical compositions that comprise one or more of the compounds described herein or a pharmaceutically acceptable salt, a stereoisomer, or a mixture of stereoisomers thereof, and one or more pharmaceutically acceptable excipients. Suitable pharmaceutically acceptable excipients may include, for example, inert solid diluents and fillers, liquid diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In some embodiments, the pharmaceutical composition comprises a compound of Formula I, or a pharmaceutically acceptable salt thereof, and a pharmaceutically acceptable excipient. In some embodiments, the pharmaceutical composition comprises a compound of Formula Ia, or a pharmaceutically acceptable salt thereof, and a pharmaceutically acceptable excipient. In some embodiments, the pharmaceutical composition comprises a compound of Formula Ib, or a pharmaceutically acceptable salt thereof, and a pharmaceutically acceptable excipient. In some embodiments, the pharmaceutical composition comprises a compound of Formula Ic, or a pharmaceutically acceptable salt thereof, and a pharmaceutically acceptable excipient. In some embodiments, the pharmaceutical composition comprises a compound of Table 1, or a pharmaceutically acceptable salt thereof, and a pharmaceutically acceptable excipient.

The pharmaceutical compositions may be administered in either single or multiple doses. The pharmaceutical composition may be administered by various methods including, for example, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or tablet, such as enteric coated tablets. In making the pharmaceutical compositions that include at least one compound described herein or a pharmaceutically acceptable salt, a stereoisomer, or a mixture of stereoisomers thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propythydroxy-benzoates; sweetening agents; and flavoring agents.

The compositions that include at least one compound described herein or a pharmaceutically acceptable salt, a stereoisomer, or a mixture of stereoisomers thereof can be formulated so as to provide quick, sustained, or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, a stereoisomer, or a mixture of stereoisomers thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

### IV. Methods of Treatment

Described herein are methods for promoting myelination of central nervous system neurons in a subject suffering from a myelin-related disorder, the method comprising administering to the subject a therapeutically effective amount of a compound of Formula I, or a pharmaceutical composition comprising the same. In certain embodiments, the subject matter disclosed herein is directed to a compound of Formula I or a pharmaceutically acceptable salt thereof for promoting myelination of central nervous system neurons in a subject suffering from a myelin-related disorder. In another embodiment, the subject matter described herein is directed to the use of a compound of Formula I or a pharmaceutically acceptable salt thereof for the manufacture of a medicament for promoting myelination of central nervous system neurons in a subject suffering from a myelin-related disorder. In some embodiments, the compound of Formula I is a compound of Table 1, or a pharmaceutically acceptable salt thereof.

In certain embodiments, in the methods for promoting myelination of central nervous system neurons in a subject suffering from a myelin-related disorder, the compound of Formula I, or a pharmaceutically acceptable salt thereof, or a pharmaceutical composition comprising the same, inhibits enzyme mediated synthesis of one or more sterol intermediates in the cholesterol biosynthesis pathway.

In certain embodiments, in the methods for promoting myelination of central nervous system neurons in a subject suffering from a myelin-related disorder, the compound of Formula I, or a pharmaceutically acceptable salt thereof, or a pharmaceutical composition comprising the same, promotes accumulation of Δ8,9-unsaturated sterol intermediates in the cholesterol biosynthesis pathway.

In certain embodiments, in the methods for promoting myelination of central nervous system neurons in a subject suffering from a myelin-related disorder, the compound of Formula I, or a pharmaceutically acceptable salt thereof, or a pharmaceutical composition comprising the same, inhibits one or more of CYP51, sterol-14-reductase, or EBP enzyme mediated synthesis of sterol intermediates in the cholesterol biosynthesis pathway.

In certain embodiments, in the methods for promoting myelination of central nervous system neurons in a subject suffering from a myelin-related disorder, the compound of Formula I, or a pharmaceutically acceptable salt thereof, or a pharmaceutical composition comprising the same, induces, promotes, and/or modulates oligodendrocyte precursor cell (OPC) differentiation, proliferation and/or maturation. In certain embodiments, the induction of OPC differentiation is characterized by an increase in myelin basic protein (MBP) expression.

In certain embodiments, the subject matter described herein is directed to a method of treating a disorder in a subject in need thereof, the method comprising administering to the subject in need thereof a therapeutically effective amount of a compound of Formula I or a pharmaceutically acceptable salt thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof. In some embodiments, the compound of Formula I is a compound of Table 1, or a pharmaceutically acceptable salt thereof.

In certain embodiments, the subject matter disclosed herein is directed to a compound of Formula I or a pharmaceutically acceptable salt thereof, for use in treating a disorder in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof. In some embodiments, the compound of Formula I is a compound of Table 1, or a pharmaceutically acceptable salt thereof.

In certain embodiments, the subject matter disclosed herein is directed to the use of a compound of Formula I, or a pharmaceutically acceptable salt thereof, in the manufacture of a medicament for treating a disorder in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof. In some embodiments, the compound of Formula I is a compound of Table 1, or a pharmaceutically acceptable salt thereof.

In certain embodiments, the subject matter disclosed herein is directed to a method of promoting myelination in a subject in need thereof, comprising administering to the subject a therapeutically effective amount of a compound of Formula I, or a pharmaceutically acceptable salt thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof.

In certain embodiments, the subject matter disclosed herein is directed to a compound of Formula I, or a pharmaceutically acceptable salt thereof, or a pharmaceutical composition comprising the compound, for use in promoting myelination in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof. In some embodiments, the compound of Formula I is a compound of Table 1, or a pharmaceutically acceptable salt thereof.

In certain embodiments, the subject matter disclosed herein is directed to use of a compound of Formula I, or a pharmaceutically acceptable salt thereof, or a pharmaceutical composition comprising the compound, in the manufacture of a medicament for promoting myelination in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof. In some embodiments, the compound of Formula I is a compound of Table 1, or a pharmaceutically acceptable salt thereof.

In certain embodiments, the subject matter disclosed herein is directed to a method of inducing endogenous oligodendrocyte precursor cell (OPC) differentiation in a subject in need thereof, the method comprising administering to the subject a therapeutically effective amount of a compound of Formula I, or a pharmaceutically acceptable salt thereof, or a pharmaceutical composition comprising the same. In certain embodiments, the subject is suffering from a myelin-related disorder. In certain embodments, the myelin-related disorder is multiple sclerosis.

Such myelin-related disorders include, but are not limited to, multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy and radiation-induced demyelination.

The compound of Formula I or a pharmaceutically acceptable salt thereof can be administered alone or in combination with another agent to a subject suffering from a myelin-related disorder to promote myelination of neurons (e.g., neuronal axons). A myelin-related disorder can include any disease, condition (e.g., those occurring from traumatic spinal cord injury and cerebral infarction), or disorderresulting in abnormalities of the myelin sheath. Abnormalities can be caused by loss of myelin referred to as demyelination, dysfunctional myelin referred to as dysmyelination or failure to form enough myelin referred to as hypomyelination. A myelin related disorder as described herein can arise from a genetic disorder or from one or more of a variety of neurotoxic insults. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof. In other embodiments, the compound of Formula I is a compound of Formula Ic, or a pharmaceutically acceptable salt thereof. In some embodiments, the compound of Formula I is a compound of Table 1, or a pharmaceutically acceptable salt thereof.

“Demyelination” as used herein, refers to the act of demyelinating, or the damage or loss of part or all of the myelin sheath insulating the nerves, and is the hallmark of myelin-related disorders. In certain embodiments, demyelination refers to the damage or loss of part or all of the myelin sheath insulating a subset of nerves in an individual, such as, for example, one or more nerves localized in a particular area of the body (e.g., neurons in the brain or spinal cord, or both brain and spinal cord; or the optic nerve).

Myelination of neurons requires oligodendrocytes. The term “myelination”, as used herein, refers to the generation of the nerve's myelin sheath by replacing myelin producing cells or restoring their function. The neurons that undergo remyelination may be in the brian, spinal cord, or both the brain and spinal cord. Restoring the function of a myelin producing cell may include, for example, increasing the rate of myelin production in a cell (or cells) with a less-than-average production level. Such increase may encompass raising the rate of myelin production up to or exceeding average production level; but also may encompass raising the rate of myelin production to a level that is still less than average, but higher than the previous level.

“Promoting Myelination” as used herein refers to increasing the rate of myelin production rather than a mere net increase in the amount of myelin as compared to a baseline level of myelin production rate in a subject. An increase in the rate of myelin production can be determined using imaging techniques or functional measurements. In some embodiments, myelination is promoted by increasing the differentiation of OPCs, increasing the accumulation of 8,9-unsaturated sterol intermediates in the biosynthetic pathway, increasing the formation of OPCs, or any combinations thereof. Such activities may be evaluated, for example, using one or more in vitro assays, such as those described herein or known to one of skill in the art.

A “baseline level of myelin production rate” as used herein, refers to the rate of myelin production in subject being treated before the onset of treatment.

### V. Methods of Preparing Compounds of Formula I and Pharmaceutically Acceptable Salts Thereof

Compounds can be synthesized by synthetic routes that include processes analogous to those well-known in the chemical arts, particularly in light of the description contained herein, and those for other heterocycles described in: Comprehensive Heterocyclic Chemistry II, Editors Katritzky and Rees, Elsevier, 1997, e.g., Volume 3; Liebigs Annalen der Chemie, (9):1910-16, (1985); Helvetica Chimica Acta, 41:1052-60, (1958); Arzneimittel-Forschung, 40(12):1328-31, (1990), each of which are expressly incorporated by reference. Starting materials are generally available from commercial sources such as Aldrich Chemicals (Milwaukee, WI) or are readily prepared using methods well known to those skilled in the art (e.g., prepared by methods generally described in Louis F. Fieser and Mary Fieser, Reagents for Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database).

Synthetic chemistry transformations and protecting group methodologies (protection and deprotection) useful in synthesizing compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, _{3rd}Ed., John Wiley and Sons (1999); and L. Paquette, ed., Encyclopedia of Reagents for Organic Synthesis, John Wiley and Sons (1995) and subsequent editions thereof.

Compounds may be prepared singly or as compound libraries comprising at least 2, for example 5 to 1,000 compounds, or 10 to 100 compounds. Libraries of compounds of Formula I, or pharmaceutically acceptable salts thereof, may be prepared by a combinatorial ‘split and mix’ approach or by multiple parallel syntheses using either solution phase or solid phase chemistry, by procedures known to those skilled in the art. Thus, according to a further aspect, there is provided a compound library comprising at least 2 compounds, or pharmaceutically acceptable salts thereof.

### EXAMPLES

The Examples provide exemplary methods for preparing compounds. Those skilled in the art will appreciate that other synthetic routes may be used to synthesize the compounds. Although specific starting materials and reagents are depicted and discussed in the Schemes, General Procedures, and Examples, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art. The asterix (*) indicates an isolated isomer or isolated group of isomers, but that the stereochemistry has not been assigned.

### Synthesis of Intermediates

### Intermediate Example I-1: (1R,5S,6r)-6-(3-Iodo-1-isopropyl-1H-pyrazol-5-yl)bicyclo[3.1.0]hexan-3-one

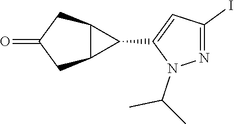 [SMILES: CC(C)n1nc(I)cc1[C@H]1[C@@H]2CC(=O)C[C@@H]21]

### Step 1: Synthesis of ethyl 4-(3-((tert-butyldiphenylsilyl)oxy)bicyclo[3.1.0]hexan-6-yl)-2,4-dioxobutanoate

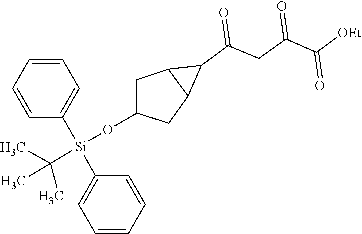 [SMILES: CCOC(=O)C(=O)CC(=O)C1C2CC(O[Si](c3ccccc3)(c3ccccc3)C(C)(C)C)CC21]

To a solution of 1-(3-((tert-butyldiphenylsilyl)oxy)bicyclo[3.1.0]hexan-6-yl)ethanone (27 g, 71 mmol) in anhydrous tetrahydrofuran (300 mL) was added lithium bis(trimethylsilyl)amide (106 mL, 106 mmol, 1 M in tetrahydrofuran) slowly at −78° C. under nitrogen. After 0.5 h, diethyl oxalate (15.63 g, 107.0 mmol) was added, and the reaction mixture was warmed to room temperature. After 6 h, the reaction mixture was quenched with 3 M aqueous hydrochloric acid until the solution reached pH˜3. The mixture was extracted with ethyl acetate (2×200 mL). The combined organic layers were washed with saturated aqueous sodium chloride solution, dried over anhydrous sodium sulfate, filtered, and concentrated to provided ethyl 4-(3-((tert-butyldiphenylsilyl)oxy)bicyclo[3.1.0]hexan-6-yl)-2,4-dioxobutanoate (40 g), which was used without further purification. LCMS: [M+H]⁺ 479.0, [M+Na]⁺ 501.0, [M+Na+CH₃CN]⁺ 542.0

### Step 2: Synthesis of ethyl 5-(3-((tert-butyldiphenylsilyl)oxy)bicyclo[3.1.0]hexan-6-yl)-1-isopropyl-1H-pyrazole-3-carboxylate

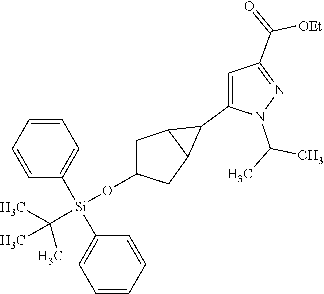 [SMILES: CCOC(=O)c1cc(C2C3CC(O[Si](c4ccccc4)(c4ccccc4)C(C)(C)C)CC32)n(C(C)C)n1]

To a solution of ethyl 4-(3-((tert-butyldiphenylsilyl)oxy)bicyclo[3.1.0]hexan-6-yl)-2,4-dioxobutanoate (0.40 kg, 84 mmol) in ethanol (500 mL) was added N-isopropylhydrazine hydrochloride (9.7 g, 84 mmol) at room temperature. After 16 h, triethylamine was added, and the resulting mixture was concentrated in vacuo. Purification of the residue by flash column chromatography (9% ethyl acetate in petroleum ether) afforded ethyl 5-(3-((tert-butyldiphenylsilyl)oxy)bicyclo[3.1.0]hexan-6-yl)-1-isopropyl-1H-pyrazole-3-carboxylate (19.5 g, 45%). LRMS: [M+H]⁺=517.1.

### Step 3: Synthesis of 5-(3-((tert-butyldiphenylsilyl)oxy)bicyclo[3.1.0]hexan-6-yl)-1-isopropyl-1H-pyrazole-3-carboxylic acid

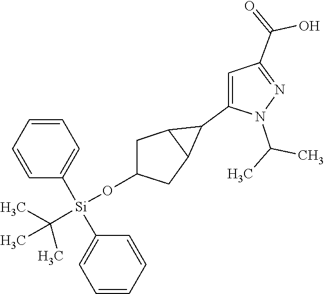 [SMILES: CC(C)n1nc(C(=O)O)cc1C1C2CC(O[Si](c3ccccc3)(c3ccccc3)C(C)(C)C)CC21]

To a stirred solution of ethyl 5-(3-((tert-butyldiphenylsilyl)oxy)bicyclo[3.1.0]hexan-6-yl)-1-isopropyl-1H-pyrazole-3-carboxylate (19.5 g, 37.7 mmol) in ethanol (200 mL) was added a solution of sodium hydroxide (6.30 g, 151 mmol) in water (50 mL) at room temperature. After 6 h, the reaction mixture was concentrated in vacuo, and the resulting aqueous solution was diluted with water (10 mL). 2 M aqueous hydrochloric acid was added until the solution reached pH˜3. The aqueous mixture was extracted with ethyl acetate (2×100 mL). The combined organic extracts were washed with saturated aqueous sodium chloride solution, dried over anhydrous sodium sulfate, filtered, and concentrated to provide crude 5-(3-((tert-butyldiphenylsilyl)oxy)bicyclo[3.1.0]hexan-6-yl)-1-isopropyl-1H-pyrazole-3-carboxylic (18 g, 92%).

### Step 4: Synthesis of benzyl (5-(3-((tert-butyldiphenylsilyl)oxy)bicyclo[3.1.0]hexan-6-yl)-1-isopropyl-1H-pyrazol-3-yl)carbamate

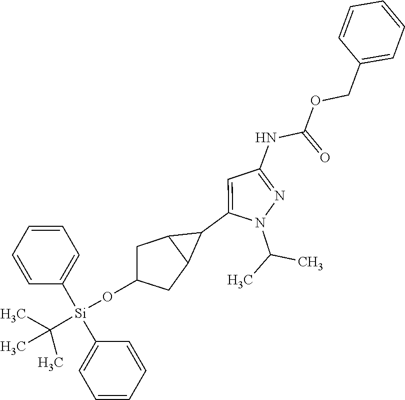 [SMILES: CC(C)n1nc(NC(=O)OCc2ccccc2)cc1C1C2CC(O[Si](c3ccccc3)(c3ccccc3)C(C)(C)C)CC21]

A 500 mL three-neck flask was charged with 5-(3-((tert-butyldiphenylsilyl)oxy) bicyclo[3.1.0]hexan-6-yl)-1-isopropyl-1H-pyrazole-3-carboxylic acid (26 g, 53 mmol), diisopropylethylamine (14 mL, 0.080 mol), benzyl alcohol (17.26 g, 159.6 mmol) and anhydrous toluene (300 mL). The reaction mixture was purged with nitrogen for 2 min and heated to 100° C. Diphenyl phosphorazidate (17.2 mL, 79.85 mmol) was added dropwise to the reaction mixture, and the reaction was maintained at 100° C. After 16 h, the reaction mixture was concentrated in vacuo. The resulting residue was purified by flash column chromatography (20:1 petroleum ether/ethyl acetate) to afford benzyl (5-(3-((tert-butyldiphenylsilyl) oxy) bicyclo[3.1.0]hexan-6-yl)-1-isopropyl-1H-pyrazol-3-yl)carbamate (28 g, 89%). LRMS: [M+H]⁺594.0.

### Step 5: Synthesis of 5-(3-((tert-butyldiphenylsilyl)oxy)bicyclo[3.1.0]hexan-6-yl)-3-iodo-1-isopropyl-1H-pyrazole

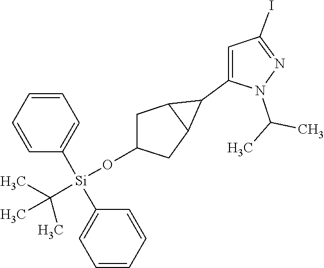 [SMILES: CC(C)n1nc(I)cc1C1C2CC(O[Si](c3ccccc3)(c3ccccc3)C(C)(C)C)CC21]

To a solution of benzyl (5-(3-((tert-butyldiphenylsilyl)oxy)bicyclo[3.1.0]hexan-6-yl)-1-isopropyl-1H-pyrazol-3-yl)carbamate (28 g, 47 mmol) in methanol (200 mL) was added 10% palladium on carbon (2.8 g). The reaction mixture was stirred at room temperature under 1 atm of hydrogen. After 16 h, the reaction mixture was filtered through Celite. The filtrate was concentrated in vacuo to provide crude 5-(3-((tert-butyldiphenylsilyl)oxy)bicyclo[3.1.0]hexan-6-yl)-1-isopropyl-1H-pyrazol-3-amine which was dissolved in acetonitrile (200 mL). A solution of 4-methylbenzenesulfonic acid monohydrate (22.34 g, 117.5 mmol) in water (25 mL) was added at room temperature. After 30 min at room temperature, the reaction mixture was cooled to 0° C. A solution of sodium nitrite (5.4 g, 78 mmol) and sodium iodide (1174 g, 78.31 mmol) in water (25 mL) was added dropwise to the reaction mixture. After 30 min, saturated aqueous sodium sulfite was added to the reaction, and the resulting aqueous mixture was extracted with ethyl acetate (2×100 mL). The combined organic layers were washed with saturated aqueous sodium chloride, dried over anhydrous sodium sulfate, filtered, and concentrated. Purified of the residue by flash column chromatography (3-5% ethyl acetate in petroleum ether) afforded 5-(3-((tert-butyldiphenylsilyl)oxy)bicyclo[3.1.0]hexan-6-yl)-3-iodo-1-isopropyl-1H-pyrazole (11.2 g, 50%). LRMS: [M+H]⁺ 570.9.

### Step 6: Synthesis of 6-(3-iodo-1-isopropyl-1H-pyrazol-5-yl)bicyclo[3.1.0]hexan-3-ol

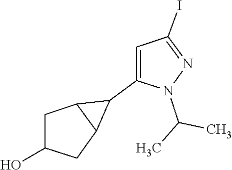 [SMILES: CC(C)n1nc(I)cc1C1C2CC(O)CC21]

A solution of 5-(3-((tert-butyldiphenylsilyl)oxy)bicyclo[3.1.0]hexan-6-yl)-3-iodo-1-isopropyl-1H-pyrazole (11.2 g, 19.7 mmol) and triethylamine trihydrofluoride (63 g, 391 mmol) in anhydrous tetrahydrofuran (100 mL) was heated at 70° C. for 6 h. Saturated aqueous sodium bicarbonate solution was added until the solution reached pH=7. The resulting aqueous mixture was extracted with ethyl acetate (2×100 mL). The combined organic layers were washed with saturated aqueous sodium chloride solution, dried over anhydrous sodium sulfate, filtered, and concentrated. Purification by flash column chromatography (5:1 petroleum either/ethyl acetate) afforded crude 6-(3-iodo-1-isopropyl-1H-pyrazol-5-yl)bicyclo[3.1.0]hexan-3-ol (6.6 g, 100%). LRMS: [M+H]⁺ 332.9.

Step 7: Synthesis of the title compound. To a solution of 6-(3-iodo-1-isopropyl-1H-pyrazol-5-yl)bicyclo[3.1.0]hexan-3-ol (6.0 g, 18 mmol) in dichloromethane (200 mL) was added Dess-Martin periodinane (11.5 g, 27.1 mmol) at room temperature. After 2 h, saturated aqueous sodium bicarbonate solution (100 mL) and saturated aqueous sodium sulfite solution (100 mL) were added sequentially to the reaction mixture. The heterogeneous solution was stirred for 0.5 h. The organic layer was separated and the aqueous layer was extracted with dichloromethane (2×100 mL). The combined organic layers were washed with saturated aqueous sodium chloride solution, dried over anhydrous sodium sulfate, filtered, and concentrated. Purified by flash column chromatography (5:1 petroleum ether/ethyl acetate) afforded (1R,5S,6r)-6-(3-iodo-1-isopropyl-1H-pyrazol-5-yl)bicyclo[3.1.0]hexan-3-one (3.5 g). LRMS: [M+H]⁺ 330.7; ¹H NMR (400 MHz, CDCl₃): δ 6.03 (s, 1H), 4.59-4.49 (m, 1H), 2.78-2.72 (m, 2H), 2.42 (s, 1H), 2.37 (s, 1H), 1.89 (t, J=3.6 Hz, 2H), 1.49 (s, 6H), 1.33 (t, J=3.2 Hz, 1H). (1R,5S,6s)-6-(3-iodo-1-isopropyl-1H-pyrazol-5-yl)bicyclo[3.1.0]hexan-3-one (1.5 g) was also isolated.

### Intermediate Example I-2: 1-((1R,5S,6r)-3-((tert-Butyldiphenylsilyl)oxy) bicyclo[3.1.0]hexan-6-yl)ethan-1-one

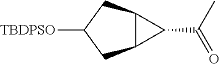

### Step 1: Synthesis of tert-butyl(cyclopent-3-en-1-yloxy)diphenylsilane

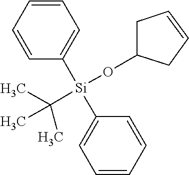 [SMILES: CC(C)(C)[Si](OC1CC=CC1)(c1ccccc1)c1ccccc1]

To an ice-cooled solution of 4-hydroxycyclopentene (50.0 g, 0.594 mol) and imidazole (80.9 g, 1.19 mol) in N,N-dimethylformamide (300 mL) was slowly added tert-butyldiphenylsilyl chloride (180 g, 0.65 mmol). The reaction mixture was warmed to room temperature. After 16 h, the reaction mixture was diluted with water (1 L) and ethyl acetate (500 mL). The aqueous layer was extracted with ethyl acetate (2×200 mL). The combined organic layers were washed sequentially with water (3×300 mL) and saturated aqueous sodium chloride solution (2×200 mL). The collected organic was dried over anhydrous sodium sulfate, filtered, and concentrated. Purification by flash column chromatography (15:1 petroleum ether/ethyl acetate) provided tert-butyl(cyclopent-3-en-1-yloxy)diphenylsilane (188 g, 98%). ¹H NMR (400 MHz, CDCl₃): δ 7.69-7.66 (m, 4H), 7.43-7.38 (m, 6H), 5.63-5.60 (m, 2H), 4.58-4.53 (m, 1H), 2.46-2.38 (m, 4H), 1.61 (s, 9H).

### Step 2: Synthesis of ethyl 3-((tert-butyldiphenylsilyl)oxy)bicyclo[3.1.0]hexane-6-carboxylate

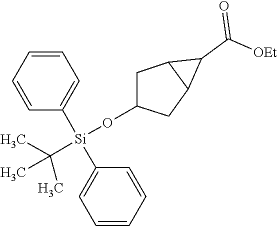 [SMILES: CCOC(=O)C1C2CC(O[Si](c3ccccc3)(c3ccccc3)C(C)(C)C)CC21]

To a stirred solution of tert-butyl(cyclopent-3-en-1-yloxy)diphenylsilane (0.100 kg, 310 mmol) and rhodium acetate dimer (1.37 g, 3.10 mmol) in anhydrous dichloromethane (1.2 L) at room temperature was added a solution of ethyl 2-diazoacetate (63.68 mmol) in dichloromethane (300 mL) over 8 h. After an additional 12 h. The reaction mixture was filtered through Celite. Concentration of the filtrate afforded crude ethyl 3-((tert-butyldiphenylsilyl)oxy)bicyclo[3.1.0]hexane-6-carboxylate (140 g) which was used without further purification.

### Step 3: Synthesis of 3-((tert-butyldiphenylsilyl)oxy)-N-methoxy-N-methylbicyclo[3.1.0]hexane-6-carboxamide

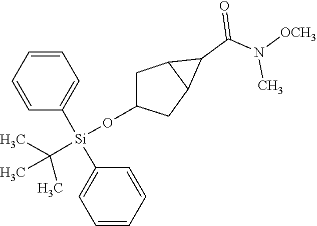 [SMILES: CON(C)C(=O)C1C2CC(O[Si](c3ccccc3)(c3ccccc3)C(C)(C)C)CC21]

To a solution of ethyl 3-((tert-butyldiphenylsilyl)oxy)bicyclo[3.1.0]hexane-6-carboxylate (70.0 g, 171 mmol) in ethanol (400 mL) was slowly added a solution of sodium hydroxide (20.56 g, 513.94 mmol) in water (100 mL). After 20 h, the reaction mixture was concentrated and the resulting residue was diluted with water (200 mL). The aqueous solution was adjusted to pH=3 by dropwise addition of 3 M aqueous hydrochloric acid. The aqueous mixture was extracted with ethyl acetate (2×200 mL). The combined organic layers were washed with saturated aqueous sodium chloride (200 mL), dried over anhydrous sodium sulfate, filtered, and concentrated to yield 3-((tert-butyldiphenylsilyl)oxy)bicyclo[3.1.0]hexane-6-carboxylic acid (53 g). To an ice-cooled suspension of crude acid in dichloromethane (600 mL) was added carbonyldiimidazole (25.5 g, 158 mmol). After 2 h, N,O-dimethylhydroxylamine hydrochloride (32 g, 0.33 mmol) was added. After 3 h, the reaction mixture was filtered, the filtrate was concentrated and purified by flash column chromatography (6:1 petroleum ether/ethyl acetate) to afford 3-((tert-butyldiphenylsilyl)oxy)-N-methoxy-N-methylbicyclo[3.1.0]hexane-6-carboxamide (37 g, 60%). ¹H NMR (400 MHz, CDCl₃): δ 7.63-7.61 (m, 4H), 7.42-7.33 (m, 6H), 4.33-4.31 (m, 1H), 3.74 (s, 2H), 3.57 (s, 1H), 3.21 (s, 2H), 3.10 (s, 1H), 2.21-2.18 (m, 1H), 2.00-1.80 (m, 6H), 1.06-1.01 (m, 9H).

Step 4: Synthesis of the title compound. To an ice-cooled solution of 3-((tert-butyldiphenylsilyl)oxy)-N-methoxy-N-methylbicyclo[3.1.0]hexane-6-carboxamide (37 g, 87 mmol) in anhydrous tetrahydrofuran (500 mL) was added dropwise methylmagnesium bromide (87 mL, 262 mmol, 3.0 M in diethyl ether). After 3 h, saturated aqueous ammonium chloride was added to the reaction mixture. The resulting aqueous solution was extracted with ethyl acetate (3×100 mL). The combined organic layers were washed with saturated aqueous sodium chloride solution, dried over anhydrous sodium sulfate, filtered, and concentrated to yield crude 1-(3-((tert-butyldiphenylsilyl)oxy)bicyclo[3.1.0]hexan-6-yl)ethanone (30.0 g, 91%) which was used without further purification.

### Synthesis of Compounds of Formula I

### Example A: 6-((1R,3r,5S,6r)-6-(1-Isopropyl-3-(3-(trifluoromethyl)phenyl)-1H-pyrazol-5-yl)bicyclo[3.1.0]hexan-3-yl)-2-thia-6-azaspiro[3.4]octane 2,2-dioxide (Compound 1), and 6-((1R,3s,5S,6r)-6-(1-isopropyl-3-(3-(trifluoromethyl)phenyl)-1H-pyrazol-5-yl)bicyclo[3.1.0]hexan-3-yl)-2-thia-6-azaspiro[3.4]octane 2,2-dioxide (Compound 2)

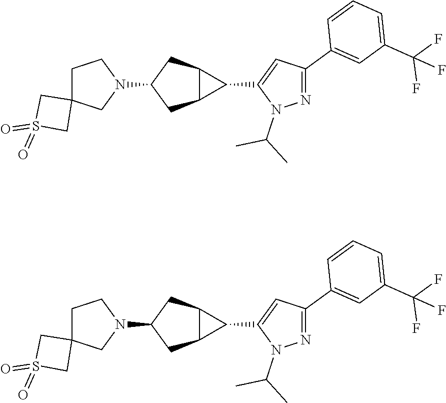 [SMILES: CC(C)n1nc(-c2cccc(C(F)(F)F)c2)cc1[C@H]1[C@@H]2C[C@@H](N3CCC4(C3)CS(=O)(=O)C4)C[C@@H]21 ; CC(C)n1nc(-c2cccc(C(F)(F)F)c2)cc1[C@H]1[C@@H]2C[C@H](N3CCC4(C3)CS(=O)(=O)C4)C[C@@H]21]

The title compounds were synthesized generally following the procedure described for Compound 3 and Compound 4 using 4,4,5,5-tetramethyl-2-(3-(trifluoromethyl)phenyl)-1,3,2-dioxaborolane. The crude mixture was purified by reverse phase chromatography (acetonitrile/water gradient with 0.05% NH₃+10 mM NH₄HCO₃) to afford compound 1 as the first eluting peak and compound 2 as the second eluting peak. The relative stereochemistry was assigned based on ¹H NMR analysis.

Compound 1: 6-((1R,3r,5S,6r)-6-(1-isopropyl-3-(3-(trifluoromethyl)phenyl)-1H-pyrazol-5-yl)bicyclo[3.1.0]hexan-3-yl)-2-thia-6-azaspiro[3.4]octane 2,2-dioxide LCMS (ESI) [M+H]⁺=494.1. ¹H NMR (400 MHz, CD₃OD) δ 8.01 (s, 1H), 7.98-7.90 (m, 1H), 7.54 (d, J=5.2 Hz, 2H), 6.25 (s, 1H), 4.76-4.73 (m, 1H), 4.09 (s, 4H), 3.06-2.93 (m, 1H), 2.88-2.80 (m, 1H), 2.84 (s, 1H), 2.72 (t, J=7.2 Hz, 2H), 2.25-2.11 (m, 5H), 1.95 (d, J=4.4 Hz, 1H), 1.92 (d, J=4.4 Hz, 1H), 1.74-1.70 (m, 2H), 1.54 (d, J=6.8 Hz, 6H).

Compound 2: 6-((1R,3s,5S,6r)-6-(1-isopropyl-3-(3-(trifluoromethyl)phenyl)-1H-pyrazol-5-yl)bicyclo[3.1.0]hexan-3-yl)-2-thia-6-azaspiro[3.4]octane 2,2-dioxide LCMS (ESI) [M+H]⁺=494.1. ¹H NMR (400 MHz, CD₃OD) δ 8.02 (s, 1H), 7.98-7.93 (m, 1H), 7.54 (d, J=5.2 Hz, 2H), 6.29 (s, 1H), 4.80-4.72 (m, 1H), 4.18-4.07 (m, 4H), 2.86 (s, 2H), 2.71 (t, J=7.2 Hz, 2H), 2.62-2.50 (m, 1H), 2.31-2.16 (m, 4H), 1.91-1.81 (m, 2H), 1.78-1.70 (m, 3H), 1.53 (d, J=6.8 Hz, 6H).

### Example B: 6-((1R,3s,5S,6r)-6-(1-isopropyl-3-(5-(trifluoromethyl)pyridin-3-yl)-1H-pyrazol-5-yl)bicyclo[3.1.0]hexan-3-yl)-2-thia-6-azaspiro[3.4]octane 2,2-dioxide (Compound 3), and 6-((1R,3r,5S,6r)-6-(1-isopropyl-3-(5-(trifluoromethyl)pyridin-3-yl)-1H-pyrazol-5-yl)bicyclo[3.1.0]hexan-3-yl)-2-thia-6-azaspiro[3.4]octane 2,2-dioxide (Compound 4)

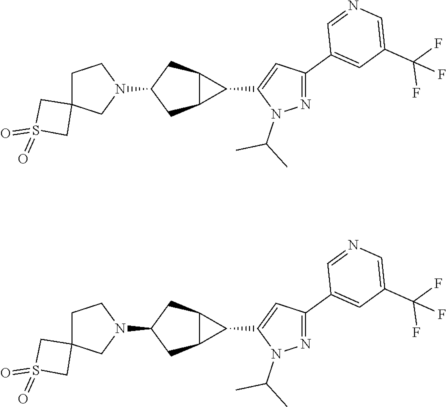 [SMILES: CC(C)n1nc(-c2cncc(C(F)(F)F)c2)cc1[C@H]1[C@@H]2C[C@@H](N3CCC4(C3)CS(=O)(=O)C4)C[C@@H]21 ; CC(C)n1nc(-c2cncc(C(F)(F)F)c2)cc1[C@H]1[C@@H]2C[C@H](N3CCC4(C3)CS(=O)(=O)C4)C[C@@H]21]

### Step 1: (1R,5S,6r)-6-(1-Isopropyl-3-(5-(trifluoromethyl)pyridin-3-yl)-1H-pyrazol-5-yl)bicyclo[3.1.0]hexan-3-one

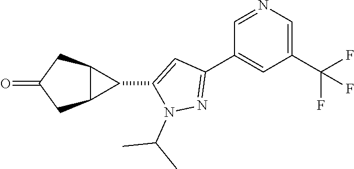 [SMILES: CC(C)n1nc(-c2cncc(C(F)(F)F)c2)cc1[C@H]1[C@@H]2CC(=O)C[C@@H]21]

To a mixture of (1R,5S,6r)-6-(3-iodo-1-isopropyl-1H-pyrazol-5-yl)bicyclo[3.1.0]hexan-3-one (2.0 g, 6.1 mmol) and 3-(4,4,5,5-tetramethyl-1,3,2-dioxaborolan-2-yl)-5-(trifluoromethyl)pyridine (2.5 g, 9.2 mmol) in 1,4-dioxane (32 mL) and water (8 mL) were added Cs₂CO₃ (6 g, 18.4 mmol) and bis(di-tert-butyl(4-dimethylaminophenyl)phosphine)dichloropalladium(II) (460 mg, 0.65 mmol). The reaction mixture was placed under a nitrogen atmosphere and stirred at 100° C. for 4 h. The reaction was quenched with water (30 mL) and extracted with ethyl acetate (100 mL×2). The combined organic layer was washed with brine (20 mL), dried over Na₂SO₄, filtered and concentrated. The residue was purified by silica column chromatography (0-30% ethyl acetate in petroleum ether) to provide the title compound (2.1 g, 96.3% yield). LCMS (ESI) [M+H]⁺=350.2.

### Step 2: 6-((1R,5S,6r)-6-(1-isopropyl-3-(5-(trifluoromethyl)pyridin-3-yl)-1H-pyrazol-5-yl)bicyclo[3.1.0]hexan-3-yl)-2-thia-6-azaspiro[3.4]octane 2,2-dioxide

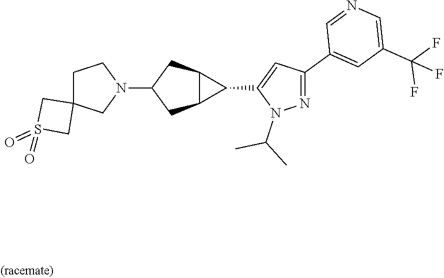 [SMILES: CC(C)n1nc(-c2cncc(C(F)(F)F)c2)cc1[C@H]1[C@@H]2CC(N3CCC4(C3)CS(=O)(=O)C4)C[C@@H]21]

To a solution of (1R,5S,6r)-6-(1-isopropyl-3-(5-(trifluoromethyl)pyridin-3-yl)-1H-pyrazol-5-yl)bicyclo[3.1.0]hexan-3-one (60 mg, 0.171 mmol) and 2-thia-6-azaspiro[3.4]octane 2,2-dioxide hydrochloride (50 mg, 0.25 mmol) in anhydrous methanol (2 mL) was added NaBH₃CN (55 mg, 0.88 mmol) at 20° C. The reaction mixture was heated to 70° C. and stirred for 16 h. The reaction was quenched with an aq. saturated NaHCO₃ solution (5 mL), and extracted with dichloromethane (30 mL×3). The combined organic phase was washed with brine, dried over anhydrous Na₂SO₄, filtered and concentrated in vacuo.

The residue was purified by reverse phase chromatography (acetonitrile/water gradient with 0.05% NH₃+10 mM NH₄HCO₃) to provide the first eluting peak, compound 3, as a pure single stereoisomer of the title compound (37.9 mg, 44.6% yield). The second eluting peak, compound 4, was obtained as a pure single stereoisomer of the title compound (33.0 mg, 32.7% yield).

Compound 3: 6-((1R,3s,5S,6r)-6-(1-isopropyl-3-(5-(trifluoromethyl)pyridin-3-yl)-1H-pyrazol-5-yl)bicyclo[3.1.0]hexan-3-yl)-2-thia-6-azaspiro[3.4]octane 2,2-dioxide LCMS (ESI) [M+H]⁺=495.4. ¹H NMR (400 MHz, CDCl₃) δ 9.11 (s, 1H), 8.75 (s, 1H), 8.29 (s, 1H), 6.19 (s, 1H), 4.68-4.60 (m, 1H), 4.09 (s, 4H), 2.93-2.69 (m, 4H), 2.46 (brs, 1H), 2.25-2.11 (m, 4H), 1.92-1.84 (m, 2H), 1.72-1.66 (m, 3H), 1.55 (d, J=6.4 Hz, 6H).

Compound 4: 6-((1R,3r,5S,6r)-6-(1-isopropyl-3-(5-(trifluoromethyl)pyridin-3-yl)-1H-pyrazol-5-yl)bicyclo[3.1.0]hexan-3-yl)-2-thia-6-azaspiro[3.4]octane 2,2-dioxide. LCMS (ESI) [M+H]⁺=495.4. ¹H NMR (400 MHz, CDCl₃) δ 9.11 (s, 1H), 8.74 (s, 1H), 8.29 (s, 1H), 6.14 (s, 1H), 4.72-4.55 (m, 1H), 4.10-3.98 (m, 4H), 2.93 (brs, 1H), 2.81 (s, 2H), 2.65-2.75 (m, 2H), 2.20-2.10 (m, 4H), 2.08 (s, 1H), 1.98-1.86 (m, 2H), 1.72-1.66 (m, 2H), 1.55 (d, J=6.8 Hz, 6H).

### Example C: 6-((1R,3r,5S,6r)-6-(1-Isopropyl-3-(4-(trifluoromethyl)phenyl)-1H-pyrazol-5-yl)bicyclo[3.1.0]hexan-3-yl)-2-thia-6-azaspiro[3.4]octane 2,2-dioxide (Compound 5)

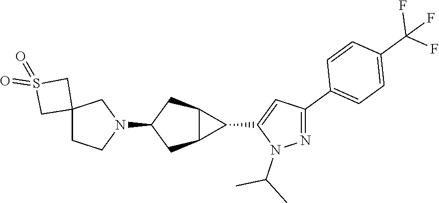 [SMILES: CC(C)n1nc(-c2ccc(C(F)(F)F)cc2)cc1[C@H]1[C@@H]2C[C@H](N3CCC4(C3)CS(=O)(=O)C4)C[C@@H]21]

### (Relative Stereochemistry was Assigned Based on ¹H NMR Analysis)

### Step 1: 6-((1R,3r,5S,6r)-6-(3-Iodo-1-isopropyl-1H-pyrazol-5-yl)bicyclo[3.1.0]hexan-3-yl)-2-thia-6-azaspiro[3.4]octane 2,2-dioxide

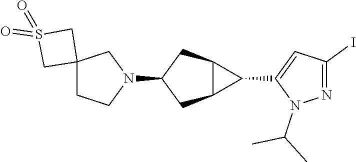 [SMILES: CC(C)n1nc(I)cc1[C@H]1[C@@H]2C[C@H](N3CCC4(C3)CS(=O)(=O)C4)C[C@@H]21]

To (1R,5S,6r)-6-(3-iodo-1-isopropyl-1H-pyrazol-5-yl)bicyclo[3.1.0]hexan-3-one (750 mg, 2.27 mmol) in m-xylene (9.1 mL) was added 2-thia-6-azaspiro[3.4]octane 2,2-dioxide hydrochloride (674 mg, 3.41 mmol), sodium triacetoxyborohydride (1444 mg, 6.82 mmol) and acetic acid (1.56 mL, 27.26 mmol). The reaction mixture was stirred at 60° C. for 18 h. Additional 2-thia-6-azaspiro[3.4]octane 2,2-dioxide hydrochloride (209 mg, 1.07 mmol) was added and the reaction mixture was stirred at 75° C. for 18 h. Additional 2-thia-6-azaspiro[3.4]octane 2,2-dioxide hydrochloride (209 mg, 1.07 mmol) and sodium triacetoxyborohydride (720 mg, 3.40 mmol) was added and the reaction mixture was stirred at 75° C. for 6 h. The reaction mixture was diluted with 1N aq. NH₄Cl (5 mL), 1N aq. NaHCO₃ (40 mL) and 10% methanol in dichloromethane (25 mL). The aqueous layer was extracted with 10% methanol in dichloromethane (2×25 mL). The combined organic layer was concentrated under reduced pressure. The mixture was diluted with DMSO and purified by prep-HPLC (acetonitrile/water gradient with 0.1% NH₄OH) to give the second eluting peak as a pure single stereoisomer of the title compound (110 mg, 10% yield). LCMS (ESI) [M+H]⁺=476.1. ¹H NMR (400 MHz, DMSO-d₆) δ 6.00 (s, 1H), 4.64-4.52 (m, 1H), 4.16-4.03 (m, 4H), 2.84-2.76 (m, 1H), 2.69 (s, 2H), 2.56 (t, J=7.3 Hz, 2H), 2.18 (t, J=3.4 Hz, 1H), 2.05 (t, J=7.2 Hz, 2H), 2.02-1.93 (m, 2H), 1.89 (dd, J=14.0, 3.3 Hz, 2H), 1.59-1.53 (m, 2H), 1.36 (d, J=6.5 Hz, 6H).

Step 2: Synthesis of the title compound. A solution of 6-((1R,3r,5S,6r)-6-(3-iodo-1-isopropyl-1H-pyrazol-5-yl)bicyclo[3.1.0]hexan-3-yl)-2-thia-6-azaspiro[3.4]octane 2,2-dioxide (35.7 mg, 0.075 mmol), potassium phosphate (32 mg, 0.15 mmol), SPhos Pd G₃ (2.9 mg, 0.004 mmol) and (4-(trifluoromethyl)phenyl)boronic acid (21 mg, 0.11 mmol) in 1,4-dioxane (0.38 mL) and water (0.094 mL) was stirred at 60° C. for 18 h. The reaction mixture was diluted with 1N aq. NH₄Cl (0.5 mL) and DMSO, filtered and purified by prep-HPLC (acetonitrile/water gradient with 0.1% TFA) to give the title compound (31.2 mg, 84% yield).

Compound 5: LCMS (ESI) [M+H]⁺=494.2. ¹H NMR (400 MHz, DMSO-d₆) δ 7.93 (d, J=8.0 Hz, 2H), 7.71 (d, J=8.2 Hz, 2H), 6.41 (s, 1H), 4.74-4.60 (m, 1H), 4.18-4.05 (m, 4H), 2.88-2.80 (m, 1H), 2.72 (s, 2H), 2.59 (t, J=7.2 Hz, 2H), 2.24 (t, J=3.4 Hz, 1H), 2.11-1.98 (m, 4H), 1.92 (dd, J=13.6, 3.3 Hz, 2H), 1.68-1.61 (m, 2H), 1.46 (d, J=6.6 Hz, 6H).

### Example D: 6-((1R,3r,5S,6r)-6-(1-Isopropyl-3-(2-(trifluoromethyl)pyrimidin-5-yl)-1H-pyrazol-5-yl)bicyclo[3.1.0]hexan-3-yl)-2-thia-6-azaspiro[3.4]octane 2,2-dioxide (Compound 6)

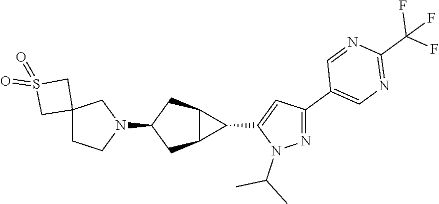 [SMILES: CC(C)n1nc(-c2cnc(C(F)(F)F)nc2)cc1[C@H]1[C@@H]2C[C@H](N3CCC4(C3)CS(=O)(=O)C4)C[C@@H]21]

The title compound was synthesized generally following the procedure described for Compound 5 using 5-(4,4,5,5-tetramethyl-1,3,2-dioxaborolan-2-yl)-2-(trifluoromethyl)pyrimidine.

Compound 6: LCMS (ESI) [M+H]⁺=496.2. ¹H NMR (400 MHz, DMSO-d₆) δ 9.31 (s, 2H), 6.65 (s, 1H), 4.79-4.66 (m, 1H), 4.18-4.05 (m, 4H), 2.89-2.79 (m, 1H), 2.72 (s, 2H), 2.59 (t, J=7.3 Hz, 2H), 2.29 (t, J=3.3 Hz, 1H), 2.11-1.99 (m, 4H), 1.95 (dd, J=13.8, 3.2 Hz, 2H), 1.68-1.62 (m, 2H), 1.48 (d, J=6.6 Hz, 6H).

### Example E—Compounds 7*-10*: (R)-7-((1R,3s,5S,6R)-6-(1-isopropyl-3-(5-(trifluoromethyl)pyridin-3-yl)-1H-pyrazol-5-yl)bicyclo[3.1.0]hexan-3-yl)-2-thia-7-azaspiro[4.4]nonane 2,2-dioxide; (S)-7-((1R,3s,5S,6S)-6-(1-isopropyl-3-(5-(trifluoromethyl)pyridin-3-yl)-1H-pyrazol-5-yl)bicyclo[3.1.0]hexan-3-yl)-2-thia-7-azaspiro[4.4]nonane 2,2-dioxide; (R)-7-((1R,3r,5S,6R)-6-(1-isopropyl-3-(5-(trifluoromethyl)pyridin-3-yl)-1H-pyrazol-5-yl)bicyclo[3.1.0]hexan-3-yl)-2-thia-7-azaspiro[4.4]nonane 2,2-dioxide; and (S)-7-((1R,3r,5S,6S)-6-(1-isopropyl-3-(5-(trifluoromethyl)pyridin-3-yl)-1H-pyrazol-5-yl)bicyclo[3.1.0]hexan-3-yl)-2-thia-7-azaspiro[4.4]nonane 2,2-dioxide

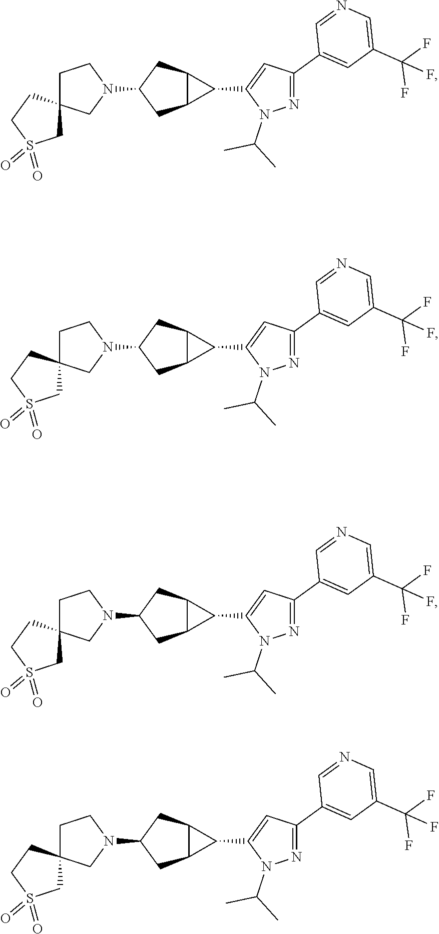 [SMILES: CC(C)n1nc(-c2cncc(C(F)(F)F)c2)cc1[C@H]1[C@@H]2C[C@@H](N3CC[C@@]4(CCS(=O)(=O)C4)C3)C[C@@H]21 ; CC(C)n1nc(-c2cncc(C(F)(F)F)c2)cc1[C@H]1[C@@H]2C[C@@H](N3CC[C@]4(CCS(=O)(=O)C4)C3)C[C@@H]21 ; CC(C)n1nc(-c2cncc(C(F)(F)F)c2)cc1[C@H]1[C@@H]2C[C@H](N3CC[C@@]4(CCS(=O)(=O)C4)C3)C[C@@H]21 ; CC(C)n1nc(-c2cncc(C(F)(F)F)c2)cc1[C@H]1[C@@H]2C[C@H](N3CC[C@]4(CCS(=O)(=O)C4)C3)C[C@@H]21]

### Step 1: 7-((1R,5S,6r)-6-(1-isopropyl-3-(5-(trifluoromethyl)pyridin-3-yl)-H-pyrazol-5-yl)bicyclo[3.1.0]hexan-3-yl)-2-thia-7-azaspiro[4.4]nonane 2,2-dioxide

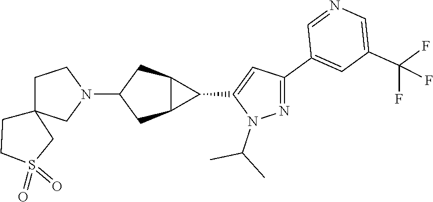

7-((1R,5S,6r)-6-(1-isopropyl-3-(5-(trifluoromethyl)pyridin-3-yl)-1H-pyrazol-5-yl)bicyclo[3.1.0]hexan-3-yl)-2-thia-7-azaspiro[4.4]nonane 2,2-dioxide was synthesized following a procedure similar to Compound 3, using 2-thia-7-azaspiro[4.4]nonane 2,2-dioxide in step 2.

Compound 7*: The crude mixture was purified by chiral Prep-HPLC (Daicel Chiralpak AS, 0.1% NH₃ in H₂O/EtOH 95:5 to 75:25) to provide the first eluting peak as a pure single undefined enantiomer of the title compound (10.8 mg, 6% yield). LCMS (ESI) [M+H]⁺=509.2. ¹H NMR (400 MHz, CD₃OD) δ 9.15 (s, 1H), 8.74 (s, 1H), 8.41 (s, 1H), 6.39 (s, 1H), 4.82-4.74 (m, 1H), 3.26-3.07 (m, 4H), 3.00-2.90 (m, 1H), 2.89-2.84 (m, 1H), 2.82-2.75 (m, 1H), 2.66-2.56 (m, 1H), 2.54-2.46 (m, 1H), 2.33-2.12 (m, 5H), 2.06-1.83 (m, 4H), 1.79-1.64 (m, 2H), 1.55 (d, J=4.8 Hz, 6H).

Compound 8*: The crude mixture was purified by chiral Prep-HPLC (Daicel Chiralpak AS, 0.1% NH₃ in H₂O/EtOH 95:5 to 75:25) to provide the second eluting peak as a pure single undefined enantiomer of the title compound (7.7 mg, 4.2% yield). LCMS (ESI) [M+H]⁺=509.2. ¹H NMR (400 MHz, CD₃OD) δ 9.15 (s, 1H), 8.73 (s, 1H), 8.41 (s, 1H), 6.39 (s, 1H), 4.83-4.76 (m, 1H), 3.26-3.06 (m, 4H), 2.95-2.87 (m, 1H), 2.85-2.81 (m, 1H), 2.79-2.73 (m, 1H), 2.63-2.54 (m, 1H), 2.51-2.45 (m, 1H), 2.30-2.13 (m, 5H), 2.03-1.85 (m, 4H), 1.80-1.65 (m, 2H), 1.55 (d, J=6.4 Hz, 6H).

Compound 9*: The crude mixture was purified by chiral Prep-HPLC (Daicel Chiralpak AS, 0.1% NH₃ in H₂O/EtOH 95:5 to 75:25) to provide the third eluting peak as a pure single undefined enantiomer of the title compound (12.9 mg, 7.2% yield). LCMS (ESI) [M+H]⁺=509.2. ¹H NMR (400 MHz, CD₃OD) δ 9.15 (s, 1H), 8.74 (s, 1H), 8.41 (s, 1H), 6.44 (s, 1H), 4.82-4.77 (m, 1H), 3.25-3.09 (m, 4H), 2.81-2.76 (m, 1H), 2.76-2.70 (m, 1H), 2.69-2.63 (m, 1H), 2.61-2.56 (m, 1H), 2.54-2.46 (m, 1H), 2.33-2.19 (m, 4H), 2.08-1.98 (m, 1H), 1.94-1.85 (m, 3H), 1.80-1.75 (m, 3H), 1.53 (d, J=6.8 Hz, 6H).

Compound 10*: The crude mixture was purified by chiral Prep-HPLC (Daicel Chiralpak AS, 0.1% NH₃ in H₂O/EtOH 95:5 to 75:25) to provide the fourth eluting peak as a pure single undefined enantiomer of the title compound (15.3 mg, 8.5% yield). LCMS (ESI) [M+H]⁺=509.2. ¹H NMR (400 MHz, CD₃OD) δ 9.15 (s, 1H), 8.74 (s, 1H), 8.41 (s, 1H), 6.45 (s, 1H), 4.82-4.75 (m, 1H), 3.25-3.10 (m, 4H), 2.81-2.70 (m, 2H), 2.69-2.60 (m, 1H), 2.59-2.55 (m, 1H), 2.53-2.47 (m, 1H), 2.34-2.20 (m, 4H), 2.08-1.98 (m, 1H), 1.96-1.87 (m, 3H), 1.80-1.75 (m, 3H), 1.53 (d, J=6.4 Hz, 6H).

### Example F: 6-((1R,3s,5S,6r)-6-(1-Isopropyl-3-(6-(trifluoromethyl)pyridin-2-yl)-1H-pyrazol-5-yl)bicyclo[3.1.0]hexan-3-yl)-2-thia-6-azaspiro[3.4]octane 2,2-dioxide (Compound 11), and 6-((1R,3r,5S,6r)-6-(1-Isopropyl-3-(6-(trifluoromethyl)pyridin-2-yl)-1H-pyrazol-5-yl)bicyclo[3.1.0]hexan-3-yl)-2-thia-6-azaspiro[3.4]octane 2,2-dioxide (Compound 12)

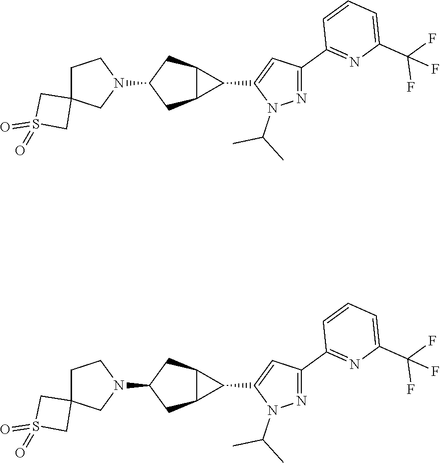 [SMILES: CC(C)n1nc(-c2cccc(C(F)(F)F)n2)cc1[C@H]1[C@@H]2C[C@@H](N3CCC4(C3)CS(=O)(=O)C4)C[C@@H]21 ; CC(C)n1nc(-c2cccc(C(F)(F)F)n2)cc1[C@H]1[C@@H]2C[C@H](N3CCC4(C3)CS(=O)(=O)C4)C[C@@H]21]

### Step 1: 1-((1R,5S,6r)-3-((tert-Butyldiphenylsilyl)oxy)bicyclo[3.1.0]hexan-6-yl)-3-(6-(trifluoromethyl)pyridin-2-yl)propane-1,3-dione

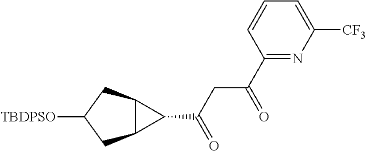

To a solution of 1-((1R,5S,6r)-3-((tert-butyldiphenylsilyl)oxy)bicyclo[3.1.0]hexan-6-yl)ethan-1-one (25.0 g, 66.0 mmol) in tetrahydrofuran (300 mL) was added NaH (4.0 g, 100 mmol, 60% in mineral oil) in portions at 0° C. under nitrogen and stirred for 1 h. A solution of methyl 6-(trifluoromethyl)picolinate (25.0 g, 121.9 mmol) in THF (100 mL) was added at 0° C. under nitrogen. Then the reaction mixture was stirred at 20° C. for 2 h. The reaction mixture was quenched with a saturated aq. NH₄Cl solution (80 mL), extracted with ethyl acetate (500 mL×3). The combined organic layer was washed with brine (50 mL), dried over Na₂SO₄, filtered and concentrated. The residue was purified by silica column chromatography (0-10% ethyl acetate in petroleum ether) to provide the title compound (35 g, 59.64 mmol, 90.3% yield). LCMS (ESI) [M+H]⁺=552.2.

### Step 2: 2-(5-((1R,5S,6r)-3-((tert-Butyldiphenylsilyl)oxy)bicyclo[3.1.0]hexan-6-yl)-1-isopropyl-1H-pyrazol-3-yl)-6-(trifluoromethyl)pyridine

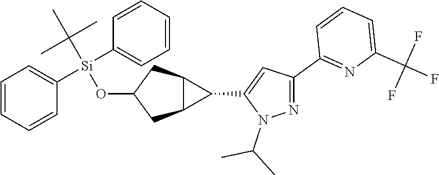 [SMILES: CC(C)n1nc(-c2cccc(C(F)(F)F)n2)cc1[C@H]1[C@@H]2CC(O[Si](c3ccccc3)(c3ccccc3)C(C)(C)C)C[C@@H]21]

To a solution of 1-((1R,5S,6r)-3-((tert-butyldiphenylsilyl)oxy)bicyclo[3.1.0]hexan-6-yl)-3-(6-(trifluoromethyl)pyridin-2-yl)propane-1,3-dione (26.0 g, 47.13 mmol) in ethanol (250 mL) was added isopropylhydrazine hydrochloride (5.44 g, 49.22 mmol) and triethylamine (6.83 mL, 49.1 mmol) dropwise. The mixture was stirred at 25° C. for 15 h. The mixture was concentrated, diluted with ethyl acetate (100 mL), and washed with brine (60 mL). The organic layer was dried over anhydrous sodium sulfate, filtered and concentrated under vacuum. The residue was purified by silica column chromatography (0-10% EtOAc in petroleum ether) to afford a mixture of the title compound and its pyrazole regioisomer (24 g, 86.4% yield). LCMS (ESI) [M+H]⁺=590.2.

### Step 3: (1R,5S,6r)-6-(1-Isopropyl-3-(6-(trifluoromethyl)pyridin-2-yl)-1H-pyrazol-5-yl)bicyclo[3.1.0]hexan-3-ol

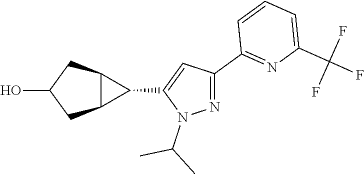 [SMILES: CC(C)n1nc(-c2cccc(C(F)(F)F)n2)cc1[C@H]1[C@@H]2CC(O)C[C@@H]21]

To 2-(5-((1R,5S,6r)-3-((tert-butyldiphenylsilyl)oxy)bicyclo[3.1.0]hexan-6-yl)-1-isopropyl-1H-pyrazol-3-yl)-6-(trifluoromethyl)pyridine (24.0 g, 40.69 mmol) was added 1 M tetrabutylammonium fluoride in tetrahydrofuran (212.5 mL, 212.5 mmol), the mixture was stirred at 25° C. for 15 h. The reaction was quenched with a saturated aq. NH₄Cl solution (100 mL) and extracted with ethyl acetate (350 mL×3). The combined organic layers were washed with brine (100 mL×2), dried over Na₂SO₄, filtered and concentrated under reduced pressure. The residue was purified by silica column chromatography (0-50% ethyl acetate in petroleum ether) to provide a mixture the title compound and its pyrazole regioisomer (13.0 g, 90.9% yield). LCMS (ESI) [M+H]⁺=352.1

### Step 4: (1R,5S,6r)-6-(1-Isopropyl-3-(6-(trifluoromethyl)pyridin-2-yl)-1H-pyrazol-5-yl)bicyclo[3.1.0]hexan-3-one

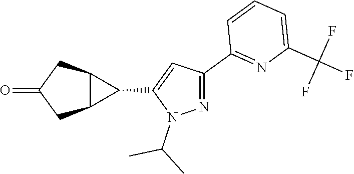 [SMILES: CC(C)n1nc(-c2cccc(C(F)(F)F)n2)cc1[C@H]1[C@@H]2CC(=O)C[C@@H]21]

To a solution of (1R,5S,6r)-6-(1-isopropyl-3-(6-(trifluoromethyl)pyridin-2-yl)-1H-pyrazol-5-yl)bicyclo[3.1.0]hexan-3-ol (10.5 g, 29.88 mmol) in anhydrous dichloromethane (70 mL) was added DMP (25.35 g, 59.77 mmol) at 0° C. under nitrogen. The reaction mixture was stirred at 25° C. for 1 h. The mixture was filtered and washed with dichloromethane (100 mL). The organic layer was washed with brine (50 mL×3), dried over Na₂SO₄, filtered and concentrated under reduced pressure. The residue was purified by silica column chromatography (10-20% ethyl acetate in petroleum ether) to provide the titled compound as the first eluting peak (5000 mg, 48% yield). LCMS (ESI) [M+H]⁺=350.1. ¹H NMR (400 MHz, CD₃OD) δ 8.17 (d, J=8.0 Hz, 1H), 7.98 (t, J=7.6 Hz, 1H), 7.63 (d, J=7.6 Hz, 1H), 6.61 (s, 1H), 4.83-4.76 (m, 1H), 2.80-2.71 (m, 2H), 2.42-2.35 (m, 2H), 2.02-1.99 (m, 2H), 1.61-1.56 (m, 1H), 1.48 (d, J=6.4 Hz, 6H).

### Step 5: 6-((1R,3s,5S,6r)-6-(1-Isopropyl-3-(6-(trifluoromethyl)pyridin-2-yl)-1H-pyrazol-5-yl)bicyclo[3.1.0]hexan-3-yl)-2-thia-6-azaspiro[3.4]octane 2,2-dioxide (Compound 11)

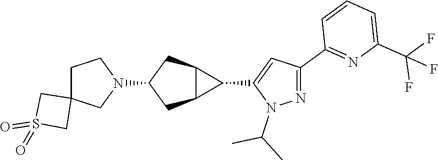 [SMILES: CC(C)n1nc(-c2cccc(C(F)(F)F)n2)cc1[C@H]1[C@@H]2C[C@@H](N3CCC4(C3)CS(=O)(=O)C4)C[C@@H]21]

To a solution of 6-(1-isopropyl-3-(6-(trifluoromethyl)pyridin-2-yl)-1H-pyrazol-5-yl)bicyclo[3.1.0]hexan-3-one (5 g, 14.3 mmol) and 2-thia-6-azaspiro[3.4]octane 2,2-dioxide (3 g, 18.6 mmol) in anhydrous methanol (100 mL) was added NaBH₃CN (3.2 g, 50.9 mmol) and acetic acid (2.4 mL, 41.9 mmol) dropwise at 20° C. The reaction mixture was heated to 50° C. and stirred for 2 h. The reaction was quenched with a saturated aq. NaHCO₃ solution (20 mL) and extracted with dichloromethane (300 mL×3). The combined organic phase was washed with brine, dried over Na₂SO₄, filtered and concentrated under reduced pressure. The residue was purified by reverse phase chromatography (acetonitrile/water gradient with 0.05% NH₃+10 mM NH₄HCO₃) to afford the second eluting peak as a pure single stereoisomer of the title compound (2.88 g, 39.8% yield).

Compound 11: LCMS (ESI) [M+H]⁺=495.1. ¹H NMR (400 MHz, CDCl₃) δ 8.17 (d, J=8.0 Hz, 1H), 7.84-7.80 (m, 1H), 7.51 (d, J=7.2 Hz, 1H), 6.56 (s, 1H), 4.70-4.59 (m, 1H), 4.08 (s, 4H), 2.78 (s, 2H), 2.68-2.66 (m, 2H), 2.42-2.35 (m, 1H), 2.21-2.16 (m, 4H), 1.86-1.76 (m, 2H), 1.73 (s, 2H), 1.58-1.56 (m, 1H), 1.54 (d, J=6.4 Hz, 6H). The relative stereochemistry was assigned based on ¹H NMR analysis.

### 6-((1R,3r,5S,6r)-6-(1-Isopropyl-3-(6-(trifluoromethyl)pyridin-2-yl)-1H-pyrazol-5-yl)bicyclo[3.1.0]hexan-3-yl)-2-thia-6-azaspiro[3.4]octane 2,2-dioxide (Compound 12)

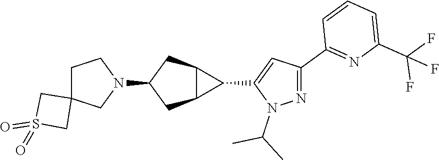 [SMILES: CC(C)n1nc(-c2cccc(C(F)(F)F)n2)cc1[C@H]1[C@@H]2C[C@H](N3CCC4(C3)CS(=O)(=O)C4)C[C@@H]21]

The residue from step 6 was purified by preparative reverse phase HPLC (acetonitrile/water gradient with 0.05% NH₃+10 mM NH₄HCO₃) to afford the first eluting peak as a pure single stereoisomer of the title compound (3.60 g, 49.4% yield).

Compound 12: LCMS (ESI) [M+H]⁺=495.1. ¹H NMR (400 MHz, CDCl₃) δ 8.17 (d, J=8.0 Hz, 1H), 7.84-7.80 (m, 1H), 7.51 (d, J=7.2 Hz, 1H), 6.52 (s, 1H), 4.70-4.60 (m, 1H), 4.05 (s, 4H), 3.00-2.92 (m, 1H), 2.79 (s, 2H), 2.69 (t, J=7.2 Hz, 2H), 2.19-2.10 (m, 4H), 2.04 (t, J=3.2 Hz, 1H), 1.94-1.85 (m, 2H), 1.69 (s, 2H), 1.55 (d, J=6.8 Hz, 6H). The relative stereochemistry was assigned based on ¹H NMR analysis.

### Example G: 6-((1R,3s,5S,6r)-6-(1-isopropyl-3-(6-(trifluoromethyl)pyridin-3-yl)-1H-pyrazol-5-yl)bicyclo[3.1.0]hexan-3-yl)-2-thia-6-azaspiro[3.4]octane 2,2-dioxide (Compound 13), and 6-((1R,3r,5S,6r)-6-(1-isopropyl-3-(6-(trifluoromethyl)pyridin-3-yl)-1H-pyrazol-5-yl)bicyclo[3.1.0]hexan-3-yl)-2-thia-6-azaspiro[3.4]octane 2,2-dioxide (Compound 14)

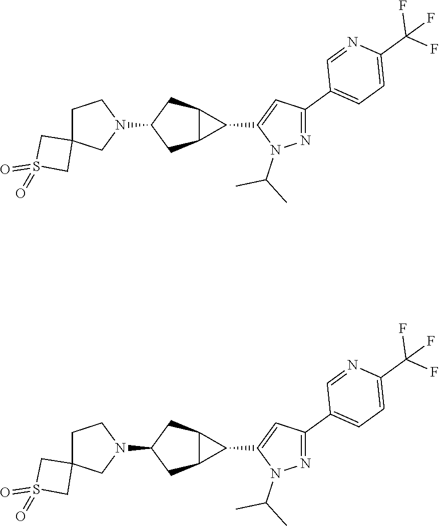 [SMILES: CC(C)n1nc(-c2ccc(C(F)(F)F)nc2)cc1[C@H]1[C@@H]2C[C@@H](N3CCC4(C3)CS(=O)(=O)C4)C[C@@H]21 ; CC(C)n1nc(-c2ccc(C(F)(F)F)nc2)cc1[C@H]1[C@@H]2C[C@H](N3CCC4(C3)CS(=O)(=O)C4)C[C@@H]21]

6-((1R,5S,6r)-6-(1-isopropyl-3-(6-(trifluoromethyl)pyridin-3-yl)-1H-pyrazol-5-yl)bicyclo[3.1.0]hexan-3-yl)-2-thia-6-azaspiro[3.4]octane 2,2-dioxide was synthesized following a procedure similar for Compounds 3 and 4 using 5-(4,4,5,5-tetramethyl-1,3,2-dioxaborolan-2-yl)-2-(trifluoromethyl)pyridine. The relative stereochemistry was assigned based on ¹H NMR analysis.

The crude mixture was purified by reverse phase chromatography (acetonitrile/water gradient with 0.05% NH₃+10 mM NH₄HCO₃) to afford compound 13 as the first eluting peak and compound 14 as the second eluting peak.

Compound 13: 6-((1R,3s,5S,6r)-6-(1-isopropyl-3-(6-(trifluoromethyl)pyridin-3-yl)-1H-pyrazol-5-yl)bicyclo[3.1.0]hexan-3-yl)-2-thia-6-azaspiro[3.4]octane 2,2-dioxide. LCMS (ESI) [M+H]⁺=495.2. ¹H NMR (400 MHz, CDCl₃) δ 9.03 (s, 1H), 8.23 (d, J=8.4 Hz, 1H), 7.66 (d, J=8.0 Hz, 1H), 6.19 (s, 1H), 4.68-4.61 (m, 1H), 4.08 (s, 4H), 2.79 (s, 2H), 2.66 (d, J=6.8 Hz, 2H), 2.45-2.42 (m, 1H), 2.24-2.16 (m, 4H), 1.88-1.86 (m, 2H), 1.71 (brs, 2H), 1.61-1.59 (m, 1H), 1.56 (d, J=6.8 Hz, 6H).

Compound 14: 6-((1R,3r,5S,6r)-6-(1-isopropyl-3-(6-(trifluoromethyl)pyridin-3-yl)-1H-pyrazol-5-yl)bicyclo[3.1.0]hexan-3-yl)-2-thia-6-azaspiro[3.4]octane 2,2-dioxide. LCMS (ESI) [M+H]⁺=495.2. ¹H NMR (400 MHz, CDCl₃) δ 9.02 (s, 1H), 8.24 (d, J=8.4 Hz, 1H), 7.66 (d, J=8.0 Hz, 1H), 6.15 (s, 1H), 4.68-4.61 (m, 1H), 4.09-4.00 (m, 4H), 2.98-2.89 (m, 1H), 2.80 (s, 2H), 2.70-2.68 (m, 2H), 2.17-2.08 (m, 5H), 1.94-1.90 (m, 2H), 1.67 (s, 2H), 1.55 (d, J=6.8 Hz, 6H).

### Example H: 6-((1R,3r,5S,6r)-6-(1-Isopropyl-3-(2-(trifluoromethyl)pyrimidin-4-yl)-1H-pyrazol-5-yl)bicyclo[3.1.0]hexan-3-yl)-2-thia-6-azaspiro[3.4]octane 2,2-dioxide (Compound 15)

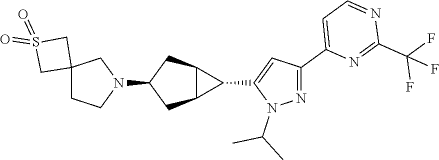 [SMILES: CC(C)n1nc(-c2ccnc(C(F)(F)F)n2)cc1[C@H]1[C@@H]2C[C@H](N3CCC4(C3)CS(=O)(=O)C4)C[C@@H]21]

The title compound was synthesized following a procedure similar for Compound 5 using (2-(trifluoromethyl)pyrimidin-4-yl)boronic acid. The relative stereochemistry was assigned based on ¹H NMR analysis.

Compound 15: LCMS (ESI) [M+H]⁺=496.2. ¹H NMR (400 MHz, DMSO-d₆) δ 8.95 (d, J=5.3 Hz, 1H), 8.11 (d, J=5.3 Hz, 1H), 6.56 (s, 1H), 4.82-4.67 (m, 1H), 4.19-4.04 (m, 4H), 2.90-2.78 (m, 1H), 2.72 (s, 2H), 2.59 (t, J=7.3 Hz, 2H), 2.30 (t, J=3.3 Hz, 1H), 2.11-1.97 (m, 4H), 1.94 (dd, J=13.7, 3.2 Hz, 2H), 1.73-1.67 (m, 2H), 1.49 (d, J=6.6 Hz, 6H). The relative stereochemistry was assigned based on ¹H NMR analysis.

### Example I: 6-((1R,3s,5S,6r)-6-(1-Isopropyl-3-(5-(trifluoromethyl)pyridin-3-yl)-1H-1,2,4-triazol-5-yl)bicyclo[3.1.0]hexan-3-yl)-2-thia-6-azaspiro[3.4]octane 2,2-dioxide (Compound 16), and 6-((1R,3r,5S,6r)-6-(1-Isopropyl-3-(5-(trifluoromethyl)pyridin-3-yl)-1H-1,2,4-triazol-5-yl)bicyclo[3.1.0]hexan-3-yl)-2-thia-6-azaspiro[3.4]octane 2,2-dioxide (Compound 17)

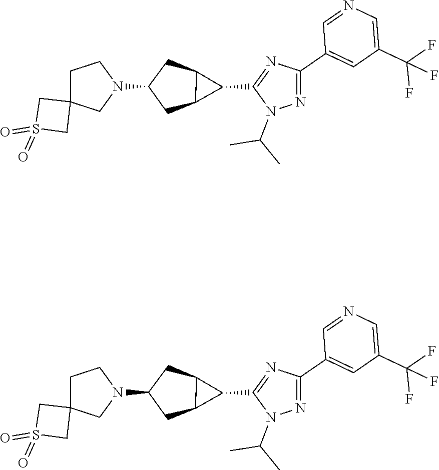 [SMILES: CC(C)n1nc(-c2cncc(C(F)(F)F)c2)nc1[C@H]1[C@@H]2C[C@@H](N3CCC4(C3)CS(=O)(=O)C4)C[C@@H]21 ; CC(C)n1nc(-c2cncc(C(F)(F)F)c2)nc1[C@H]1[C@@H]2C[C@H](N3CCC4(C3)CS(=O)(=O)C4)C[C@@H]21]

### Step 1: (E)-Benzyl (amino(methylthio)methylene)carbamate

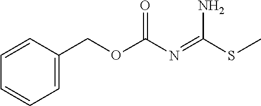

To an ice cold mixture of 2-methylisothiourea sulfurous acid (10.0 g, 58.06 mmol) and 2N aq. sodium hydroxide (34.8 mL, 69.6 mmol) in dichloromethane (100 mL) was added benzyl chloroformate (7.0 mL, 52.26 mmol). The mixture was stirred at 25° C. for 1 h. The mixture was extracted with ethyl acetate (500 mL×2). The combined organic layer was washed with brine (100 mL), dried over anhydrous Na₂SO₄, filtered and concentrated in vacuo. The residue was purified by silica column chromatography (0-50% ethyl acetate in petroleum ether) to provide the title compound (10 g, 76.8% yield). LCMS (ESI) [M+H]⁺=225.1. ¹H NMR (400 MHz, DMSO-d₆) δ 8.74 (brs, 2H), 7.39-7.29 (m, 5H), 5.04 (s, 2H), 2.33 (s, 3H).

### Step 2: Benzyl ((Z)-((1R,5S,6r)-3-((tert-butyldiphenylsilyl)oxy)bicyclo[3.1.0]hexane-6-carboxamido)(methylthio)methylene)carbamate

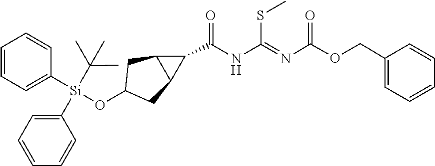

To a solution of (1R,5S,6r)-3-((tert-butyldiphenylsilyl)oxy)bicyclo[3.1.0]hexane-6-carboxylic acid (1.97 mL, 62.42 mmol), N,N-diisopropylethylamine (19.4 mL, 111.4 mmol) and (E)-Benzyl (amino(methylthio)methylene)carbamate (10.0 g, 44.59 mmol) in tetrahydrofuran (80 mL) was added HATU (25.4 g, 66.88 mmol) at 20° C. The reaction mixture was stirred at 20° C. for 2 h. The mixture was diluted with ethyl acetate (200 mL), washed with brine (50 mL). The organic layer was dried over Na₂SO₄, filtered and concentrated. The residue was purified by silica column chromatography (0-20% ethyl acetate in petroleum ether) to provide the title compound (22 g, 84.1% yield). LCMS (ESI) [M+H]⁺=587.1.

### Step 3: 5-((1R,5S,6r)-3-((tert-Butyldiphenylsilyl)oxy)bicyclo[3.1.0]hexan-6-yl)-1-isopropyl-1H-1,2,4-triazol-3-amine

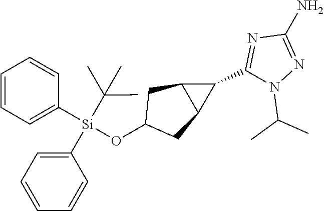

To a stirred solution of benzyl ((Z)-((1R,5S,6r)-3-((tert-butyldiphenylsilyl)oxy) bicyclo[3.1.0]hexane-6-carboxamido) (methylthio)methylene)carbamate (20.0 g, 34.08 mmol) in N,N-dimethylformamide (200 mL) was added isopropylhydrazine hydrochloride (18.9 g, 170.4 mmol) and trimethylamine (47.4 mL, 340.8 mmol). The reaction mixture was stirred at 160° C. for 2.5 h. The reaction mixture was quenched by water (200 mL) and extracted with 10% methanol in ethyl acetate (100 mL×3). The combined organic layer was washed with brine (100 mL×3), dried over Na₂SO₄, filtered and concentrated under reduced pressure. The residue was purified by silica column chromatography (0˜50% ethyl acetate in petroleum ether) to provide the title compound (10 g, 63.7% yield). LCMS (ESI) [M+H]⁺=461.3.

### Step 4: 5-((1R,5S,6r)-3-((tert-Butyldiphenylsilyl)oxy)bicyclo[3.1.0]hexan-6-yl)-3-iodo-1-isopropyl-1H-1,2,4-triazole

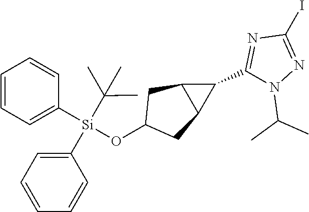

To an ice cooled solution of 5-((1R,5S,6r)-3-((tert-butyldiphenylsilyl)oxy) bicyclo[3.1.0] hexan-6-yl)-1-isopropyl-1H-1,2,4-triazol-3-amine (5.0 g, 10.85 mmol) in acetonitrile (50 mL) and water (10 mL) was added 4-methylbenzenesulfonic acid (9331 mg, 54.3 mmol) and sodium nitrite (1498 mg, 21.71 mmol) in water (5 mL). The reaction mixture was stirred at 0° C. for 30 minutes. Sodium iodide (4067.0 mg, 27.13 mmol) was added rapidly and the solution was stirred at 0° C. for 3 h. The reaction was poured into water (50 mL) and extracted with ethyl acetate (200 mL×3). The combined organic layer was washed with brine (50 mL×3), dried over Na₂SO₄, filtered and concentrated under reduced pressure. The residue was purified by silica column chromatography (0-20% ethyl acetate in petroleum ether) to provide the title compound (3800 mg, 61.3% yield). LCMS (ESI) [M+H]⁺=572.0.

### Step 5: (1R,5S,6r)-6-(3-Iodo-1-isopropyl-1H-1,2,4-triazol-5-yl)bicyclo[3.1.0]hexan-3-ol

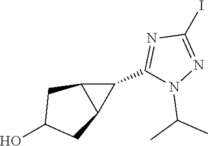

To a stirred solution of 5-((1R,5S,6r)-3-((tert-butyldiphenylsilyl)oxy) bicyclo[3.1.0]hexan-6-yl)-3-iodo-1-isopropyl-1H-1,2,4-triazole (3800.0 mg, 6.65 mmol) in tetrahydrofuran (38 mL) was added triethylamine trihydrofluoride (21.7 mL, 132.97 mmol). The reaction mixture was stirred at 70° C. for 16 h. The reaction was quenched with a saturated aq. NaHCO₃ solution (100 mL) and extracted with ethyl acetate (200 mL×3). The combined organic layer was washed with brine (100 mL), dried over sodium sulfate, filtered and concentrated under reduced pressure. The residue was purified by silica column chromatography (0-25% ethyl acetate in petroleum ether) to provide the title compound (2000 mg, 83.1% yield). LCMS (ESI) [M+H]⁺=334.0.

### Step 6: (1R,5S,6r)-6-(3-Iodo-1-isopropyl-1H-1,2,4-triazol-5-yl)bicyclo[3.1.0]hexan-3-one

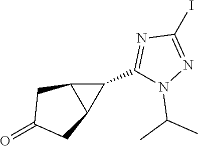 [SMILES: CC(C)n1nc(I)nc1[C@H]1[C@@H]2CC(=O)C[C@@H]21]

To a stirred solution of (1R,5S,6r)-6-(3-iodo-1-isopropyl-1H-1,2,4-triazol-5-yl)bicyclo[3.1.0]hexan-3-ol (2000 mg, 6 mmol) in dichloromethane (20 mL) was added Dess-Martin periodinane (3055 mg, 7.2 mmol). The reaction mixture was stirred at 25° C. for 16 h. The reaction was quenched with a saturated aq. NaHCO₃ solution (80 mL) and extracted with ethyl acetate (200 mL×3). The combined organic layer was washed with brine (50 mL×3), dried over sodium sulfate, filtered and concentrated under reduced pressure. The residue was purified by silica column chromatography (0˜50% ethyl acetate in petroleum ether) to provide the title compound (1200 mg, 58.6% yield). LCMS (ESI) [M+H]⁺=332.0.

### Step 7: (1R,5S,6r)-6-(1-Isopropyl-3-(5-(trifluoromethyl)pyridin-3-yl)-1H-1,2,4-triazol-5-yl)bicyclo[3.1.0]hexan-3-one

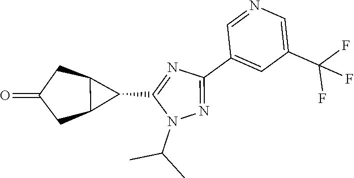 [SMILES: CC(C)n1nc(-c2cncc(C(F)(F)F)c2)nc1[C@H]1[C@@H]2CC(=O)C[C@@H]21]

A suspension of K₂CO₃ (626 mg, 4.53 mmol), bis(di-tert-butyl(4-dimethylaminophenyl)phosphine)dichloropalladium(II) (106.9 mg, 0.15 mmol), (1R,5S,6r)-6-(3-iodo-1-isopropyl-1H-1,2,4-triazol-5-yl)bicyclo[3.1.0]hexan-3-one (500 mg, 1.51 mmol) and 3-(4,4,5,5-tetramethyl-1,3,2-dioxaborolan-2-yl)-5-(trifluoromethyl)pyridine (495 mg, 1.81 mmol) in 1,4-dioxane (10 mL) and water (2 mL) was stirred at 80° C. under a N₂ atmosphere for 2 h. The mixture was diluted with water (25 mL) and extracted with ethyl acetate (40 mL×3). The combined organic layer was washed with brine (20 mL×3), dried over Na₂SO₄, filtered and concentrated under reduced pressure. The residue was purified by silica column chromatography (0-50% ethyl acetate in petroleum ether) to provide the title compound (200 mg, 26.5% yield). LCMS (ESI) [M+H]⁺=351.1.

### Step 8: 6-((1R,5S,6r)-6-(1-isopropyl-3-(5-(trifluoromethyl)pyridin-3-yl)-1H-1,2,4-triazol-5-yl)bicyclo[3.1.0]hexan-3-yl)-2-thia-6-azaspiro[3.4]octane 2,2-dioxide

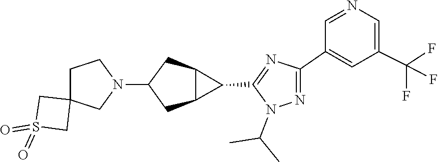

To a solution of (1R,5S,6r)-6-(1-isopropyl-3-(5-(trifluoromethyl)pyridin-3-yl)-1H-1,2,4-triazol-5-yl)bicyclo[3.1.0]hexan-3-one (50 mg, 0.14 mmol) and 2-thia-6-azaspiro[3.4]octane 2,2-dioxide hydrochloride (28.2 mg, 0.14 mmol) in methanol (3 mL) was added NaBH₃CN (45 mg, 0.71 mmol) at 25° C. The reaction mixture was stirred at 80° C. for 4 h. The reaction mixture was concentrated and the residue was purified by preparative reverse phase HPLC (acetonitrile/water gradient with 0.05% NH₃+10 mM NH₄HCO₃) to afford the first eluting peak as a pure single stereoisomer of the title compound (27.5 mg, 39% yield) (Compound 16).

### Compound 16: 6-((1R,3s,5S,6r)-6-(1-Isopropyl-3-(5-(trifluoromethyl)pyridin-3-yl)-1H-1,2,4-triazol-5-yl)bicyclo[3.1.0]hexan-3-yl)-2-thia-6-azaspiro[3.4]octane 2,2-dioxide

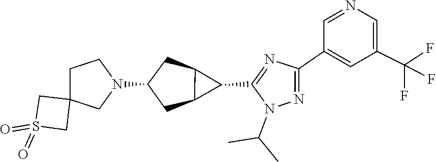 [SMILES: CC(C)n1nc(-c2cncc(C(F)(F)F)c2)nc1[C@H]1[C@@H]2C[C@@H](N3CCC4(C3)CS(=O)(=O)C4)C[C@@H]21]

LCMS (ESI) [M+H]⁺=496.1. ¹H NMR (400 MHz, CD₃OD) δ 9.33 (d, J=1.6 Hz, 1H), 8.83 (d, J=1.2 Hz, 1H), 8.55 (s, 1H), 4.79-4.72 (m, 1H), 4.08-4.05 (m, 4H), 2.94-2.88 (m, 1H), 2.71-2.66 (m, 2H), 2.44-2.43 (m, 1H), 2.19-2.12 (m, 4H), 2.02-1.96 (m, 4H), 1.85 (s, 2H), 1.53 (d, J=6.8 Hz, 6H). The relative stereochemistry was assigned based on ¹H NMR analysis.

### Compound 17: 6-((1R,3r,5S,6r)-6-(1-Isopropyl-3-(5-(trifluoromethyl)pyridin-3-yl)-1H-1,2,4-triazol-5-yl)bicyclo[3.1.0]hexan-3-yl)-2-thia-6-azaspiro[3.4]octane 2,2-dioxide

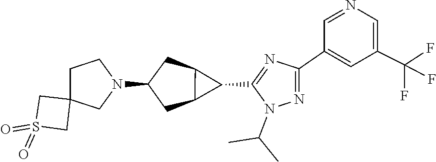 [SMILES: CC(C)n1nc(-c2cncc(C(F)(F)F)c2)nc1[C@H]1[C@@H]2C[C@H](N3CCC4(C3)CS(=O)(=O)C4)C[C@@H]21]

The residue from step 8 was purified by preparative reverse phase HPLC (acetonitrile/water gradient with 0.05% NH₃+10 mM NH₄HCO₃) to afford the second eluting peak as a pure single stereoisomer of the title compound (24.4 mg, 35% yield). LCMS (ESI) [M+H]⁺=496.1. ¹H NMR (400 MHz, CD₃OD) δ 9.34 (d, J=1.2 Hz, 1H), 8.83 (d, J=1.2 Hz, 1H), 8.55 (s, 1H), 4.21-4.12 (m, 1H), 4.11-4.07 (m, 4H), 3.58-3.55 (m, 1H), 3.26-3.07 (m, 1H), 2.85 (s, 1H), 2.71-2.69 (m, 2H), 2.66-2.58 (m, 1H), 2.29-2.15 (m, 5H), 2.06-1.96 (m, 3H), 1.51 (d, J=6.8 Hz, 6H). The relative stereochemistry was assigned based on ¹H NMR analysis.

### Example J: 6-((1R,3s,5S,6r)-6-(1-isopropyl-3-(2-(trifluoromethyl)pyridin-4-yl)-1H-pyrazol-5-yl)bicyclo[3.1.0]hexan-3-yl)-2-thia-6-azaspiro[3.4]octane 2,2-dioxide (Compound 18), and 6-((1R,3r,5S,6r)-6-(1-isopropyl-3-(2-(trifluoromethyl)pyridin-4-yl)-1H-pyrazol-5-yl)bicyclo[3.1.0]hexan-3-yl)-2-thia-6-azaspiro[3.4]octane 2,2-dioxide (Compound 19)

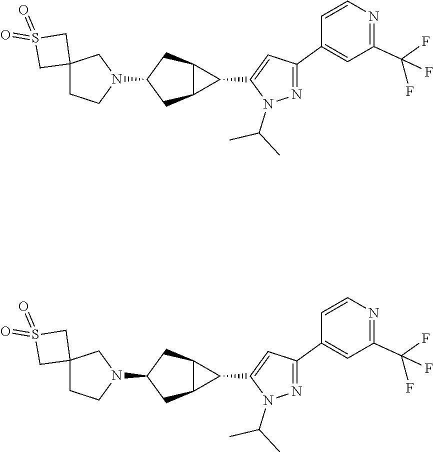 [SMILES: CC(C)n1nc(-c2ccnc(C(F)(F)F)c2)cc1[C@H]1[C@@H]2C[C@@H](N3CCC4(C3)CS(=O)(=O)C4)C[C@@H]21 ; CC(C)n1nc(-c2ccnc(C(F)(F)F)c2)cc1[C@H]1[C@@H]2C[C@H](N3CCC4(C3)CS(=O)(=O)C4)C[C@@H]21]

The title compounds were synthesized following a procedure similar for Compounds 3 and 4 using 5-(4,4,5,5-tetramethyl-1,3,2-dioxaborolan-2-yl)-2-(trifluoromethyl)pyridine. The crude mixture was purified by reverse phase chromatography (acetonitrile/water gradient with 0.05% NH₃+10 mM NH₄HCO₃) to afford compound 19 as the first eluting peak and compound 18 as the second eluting peak.

Compound 18: 6-((1R,3s,5S,6r)-6-(1-isopropyl-3-(2-(trifluoromethyl)pyridin-4-yl)-1H-pyrazol-5-yl)bicyclo[3.1.0]hexan-3-yl)-2-thia-6-azaspiro[3.4]octane 2,2-dioxide. LCMS (ESI) [M+H]⁺=495.2. ¹H NMR (400 MHz, CDCl₃) δ 8.66 (d, J=5.2 Hz, 1H), 8.03 (s, 1H), 7.79 (d, J=5.2 Hz, 1H), 6.24 (s, 1H), 4.72-4.60 (m, 1H), 4.11-4.05 (m, 4H), 2.78 (s, 2H), 2.66 (t, J=7.2 Hz, 2H), 2.47-2.39 (m, 1H), 2.22-2.15 (m, 4H), 1.88-1.65 (m, 4H), 1.59 (t, J=3.2 Hz, 1H), 1.55 (d, J=6.4 Hz, 6H).

Compound 19: 6-((1R,3r,5S,6r)-6-(1-isopropyl-3-(2-(trifluoromethyl)pyridin-4-yl)-1H-pyrazol-5-yl)bicyclo[3.1.0]hexan-3-yl)-2-thia-6-azaspiro[3.4]octane 2,2-dioxide. LCMS (ESI) [M+H]⁺=495.2. ¹H NMR (400 MHz, CDCl₃) δ 8.66 (d, J=5.2 Hz, 1H), 8.03 (s, 1H), 7.80 (d, J=5.2 Hz, 1H), 6.20 (s, 1H), 4.70-4.60 (m, 1H), 4.08-4.00 (m, 4H), 2.96-2.90 (m, 1H), 2.80 (s, 2H), 2.70 (t, J=7.2 Hz, 2H), 2.20-2.12 (m, 4H), 2.08 (t, J=3.2 Hz, 1H), 1.94-1.90 (m, 2H), 1.75-1.60 (m, 2H), 1.55 (d, J=6.8 Hz, 6H).

### Example K: 6-((1R,3s,5S,6r)-6-(1-isopropyl-3-(4-(trifluoromethyl)pyridin-2-yl)-1H-pyrazol-5-yl)bicyclo[3.1.0]hexan-3-yl)-2-thia-6-azaspiro[3.4]octane 2,2-dioxide (Compound 20), and 6-((1R,3r,5S,6r)-6-(1-isopropyl-3-(4-(trifluoromethyl)pyridin-2-yl)-1H-pyrazol-5-yl)bicyclo[3.1.0]hexan-3-yl)-2-thia-6-azaspiro[3.4]octane 2,2-dioxide (Compound 21)

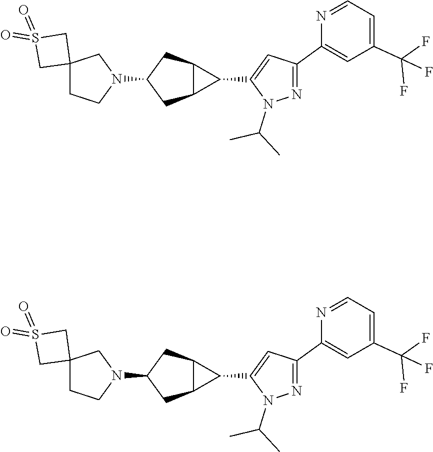 [SMILES: CC(C)n1nc(-c2cc(C(F)(F)F)ccn2)cc1[C@H]1[C@@H]2C[C@@H](N3CCC4(C3)CS(=O)(=O)C4)C[C@@H]21 ; CC(C)n1nc(-c2cc(C(F)(F)F)ccn2)cc1[C@H]1[C@@H]2C[C@H](N3CCC4(C3)CS(=O)(=O)C4)C[C@@H]21]

### Step 1: 6-((1R,5S,6r)-6-(1-isopropyl-3-(4-(trifluoromethyl)pyridin-2-yl)-1H-pyrazol-5-yl)bicyclo[3.1.0]hexan-3-yl)-2-thia-6-azaspiro[3.4]octane 2,2-dioxide

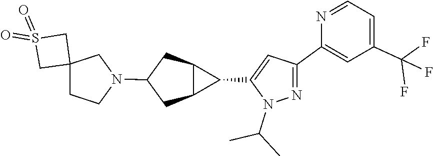 [SMILES: CC(C)n1nc(-c2cc(C(F)(F)F)ccn2)cc1[C@H]1[C@@H]2CC(N3CCC4(C3)CS(=O)(=O)C4)C[C@@H]21]

6-((1R,5S,6r)-6-(1-isopropyl-3-(4-(trifluoromethyl)pyridin-2-yl)-1H-pyrazol-5-yl)bicyclo[3.1.0]hexan-3-yl)-2-thia-6-azaspiro[3.4]octane 2,2-dioxide was synthesized following a procedure similar for Compounds 3 and 4 using 2-(4,4,5,5-tetramethyl-1,3,2-dioxaborolan-2-yl)-4-(trifluoromethyl)pyridine. The crude mixture was purified by chiral SFC (Daicel Chiralcel OD, 0.1% NH₃ in H₂O/EtOH 65:45) to afford compound 20 as the first eluting peak and compound 21 as the second eluting peak. The relative stereochemistry for both compounds was assigned based on ¹H NMR analysis.

Compound 20: 6-((1R,3s,5S,6r)-6-(1-isopropyl-3-(4-(trifluoromethyl)pyridin-2-yl)-1H-pyrazol-5-yl)bicyclo[3.1.0]hexan-3-yl)-2-thia-6-azaspiro[3.4]octane 2,2-dioxide. LCMS (ESI) [M+H]⁺=495.1. ¹H NMR (400 MHz, CD₃OD) δ 8.68 (d, J=5.2 Hz, 1H), 8.13 (s, 1H), 7.49 (d, J=4.0 Hz, 1H), 6.49 (s, 1H), 4.76-4.73 (m, 1H), 4.08-4.05 (m, 4H), 2.83 (s, 2H), 2.70-2.66 (m, 2H), 2.49-2.42 (m, 1H), 2.25-2.15 (m, 4H), 1.85-1.69 (m, 5H), 1.50 (d, J=6.4 Hz, 6H).

Compound 21: 6-((1R,3r,5S,6r)-6-(1-isopropyl-3-(4-(trifluoromethyl)pyridin-2-yl)-1H-pyrazol-5-yl)bicyclo[3.1.0]hexan-3-yl)-2-thia-6-azaspiro[3.4]octane 2,2-dioxide. LCMS (ESI) [M+H]⁺=495.1. ¹H NMR (400 MHz, CD₃OD) δ 8.68 (d, J=5.2 Hz, 1H), 8.13 (s, 1H), 7.49 (d, J=4.4 Hz, 1H), 6.44 (s, 1H), 4.77-4.72 (m, 1H), 4.09-4.06 (m, 4H), 2.95-2.93 (m, 1H), 2.79 (s, 2H), 2.70-2.66 (m, 2H), 2.15-2.11 (m, 5H), 1.92-1.90 (m, 2H), 1.67-1.66 (m, 2H), 1.51 (d, J=6.4 Hz, 6H).

### Example L: 6-((1R,3s,5S,6r)-6-(1-Isopropyl-3-(trifluoromethyl)-1H-pyrazol-5-yl)bicyclo[3.1.0]hexan-3-yl)-2-thia-6-azaspiro[3.4]octane 2,2-dioxide (Compound 22), and 6-((1R,3r,5S,6r)-6-(1-Isopropyl-3-(trifluoromethyl)-1H-pyrazol-5-yl)bicyclo[3.1.0]hexan-3-yl)-2-thia-6-azaspiro[3.4]octane 2,2-dioxide (Compound 23)

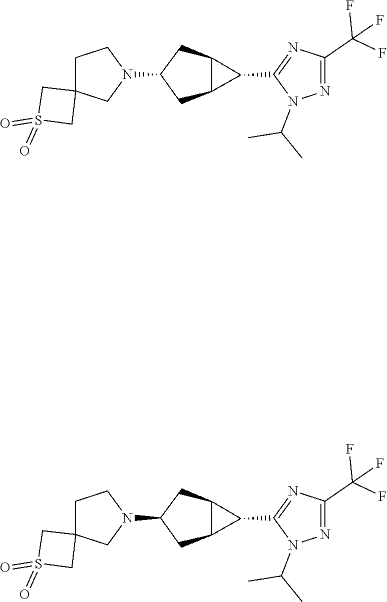 [SMILES: CC(C)n1nc(C(F)(F)F)nc1[C@H]1[C@@H]2C[C@@H](N3CCC4(C3)CS(=O)(=O)C4)C[C@@H]21 ; CC(C)n1nc(C(F)(F)F)nc1[C@H]1[C@@H]2C[C@H](N3CCC4(C3)CS(=O)(=O)C4)C[C@@H]21]

### Step 1: 1-((1R,5S,6r)-3-((tert-Butyldiphenylsilyl)oxy)bicyclo[3.1.0]hexan-6-yl)-4,4,4-trifluorobutane-1,3-dione

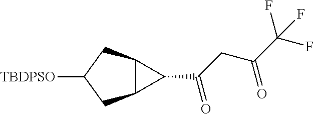

To a solution of ethyl trifluoroacetate (0.52 mL, 4.22 mmol) in THF (10 mL) was added NaH (0.19 g, 4.65 mmol) in portions at 0° C. under N₂. The reaction mixture was at 0° C. stirred for 0.5 h. A solution of 1-((1R,5S,6r)-3-((tert-butyldiphenylsilyl)oxy)bicyclo[3.1.0]hexan-6-yl)ethan-1-one (1.28 g, 3.38 mmol) in THF (20 mL) was added at 0° C. under N₂. The reaction mixture was stirred at 20-30° C. for 2 h, quenched with saturated NH₄Cl solution (50 mL) and extracted with ethyl acetate (50 mL×3). The combined organic layer was washed brine (50 mL×3), dried over Na₂SO₄, filtered and concentrated under reduced pressure. The residue was purified by silica column chromatography (0-10% ethyl acetate in petroleum ether) to provide the title compound (1.2 g, 75% yield). LCMS (ESI) [M+H]⁺=475.1.

### Step 2: 5-((1R,5S,6r)-3-((tert-Butyldiphenylsilyl)oxy)bicyclo[3.1.0]hexan-6-yl)-1-isopropyl-3-(trifluoromethyl)-1H-pyrazole

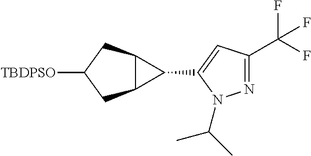

To a solution of 1-((1R,5S,6r)-3-((tert-butyldiphenylsilyl)oxy)bicyclo[3.1.0]hexan-6-yl)-4,4,4-trifluorobutane-1,3-dione (400 mg, 0.84 mmol) in ethanol (15 mL) was added isopropylhydrazine hydrochloride (93 mg, 0.84 mmol) and triethylamine (0.12 mL, 0.84 mmol). The mixture was stirred at 25° C. for 15 h. The mixture was concentrated under reduced pressure and purified by silica column chromatography (0-5% ethyl acetate in petroleum ether) to afford the title compound as a mixture of stereoisomers (350 mg, 81% yield). LCMS (ESI) [M+H]⁺=513.2.

### Step 3: (1R,5S,6r)-6-(1-Isopropyl-3-(trifluoromethyl)-1H-pyrazol-5-yl)bicyclo[3.1.0]hexan-3-ol

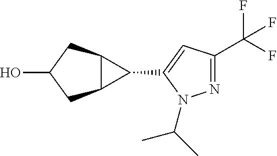 [SMILES: CC(C)n1nc(C(F)(F)F)cc1[C@H]1[C@@H]2CC(O)C[C@@H]21]

To 5-((1R,5S,6r)-3-((tert-butyldiphenylsilyl)oxy)bicyclo[3.1.0]hexan-6-yl)-1-isopropyl-3-(trifluoromethyl)-1H-pyrazole (350 mg, 0.68 mmol) was added 1M tetrabutylammoniumfluoride in tetrahydrofuran (5 mL, 5 mmol). The reaction mixture was stirred at 25° C. for 5 h. The mixture was diluted with water (20 mL) and extracted with ethyl acetate (30 mL×2). The combined organic layer was concentrated under reduced pressure. The residue was purified by silica column chromatography (0-30% ethyl acetate in petroleum ether) to afford the title compound as a mixture of stereoisomers (170 mg, 85.3% yield). LCMS (ESI) [M+H]⁺=275.1.

### Step 4: (1R,5S,6r)-6-(1-Isopropyl-3-(trifluoromethyl)-1H-pyrazol-5-yl)bicyclo[3.1.0]hexan-3-one

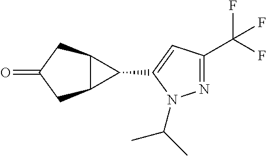 [SMILES: CC(C)n1nc(C(F)(F)F)cc1[C@H]1[C@@H]2CC(=O)C[C@@H]21]

To a solution of (1R,5S,6r)-6-(1-isopropyl-3-(trifluoromethyl)-1H-pyrazol-5-yl)bicyclo[3.1.0]hexan-3-ol (170 mg, 0.62 mmol) in anhydrous dichloromethane (6 mL) was added Dess-Martin periodinane (394 mg, 0.93 mmol). The reaction mixture was stirred at 25° C. for 4 h. The mixture was diluted with H₂O (5 mL), saturated aq. Na₂SO₃ (5 mL) and saturated aq. NaHCO₃ (5 mL). The mixture was extracted with ethyl acetate (25 mL×3). The combined organic layer was washed with brine (50 mL), dried over Na₂SO₄, filtered and concentrated under reduced pressure. The residue was purified by silica column chromatography (0-40% ethyl acetate in petroleum ether) to afford the title compound (150 mg, 74.7% yield). LCMS (ESI) [M+H]⁺=273.1. ¹H NMR (400 MHz, CD₃OD) δ 6.27 (s, 1H), 4.84-4.74 (m, 1H), 2.78-2.72 (m, 2H), 2.44-2.31 (m, 2H), 2.03-1.96 (m, 2H), 1.62 (t, J=3.6 Hz, 1H), 1.48 (d, J=6.8 Hz, 6H).

### Step 5: 6-((1R,5S,6r)-6-(1-isopropyl-3-(trifluoromethyl)-1H-pyrazol-5-yl)bicyclo[3.1.0]hexan-3-yl)-2-thia-6-azaspiro[3.4]octane 2,2-dioxide

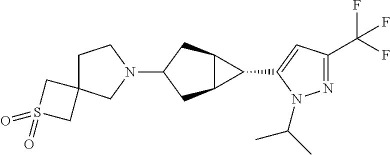

To a solution of (1R,5S,6r)-6-(1-isopropyl-3-(trifluoromethyl)-1H-pyrazol-5-yl)bicyclo[3.1.0]hexan-3-one (100 mg, 0.37 mmol) in methanol (5 mL) was added 2-thia-6-azaspiro[3.4]octane 2,2-dioxide hydrochloride (87.5 mg, 0.44 mmol), acetic acid (0.11 mL) and NaBH₃CN (115 mg, 1.84 mmol). The mixture was stirred at 70° C. for 2 h. The reaction mixture was diluted with saturated aq. sodium bicarbonate (10 mL) and extracted with dichloromethane (50 mL×3). The combined organic layer was dried over anhydrous Na₂SO₄, filtered and concentrated under reduced pressure. The residue was purified by reverse phase chromatography (acetonitrile/water gradient with 0.05% NH₃+10 mM NH₄HCO₃) to give the first eluting peak as a pure single stereoisomer of the above titled compound (30 mg, 19.5% yield) (Compound 22).

### Compound 22: 6-((1R,3s,5S,6r)-6-(1-Isopropyl-3-(trifluoromethyl)-1H-pyrazol-5-yl)bicyclo[3.1.0]hexan-3-yl)-2-thia-6-azaspiro[3.4]octane 2,2-dioxide

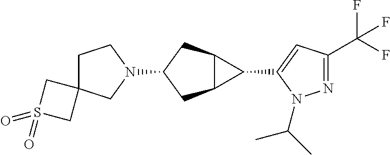 [SMILES: CC(C)n1nc(C(F)(F)F)cc1[C@H]1[C@@H]2C[C@@H](N3CCC4(C3)CS(=O)(=O)C4)C[C@@H]21]

LCMS (ESI) [M+H]⁺=418.2. ¹H NMR (400 MHz, CDCl₃) δ 6.04 (s, 1H), 4.68-4.60 (m, 1H), 4.12-4.01 (m, 4H), 2.77 (s, 2H), 2.65 (t, J=7.2 Hz, 2H), 2.45-2.36 (m, 1H), 2.20-2.14 (m, 4H), 1.87-1.81 (m, 2H), 1.66-1.65 (m, 2H), 1.56-1.55 (m, 1H), 1.51 (d, J=6.4 Hz, 6H).

### Compound 23: 6-((1R,3r,5S,6r)-6-(1-Isopropyl-3-(trifluoromethyl)-1H-pyrazol-5-yl)bicyclo[3.1.0]hexan-3-yl)-2-thia-6-azaspiro[3.4]octane 2,2-dioxide

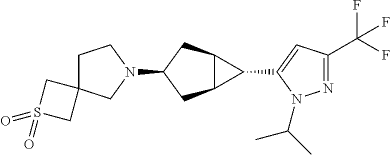 [SMILES: CC(C)n1nc(C(F)(F)F)cc1[C@H]1[C@@H]2C[C@H](N3CCC4(C3)CS(=O)(=O)C4)C[C@@H]21]

The crude mixture from step 5 was purified by preparative reverse phase HPLC (acetonitrile/water gradient with 0.05% NH₃+10 mM NH₄HCO₃) to give the second eluting peak as a pure single stereoisomer of the above titled compound (30.5 mg, 19.5% yield). ¹H NMR (400 MHz, CDCl₃) δ 6.00 (s, 1H), 4.68-4.60 (m, 1H), 4.09-3.97 (m, 4H), 2.96-2.86 (m, 1H), 2.79 (s, 2H), 2.68 (t, J=7.2 Hz, 2H), 2.18-2.10 (m, 4H), 2.07-2.06 (m, 1H), 1.92-1.86 (m, 2H), 1.62-1.61 (m, 2H), 1.51 (d, J=6.8 Hz, 6H). The relative stereochemistry was assigned based on ¹H NMR analysis.

### Example M: 6-((1R,3s,5S,6r)-6-(1-ethyl-3-(5-(trifluoromethyl)pyridin-3-yl)-1H-pyrazol-5-yl)bicyclo[3.1.0]hexan-3-yl)-2-thia-6-azaspiro[3.4]octane 2,2-dioxide (Compound 24), and 6-((1R,3r,5S,6r)-6-(1-ethyl-3-(5-(trifluoromethyl)pyridin-3-yl)-1H-pyrazol-5-yl)bicyclo[3.1.0]hexan-3-yl)-2-thia-6-azaspiro[3.4]octane 2,2-dioxide (Compound 25)

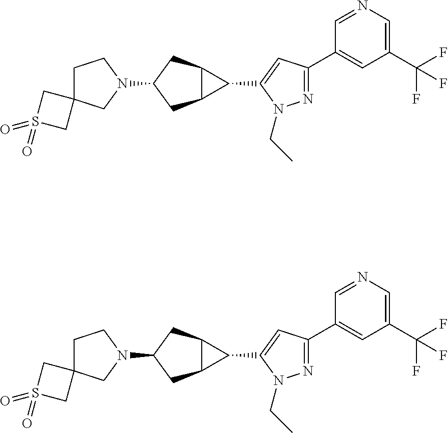 [SMILES: CCn1nc(-c2cncc(C(F)(F)F)c2)cc1[C@H]1[C@@H]2C[C@@H](N3CCC4(C3)CS(=O)(=O)C4)C[C@@H]21 ; CCn1nc(-c2cncc(C(F)(F)F)c2)cc1[C@H]1[C@@H]2C[C@H](N3CCC4(C3)CS(=O)(=O)C4)C[C@@H]21]

### Step 1: 3-(5-((1R,5S,6r)-3-((tert-Butyldiphenylsilyl)oxy)bicyclo[3.1.0]hexan-6-yl)-1H-pyrazol-3-yl)-5-(trifluoromethyl)pyridine

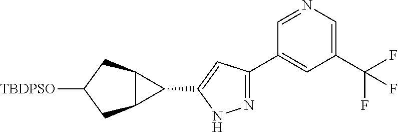

To a solution of tert-butyl-[[(1R,5S)-6-(3-iodo-1H-pyrazol-5-yl)-3-bicyclo[3.1.0]hexanyl]oxy]-diphenyl-silane (2.0 g, 3.78 mmol) and 3-(4,4,5,5-tetramethyl-1,3,2-dioxaborolan-2-yl)-5-(trifluoromethyl)pyridine (1.6 g, 5.86 mmol) in 1,4-dioxane (20 mL) and water (4 mL) were added K₂CO₃ (1.6 g, 11.58 mmol) and bis(di-tert-butyl(4-dimethylaminophenyl)phosphine)dichloropalladium(II) (0.28 g, 0.40 mmol). The reaction mixture was placed under a nitrogen atmosphere and stirred at 75° C. for 5 h. The reaction mixture was concentrated under reduced pressure and the residue was purified by silica column chromatography (0-30% ethyl acetate in petroleum ether) to provide the title compound (2.0 g, 3.32 mmol, 87.8% yield). LCMS (ESI) [M+H]⁺=548.3.

### Step 2: 3-(5-((1R,5S,6r)-3-((tert-Butyldiphenylsilyl)oxy)bicyclo[3.1.0]hexan-6-yl)-1-ethyl-1H-pyrazol-3-yl)-5-(trifluoromethyl)pyridine

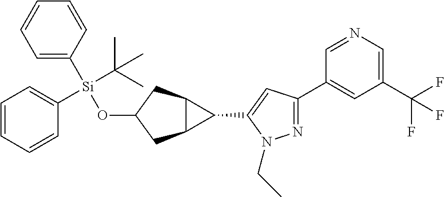 [SMILES: CCn1nc(-c2cncc(C(F)(F)F)c2)cc1[C@H]1[C@@H]2CC(O[Si](c3ccccc3)(c3ccccc3)C(C)(C)C)C[C@@H]21]

To a stirred mixture of 3-(5-((1R,5S,6r)-3-((tert-butyldiphenylsilyl)oxy)bicyclo[3.1.0] hexan-6-yl)-1H-pyrazol-3-yl)-5-(trifluoromethyl)pyridine (1.2 g, 2.19 mmol) and Cs₂CO₃ (1.4 g, 4.3 mmol) in N,N-dimethylformamide (12 mL) was added iodoethane (0.9 mL, 11.25 mmol) dropwise. The mixture was stirred at 25° C. for 3 h. The reaction was quenched with water (50 mL) and extracted with ethyl acetate (100 mL×3). The combined organic layer was washed with brine (50 mL×3), dried over Na₂SO₄, filtered and concentrated. The residue was purified by silica column chromatography (0-15% ethyl acetate in petroleum ether) to provide the title compound (890 mg, 70.6% yield). LCMS (ESI) [M+H]⁺=576.2.

### Step 3: (1R,5S,6r)-6-(1-Ethyl-3-(5-(trifluoromethyl)pyridin-3-yl)-1H-pyrazol-5-yl)bicyclo[3.1.0]hexan-3-ol

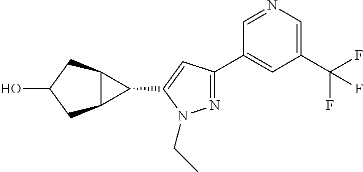 [SMILES: CCn1nc(-c2cncc(C(F)(F)F)c2)cc1[C@H]1[C@@H]2CC(O)C[C@@H]21]

To 3-(5-((1R,5S,6r)-3-((tert-butyldiphenylsilyl)oxy)bicyclo[3.1.0]hexan-6-yl)-1-ethyl-1H-pyrazol-3-yl)-5-(trifluoromethyl)pyridine (870 mg, 1.51 mmol) was added tetrabutyl ammonium fluoride in tetrahydrofuran (12.0 mL, 12 mmol, 1 M) and the mixture was stirred at 25° C. for 5 h. The reaction was quenched with a saturated NH₄Cl aqueous solution (50 mL) and extracted with ethyl acetate (100 mL×3). The combined organic layer was washed with brine (30 mL), dried over anhydrous Na₂SO₄, filtered and concentrated. The residue was purified by silica column chromatography (0-50% ethyl acetate in petroleum ether) to provide the title compound (500 mg, 98.1% yield). LCMS (ESI) [M+H]⁺=338.1.

### Step 4: (1R,5S,6r)-6-(1-Ethyl-3-(5-(trifluoromethyl)pyridin-3-yl)-1H-pyrazol-5-yl)bicyclo[3.1.0]hexan-3-one

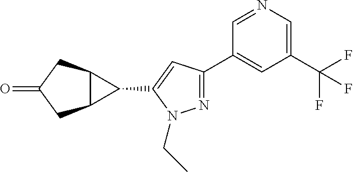 [SMILES: CCn1nc(-c2cncc(C(F)(F)F)c2)cc1[C@H]1[C@@H]2CC(=O)C[C@@H]21]

To a solution of (1R,5S,6r)-6-(1-ethyl-3-(5-(trifluoromethyl)pyridin-3-yl)-1H-pyrazol-5-yl)bicyclo[3.1.0]hexan-3-ol (500 mg, 1.48 mmol) in anhydrous dichloromethane (12 mL) was added Dess-Martin periodinane (1000 mg, 2.36 mmol). The reaction mixture was stirred at 25° C. for 4 h. The mixture was quenched with a Na₂SO₃ aqueous solution (20 mL). A saturated NaHCO₃ aqueous solution (10 mL) was added and the mixture was extracted with dichloromethane (100 mL×3). The combined organic layer was washed with brine (30 mL), dried over Na₂SO₄, filtered and concentrated. The residue was purified by silica column chromatography (0-40% ethyl acetate in petroleum ether) to provide the title compound (410 mg, 82.5% yield). LCMS (ESI) [M+H]⁺=336.3.

### Step 5: 6-((1R,5S,6r)-6-(1-ethyl-3-(5-(trifluoromethyl)pyridin-3-yl)-1H-pyrazol-5-yl)bicyclo[3.1.0]hexan-3-yl)-2-thia-6-azaspiro[3.4]octane 2,2-dioxide

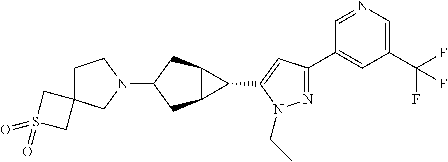

To a solution of (1R,5S,6r)-6-(1-ethyl-3-(5-(trifluoromethyl)pyridin-3-yl)-1H-pyrazol-5-yl)bicyclo[3.1.0]hexan-3-one (130 mg, 0.38 mmol) and 2-thia-6-azaspiro[3.4]octane 2,2-dioxide hydrochloride (100 mg, 0.504 mmol) in anhydrous methanol (4 mL) was added NaBH₃CN (120 mg, 1.92 mmol) and acetic acid (0.02 mL, 0.35 mmol) at 20° C. Then the reaction mixture was heated to 70° C. and stirred for 5 hours. The reaction was quenched with a saturated NaHCO₃ aqueous solution (5 mL), and extracted with dichloromethane (30 mL×3). The combined organic phase was washed with brine, dried over Na₂SO₄, filtered and concentrated under reduced pressure. The crude residue was purified by reverse phase HPLC (acetonitrile/water gradient with 0.05% NH₃+10 mM NH₄HCO₃) to afford the second eluting peak as a pure single stereoisomer of the title compound (51.5 mg, 27.6% yield) (Compound 24).

### Compound 24: 6-((1R,3s,5S,6r)-6-(1-Ethyl-3-(5-(trifluoromethyl)pyridin-3-yl)-1H-pyrazol-5-yl)bicyclo[3.1.0]hexan-3-yl)-2-thia-6-azaspiro[3.4]octane 2,2-dioxide

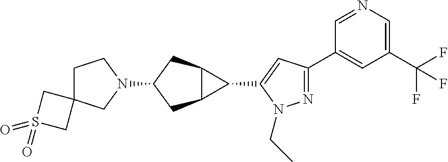 [SMILES: CCn1nc(-c2cncc(C(F)(F)F)c2)cc1[C@H]1[C@@H]2C[C@@H](N3CCC4(C3)CS(=O)(=O)C4)C[C@@H]21]

LCMS (ESI) [M+H]⁺=481.4. ¹H NMR (400 MHz, CDCl₃) δ 9.11 (d, J=1.6 Hz, 1H), 8.76 (s, 1H), 8.28 (s, 1H), 6.19 (s, 1H), 4.27-4.23 (m, 2H), 4.09 (s, 4H), 2.82-2.78 (m, 1H), 2.69-2.65 (m, 2H), 2.46-2.42 (m, 1H), 2.22-2.18 (m, 4H), 2.00-1.85 (m, 1H), 1.75-1.72 (m, 3H), 1.58 (s, 2H), 1.51 (t, J=7.2 Hz, 3H). The relative stereochemistry was assigned based on ¹H NMR analysis.

### Compound 25: 6-((1R,3r,5S,6r)-6-(1-ethyl-3-(5-(trifluoromethyl)pyridin-3-yl)-1H-pyrazol-5-yl)bicyclo[3.1.0]hexan-3-yl)-2-thia-6-azaspiro[3.4]octane 2,2-dioxide

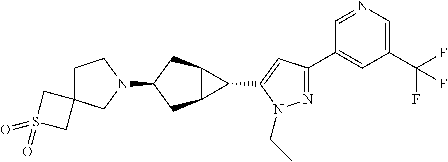 [SMILES: CCn1nc(-c2cncc(C(F)(F)F)c2)cc1[C@H]1[C@@H]2C[C@H](N3CCC4(C3)CS(=O)(=O)C4)C[C@@H]21]

The residue from step 5 was purified by preparative reverse phase HPLC (acetonitrile/water gradient with 0.05% NH₃+10 mM NH₄HCO₃) to afford the first eluting peak as a pure single stereoisomer of the title compound (46.1 mg, 25% yield). LCMS (ESI) [M+H]⁺=481.4. ¹H NMR (400 MHz, CDCl₃) δ 9.10 (s, 1H), 8.76 (s, 1H), 8.28 (s, 1H), 6.14 (s, 1H), 4.30-4.20 (m, 2H), 4.13-3.99 (m, 4H), 2.95-2.90 (m, 1H), 2.82-2.79 (m, 2H), 2.72-2.68 (m, 2H), 2.43-2.11 (m, 3H), 2.09-2.06 (m, 1H), 1.98-1.90 (m, 2H), 1.67-1.64 (m, 2H), 1.50 (t, J=7.2 Hz, 3H). The relative stereochemistry was assigned based on ¹H NMR analysis.

### Example N: 6-((1R,3s,5S,6r)-6-(1-ethyl-3-(2-(trifluoromethyl)pyridin-4-yl)-1H-pyrazol-5-yl)bicyclo[3.1.0]hexan-3-yl)-2-thia-6-azaspiro[3.4]octane 2,2-dioxide (Compound 26) and 6-((1R,3r,5S,6r)-6-(1-ethyl-3-(2-(trifluoromethyl)pyridin-4-yl)-1H-pyrazol-5-yl)bicyclo[3.1.0]hexan-3-yl)-2-thia-6-azaspiro[3.4]octane 2,2-dioxide (Compound 27)

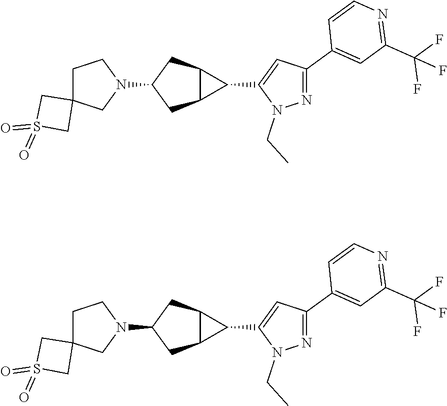 [SMILES: CCn1nc(-c2ccnc(C(F)(F)F)c2)cc1[C@H]1[C@@H]2C[C@@H](N3CCC4(C3)CS(=O)(=O)C4)C[C@@H]21 ; CCn1nc(-c2ccnc(C(F)(F)F)c2)cc1[C@H]1[C@@H]2C[C@H](N3CCC4(C3)CS(=O)(=O)C4)C[C@@H]21]

The title compounds were synthesized following a procedure similar for Compounds 24 and 25 using 4-(4,4,5,5-tetramethyl-1,3,2-dioxaborolan-2-yl)-2-(trifluoromethyl)pyridine. The relative stereochemistry for both compounds was assigned based on ¹H NMR analysis.

Compound 26: 6-((1R,3s,5S,6r)-6-(1-ethyl-3-(5-(trifluoromethyl)pyridin-3-yl)-1H-pyrazol-5-yl)bicyclo[3.1.0]hexan-3-yl)-2-thia-6-azaspiro[3.4]octane 2,2-dioxide. LCMS (ESI) [M+H]⁺=481.2. ¹H NMR (400 MHz, CDCl₃) δ 8.68 (d, J=5.2 Hz, 1H), 8.02 (s, 1H), 7.77 (d, J=5.2 Hz, 1H), 6.25 (s, 1H), 4.24 (q, J=7.2 Hz, 2H), 4.19-4.08 (m, 4H), 2.87-2.65 (m, 3H), 2.52-2.41 (m, 1H), 2.29-2.18 (m, 4H), 2.10-1.80 (m, 2H), 1.79-1.61 (m, 3H), 1.58-1.56 (m, 1H), 1.51 (t, J=7.2 Hz, 3H).

Compound 27: 6-((1R,3r,5S,6r)-6-(1-ethyl-3-(5-(trifluoromethyl)pyridin-3-yl)-1H-pyrazol-5-yl)bicyclo[3.1.0]hexan-3-yl)-2-thia-6-azaspiro[3.4]octane 2,2-dioxide. LCMS (ESI) [M+H]⁺=481.2. ¹H NMR (400 MHz, CDCl₃) δ 8.68 (d, J=5.2 Hz, 1H), 8.01 (s, 1H), 7.78 (d, J=4.4 Hz, 1H), 6.20 (s, 1H), 4.24 (q, J=7.2 Hz, 2H), 4.09-4.01 (m, 4H), 2.98-2.91 (m, 1H), 2.80 (s, 2H), 2.73-2.67 (m, 2H), 2.16-2.09 (m, 4H), 1.98-1.91 (m, 2H), 1.73-1.62 (m, 2H), 1.61-1.58 (m, 1H), 1.50 (t, J=7.2 Hz, 3H).

### Example O: 6-((1R,3s,5S,6r)-6-(1-isopropyl-3-(2-(trifluoromethyl)pyrimidin-4-yl)-1H-pyrazol-5-yl)bicyclo[3.1.0]hexan-3-yl)-2-thia-6-azaspiro[3.4]octane 2,2-dioxide (Compound 28)

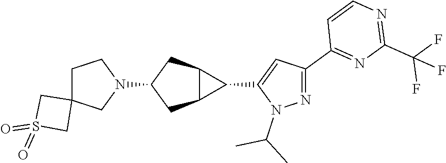 [SMILES: CC(C)n1nc(-c2ccnc(C(F)(F)F)n2)cc1[C@H]1[C@@H]2C[C@@H](N3CCC4(C3)CS(=O)(=O)C4)C[C@@H]21]

The title compound was synthesized following a procedure similar for Compound 5 using (2-(trifluoromethyl)pyrimidin-4-yl)boronic acid. The relative stereochemistry was assigned based on ¹H NMR analysis.

Compound 28: LCMS (ESI) [M+H]⁺=496.2. ¹H NMR (400 MHz, DMSO-d₆) δ 8.96 (d, J=5.3 Hz, 1H), 8.11 (d, J=5.4 Hz, 1H), 6.60 (s, 1H), 4.90-4.76 (m, 1H), 4.19-4.07 (m, 4H), 2.68 (s, 2H), 2.59-2.51 (m, 3H), 2.16-2.04 (m, 4H), 1.87-1.82 (m, 1H), 1.77-1.67 (m, 4H), 1.46 (d, J=6.5 Hz, 6H).

### Example P: 6-((1R,3r,5S,6r)-6-(1-Isopropyl-3-(3,3,3-trifluoroprop-1-en-2-yl)-1H-pyrazol-5-yl)bicyclo[3.1.0]hexan-3-yl)-2-thia-6-azaspiro[3.4]octane 2,2-dioxide (Compound 31)

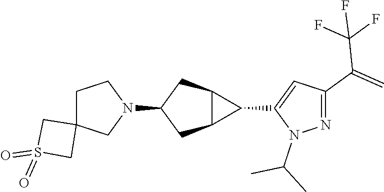 [SMILES: C=C(c1cc([C@H]2[C@@H]3C[C@H](N4CCC5(C4)CS(=O)(=O)C5)C[C@@H]32)n(C(C)C)n1)C(F)(F)F]

### Step 1: 6-((1R,3r,5S,6r)-6-(3-Iodo-1-isopropyl-1H-pyrazol-5-yl)bicyclo[3.1.0]hexan-3-yl)-2-thia-6-azaspiro[3.4]octane 2,2-dioxide

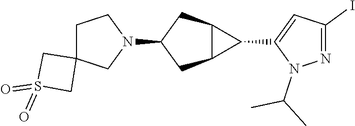 [SMILES: CC(C)n1nc(I)cc1[C@H]1[C@@H]2C[C@H](N3CCC4(C3)CS(=O)(=O)C4)C[C@@H]21]

Step 2: The title compound was prepared following the procedure described for Compound 5. A solution of 6-((1R,3r,5S,6r)-6-(3-iodo-1-isopropyl-1H-pyrazol-5-yl)bicyclo[3.1.0]hexan-3-yl)-2-thia-6-azaspiro[3.4]octane 2,2-dioxide (75 mg, 0.16 mmol), potassium phosphate (67 mg, 0.32 mmol), Cataxium Pd G4 (5.9 mg, 0.008 mmol) and 4,4,6,6-tetramethyl-2-(3,3,3-trifluoroprop-1-en-2-yl)-1,3,2-dioxaborinane (56 mg, 0.24 mmol) in 1,4-dioxane (0.79 mL) and water (0.20 mL) was stirred at 50° C. for 18 h. The reaction mixture was diluted with 1N aq. NH₄Cl (0.5 mL) and DMSO, filtered and purified by prep-HPLC (acetonitrile/water gradient with 0.1% TFA) to give the title compound (38 mg, 54% yield). The relative stereochemistry was assigned based on ¹H NMR analysis.

Compound 31: LCMS (ESI) [M+H]⁺=444.3. ¹H NMR (400 MHz, DMSO-d₆) δ 6.09 (s, 1H), 6.02 (s, 1H), 5.81 (s, 1H), 4.69-4.57 (m, 1H), 4.16-4.04 (m, 4H), 2.85-2.78 (m, 1H), 2.70 (s, 2H), 2.57 (t, J=7.2 Hz, 2H), 2.23-2.17 (m, 1H), 2.10-1.96 (m, 4H), 1.90 (dd, J=13.7, 3.2 Hz, 2H), 1.62-1.57 (m, 2H), 1.40 (d, J=6.5 Hz, 6H).

### Example Q: 6-((1R,3r,5S,6r)-6-(1-Isopropyl-3-(1-(trifluoromethyl)cyclopropyl)-1H-pyrazol-5-yl)bicyclo[3.1.0]hexan-3-yl)-2-thia-6-azaspiro[3.4]octane 2,2-dioxide (Compound 32)

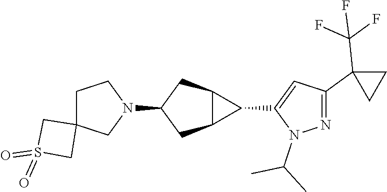 [SMILES: CC(C)n1nc(C2(C(F)(F)F)CC2)cc1[C@H]1[C@@H]2C[C@H](N3CCC4(C3)CS(=O)(=O)C4)C[C@@H]21]

To a cooled to 0° C. solution of 6-((1R,3r,5S,6r)-6-(1-isopropyl-3-(3,3,3-trifluoroprop-1-en-2-yl)-1H-pyrazol-5-yl)bicyclo[3.1.0]hexan-3-yl)-2-thia-6-azaspiro[3.4]octane 2,2-dioxide (36 mg, 0.081 mmol) and diphenyl(methyl)sulfonium tetrafluoroborate (30 mg, 0.11 mmol) in tetrahydrofuran (0.54 mL) was added sodium bis(trimethylsilyl)amide (0.13 mL, 0.13 mmol, 1 mol/L in tetrahydrofuran). The reaction mixture was stirred at 0-25° C. for 1 h. The reaction mixture was cooled to 0° C., and additional diphenyl(methyl)sulfonium tetrafluoroborate (25 mg, 0.092 mmol) and sodium bis(trimethylsilyl)amide (0.090 mL, 0.090 mmol, 1 mol/L in tetrahydrofuran) were added. The reaction mixture was stirred at 0-25° C. for 2 h. The reaction mixture was diluted with 1N aq. NH₄Cl (0.5 mL) and DMSO, filtered and purified by prep-HPLC (acetonitrile/water gradient with 0.1% TFA) to give the title compound (6.1 mg, 16% yield).

Compound 32: LCMS (ESI) [M+H]⁺=458.2. ¹H NMR (400 MHz, DMSO-d₆) δ5.82 (s, 1H), 4.63-4.48 (m, 1H), 4.16-4.02 (m, 4H), 2.85-2.75 (m, 1H), 2.69 (s, 2H), 2.56 (t, J=7.3 Hz, 2H), 2.15 (t, J=3.4 Hz, 1H), 2.09-1.93 (m, 4H), 1.88 (dd, J=13.8, 3.2 Hz, 2H), 1.58-1.52 (m, 2H), 1.35 (d, J=6.6 Hz, 6H), 1.24-1.20 (m, 2H), 1.15-1.08 (m, 2H). The relative stereochemistry was assigned based on ¹H NMR analysis.

### Example R: 7-((1R,3s,5S,6r)-6-(1-Isopropyl-3-(5-(trifluoromethyl)pyridin-3-yl)-1H-pyrazol-5-yl)bicyclo[3.1.0]hexan-3-yl)-2-thia-7-azaspiro[3.5]nonane 2,2-dioxide (Compound 38)

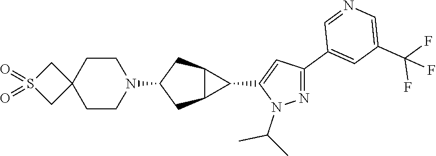 [SMILES: CC(C)n1nc(-c2cncc(C(F)(F)F)c2)cc1[C@H]1[C@@H]2C[C@@H](N3CCC4(CC3)CS(=O)(=O)C4)C[C@@H]21]

The title compound was synthesized following a procedure similar for Compound 3 using 2-thia-7-azaspiro[3.5]nonane 2,2-dioxide. The crude mixture was purified by silica column chromatography (0-12% ethanol in ethyl acetate) to afford compound 38 as the first eluting peak. The relative stereochemistry was assigned based on ¹H NMR analysis.

Compound 38: LCMS (ESI) [M+H]⁺=509.2. ¹H NMR (400 MHz, CDCl₃) δ 9.11 (d, J=2.0 Hz, 1H), 8.75 (d, J=1.2 Hz, 1H), 8.29 (s, 1H), 6.13 (s, 1H), 4.69-4.59 (m, 1H), 3.86 (s, 4H), 2.58-2.33 (m, 4H), 2.27-2.22 (m, 2H), 1.95-1.92 (m, 4H), 1.88-1.83 (m, 2H), 1.72 (s, 2H), 1.66-1.56 (m, 2H), 1.55 (d, J=6.4 Hz, 6H).

### Example S: 7-((1R,3r,5S,6r)-6-(1-Isopropyl-3-(5-(trifluoromethyl)pyridin-3-yl)-1H-pyrazol-5-yl)bicyclo[3.1.0]hexan-3-yl)-2-thia-7-azaspiro[3.5]nonane 2,2-dioxide (Compound 40)

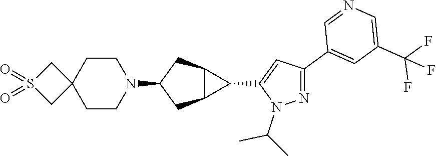 [SMILES: CC(C)n1nc(-c2cncc(C(F)(F)F)c2)cc1[C@H]1[C@@H]2C[C@H](N3CCC4(CC3)CS(=O)(=O)C4)C[C@@H]21]

The title compound was synthesized following a procedure similar to Compound 4 using 2-thia-7-azaspiro[3.5]nonane 2,2-dioxide. The crude mixture was purified by silica column chromatography (0-12% ethanol in ethyl acetate) to afford compound 40 as the second eluting peak. The relative stereochemistry was assigned based on ¹H NMR analysis.

Compound 40: LCMS (ESI) [M+H]⁺=509.2. ¹H NMR (400 MHz, CDCl₃) δ 9.10 (d, J=1.6 Hz, 1H), 8.74 (d, J=1.2 Hz, 1H), 8.28 (s, 1H), 6.13 (s, 1H), 4.72-4.62 (m, 1H), 3.85 (s, 4H), 2.96-2.88 (m, 1H), 2.56-2.25 (m, 5H), 1.92-1.89 (m, 4H), 1.71-1.64 (m, 4H), 1.61-1.58 (m, 2H), 1.55 (d, J=6.8 Hz, 6H).

### Example T—Compounds 43* and 44*: 7-((1R,3s,5S,6r)-6-(1-Isopropyl-3-(trifluoromethyl)-1H-pyrazol-5-yl)bicyclo[3.1.0]hexan-3-yl)-2-thia-7-azaspiro[3.5]nonane 2,2-dioxide, and 7-((1R,3r,5S,6r)-6-(1-Isopropyl-3-(trifluoromethyl)-1H-pyrazol-5-yl)bicyclo[3.1.0]hexan-3-yl)-2-thia-7-azaspiro[3.5]nonane 2,2-dioxide

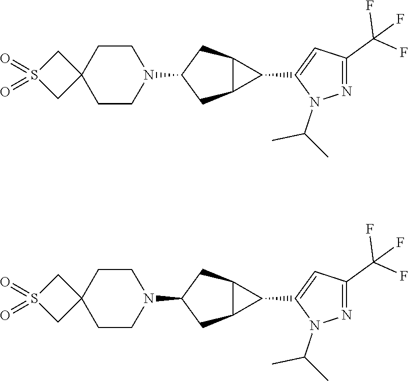 [SMILES: CC(C)n1nc(C(F)(F)F)cc1[C@H]1[C@@H]2C[C@@H](N3CCC4(CC3)CS(=O)(=O)C4)C[C@@H]21 ; CC(C)n1nc(C(F)(F)F)cc1[C@H]1[C@@H]2C[C@H](N3CCC4(CC3)CS(=O)(=O)C4)C[C@@H]21]

### Step 1: 1-(3-((tert-Butyldiphenylsilyl)oxy)bicyclo[3.1.0]hexan-6-yl)-4,4,4-trifluorobutane-1,3-dione

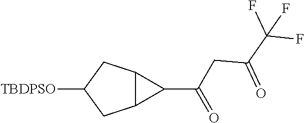

To a solution of ethyl trifluoroacetate (0.52 mL, 4.22 mmol) in THF (10 mL) was added NaH (0.19 g, 4.65 mmol) in portions at 0° C. under N₂ and stirred for 0.5 h. A solution of 1-[3-[tert-butyl(diphenyl)silyl]oxy-6-bicyclo[3.1.0]hexanyl]ethanone (1.28 g, 3.38 mmol) in THF (20 mL) was added at 0° C. under N₂. The reaction mixture was then stirred at 20-30° C. for 2 h. The reaction mixture was quenched with saturated NH₄Cl solution (50 mL) and extracted with ethyl acetate (50 mL×3). The combined organic layers were washed brine (50 mL×3), dried over anhydrous Na₂SO₄, filtered and concentrated in vacuo. The resulting residue was purified by silica flash chromatography (0-10% ethyl acetate in petroleum ether) to provide the title compound (1.2 g, 2.53 mmol, 75% yield). LCMS (ESI) [M+H]⁺=475.1.

### Step 2: 5-(3-((tert-Butyldiphenylsilyl)oxy)bicyclo[3.1.0]hexan-6-yl)-1-isopropyl-3-(trifluoromethyl)-1H-pyrazole

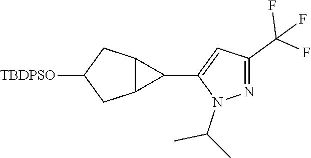

To a solution of 1-[3-[tert-butyl(diphenyl)silyl]oxy-6-bicyclo[3.1.0]hexanyl]-4,4,4-trifluoro-butane-1,3-dione (400 mg, 0.84 mmol) in ethanol (15 mL) were added isopropylhydrazine hydrochloride (93 mg, 0.84 mmol) and triethylamine (0.12 mL, 0.84 mmol). The reaction mixture was stirred at 25° C. for 15 h. The mixture was concentrated in vacuo and purified by silica flash chromatography (0-5% ethyl acetate in petroleum ether) to afford the title compound (350 mg, 0.683 mmol, 81% yield). LCMS (ESI) [M+H]⁺=513.2.

### Step 3: 6-(1-Isopropyl-3-(trifluoromethyl)-1H-pyrazol-5-yl)bicyclo[3.1.0]hexan-3-ol

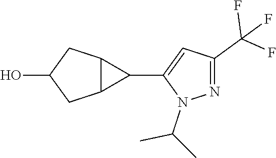 [SMILES: CC(C)n1nc(C(F)(F)F)cc1C1C2CC(O)CC21]

To tert-butyl-[[6-[2-isopropyl-5-(trifluoromethyl)pyrazol-3-yl]-3-bicyclo[3.1.0]hexanyl]oxy]-diphenyl-silane (350 mg, 0.68 mmol) in the flask was added tetrabutylammoniumfluoride in tetrahydrofuran (5 mL, 5 mmol, 1 M). The reaction mixture was stirred at 25° C. for 5 h. The mixture was diluted with water (20 mL) and extracted with ethyl acetate (30 mL×2). The combined organic layers were concentrated in vacuo. The resulting residue was purified by silica flash chromatography (0-30% ethyl acetate in petroleum ether) to afford the title compound (170 mg, 0.5826 mmol, 85.3% yield). LCMS (ESI) [M+H]⁺=275.1.

### Step 4: (1R,5S,6r)-6-(1-Isopropyl-3-(trifluoromethyl)-1H-pyrazol-5-yl)bicyclo[3.1.0]hexan-3-one

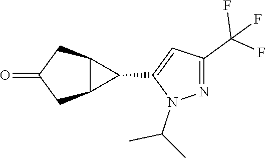 [SMILES: CC(C)n1nc(C(F)(F)F)cc1[C@H]1[C@@H]2CC(=O)C[C@@H]21]

To a solution of 6-[2-isopropyl-5-(trifluoromethyl)pyrazol-3-yl]bicyclo[3.1.0]hexan-3-ol (170 mg, 0.62 mmol) in anhydrous dichloromethane (6 mL) was added Dess-Martin periodinane (394 mg, 0.93 mmol). The reaction mixture was stirred at 25° C. for 4 h. The reaction mixture was then diluted with H₂O (5 mL), saturated Na₂SO₃ solution (5 mL) and saturated NaHCO₃ solution (5 mL). The mixture was extracted with ethyl acetate (25 mL×3). The combined organic layers were washed with brine (50 mL), dried over anhydrous Na₂SO₄, filtered and concentrated in vacuo. The resulting residue was purified by silica flash chromatography (0-40% ethyl acetate in petroleum ether) to afford the title compound (150 mg, 0.463 mmol, 75% yield). LCMS (ESI) [M+H]⁺=273.1; ¹H NMR (400 MHz, CD₃OD) δ 6.27 (s, 1H), 4.84-4.74 (m, 1H), 2.78-2.72 (m, 2H), 2.44-2.31 (m, 2H), 2.03-1.96 (m, 2H), 1.62 (t, J=3.6 Hz, 1H), 1.48 (d, J=6.8 Hz, 6H).

Step 5: Synthesis of title compounds. To a mixture of (1R, 5S)-6-[2-isopropyl-5-(trifluoromethyl)pyrazol-3-yl]bicyclo[3.1.0]hexan-3-one (600.0 mg, 2.2 mmol), 2-thia-7-azaspiro[3.5]nonane 2,2-dioxide hydrochloride (564 mg, 2.66 mmol) and acetic acid (662 mg, 11.02 mmol) in methanol (8 mL) was added sodium cyanoborohydride (692 mg, 11.0 mmol). The reaction mixture was stirred at 70° C. for 2 h, was diluted with saturated sodium bicarbonate (10 mL) and extracted with dichloromethane (50 mL×3). The combined organic layers were dried over anhydrous Na₂SO₄, filtered and concentrated under reduced pressure. The residue was purified by reverse phase chromatography (water (0.05% NH₃H₂O+10 mM NH₄HCO₃)-ACN) to provide the title compound 43* (the first peak on HPLC, 349 mg, 0.80 mmol, 36.4% yield) and the title compound 44* (the second peak on HPLC, 273 mg, 0.62 mmol, 28.2% yield). LCMS (ESI) [M+H]⁺=432.3. The relative stereochemistry was assigned for arbitrarily for both compounds.

Compound 43*: ¹H NMR (400 MHz, CDCl₃) δ 6.03 (s, 1H), 4.66-4.62 (m, 1H), 3.85 (s, 4H), 2.38-2.30 (m, 3H), 2.21-2.19 (m, 2H), 1.94-1.91 (m, 4H), 1.78-1.72 (m, 2H), 1.67 (s, 2H), 1.60-1.62 (m, 1H), 1.56-1.51 (m, 8H).

Compound 44*: ¹H NMR (400 MHz, CDCl₃) δ 5.99 (s, 1H), 4.69-4.62 (m, 1H), 3.85 (s, 4H), 2.94-2.88 (m, 1H), 2.34-2.30 (m, 5H), 1.91-1.90 (m, 4H), 1.68 (s, 1H), 1.62 (s, 3H), 1.57-1.50 (m, 8H).

### Example U—Compounds 45* and 46*: 7-((1R,3s,5S,6r)-6-(3-(5-(Difluoromethoxy)pyridin-3-yl)-1-isopropyl-1H-pyrazol-5-yl)bicyclo[3.1.0]hexan-3-yl)-2-thia-7-azaspiro[3.5]nonane 2,2-dioxide, and 7-((1R,3r,5S,6r)-6-(3-(5-(difluoromethoxy)pyridin-3-yl)-1-isopropyl-1H-pyrazol-5-yl)bicyclo[3.1.0]hexan-3-yl)-2-thia-7-azaspiro[3.5]nonane 2,2-dioxide

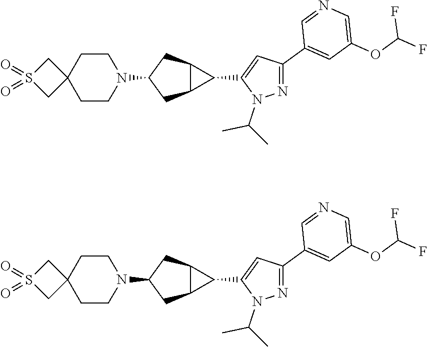 [SMILES: CC(C)n1nc(-c2cncc(OC(F)F)c2)cc1[C@H]1[C@@H]2C[C@@H](N3CCC4(CC3)CS(=O)(=O)C4)C[C@@H]21 ; CC(C)n1nc(-c2cncc(OC(F)F)c2)cc1[C@H]1[C@@H]2C[C@H](N3CCC4(CC3)CS(=O)(=O)C4)C[C@@H]21]

### Step 1: (5-(Difluoromethoxy)pyridin-3-yl)boronic acid

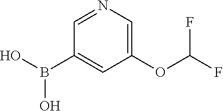 [SMILES: OB(O)c1cncc(OC(F)F)c1]

A solution of potassium acetate (657 mg, 6.7 mmol), Pd(dppf)Cl₂ (163 mg, 0.22 mmol), 3-bromo-5-(difluoromethoxy)pyridine (500 mg, 2.23 mmol), bis(pinacolato)diboron (2.3 g, 8.93 mmol) in 1,4-dioxane (20 mL) was stirred at 100° C. under N₂ atmosphere for 4 h. The reaction mixture was used directly for next step, without further purification. LCMS (ESI) [M+H]⁺=190.1

### Step 2: (1R,5S,6r)-6-(3-(5-(Difluoromethoxy)pyridin-3-yl)-1-isopropyl-1H-pyrazol-5-yl)bicyclo[3.1.0]hexan-3-one

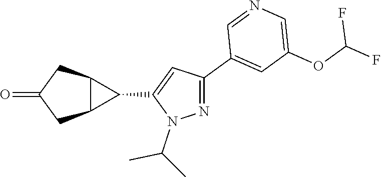 [SMILES: CC(C)n1nc(-c2cncc(OC(F)F)c2)cc1[C@H]1[C@@H]2CC(=O)C[C@@H]21]

To a mixture of potassium carbonate (500 mg, 3.62 mmol) and bis(di-tert-butyl(4-dimethylaminophenyl)phosphine)dichloropalladium(II) (85 mg, 0.12 mmol) in 1,4-dioxane (20 mL) and water (8 mL) were added (1R,5S)-6-(5-iodo-2-isopropyl-1,2,4-triazol-3-yl)bicyclo[3.1.0]hexan-3-one (400 mg, 1.21 mmol) and 3-(difluoromethoxy)-5-(4,4,5,5-tetramethyl-1,3,2-dioxaborolan-2-yl)pyridine (655 mg, 2.42 mmol). The reaction mixture was then stirred at 75° C. under nitrogen atmosphere for 3 h. The mixture was extracted with ethyl acetate (20 mL×3). The combined organic layers were dried over anhydrous Na₂SO₄, filtered and concentrated in vacuo. The residue was purified by silica flash chromatography (0-50% of ethyl acetate in petroleum ether) to provide the title compound (420 mg, 1.21 mmol, 99.8% yield). LCMS (ESI) [M+H]⁺=348.2.

Step 5: Synthesis of title compounds. To a mixture of 2-thia-7-azaspiro[3.5]nonane 2,2-dioxide hydrochloride (62 mg, 0.35 mmol), acetic acid (0.1 mL) and sodium cyanoborohydride (72 mg, 1.15 mmol) in methanol (5 mL) was added (1S,5R)-6-[5-[5-(difluoromethoxy)-3-pyridyl]-2-isopropyl-pyrazol-3-yl]bicyclo[3.1.0]hexan-3-one (100 mg, 0.29 mmol). The reaction mixture was stirred at 60° C. for 6 h. The mixture was concentrated in vacuo and the residue was diluted with ethyl acetate (50 mL), washed with water (30 mL) and brine (30 mL*2). The combined organic layers were dried over anhydrous sodium sulfate, filtered and concentrated in vacuo. The residue was purified by reverse phase chromatography (acetonitrile 30-60%/0.1% NH₄OH in water) to provide the title compound 45* (the first peak on HPLC (basic), 40.6 mg, 0.0793 mmol, 27.6% yield) and the title compound 46* (the second peak on HPLC (basic), 30.3 mg, 0.0562 mmol, 19.5% yield). LCMS (ESI) [M+H]⁺=507.3. The relative stereochemistry was assigned for arbitrarily for both compounds.

Compound 45*: ¹H NMR (400 MHz, CD₃OD) δ 8.78 (d, J=1.6 Hz, 1H), 8.30 (d, J=2.4 Hz, 1H), 7.96 (d, J=2.0 Hz, 1H), 6.99 (t, J=33.2 Hz, 1H), 6.37 (s, 1H), 4.80-4.74 (m, 1H), 3.91 (s, 4H), 2.49-2.08 (m, 7H), 1.97-1.91 (m, 4H), 1.87-1.79 (m, 2H), 1.75-1.74 (m, 3H), 1.52 (d, J=6.8 Hz, 6H).

Compound 46*: ¹H NMR (400 MHz, CD₃OD) δ 8.77 (s, 1H), 8.30 (d, J=2.8 Hz, 1H), 7.95 (s, 1H), 6.99 (t, J=33.2 Hz, 1H), 6.32 (s, 1H), 4.82-4.77 (m, 1H), 3.91 (s, 4H), 3.04-3.02 (m, 1H), 2.49-2.38 (m, 6H), 1.94-1.89 (m, 4H), 1.75-1.72 (m, 3H), 1.62-1.57 (m, 2H), 1.54 (d, J=6.5 Hz, 6H).

### Example V: 7-((1R,3s,5S,6r)-6-(1-Isopropyl-3-(6-(trifluoromethyl)pyridin-2-yl)-1H-pyrazol-5-yl)bicyclo[3.1.0]hexan-3-yl)-2-thia-7-azaspiro[3.5]nonane 2,2-dioxide (Compound 47), and 7-((1R,3r,5S,6r)-6-(1-Isopropyl-3-(6-(trifluoromethyl)pyridin-2-yl)-1H-pyrazol-5-yl)bicyclo[3.1.0]hexan-3-yl)-2-thia-7-azaspiro[3.5]nonane 2,2-dioxide (Compound 48)

The title compounds were synthesized similarly to Compound 45 using (6-(trifluoromethyl)pyridin-2-yl)boronic acid.

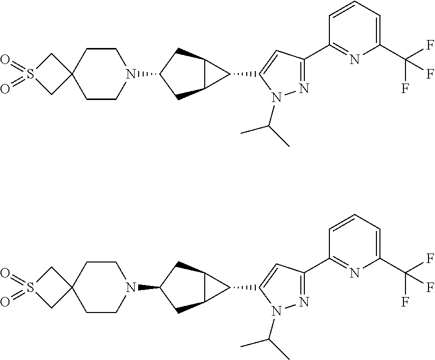 [SMILES: CC(C)n1nc(-c2cccc(C(F)(F)F)n2)cc1[C@H]1[C@@H]2C[C@@H](N3CCC4(CC3)CS(=O)(=O)C4)C[C@@H]21 ; CC(C)n1nc(-c2cccc(C(F)(F)F)n2)cc1[C@H]1[C@@H]2C[C@H](N3CCC4(CC3)CS(=O)(=O)C4)C[C@@H]21]

The isomers were purified by pre-HPLC (water (0.05% FA)-ACN, 40-60%) to afford the title compound 47 (the first peak on SFC, 48.1 mg, 0.0936 mmol, 23.4% yield), and the title compound 48 (the second peak on SFC, 50.7 mg, 0.0986 mmol, 24.6% yield). LCMS (ESI) [M+H]⁺=509.2. The relative stereochemistry was assigned based on ¹H NMR analysis.

Compound 47: ¹H NMR (400 MHz, CD₃OD) δ 8.16 (d, J=8.0 Hz, 1H), 7.98 (t, J=8.0 Hz, 1H), 7.63 (d, J=8.0 Hz, 1H), 6.56 (s, 1H), 4.82-4.73 (m, 1H), 4.04 (s, 4H), 3.30-3.25 (m, 2H), 3.14 (brs, 3H), 2.52 (dd, J=7.6, 12.8 Hz, 2H), 2.23-2.02 (m, 6H), 1.88 (s, 2H), 1.82-1.77 (m, 1H), 1.54 (d, J=6.4 Hz, 6H).

Compound 48: ¹H NMR (400 MHz, CD₃OD) δ 8.16 (d, J=8.0 Hz, 1H), 7.97 (d, J=8.0 Hz, 1H), 7.62 (d, J=8.0 Hz, 1H), 6.50 (s, 1H), 4.85-4.75 (m, 1H), 4.02 (s, 4H), 3.63-3.52 (m, 1H), 3.21-2.69 (m, 4H), 2.68-2.53 (m, 2H), 2.10-2.04 (m, 4H), 1.88-1.72 (m, 5H), 1.56 (d, J=7.2 Hz, 6H).

### Example W: 7-((1R,3s,5S,6r)-6-(1-Isopropyl-3-(2-(trifluoromethyl)pyrimidin-4-yl)-1H-pyrazol-5-yl)bicyclo[3.1.0]hexan-3-yl)-2-thia-7-azaspiro[3.5]nonane 2,2-dioxide (Compound 49), and 7-((1R,3r,5S,6r)-6-(1-isopropyl-3-(2-(trifluoromethyl)pyrimidin-4-yl)-1H-pyrazol-5-yl)bicyclo[3.1.0]hexan-3-yl)-2-thia-7-azaspiro[3.5]nonane 2,2-dioxide (Compound 50)

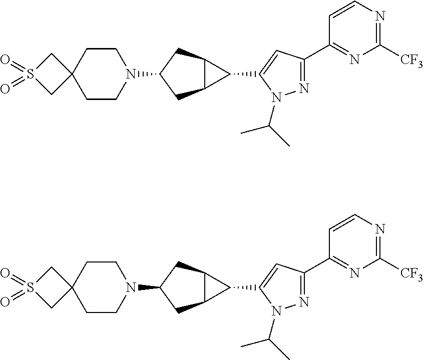 [SMILES: CC(C)n1nc(-c2ccnc(C(F)(F)F)n2)cc1[C@H]1[C@@H]2C[C@@H](N3CCC4(CC3)CS(=O)(=O)C4)C[C@@H]21 ; CC(C)n1nc(-c2ccnc(C(F)(F)F)n2)cc1[C@H]1[C@@H]2C[C@H](N3CCC4(CC3)CS(=O)(=O)C4)C[C@@H]21]

### Step 1: 1-[3-[tert-Butyl(diphenyl)silyl]oxy-6-bicyclo[3.1.0]hexanyl]-3-[2-(trifluoromethyl)pyrimidin-4-yl]propane-1,3-dione

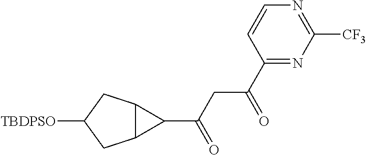

To a solution of 1-[3-[tert-butyl(diphenyl)silyl]oxy-6-bicyclo[3.1.0]hexanyl]ethanone (500 mg, 1.32 mmol) in THF (10 mL) was added NaH (60% in mineral oil, 79 mg, 1.98 mmol) portionwise at 0° C. under N₂. The reaction mixture was stirred for 0.5 h. A solution of methyl 2-(trifluoromethyl) pyrimidine-4-carboxylate (327 mg, 1.58 mmol) in THF (1 mL) was then added at 0° C. under N₂. The resulting mixture was stirred at 20° C. for 2 h. The reaction mixture was quenched with saturated aqueous solution of NH₄Cl (20 mL), diluted with water (30 mL), and extracted with ethyl acetate (30 mL×3). The combined organic layers were concentrated in vacuo. The residue was purified by silica flash chromatography (petroleum ether) to afford the title compound (0.530 g, 73% yield). LCMS (ESI) [M+H]⁺=553.1.

### Step 2: tert-Butyl-[[6-[2-isopropyl-5-[2-(trifluoromethyl)pyrimidin-4-yl]pyrazol-3-yl]-3-bicyclo[3.1.0]hexanyl]oxy]-diphenyl-silane and tert-butyl-[[6-[1-isopropyl-5-[2-(trifluoromethyl)pyrimidin-4-yl]pyrazol-3-yl]-3-bicyclo[3.1.0]hexanyl]oxy]-diphenyl-silane

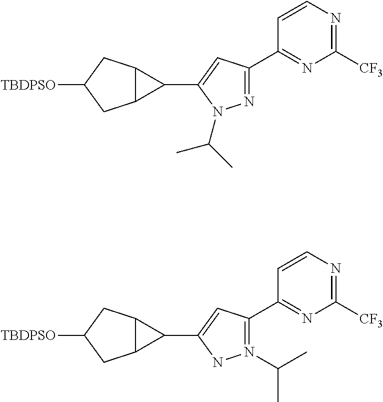

To a solution of 1-[3-[tert-butyl(diphenyl)silyl]oxy-6-bicyclo[3.1.0]hexanyl]-3-[2-(trifluoromethyl)pyrimidin-4-yl]propane-1,3-dione (1.2 g, 2.17 mmol) in ethanol (40 mL) were added isopropyl hydrazine hydrochloride (240 mg, 2.39 mmol) and triethylamine (0.35 mL, 2.39 mmol). The reaction mixture was stirred at 25° C. for 15 h. The mixture was concentrated in vacuo and the residue was then purified by silica flash chromatography (0-5% ethyl acetate in petroleum ether) to afford the title compound (first peak, less polar) (330 mg, 26% yield). LCMS (ESI) [M+H]⁺=591.2; ¹H NMR (400 MHz, CDCl₃) δ 8.78 (d, J=5.2 Hz, 1H), 8.11 (d, J=5.2 Hz, 1H), 7.70-7.61 (m, 4H), 7.49-7.36 (m, 6H), 6.63 (s, 1H), 4.89-4.74 (m, 1H), 4.46-4.40 (m, 1H), 2.43 (t, J=3.2 Hz, 1H), 2.10-1.97 (m, 4H), 1.69-1.66 (m, 2H), 1.62-1.52 (m, 8H), 1.09 (s, 9H). 2D NMR confirmed the regioselectivity of the pyrazole.

The other isomer (second peak, more polar) (400 mg, 31% yield). LCMS (ESI) [M+H]⁺=591.2; ¹H NMR (400 MHz, CDCl₃) δ 8.75 (d, J=4.4 Hz, 1H), 8.05 (d, J=4.8 Hz, 1H), 7.74-7.63 (m, 4H), 7.48-7.35 (m, 7H), 6.62-6.55 (m, 1H), 4.62-4.42 (m, 1H), 4.09-4.02 (m, 1H), 2.18-2.13 (m, 2H), 2.07-1.95 (m, 2H), 1.65-1.62 (m, 2H), 1.46-1.44 (m, 6H), 1.05 (d, J=1.2 Hz, 9H).

### Step 2: 6-[2-Isopropyl-5-[2-(trifluoromethyl)pyrimidin-4-yl]pyrazol-3-yl]bicyclo[3.1.0]hexan-3-ol

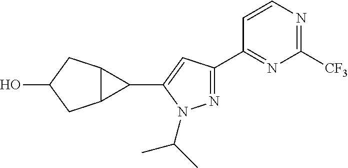 [SMILES: CC(C)n1nc(-c2ccnc(C(F)(F)F)n2)cc1C1C2CC(O)CC21]

To a solution of tert-butyl-[[6-[2-isopropyl-5-[2-(trifluoromethyl)pyrimidin-4-yl]pyrazol-3-yl]-3-bicyclo[3.1.0]hexanyl]oxy]-diphenyl-silane (330 mg, 0.56 mmol) in tetrahydrofuran (10 mL) was added triethylamine trihydrofluoride (2.2 mL). The reaction mixture was stirred at 70° C. for 15 h. The reaction was concentrated in vacuo and the residue was quenched by aqueous NaOH solution (2 N). The resulting mixture was extracted with ethyl acetate (50 mL×3). The combined organic layers were concentrated in vacuo and the resulting residue was purified by silica flash chromatography (0-50% ethyl acetate in petroleum ether) to afford the title compound (150 mg, 76% yield). LCMS (ESI) [M+H]⁺=353.1.

### Step 3: 6-[2-Isopropyl-5-[2-(trifluoromethyl)pyrimidin-4-yl]pyrazol-3-yl]bicycle [3.1.0]hexan-3-one

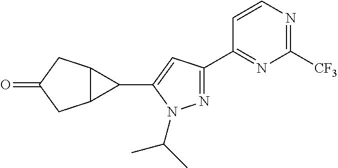 [SMILES: CC(C)n1nc(-c2ccnc(C(F)(F)F)n2)cc1C1C2CC(=O)CC21]

To a solution of 6-[2-isopropyl-5-[2-(trifluoromethyl)pyrimidin-4-yl]pyrazol-3-yl]bicyclo[3.1.0]hexan-3-ol (150 mg, 0.43 mmol) in anhydrous dichloromethane (7 mL) was added Dess-Martin periodinane (270 mg, 0.64 mmol) at 0° C. and the reaction mixture was stirred at 25° C. for 4 h. The reaction mixture was diluted with aq. Na₂SO₃ solution (10 mL) and aq. NaHCO₃ solution (5 mL). The mixture was then extracted with ethyl acetate (30 mL×3). The combined organic layers were dried over anhydrous Na₂SO₄, filtered, and concentrated in vacuo. The residue was purified by silica flash chromatography (0-40% ethyl acetate in petroleum ether) to afford the title compound (125 mg, 84% yield). ¹H NMR (400 MHz, CDCl₃) δ 8.80 (d, J=5.2 Hz, 1H), 8.11 (d, J=5.2 Hz, 1H), 6.75 (s, 1H), 4.72-4.60 (m, 1H), 2.86-2.72 (m, 2H), 2.46 (s, 1H), 2.41 (s, 1H), 2.04-2.00 (m, 2H), 1.60-1.52 (m, 6H), 1.41 (t, J=3.6 Hz, 1H).

Step 3: 7-((1R,3s,5S,6r)-6-(1-Isopropyl-3-(2-(trifluoromethyl)pyrimidin-4-yl)-1H-pyrazol-5-yl)bicyclo[3.1.0]hexan-3-yl)-2-thia-7-azaspiro[3.5]nonane 2,2-dioxide (Compound 49) and 7-((1R,3r,5S,6r)-6-(1-isopropyl-3-(2-(trifluoromethyl)pyrimidin-4-yl)-1H-pyrazol-5-yl)bicyclo[3.1.0]hexan-3-yl)-2-thia-7-azaspiro[3.5]nonane 2,2-dioxide (Compound 50). To a solution of 6-[2-isopropyl-5-[2-(trifluoromethyl)pyrimidin-4-yl]pyrazol-3-yl]bicyclo[3.1.0]hexan-3-one (110 mg, 0.31 mmol) and 2-thia-7-azaspiro[3.5]nonane 2,2-dioxide hydrochloride (100 mg, 0.47 mmol) in anhydrous methanol (6 mL) was added acetic acid (57 mg, 0.94 mmol) and sodium cyanoborohydride (59 mg, 0.94 mmol) at 20° C. and then the reaction mixture was stirred at 60° C. for 15 h. The reaction was purified by pre-HPLC (acetonitrile 25-55%/0.05% NH₄OH in water) to afford the title compound 49 (second, 56.25 mg, 34% yield), and the title compound 50 (first peak, 39.7 mg, 24% yield). LCMS (ESI) [M+H]⁺=510.3. The relative stereochemistry was assigned based on ¹H NMR analysis.

Compound 49: ¹H NMR (400 MHz, CD₃OD) δ 8.84 (d, J=5.2 Hz, 1H), 8.13 (d, J=5.2 Hz, 1H), 6.64 (s, 1H), 4.85-4.78 (m, 1H), 3.91 (s, 4H), 2.85-2.12 (m, 6H), 1.98-1.89 (m, 4H), 1.87-1.72 (m, 5H), 1.54 (d, J=6.4 Hz, 6H).

Compound 50: ¹H NMR (400 MHz, CD₃OD) δ 8.86 (d, J=5.2 Hz, 1H), 8.15 (d, J=5.2 Hz, 1H), 6.61 (s, 1H), 4.89-4.82 (m, 1H), 3.93 (s, 4H), 3.08-2.99 (m, 1H), 2.96-2.03 (m, 6H), 1.98-1.88 (m, 4H), 1.83-1.78 (m, 1H), 1.77-1.72 (m, 2H), 1.66-1.59 (m, 1H), 1.58 (d, J=6.4 Hz, 6H).

### Example X: 6-((1R,3s,5S,6r)-6-(1-Isopropyl-3-(3-(trifluoromethyl)phenyl)-1H-1,2,4-triazol-5-yl)bicyclo[3.1.0]hexan-3-yl)-2-thia-6-azaspiro[3.4]octane 2,2-dioxide (Compound 51), and 6-((1R,3r,5S,6r)-6-(1-isopropyl-3-(3-(trifluoromethyl)phenyl)-1H-1,2,4-triazol-5-yl)bicyclo[3.1.0]hexan-3-yl)-2-thia-6-azaspiro[3.4]octane 2,2-dioxide (Compound 52)

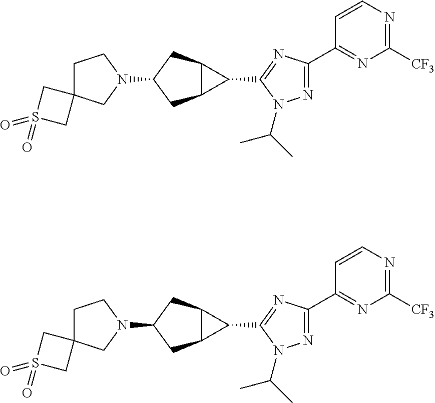 [SMILES: CC(C)n1nc(-c2ccnc(C(F)(F)F)n2)nc1[C@H]1[C@@H]2C[C@@H](N3CCC4(C3)CS(=O)(=O)C4)C[C@@H]21 ; CC(C)n1nc(-c2ccnc(C(F)(F)F)n2)nc1[C@H]1[C@@H]2C[C@H](N3CCC4(C3)CS(=O)(=O)C4)C[C@@H]21]

### Step 1: 5-((1R,5S,6r)-3-((tert-Butyldiphenylsilyl)oxy)bicyclo[3.1.0]hexan-6-yl)-1-isopropyl-3-(3-(trifluoromethyl)phenyl)-1H-1,2,4-triazole

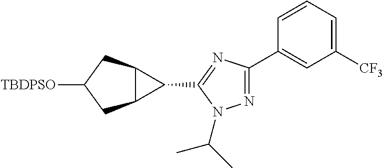

To a solution of (1R,5S)-3-[tert-butyl(diphenyl)silyl]oxybicyclo[3.1.0]hexane-6-carboxylic acid (2.0 g, 5.26 mmol) and 3-(trifluoromethyl)benzamidine (1.48 g, 7.88 mmol) in N,N-dimethylformamide (20 mL) were added HATU (2.2 g, 5.78 mmol) and N,N-diisopropylethylamine (2.75 mL, 15.8 mmol). The reaction mixture was stirred at 20° C. for 1 h. To the reaction mixture were added isopropylhydrazine hydrochloride (872 mg, 7.88 mmol) and acetic acid (3156 mg, 52.6 mmol). The reaction mixture was then stirred at 80° C. for 1.5 h. The reaction mixture was diluted with ethyl acetate (200 mL) and washed with saturated NaHCO₃ (50 mL) and brine (50 mL×3). The combined organic layer was dried over anhydrous MgSO₄, filtered and concentrated in vacuo. The crude residue was purified by silica flash chromatography (0-10% ethyl acetate in petroleum ether) to afford the title compound (2780 mg, 4.71 mmol, 89.7% yield). LCMS (ESI) [M+H]⁺=590.3.

### Step 2: (1R,5S,6r)-6-(1-Isopropyl-3-(3-(trifluoromethyl)phenyl)-1H-1,2,4-triazol-5-yl)bicyclo[3.1.0]hexan-3-ol

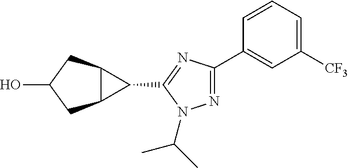

To a stirred solution of tert-butyl-[[(1R,5S)-6-[2-isopropyl-5-[3-(trifluoromethyl)phenyl]-1,2,4-triazol-3-yl]-3-bicyclo[3.1.0]hexanyl]oxy]-diphenyl-silane (2780 mg, 4.71 mmol) in tetrahydrofuran (23 mL) was added triethylamine trihydrofluoride (23 mL, 142 mmol). The reaction mixture was stirred at 70° C. for 16 h. The reaction was quenched by sat. aqueous NaOH solution and extracted with ethyl acetate (50 mL×3). The combined organic layers were washed by brine (20 mL×2), dried over anhydrous sodium sulfate, filtered and concentrated in vacuo. The residue was purified by silica flash chromatography (10% ethyl acetate in petroleum ether) to give the title compound (1.06 g, 3.017 mmol, 64% yield). LCMS (ESI) [M+H]⁺=352.3.

### Step 3: (1R,5S,6r)-6-(1-isopropyl-3-(3-(trifluoromethyl)phenyl)-1H-1,2,4-triazol-5-yl)bicyclo[3.1.0]hexan-3-one

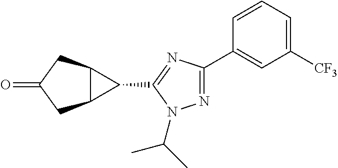 [SMILES: CC(C)n1nc(-c2cccc(C(F)(F)F)c2)nc1[C@H]1[C@@H]2CC(=O)C[C@@H]21]

To a solution of (1R,5S)-6-[2-isopropyl-5-[3-(trifluoromethyl)phenyl]-1,2,4-triazol-3-yl]bicyclo[3.1.0]hexan-3-ol (1.06 mg, 3.02 mmol) in anhydrous dichloromethane (20 mL) was added Dess-Martin periodinane (1.93 g, 4.54 mmol) at 0° C. The reaction mixture was stirred at 25° C. for 16 h. The reaction mixture was then diluted with water (10 mL), then with aq. Na₂SO₃ solution (50 mL) and aq. NaHCO₃ solution (50 mL). The resulting residue was extracted with ethyl acetate (100 mL×3). The combined organic layers were dried over anhydrous Na₂SO₄, filtered, and concentrated in vacuo. The resulting residue was purified by silica flash chromatography (0-30% ethyl acetate in petroleum ether) to afford the title compound (950 mg, 2.72 mmol, 90% yield). LCMS (ESI) [M+H]⁺=350.1.

Step 4: 6-((1R,3s,5S,6r)-6-(1-Isopropyl-3-(3-(trifluoromethyl)phenyl)-1H-1,2,4-triazol-5-yl)bicyclo[3.1.0]hexan-3-yl)-2-thia-6-azaspiro[3.4]octane 2,2-dioxide (Compound 51) and 6-((1R,3r,5S,6r)-6-(1-isopropyl-3-(3-(trifluoromethyl)phenyl)-1H-1,2,4-triazol-5-yl)bicyclo[3.1.0]hexan-3-yl)-2-thia-6-azaspiro[3.4]octane 2,2-dioxide (Compound 52). To a solution of (1R,5S)-6-[2-isopropyl-5-[3-(trifluoromethyl)phenyl]-1,2,4-triazol-3-yl]bicyclo[3.1.0]hexan-3-one (100 mg, 0.28 mmol) in methanol (4 mL) were added acetic acid (0.12 mL, 1.44 mmol), 2-thia-7-azaspiro[3.5]nonane 2,2-dioxide hydrochloride (60 mg, 0.38 mmol) and sodium cyanoborohydride (90 mg, 1.44 mmol). The reaction mixture was stirred at 70° C. for 6 h. The mixture was diluted with ethyl acetate (40 mL×2), and the combined organic layers were washed with aquNaHCO₃ (15 mL×3). The organic phase was dried over anhydrous Na₂SO₄, filtered and concentrated. The residue was purified by reverse phase chromatography (water (0.05% NH₃H₂O+10 nM NH₄HCO₃)-ACN) to afford the title compound 51 (43.32 mg, 0.085 mmol, 30% yield) and the compound 52 (52.6 mg, 0.104 mmol, 36.5% yield). LCMS (ESI): [M+H]⁺=495.3. The relative stereochemistry was assigned based on ¹H NMR analysis.

Compound 51: ¹H NMR (400 MHz, CDCl₃) δ 8.31 (s, 1H), 8.22 (d, J=7.2 Hz, 1H), 7.59 (d, J=8.4 Hz, 1H), 7.52-7.49 (m, 1H), 4.67-4.61 (m, 1H), 4.08 (s, 4H), 2.79 (s, 2H), 2.67 (t, J=7.2 Hz, 2H), 2.48-2.40 (m, 1H), 2.23-2.16 (m, 4H), 2.09 (brs, 2H), 1.90 (t, J=9.6 Hz, 2H), 1.69 (t, J=2.8 Hz, 1H), 1.57 (d, J=6.4 Hz, 6H).

Compound 52: ¹H NMR (400 MHz, CDCl₃) δ 8.30 (s, 1H), 8.22 (d, J=7.2 Hz, 1H), 7.58 (d, J=6.8 Hz, 1H), 7.52-7.48 (m, 1H), 4.62-4.59 (m, 1H), 4.09-4.05 (m, 4H), 2.93 (s, 1H), 2.81 (s, 2H), 2.70 (t, J=6.8 Hz, 2H), 2.22-2.15 (m, 5H), 2.04 (s, 2H), 1.97-1.94 (m, 2H). 1.58 (d, J=6.4 Hz, 6H).

### Example Y—Compounds 53* and 54*: 7-((1R,3s,5S,6r)-6-(1-Ethyl-3-(5-(trifluoromethyl)pyridin-3-yl)-1H-pyrazol-5-yl)bicyclo[3.1.0]hexan-3-yl)-2-thia-7-azaspiro[3.5]nonane 2,2-dioxide, and 7-((1R,3r,5S,6r)-6-(1-ethyl-3-(5-(trifluoromethyl)pyridin-3-yl)-1H-pyrazol-5-yl)bicyclo[3.1.0]hexan-3-yl)-2-thia-7-azaspiro[3.5]nonane 2,2-dioxide

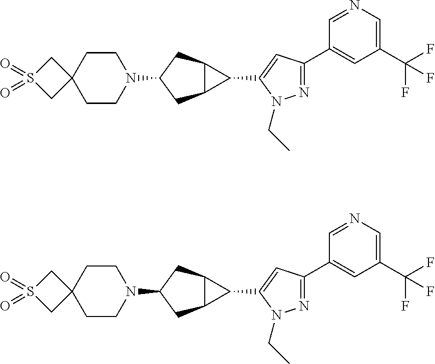 [SMILES: CCn1nc(-c2cncc(C(F)(F)F)c2)cc1[C@H]1[C@@H]2C[C@@H](N3CCC4(CC3)CS(=O)(=O)C4)C[C@@H]21 ; CCn1nc(-c2cncc(C(F)(F)F)c2)cc1[C@H]1[C@@H]2C[C@H](N3CCC4(CC3)CS(=O)(=O)C4)C[C@@H]21]

### Step 1: 3-(5-((1R, 5S, 6r)-3-((tert-Butyldiphenylsilyl)oxy)bicyclo[3.1.0]hexan-6-yl)-1H-pyrazol-3-yl)-5-(trifluoromethyl)pyridine

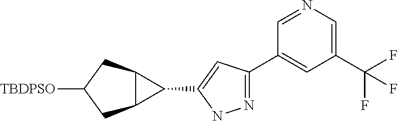

To a mixture of tert-butyl-[[(1R,5S)-6-(3-iodo-1H-pyrazol-5-yl)-3-bicyclo[3.1.0]hexanyl]oxy]-diphenyl-silane (2 g, 3.78 mmol) and 3-(4,4,5,5-tetramethyl-1,3,2-dioxaborolan-2-yl)-5-(trifluoromethyl)pyridine (1.55 g, 5.68 mmol) in 1,4-dioxane (16 mL) and water (4 mL) were added K₂CO₃ (1.57 g, 11.35 mmol) and Pd(dppf)Cl₂ (280 mg, 0.40 mmol). The reaction mixture was stirred under nitrogen atmosphere at 75° C. for 5 h. The reaction mixture was diluted with water (20 mL) and extracted with ethyl acetate (50 mL×2). The combined organic layers were dried over anhydrous Na₂SO₄, filtered and concentrated in vacuo. The residue was purified by silica flash chromatography (0-30% ethyl acetate in petroleum ether) to afford the title compound (1.9 g, 3.47 mmol, 92% yield). LCMS (ESI) [M+H]⁺=548.2.

### Step 2: 3-(5-((1R,5S,6r)-3-((tert-Butyldiphenylsilyl)oxy)bicyclo[3.1.0]hexan-6-yl)-1-ethyl-1H-pyrazol-3-yl)-5-(trifluoromethyl)pyridine

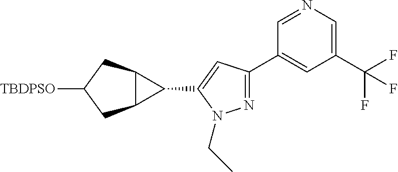

To a solution of 3-(5-((1R, 5S, 6r)-3-((tert-butyldiphenylsilyl)oxy)bicyclo[3.1.0]hexan-6-yl)-1H-pyrazol-3-yl)-5-(trifluoromethyl)pyridine (1.9 g, 3.47 mmol), Cs₂CO₃ (2.26 g, 6.94 mmol) in DMF (15 mL) was added iodoethane (1.39 mL, 17.35 mmol) dropwise. The reaction mixture was stirred at 25° C. for 3 h. The reaction was quenched by water (30 mL) and extracted with ethyl acetate (60 mL×2). The combined organic layers were washed with brine (40 mL×2), dried over anhydrous Na₂SO₄, filtered and concentrated in vacuo. The residue was purified by silica flash chromatography (0-15% ethyl acetate in petroleum ether) to afford the title compound (1.7 g, 2.95 mmol, 85% yield). LCMS (ESI) [M+H]⁺=576.2.

### Step 3: (1R, 5S, 6r)-6-(1-Ethyl-3-(5-(trifluoromethyl)pyridin-3-yl)-1H-pyrazol-5-yl)bicyclo[3.1.0]hexan-3-ol

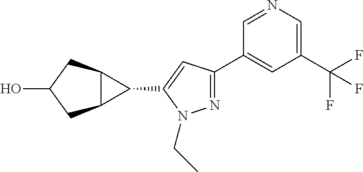

To 3-(5-((1R,5S,6r)-3-((tert-butyldiphenylsilyl)oxy)bicyclo[3.1.0]hexan-6-yl)-1-ethyl-1H-pyrazol-3-yl)-5-(trifluoromethyl)pyridine (1.7 g, 2.95 mmol) was added tetrabutylammoniumfluoride (23.6 mL, 1M in THF, 23.6 mmol). The reaction mixture was stirred at 25° C. for 5 h. The mixture was diluted with water (50 mL) and extracted with ethyl acetate (50 mL×3). The combined organic layers were dried over anhydrous Na₂SO₄, filtered and concentrated in vacuo. The residue was purified by silica flash chromatography (0-50% ethyl acetate in petroleum ether) to afford the title compound (830 mg, 2.46 mmol, 83% yield). LCMS (ESI) [M+H]⁺=338.1.

### Step 4: (1R,5S,6r)-6-(1-Ethyl-3-(5-(trifluoromethyl)pyridin-3-yl)-1H-pyrazol-5-yl)bicyclo[3.1.0]hexan-3-one

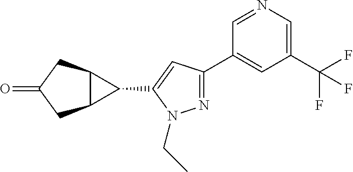 [SMILES: CCn1nc(-c2cncc(C(F)(F)F)c2)cc1[C@H]1[C@@H]2CC(=O)C[C@@H]21]

To a solution of (1R,5S,6r)-6-[2-ethyl-5-[5-(trifluoromethyl)-3-pyridyl]pyrazol-3-yl]bicyclo[3.1.0]hexan-3-ol (830 mg, 2.46 mmol) in anhydrous dichloromethane (15 mL) was added Dess-martin periodinane (1.57 g, 3.69 mmol) and stirred at 25 C for 16 hours. Then the mixture was diluted with H₂O (20 mL), followed by aq. Na₂SO₃ solution (20 mL) and aq. NaHCO₃ solution (20 mL). The resulting mixture was extracted with dichloromethane (50 mL×3). The combined organic layers were dried over anhydrous Na₂SO₄, filtered, and concentrated in vacuo to afford the title compound (600 mg, 1.79 mmol, 73% yield). ¹H NMR (400 MHz, CDCl₃) δ 9.12 (d, J=1.6 Hz, 1H), 8.77 (d, J=1.2 Hz, 1H), 8.30 (s, 1H), 6.28 (s, 1H), 4.25 (m, 2H), 2.85-2.74 (m, 2H), 2.49-2.39 (m, 2H), 2.00 (s, 2H), 1.51 (t, J=7.2 Hz, 3H), 1.40 (t, J=3.6 Hz, 1H).

Step 5: 7-((1R,3s,5S,6r)-6-(1-Ethyl-3-(5-(trifluoromethyl)pyridin-3-yl)-1H-pyrazol-5-yl)bicyclo[3.1.0]hexan-3-yl)-2-thia-7-azaspiro[3.5]nonane 2,2-dioxide (Compound 53) and 7-((1R,3r,5S,6r)-6-(1-ethyl-3-(5-(trifluoromethyl)pyridin-3-yl)-1H-pyrazol-5-yl)bicyclo[3.1.0]hexan-3-yl)-2-thia-7-azaspiro[3.5]nonane 2,2-dioxide (Compound 54). To a solution of 2-thia-7-azaspiro[3.5]nonane 2,2-dioxide hydrochloride (57 mg, 0.27 mmol) and (1R,5S,6r)-6-(1-ethyl-3-(5-(trifluoromethyl)pyridin-3-yl)-1H-pyrazol-5-yl)bicyclo[3.1.0]hexan-3-one (60 mg, 0.18 mmol) in anhydrous methyl alcohol (3 mL) were added NaBH₃CN (34 mg, 0.54 mmol) and acetic acid (0.01 mL, 0.18 mmol) at 20° C. The reaction mixture was stirred at 70° C. for 5 h. The reaction mixture was diluted with NaHCO₃ (10 mL) and extracted with dichloromethane (20 mL×3). The combined organic layers were dried over anhydrous Na₂SO₄, filtered and concentrated in vacuo to afford crude product (100 mg). The crude was purified by reverse phase chromatography (acetonitrile 30 to 60%/0.05% ammonia hydroxide in water) to provide the title compound 53* (second peak, 45.56 mg, 0.09 mmol, 50.5% yield), and the title compound 54* (first peak, 34.31 mg, 0.068 mmol, 38% yield). LCMS (ESI) [M+H]⁺=495.1. The relative stereochemistry was arbitrarily assigned.

Compound 53*: ¹H NMR (400 MHz, CD₃OD) δ 9.15 (d, J=2.0 Hz, 1H), 8.76 (d, J=1.2 Hz, 1H), 8.41 (s, 1H), 6.46 (s, 1H), 4.28 (q, J=7.2 Hz, 2H), 3.91 (s, 4H), 2.61-2.34 (m, 4H), 2.29-2.27 (m, 2H), 1.97-1.79 (m, 7H), 1.77-1.74 (m, 3H), 1.46 (t, J=7.2 Hz, 3H).

Compound 54*: ¹H NMR (400 MHz, CD₃OD) δ 9.14 (d, J=1.6 Hz, 1H), 8.76 (d, J=1.2 Hz, 1H), 8.41 (s, 1H), 6.41 (s, 1H), 4.32 (q, J=7.2 Hz, 2H), 3.91 (s, 4H), 3.07-3.03 (m, 1H), 2.49-2.04 (m, 6H), 1.94-1.89 (m, 4H), 1.79-1.72 (m, 3H), 1.64-1.56 (m, 2H), 1.48 (t, J=7.6 Hz, 3H).

### Example Z: 7-((1R,3s,5S,6r)-6-(1-(tert-Butyl)-3-(trifluoromethyl)-1H-pyrazol-5-yl)bicyclo[3.1.0]hexan-3-yl)-2-thia-7-azaspiro[3.5]nonane 2,2-dioxide (Compound 55), and 7-((1R,3r,5S,6r)-6-(1-(tert-butyl)-3-(trifluoromethyl)-1H-pyrazol-5-yl)bicyclo[3.1.0]hexan-3-yl)-2-thia-7-azaspiro[3.5]nonane 2,2-dioxide (Compound 56)

The title compounds were synthesized similarly to Compound 43 using.

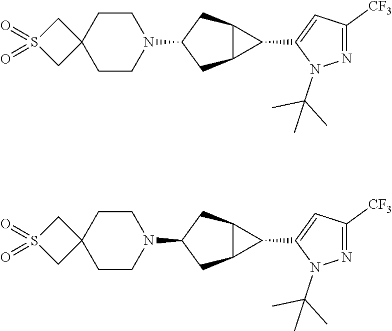 [SMILES: CC(C)(C)n1nc(C(F)(F)F)cc1[C@H]1[C@@H]2C[C@@H](N3CCC4(CC3)CS(=O)(=O)C4)C[C@@H]21 ; CC(C)(C)n1nc(C(F)(F)F)cc1[C@H]1[C@@H]2C[C@H](N3CCC4(CC3)CS(=O)(=O)C4)C[C@@H]21]

The mixture of the diastereoisomers were purified by reverse phase chromatography (acetonitrile 30-60%/0.1% NH₄OH in water) to provide the title compound 55 (first peak on HPLC, 38.79 mg, 0.0845 mmol, 24.2% yield)) and the title compound 56 (second peak on HPLC, 30.9 mg, 0.0672 mmol, 19.2% yield). LCMS (ESI) [M+H]⁺=446.1. The relative stereochemistry was assigned based on ¹H NMR analysis.

Compound 55: ¹H NMR (400 MHz, CD₃OD) δ 6.22 (s, 1H), 3.91 (s, 4H), 2.45-2.33 (m, 4H), 2.29-2.24 (m, 2H), 1.92-1.89 (m, 4H), 1.88-1.72 (m, 6H), 1.69 (s, 9H).

Compound 56: ¹H NMR (400 MHz, CD₃OD) δ 6.14 (s, 1H), 3.90 (s, 4H), 3.00-2.93 (m, 1H), 2.45-2.26 (m, 5H), 2.07-2.05 (m, 1H), 1.91-1.89 (m, 4H), 1.72 (s, 9H), 1.70-1.64 (m, 5H).

### Example AA: 6-((1R,3s,5S,6r)-6-(1-(tert-Butyl)-3-(trifluoromethyl)-1H-pyrazol-5-yl)bicyclo[3.1.0]hexan-3-yl)-2-thia-6-azaspiro[3.4]octane 2,2-dioxide (Compound 57), and 6-((1R,3r,5S,6r)-6-(1-(tert-butyl)-3-(trifluoromethyl)-1H-pyrazol-5-yl)bicyclo[3.1.0]hexan-3-yl)-2-thia-6-azaspiro[3.4]octane 2,2-dioxide (Compound 58)

The title compounds were synthesized similarly to Compound 43 using tert-butylhydrazine hydrochloride.

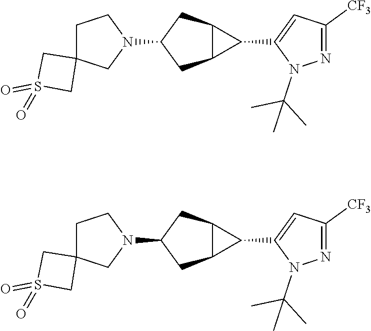 [SMILES: CC(C)(C)n1nc(C(F)(F)F)cc1[C@H]1[C@@H]2C[C@@H](N3CCC4(C3)CS(=O)(=O)C4)C[C@@H]21 ; CC(C)(C)n1nc(C(F)(F)F)cc1[C@H]1[C@@H]2C[C@H](N3CCC4(C3)CS(=O)(=O)C4)C[C@@H]21]

The mixture of the diastereoisomers were purified by reverse phase chromatography to provide the title compound 57* (first peak on HPLC, 23.3 mg, 0.050 mmol, 18% yield) and the title compound 58* (second peak on HPLC, 43.11 mg, 0.094 mmol, 34% yield). LCMS (ESI) [M+H]⁺=432.1. The relative stereochemistry was assigned based on ¹H NMR analysis.

Compound 57: ¹H NMR (400 MHz, CD₃OD) δ 6.22 (s, 1H), 4.08 (s, 4H), 2.85 (s, 2H), 2.72-2.70 (m, 2H), 2.58-2.48 (m, 1H), 2.27-2.15 (m, 4H), 1.92-1.81 (m, 3H), 1.78-1.73 (m, 2H), 1.70 (s, 9H).

Compound 58: ¹H NMR (400 MHz, CD₃OD) δ 6.13 (s, 1H), 4.08 (s, 4H), 2.90-2.86 (m, 1H), 2.82 (s, 2H), 2.71-2.69 (m, 2H), 2.58-2.54 (m, 1H), 2.18-2.12 (m, 4H), 2.06-2.01 (m, 2H), 1.71 (s, 9H), 1.70-1.69 (m, 2H).

### Example BB—Compounds 62* and 63*: 7-((1R,3s,5S,6r)-6-(1-(2,2-Difluoroethyl)-3-(trifluoromethyl)-1H-pyrazol-5-yl)bicyclo[3.1.0]hexan-3-yl)-2-thia-7-azaspiro[3.5]nonane 2,2-dioxide, and 7-((1R,3r,5S,6r)-6-(1-(2,2-difluoroethyl)-3-(trifluoromethyl)-1H-pyrazol-5-yl)bicyclo[3.1.0]hexan-3-yl)-2-thia-7-azaspiro[3.5]nonane 2,2-dioxide

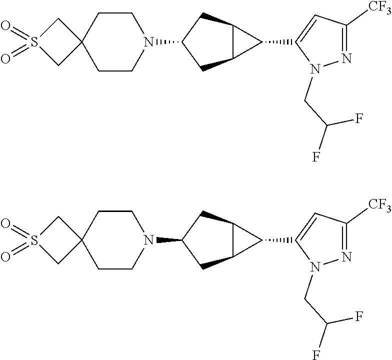 [SMILES: O=S1(=O)CC2(CCN([C@@H]3C[C@@H]4[C@H](C3)[C@H]4c3cc(C(F)(F)F)nn3CC(F)F)CC2)C1 ; O=S1(=O)CC2(CCN([C@H]3C[C@@H]4[C@H](C3)[C@H]4c3cc(C(F)(F)F)nn3CC(F)F)CC2)C1]

### Step 1: 5-(3-((tert-Butyldiphenylsilyl)oxy)bicyclo[3.1.0]hexan-6-yl)-3-(trifluoromethyl)-1H-pyrazole

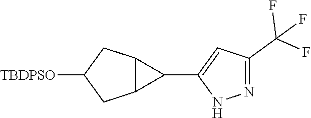

To a solution of hydrazine (140.0 mg, 4.37 mmol) in ethanol (10 mL) were added 1-[3-[tert-butyl(diphenyl)silyl]oxy-6-bicyclo[3.1.0]hexanyl]-4,4,4-trifluoro-butane-1,3-dione (2000 mg, 4.21 mmol) and triethylamine (0.63 mL, 4.39 mmol). The reaction mixture was stirred at 80° C. for 15 h. The mixture was concentrated in vacuo and the residue was purified by silica flash chromatography (0-10% ethyl acetate in petroleum ether) to afford the title compound (1.63 g, 3.46 mmol, 82% yield). LCMS (ESI), [M+H]⁺=471.3.

### Step 2: 5-(3-((tert-Butyldiphenylsilyl)oxy)bicyclo[3.1.0]hexan-6-yl)-1-(2,2-difluoroethyl)-3-(trifluoromethyl)-1H-pyrazole

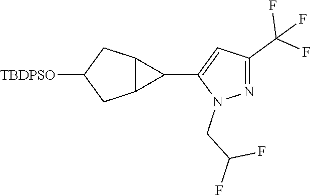

To a solution of tert-butyl-diphenyl-[[6-[3-(trifluoromethyl)-1H-pyrazol-5-yl]-3-bicyclo[3.1.0]hexanyl]oxy]silane (1500 mg, 3.19 mmol) in N,N-dimethylformamide (15 mL) were added cesium carbonate (3116 mg, 9.56 mmol) and 2,2-difluoroethyltrifluoromethanesulfonate (1024 mg, 4.78 mmol). The reaction mixture was stirred at 25° C. for 12 h. Ethyl acetate (120 mL) was added to the reaction mixture and the resulting mixture was washed with brine (30 mL×3). The organic layers were dried over anhydrous Na₂SO₄, filtered and the filtrate was concentrated under reduced pressure. The residue was purified by silica flash chromatography (0-10% ethyl acetate in petroleum ether) to afford the title compound (1280 mg, 2.39 mmol, 75.1% yield). LCMS (ESI), [M+H]⁺=535.1.

### Step 3: 6-(1-(2,2-Difluoroethyl)-3-(trifluoromethyl)-1H-pyrazol-5-yl)bicyclo[3.1.0]hexan-3-ol

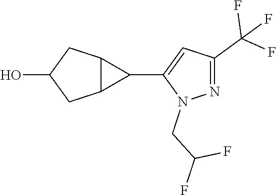 [SMILES: OC1CC2C(C1)C2c1cc(C(F)(F)F)nn1CC(F)F]

To a stirred solution of tert-butyl-[[6-[2-(2,2-difluoro-1-methyl-ethyl)-5-(trifluoromethyl)pyrazol-3-yl]-3-bicyclo[3.1.0]hexanyl]oxy]-diphenyl-silane (1.22 g, 2.22 mmol) in THF (10 mL) was added triethylamine trihydrofluoride (5.46 mL, 33.52 mmol). The reaction mixture was stirred at 70° C. for 16 h. The reaction mixture was quenched by sat. NaOH solution and extracted with ethyl acetate (50 mL×3). The combined organic layers were washed by brine (20 mL×2), dried over anhydrous sodium sulfate, filtered, concentrated in vacuo. The residue was purified by silica flash chromatography (10% ethyl acetate in petroleum ether) to give the title compound (620 mg, 1.998 mmol, 90% yield). LCMS (ESI), [M+H]⁺=297.1.

### Step 4: (1R,5S,6r)-6-(1-(2,2-Difluoroethyl)-3-(trifluoromethyl)-1H-pyrazol-5-yl)bicyclo[3.1.0]hexan-3-one

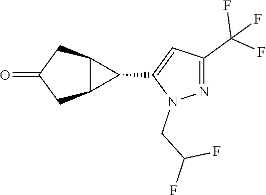 [SMILES: O=C1C[C@@H]2[C@H](C1)[C@H]2c1cc(C(F)(F)F)nn1CC(F)F]

To a solution of 6-[2-(2,2-difluoroethyl)-5-(trifluoromethyl)pyrazol-3-yl]bicyclo[3.1.0]hexan-3-ol (520 mg, 1.76 mmol) in anhydrous dichloromethane (10 mL) was added Dess-Martin periodinane (1121 mg, 2.64 mmol) at 0° C. under N₂ and stirred at 25° C. for 16 h. The reaction mixture was diluted with H₂O (10 mL), then with aq. Na₂SO₃ solution (100 mL) and aq. NaHCO₃ solution (100 mL) and extracted with ethyl acetate (100 mL×3). The combined organic layers were dried over anhydrous Na₂SO₄, filtered, and concentrated to dryness. The residue was purified by silica flash chromatography (0-30% ethyl acetate in petroleum ether) to afford the title compound (250 mg, 0.85 mmol, 48.4% yield). LCMS (ESI), [M+H]⁺=295.0.

Step 5: 7-((1R,3s,5S,6r)-6-(1-(2,2-Difluoroethyl)-3-(trifluoromethyl)-1H-pyrazol-5-yl)bicyclo[3.1.0]hexan-3-yl)-2-thia-7-azaspiro[3.5]nonane 2,2-dioxide (Compound 62) and 7-((1R,3r,5S,6r)-6-(1-(2,2-difluoroethyl)-3-(trifluoromethyl)-1H-pyrazol-5-yl)bicyclo[3.1.0]hexan-3-yl)-2-thia-7-azaspiro[3.5]nonane 2,2-dioxide (Compound 63). To a solution of 6-[2-(2,2-difluoroethyl)-5-(trifluoromethyl)pyrazol-3-yl]bicyclo[3.1.0]hexan-3-one (80 mg, 0.27 mmol) in methanol (4 mL) were added acetic acid (0.11 mL, 1.36 mmol), 2-thia-7-azaspiro[3.5]nonane 2,2-dioxide hydrochloride (69 mg, 0.33 mmol) and sodium cyanoborohydride (85 mg, 1.36 mmol) and stirred at 70° C. for 12 h.

The mixture was diluted with ethyl acetate (40 mL) and washed with NaHCO₃ (15 mL×3). The organic layers were dried over anhydrous Na₂SO₄, filtered and concentrated in vacuo. The residue was purified by reverse phase chromatography (water (0.05% NH₃H₂O+10 mM NH₄HCO₃)-ACN) to afford the title compound 62* (31.84 mg, 0.0667 mmol, 24.5% yield) and the title compound 63* (31.57 mg, 0.0675 mmol, 24.8% yield). LCMS (ESI), [M+H]⁺=454.3. The relative stereochemistry for both compounds was arbitrarily assigned.

Compound 62*: ¹H NMR (400 MHz, DMSO-d₆) δ 6.53-6.24 (m, 2H), 4.79-4.72 (m, 2H), 3.91 (s, 4H), 2.44-2.29 (m, 4H), 2.11-2.07 (m, 3H), 1.86 (s, 1H), 1.76-1.75 (m, 4H), 1.66-1.61 (m, 4H).

Compound 63*: ¹H NMR (400 MHz, DMSO-d₆) δ 6.55-6.26 (m, 2H), 4.79-4.72 (m, 2H), 3.88 (s, 4H), 2.81-2.76 (m, 1H), 2.36-2.21 (m, 3H), 2.13-2.07 (m, 3H), 1.98-1.97 (m, 1H), 1.74-1.60 (m, 8H).

### Example CC—Compounds 64* and 65*: 6-((1R,3s,5S,6r)-6-(1-Ethyl-3-(pyridin-3-yl)-1H-pyrazol-5-yl)bicyclo[3.1.0]hexan-3-yl)-2-thia-6-azaspiro[3.4]octane 2,2-dioxide, and 6-((1R,3r,5S,6r)-6-(1-ethyl-3-(pyridin-3-yl)-1H-pyrazol-5-yl)bicyclo[3.1.0]hexan-3-yl)-2-thia-6-azaspiro[3.4]octane 2,2-dioxide

The title compounds were synthesized similarly to Compounds 53 and 54 using 3-(4,4,5,5-tetramethyl-1,3,2-dioxaborolan-2-yl)pyridine.

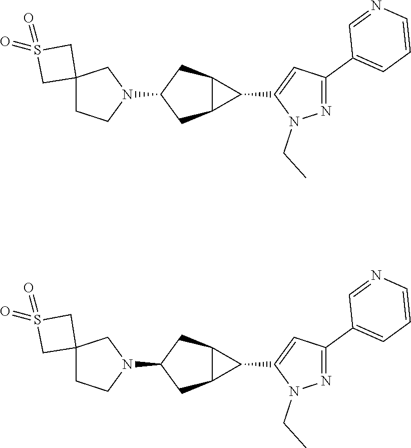 [SMILES: CCn1nc(-c2cccnc2)cc1[C@H]1[C@@H]2C[C@@H](N3CCC4(C3)CS(=O)(=O)C4)C[C@@H]21 ; CCn1nc(-c2cccnc2)cc1[C@H]1[C@@H]2C[C@H](N3CCC4(C3)CS(=O)(=O)C4)C[C@@H]21]

The mixture of the isomers were purified by reverse phase chromatography (acetonitrile 30 to 60%/0.05% ammonia hydroxide in water) to provide the title compound 64* (second peak) and the title compound 65* (first peak). LCMS (ESI) [M+H]⁺=413.2. The relative stereochemistry for both compounds was arbitrarily assigned.

Compound 64*: ¹H NMR (400 MHz, CD₃OD) δ 8.89 (s, 1H), 8.43 (d, J=3.6 Hz, 1H), 8.16-8.14 (m, 1H), 7.44 (dd, J=8.0, 5.2 Hz, 1H), 6.34 (s, 1H), 4.27 (q, J=7.2 Hz, 2H), 4.17-4.07 (m, 4H), 2.86 (s, 2H), 2.71 (t, J=7.2 Hz, 2H), 2.63-2.51 (m, 1H), 2.31-2.15 (m, 4H), 1.91-1.81 (m, 2H), 1.79-1.72 (m, 3H), 1.46 (t, J=7.2 Hz, 3H).

Compound 65*: ¹H NMR (400 MHz, CD₃OD) δ 8.89 (s, 1H), 8.43 (d, J=3.6 Hz, 1H), 8.15-8.13 (m, 1H), 7.44 (dd, J=7.6, 5.2 Hz, 1H), 6.29 (s, 1H), 4.27 (q, J=7.2 Hz, 2H), 4.14-4.05 (m, 4H), 2.96-2.92 (m, 1H), 2.82 (s, 2H), 2.71 (t, J=7.2 Hz, 2H), 2.24-2.13 (m, 5H), 1.97-1.91 (m, 2H), 1.71 (brs, 2H), 1.46 (t, J=7.2 Hz, 3H).

### Example DD—Compounds 66* and 67*: 7-((1R,5S,6r)-6-(1-Ethyl-3-(5-fluoropyridin-3-yl)-1H-pyrazol-5-yl)bicyclo[3.1.0]hexan-3-yl)-2-thia-7-azaspiro[3.5]nonane 2,2-dioxide, and 7-((1R,5S,6r)-6-(1-ethyl-3-(5-fluoropyridin-3-yl)-1H-pyrazol-5-yl)bicyclo[3.1.0]hexan-3-yl)-2-thia-7-azaspiro[3.5]nonane 2,2-dioxide

The title compounds were synthesized similarly to Compounds 53 and 54 using 3-fluoro-5-(4,4,5,5-tetramethyl-1,3,2-dioxaborolan-2-yl)pyridine.

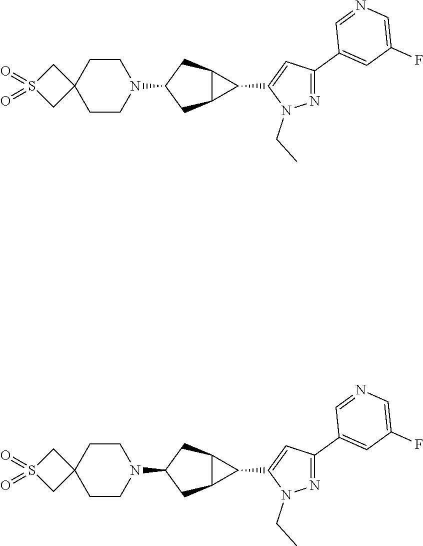 [SMILES: CCn1nc(-c2cncc(F)c2)cc1[C@H]1[C@@H]2C[C@@H](N3CCC4(CC3)CS(=O)(=O)C4)C[C@@H]21 ; CCn1nc(-c2cncc(F)c2)cc1[C@H]1[C@@H]2C[C@H](N3CCC4(CC3)CS(=O)(=O)C4)C[C@@H]21]

The mixture of the isomers were purified by reverse phase chromatography (acetonitrile 30 to 60%/0.05% ammonia hydroxide in water) to provide the title Compound 66* (second peak 39.1 mg, 0.0862 mmol, 31% yield), and the title Compound 67* (first peak, 43.58 mg, 0.097 mmol, 35% yield). LCMS (ESI) [M+H]⁺=445.2. The relative stereochemistry for both compounds was arbitrarily assigned.

Compound 66*: ¹H NMR (400 MHz, CD₃OD) δ 8.76 (s, 1H), 8.35 (s, 1H), 7.93 (d, J=9.6 Hz, 1H), 6.39 (s, 1H), 4.27 (q, J=7.2 Hz, 2H), 3.91 (s, 4H), 2.68-2.48 (m, 4H), 2.34-2.29 (m, 2H), 1.96-1.70 (m, 10H), 1.45 (t, J=7.2 Hz, 3H).

Compound 67*: ¹H NMR (400 MHz, CD₃OD) δ 8.76 (s, 1H), 8.35 (s, 1H), 7.93 (d, J=9.6 Hz, 1H), 6.33 (s, 1H), 4.30 (q, J=7.2 Hz, 2H), 3.91 (s, 4H), 3.06-3.02 (m, 1H), 2.66-2.40 (m, 6H), 1.93-1.91 (m, 4H), 1.79-1.73 (m, 3H), 1.62-1.60 (m, 2H), 1.47 (t, J=7.2 Hz, 3H).

### Example EE—Compounds 68* and 69*: 6-((1R,3s,5S,6r)-6-(1-Ethyl-3-(5-fluoropyridin-3-yl)-1H-pyrazol-5-yl)bicyclo[3.1.0]hexan-3-yl)-2-thia-6-azaspiro[3.4]octane 2,2-dioxide, and 6-((1R,3r,5S,6r)-6-(1-ethyl-3-(5-fluoropyridin-3-yl)-1H-pyrazol-5-yl)bicyclo[3.1.0]hexan-3-yl)-2-thia-6-azaspiro[3.4]octane 2,2-dioxide

The title compounds were synthesized similarly to Compounds 53 and 54 using 3-fluoro-5-(4,4,5,5-tetramethyl-1,3,2-dioxaborolan-2-yl)pyridine and 2-thia-6-azaspiro[3.4]octane 2,2-dioxide.

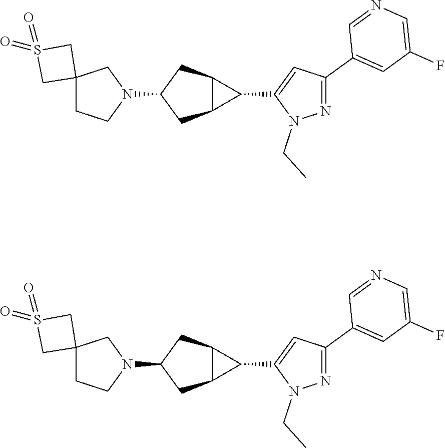 [SMILES: CCn1nc(-c2cncc(F)c2)cc1[C@H]1[C@@H]2C[C@@H](N3CCC4(C3)CS(=O)(=O)C4)C[C@@H]21 ; CCn1nc(-c2cncc(F)c2)cc1[C@H]1[C@@H]2C[C@H](N3CCC4(C3)CS(=O)(=O)C4)C[C@@H]21]

The mixture of the isomers were purified by reverse phase chromatography (acetonitrile 30-60%/0.05% ammonia hydroxide in water) to provide the title compound 68* (second peak, 49.4 mg, 0.112 mmol, 40.1% yield), and the title compound 69* (first peak, 40.9 mg, 0.090 mmol, 32.2% yield). LCMS (ESI) [M+H]⁺=431.1. The relative stereochemistry for both compounds was arbitrarily assigned.

Compound 68*: ¹H NMR (400 MHz, CD₃OD) δ 8.76 (s, 1H), 8.35 (d, J=2.4 Hz, 1H), 7.95-7.92 (m, 1H), 6.39 (s, 1H), 4.27 (q, J=7.2 Hz, 2H), 4.17-4.07 (m, 4H), 2.86 (s, 2H), 2.71 (t, J=7.2 Hz, 2H), 2.62-2.52 (m, 1H), 2.30-2.20 (m, 4H), 1.92-1.86 (m, 2H), 1.80-1.73 (m, 3H), 1.46 (t, J=7.2 Hz, 3H).

Compound 69*: ¹H NMR (400 MHz, CD₃OD) δ 8.76 (s, 1H), 8.35 (d, J=2.4 Hz, 1H), 7.97-7.88 (m, 1H), 6.33 (s, 1H), 4.27 (q, J=7.2 Hz, 2H), 4.16-4.04 (m, 4H), 2.96-2.93 (m, 1H), 2.83 (s, 2H), 2.71 (t, J=7.2 Hz, 2H), 2.25-2.13 (m, 5H), 1.98-1.95 (m, 2H), 1.71 (brs, 2H), 1.47 (t, J=7.2 Hz, 3H).

### Example FF—Compounds 70* and 71*: 7-((1R,3R,5S,6r)-6-(1-((S)-1,1-Difluoropropan-2-yl)-3-(trifluoromethyl)-1H-pyrazol-5-yl)bicyclo[3.1.0]hexan-3-yl)-2-thia-7-azaspiro[3.5]nonane 2,2-dioxide; 7-((1R,3S,5S,6r)-6-(1-((R)-1,1-difluoropropan-2-yl)-3-(trifluoromethyl)-1H-pyrazol-5-yl)bicyclo[3.1.0]hexan-3-yl)-2-thia-7-azaspiro[3.5]nonane 2,2-dioxide; 7-((1R,3S,5S,6r)-6-(1-((S)-1,1-difluoropropan-2-yl)-3-(trifluoromethyl)-1H-pyrazol-5-yl)bicyclo[3.1.0]hexan-3-yl)-2-thia-7-azaspiro[3.5]nonane 2,2-dioxide, and 7-((1R,3R,5S,6r)-6-(1-((R)-1,1-difluoropropan-2-yl)-3-(trifluoromethyl)-1H-pyrazol-5-yl)bicyclo[3.1.0]hexan-3-yl)-2-thia-7-azaspiro[3.5]nonane 2,2-dioxide

The title compounds were synthesized similarly to Compound 62 using 1,1-difluoropropan-2-yl trifluoromethanesulfonate.

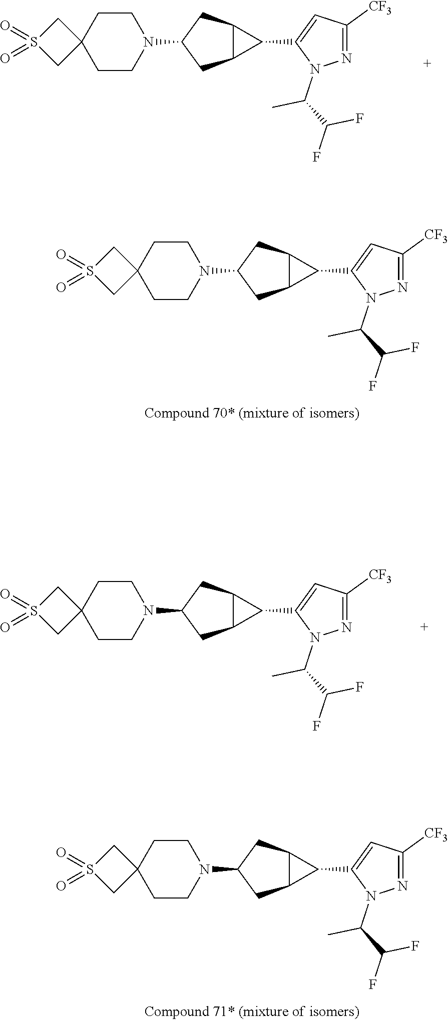 [SMILES: C[C@@H](C(F)F)n1nc(C(F)(F)F)cc1[C@H]1[C@@H]2C[C@@H](N3CCC4(CC3)CS(=O)(=O)C4)C[C@@H]21 ; C[C@H](C(F)F)n1nc(C(F)(F)F)cc1[C@H]1[C@@H]2C[C@@H](N3CCC4(CC3)CS(=O)(=O)C4)C[C@@H]21 ; C[C@@H](C(F)F)n1nc(C(F)(F)F)cc1[C@H]1[C@@H]2C[C@H](N3CCC4(CC3)CS(=O)(=O)C4)C[C@@H]21 ; C[C@H](C(F)F)n1nc(C(F)(F)F)cc1[C@H]1[C@@H]2C[C@H](N3CCC4(CC3)CS(=O)(=O)C4)C[C@@H]21]

The mixture of the isomers were purified by reverse phase chromatography (water (0.05% NH₃H₂O+10 nM NH₄HCO₃)-ACN) to afford the Compound 70* (as a mixture of isomers) (82 mg, 0.1754 mmol, 36% yield, peak 1 on HPLC) and the Compound 71* (as a mixture of isomers) (63 mg, 0.1348 mmol, 27.7% yield, peak 2 on HPLC). LCMS (ESI), [M+H]⁺=468.2.

Compound 70*: ¹H NMR (400 MHz, CDCl₃) δ 6.15-5.86 (m, 2H), 4.70-4.61 (m, 1H), 3.86 (s, 4H), 2.56-2.32 (m, 4H), 2.26-2.21 (m, 2H), 1.93-1.69 (m, 7H), 1.66-1.64 (m, 3H), 1.53 (t, J=3.2 Hz, 3H).

Compound 71*: ¹H NMR (400 MHz, CDCl₃) δ 6.15-5.85 (m, 2H), 4.70-4.61 (m, 1H), 3.85 (s, 4H), 2.90-2.87 (m, 1H), 2.68-2.44 (m, 3H), 2.30-2.13 (m, 3H), 1.90 (brs, 4H), 1.77-1.68 (m, 5H), 1.56 (t, J=3.2 Hz, 3H).

### Example GG: 6-((1R,3s,5S,6r)-6-(1-Ethyl-3-(pyridin-2-yl)-1H-pyrazol-5-yl)bicyclo[3.1.0]hexan-3-yl)-2-thia-6-azaspiro[3.4]octane 2,2-dioxide (Compound 72) and 6-((1R,3r,5S,6r)-6-(1-ethyl-3-(pyridin-2-yl)-1H-pyrazol-5-yl)bicyclo[3.1.0]hexan-3-yl)-2-thia-6-azaspiro[3.4]octane 2,2-dioxide (Compound 73)

The title compounds were synthesized similarly to Compounds 53 and 54 using 2-(4,4,5,5-tetramethyl-1,3,2-dioxaborolan-2-yl)pyridine.

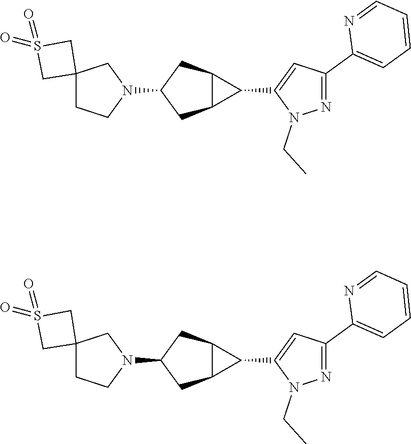 [SMILES: CCn1nc(-c2ccccn2)cc1[C@H]1[C@@H]2C[C@@H](N3CCC4(C3)CS(=O)(=O)C4)C[C@@H]21 ; CCn1nc(-c2ccccn2)cc1[C@H]1[C@@H]2C[C@H](N3CCC4(C3)CS(=O)(=O)C4)C[C@@H]21]

The mixture of the isomers were purified by reverse phase chromatography (acetonitrile 30 to 60%/0.05% ammonia hydroxide in water) to provide the title Compound 72 (Compound 72, 30.5 mg, 0.0732 mmol, 28% yield) and the title Compound 73 (Compound 73, 33.96 mg, 0.0815 mmol, 31.1% yield). LCMS (ESI) [M+H]⁺=413.2. The relative stereochemistry was assigned based on ¹H NMR analysis.

Compound 72: ¹H NMR (400 MHz, CDCl₃) δ 8.59 (d, J=4.8 Hz, 1H), 7.89 (d, J=8.0 Hz, 1H), 7.68 (t, J=2.0 Hz, 1H), 7.18-7.13 (m, 1H), 6.38 (s, 1H), 4.31-4.18 (m, 2H), 4.08 (s, 4H), 2.78 (brs, 2H), 2.71-2.62 (m, 2H), 2.45-2.41 (m, 1H), 2.18-2.14 (m, 4H), 1.88-1.83 (m, 2H), 1.76-1.71 (m, 2H), 1.57 (brs, 1H), 1.53-1.46 (m, 3H).

Compound 73: ¹H NMR (400 MHz, CDCl₃) 8.59 (d, J=4.8 Hz, 1H), 7.89 (d, J=8.0 Hz, 1H), 7.68 (t, J=2.0 Hz, 1H), 7.18-7.13 (m, 1H), 6.38 (s, 1H), 4.25 (q, J=7.2 Hz, 2H), 4.09-4.01 (m, 4H), 2.97-2.87 (m, 1H), 2.80 (s, 2H), 2.69 (t, J=7.2 Hz, 2H), 2.19-2.12 (m, 4H), 2.05 (t, J=3.2 Hz, 1H), 1.92-1.91 (m, 2H), 1.67-1.64 (m, 2H), 1.49 (t, J=7.2 Hz, 3H).

### Example HH: 7-((1R,3r,5S,6r)-6-(1-isopropyl-3-(3-(trifluoromethyl)phenyl)-1H-1,2,4-triazol-5-yl)bicyclo[3.1.0]hexan-3-yl)-2-thia-7-azaspiro[3.5]nonane 2,2-dioxide (Compound 74), and 7-((1R,3s,5S,6r)-6-(1-isopropyl-3-(3-(trifluoromethyl)phenyl)-1H-1,2,4-triazol-5-yl)bicyclo[3.1.0]hexan-3-yl)-2-thia-7-azaspiro[3.5]nonane 2,2-dioxide (Compound 79)

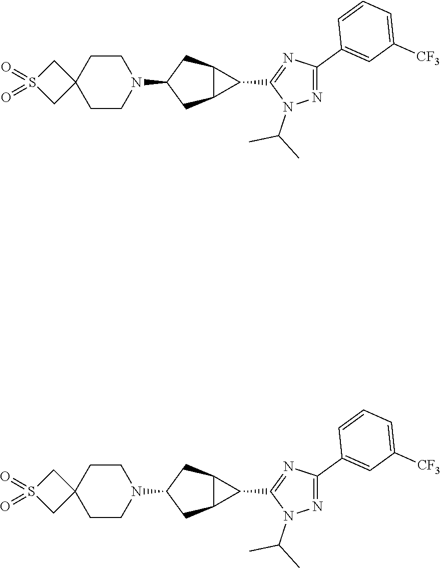 [SMILES: CC(C)n1nc(-c2cccc(C(F)(F)F)c2)nc1[C@H]1[C@@H]2C[C@H](N3CCC4(CC3)CS(=O)(=O)C4)C[C@@H]21 ; CC(C)n1nc(-c2cccc(C(F)(F)F)c2)nc1[C@H]1[C@@H]2C[C@@H](N3CCC4(CC3)CS(=O)(=O)C4)C[C@@H]21]

### Step 1: 5-((1R,5S)-3-((tert-Butyldiphenylsilyl)oxy)bicyclo[3.1.0]hexan-6-yl)-1-isopropyl-3-(3-(trifluoromethyl)phenyl)-1H-1,2,4-triazole

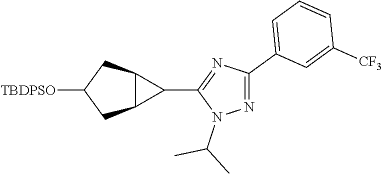

Following the general procedure in J. Org. Chem. 2011, 76, 1177, the title compound 5-((1R,5S)-3-((tert-Butyldiphenylsilyl)oxy)bicyclo[3.1.0]hexan-6-yl)-1-isopropyl-3-(3-(trifluoromethyl)phenyl)-1H-1,2,4-triazole (1.3 g, 84.5% yield) was obtained following flash column chromatography (IprOAc/heptanes). LCMS (ESI) [M+H]⁺=590.3.

### Step 2: (1R,5S)-6-(1-Isopropyl-3-(3-(trifluoromethyl)phenyl)-1H-1,2,4-triazol-5-yl)bicyclo[3.1.0]hexan-3-ol

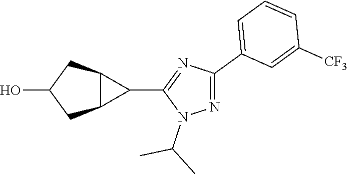

To a mixture of 5-((1R,5S)-3-((tert-butyldiphenylsilyl)oxy)bicyclo[3.1.0]hexan-6-yl)-1-isopropyl-3-(3-(trifluoromethyl)phenyl)-1H-1,2,4-triazole (1.3 g, 2.22 mmol) in THF (22 mL) was added Et₃N·3HF (7.8 mL 44.4 mmol) slowly and the reaction was then heated to 70° C. for 14 h. The reaction mixture was cooled down to room temperature, quenched with saturated NaHCO₃200 mL) and then extracted with IprOAc (100 mL×2). The combined organic layers were washed with brine (100 mL), dried over anhydrous Mg₂SO₄, filtered, concentrated and purified by siliflash chromatography (0-100% heptanes to IprOAc gradient) to give (750 mg, 96% yield). LCMS (ESI) [M+H]⁺=352.2.

### Step 3: (1R,5S,6r)-6-(1-Isopropyl-3-(3-(trifluoromethyl)phenyl)-1H-1,2,4-triazol-5-yl)bicyclo[3.1.0]hexan-3-one

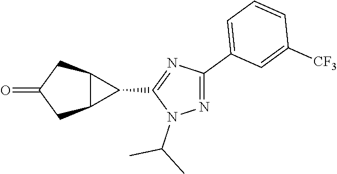 [SMILES: CC(C)n1nc(-c2cccc(C(F)(F)F)c2)nc1[C@H]1[C@@H]2CC(=O)C[C@@H]21]

To a stirred solution of (1R,5S)-6-(1-isopropyl-3-(3-(trifluoromethyl)phenyl)-1H-1,2,4-triazol-5-yl)bicyclo[3.1.0]hexan-3-ol (750 mg, 2.13 mmol) in DCM (21 mL) was added Dess-Martin reagent (1.36 g, 3.2 mmol). The reaction mixture was stirred at room temperature for 2 h. Saturated aqueous NaHCO₃ (100 mL) and sodium sulfite (100 mL) was added slowly to the reaction mixture and the resulting reaction mixture was stirred at room temperature for 0.5 h. The organic layer was separated and the aqueous layer was extracted with DCM (100 mL×2). The combined organic layers were washed with brine, over anhydrous Mg₂SO₄, filtered, concentrated in vacuo and purified by silica flash chromatography (IprOAc/Heptanes) to afford the product (710 mg, 2 mmol). LCMS (ESI) [M+H]⁺=350.1.

Step 4: 7-((1R,3r,5S,6r)-6-(1-Isopropyl-3-(3-(trifluoromethyl)phenyl)-1H-1,2,4-triazol-5-yl)bicyclo[3.1.0]hexan-3-yl)-2-thia-7-azaspiro[3.5]nonane 2,2-dioxide (Compound 74) and 7-((1R,3s,5S,6r)-6-(1-isopropyl-3-(3-(trifluoromethyl)phenyl)-1H-1,2,4-triazol-5-yl)bicyclo[3.1.0]hexan-3-yl)-2-thia-7-azaspiro[3.5]nonane 2,2-dioxide (Compound 79): To a mixture of (1R,5S,6r)-6-(1-isopropyl-3-(3-(trifluoromethyl)phenyl)-1H-1,2,4-triazol-5-yl)bicyclo[3.1.0]hexan-3-one and 2-λ-6-thia-7-azaspiro[3.5]nonane-2,2-dione hydrochloride (133 mg, 0.61 mmol) in methanol (4.1 mL) were added acetic acid (203 μL, 3.54 mmol) and sodium cyanoborohydride (51 mg, 0.81 mmol). The reaction mixture was stirred at 50° C. for 2 h, cooled then diluted with DCM, washed with an aq. saturated NaHCO₃ solution, dried over anhydrous Mg₂SO₄, filtered and concentrated in vacuo. The mixture of cis and trans isomers was separated by a mixture of reverse phase HPLC (interchim HPLC, solvent A: 0.1% formic acid in water, solvent B: acetonitrile, column: XSelect CSH Prep C18, 50×30 mm (5 μm), Method: 5-50% B over 10 min @ 60 mL/min) and chiral SFC (PIC 200 Chiral, solvent B: 0.1% ammonium hydroxide in methanol, Chiralcel OX Column 250×30 mm (5 μm), 40° C., isocratic 30% B gradient @70 mL/min over 4 min) to give the titled compounds (trans: 26 mg, 16% yield and cis: 30.6 mg, 19% yield). LCMS (ESI) [M+H]⁺=509.2. The relative stereochemistry was assigned based on ¹H NMR analysis.

Compound 74: ¹H NMR DMSO-d₆) δ 8.23-8.16 (m, 1H), 8.16-8.11 (m, 1H), 7.78-7.71 (m, 1H), 7.71-7.63 (m, 1H), 4.83 (p, J=6.6 Hz, 1H), 3.92 (s, 4H), 2.84 (p, J=7.4 Hz, 1H), 2.54 (s, 1H), 2.41-2.22 (m, 4H), 2.22-2.09 (m, 2H), 1.93-1.86 (m, 2H), 1.80-1.73 (m, 4H), 1.69 (dd, J=13.6, 6.9 Hz, 2H), 1.47 (d, J=6.5 Hz, 6H).

Compound 79: (400 MHz, DMSO-d₆) δ 8.23-8.16 (m, 1H), 8.16-8.11 (m, 1H), 7.78-7.72 (m, 1H), 7.72-7.63 (m, 1H), 4.87 (p, J=6.6 Hz, 1H), 3.92 (s, 4H), 3.28-3.17 (m, 1H), 2.48-2.19 (m, 4H), 2.12 (dd, J=12.4, 7.0 Hz, 2H), 2.05 (t, J=3.1 Hz, 1H), 1.96-1.87 (m, 2H), 1.81-1.74 (m, 4H), 1.75-1.65 (m, 2H), 1.45 (d, J=6.5 Hz, 6H).

### Example II: 6-((1R,3s,5S,6r)-6-(1-(tert-Butyl)-3-(3-(trifluoromethyl)phenyl)-1H-1,2,4-triazol-5-yl)bicyclo[3.1.0]hexan-3-yl)-2-thia-6-azaspiro[3.4]octane 2,2-dioxide (Compound 75), and 6-((1R,3s,5S,6r)-6-(1-(tert-butyl)-3-(3-(trifluoromethyl)phenyl)-1H-1,2,4-triazol-5-yl)bicyclo[3.1.0]hexan-3-yl)-2-thia-6-azaspiro[3.4]octane 2,2-dioxide (Compound 76)

The title compounds were synthesized similarly to Compound 51 using tert-butylhydrazine hydrochloride.

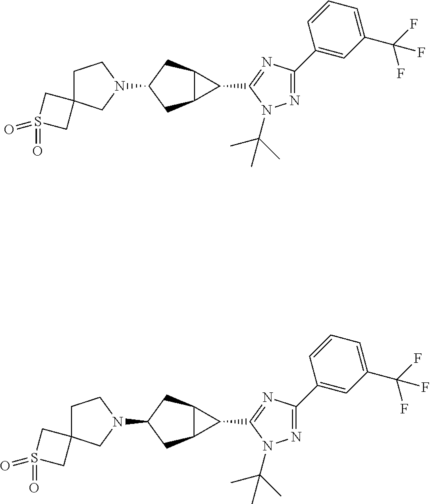 [SMILES: CC(C)(C)n1nc(-c2cccc(C(F)(F)F)c2)nc1[C@H]1[C@@H]2C[C@@H](N3CCC4(C3)CS(=O)(=O)C4)C[C@@H]21 ; CC(C)(C)n1nc(-c2cccc(C(F)(F)F)c2)nc1[C@H]1[C@@H]2C[C@H](N3CCC4(C3)CS(=O)(=O)C4)C[C@@H]21]

The mixture of the isomers were purified by reverse phase chromatography (water (0.05% NH₃H₂O+10 nM NH₄HCO₃)-ACN) to afford the title compound 75 (60.2 mg, 0.13 mmol, 37% yield), first peak) and the title compound 76 (16 mg, 0.04 mmol, 11% yield, second). LCMS (ESI) [M+H]⁺=509.2. The relative stereochemistry was assigned based on ¹H NMR analysis.

Compound 75: ¹H NMR (400 MHz, CDCl₃) δ 8.29 (s, 1H), 8.21 (d, J=8.0 Hz, 1H), 7.59 (d, J=8.4 Hz, 1H), 7.42 (d, J=8.0 Hz, 1H), 4.08 (s, 4H), 2.81 (s, 2H), 2.71 (t, J=7.2 Hz, 2H), 2.46-2.38 (m, 1H), 2.23-2.15 (m, 6H), 1.90-1.87 (m, 3H), 1.57 (s. 9H).

Compound 76: ¹H NMR (400 MHz, CDCl₃) δ 8.29 (s, 1H), 8.21 (d, J=8.0 Hz, 1H), 7.59 (d, J=8.4 Hz, 1H), 7.42 (d, J=8.0 Hz, 1H), 4.05 (s, 4H), 2.83-2.81 (m, 1H), 2.76 (s, 2H), 2.71 (t, J=7.2 Hz, 2H), 2.58-2.56 (m, 1H), 2.17-2.02 (m, 8H), 1.57 (s. 9H).

### Example JJ—Compounds 77* and 78*: 6-((1R,3s,5S,6r)-6-(1-(tert-Butyl)-3-(trifluoromethyl)-1H-1,2,4-triazol-5-yl)bicyclo[3.1.0]hexan-3-yl)-2-thia-6-azaspiro[3.4]octane 2,2-dioxide, and 6-((1R,3r,5S,6r)-6-(1-(tert-butyl)-3-(trifluoromethyl)-1H-1,2,4-triazol-5-yl)bicyclo[3.1.0]hexan-3-yl)-2-thia-6-azaspiro[3.4]octane 2,2-dioxide

The title compounds were synthesized similarly to Compound 51 using tert-butylhydrazine hydrochloride and 2,2,2-trifluoroacetimidamide.

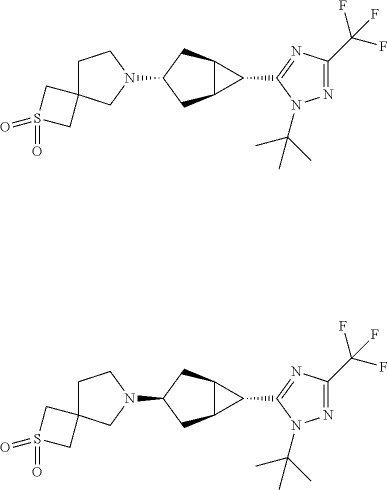 [SMILES: CC(C)(C)n1nc(C(F)(F)F)nc1[C@H]1[C@@H]2C[C@@H](N3CCC4(C3)CS(=O)(=O)C4)C[C@@H]21 ; CC(C)(C)n1nc(C(F)(F)F)nc1[C@H]1[C@@H]2C[C@H](N3CCC4(C3)CS(=O)(=O)C4)C[C@@H]21]

The mixture of the isomers were purified by reverse phase chromatography (water (0.05% NH₃H₂O+10 mM NH₄HCO₃)-ACN, 50%-80%) to afford the title Compound 77* (23.1 mg, 29.8% yield, first peak on HPLC/second peak on SFC) and the title Compound 78* (32.72 mg, 42.6% yield, second peak on HPLC/first peak on SFC). LCMS (ESI) [M+H]⁺=433.2. The relative stereochemistry for both compounds was arbitrarily assigned.

Compound 77*: ¹H NMR (400 MHz, CD₃OD) δ 4.15-4.07 (m, 4H), 2.86 (s, 2H), 2.72 (t, J=7.2 Hz, 2H), 2.63-2.57 (m, 1H), 2.27-2.18 (m, 4H), 2.07 (s, 3H), 1.88 (t, J=10.4 Hz, 2H), 1.72 (s, 9H).

Compound 78*: ¹H NMR (400 MHz, CD₃OD) δ 4.13-4.06 (m, 4H), 2.92-2.89 (m, 1H), 2.86 (t, J=3.2 Hz, 1H), 2.83 (s, 2H), 2.72 (t, J=7.2 Hz, 2H), 2.17 (t, J=7.2 Hz, 2H), 2.12 (d, J=3.6 Hz, 4H), 2.01 (br s, 2H), 1.73 (s, 9H).

### Example KK—Compounds 80* and 81*: (S)-7-((1R,3s,5S,6S)-6-(1-Isopropyl-3-(6-(trifluoromethyl)pyridin-2-yl)-1H-pyrazol-5-yl)bicyclo[3.1.0]hexan-3-yl)-2-thia-7-azaspiro[4.5]decane 2,2-dioxide, and (R)-7-((1R,3s,5S,6S)-6-(1-isopropyl-3-(6-(trifluoromethyl)pyridin-2-yl)-1H-pyrazol-5-yl)bicyclo[3.1.0]hexan-3-yl)-2-thia-7-azaspiro[4.5]decane 2,2-dioxide

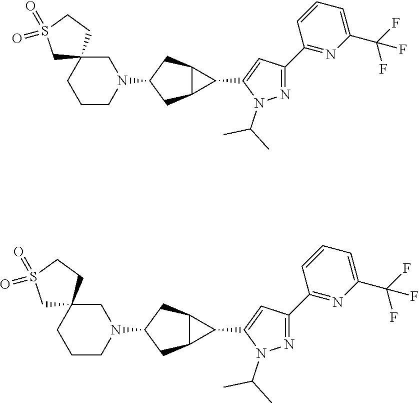 [SMILES: CC(C)n1nc(-c2cccc(C(F)(F)F)n2)cc1[C@H]1[C@@H]2C[C@@H](N3CCC[C@]4(CCS(=O)(=O)C4)C3)C[C@@H]21 ; CC(C)n1nc(-c2cccc(C(F)(F)F)n2)cc1[C@H]1[C@@H]2C[C@@H](N3CCC[C@@]4(CCS(=O)(=O)C4)C3)C[C@@H]21]

The title compounds were synthesized similarly to Compounds 3 and 4 using 2-thia-7-azaspiro[4.5]decane 2,2-dioxide and 2-(4,4,5,5-tetramethyl-1,3,2-dioxaborolan-2-yl)-6-(trifluoromethyl)pyridine. The relative stereochemistry for both compounds was arbitrarily assigned.

This mixture of isomers was purified by chiral SFC (Daicel Chiralpak AD (250 mm*50 mm, 10 μm); 0.1% NH₃H₂O; EtOH; 30%; 60 mL/min) to afford the title compound 80* (first peak, 36.2 mg, 0.0686 mmol, 45% yield) and the title compound 81* (the second peak on SFC, 40.8 mg, 0.0757 mmol, 55% yield). LCMS (ESI) [M+H]⁺=523.1.

Compound 80*: ¹H NMR (400 MHz, CD₃OD) δ 8.16 (d, J=8.0 Hz, 1H), 7.97 (t, J=8.0 Hz, 1H), 7.62 (d, J=7.6 Hz, 1H), 6.47 (s, 1H), 4.84-4.77 (m, 1H), 3.20 (dd, J=8.4, 7.2 Hz, 2H), 2.98-2.68 (m, 4H), 2.27-2.19 (m, 2H), 2.16-1.91 (m, 5H), 1.88-1.82 (m, 2H), 1.76-1.65 (m, 5H), 1.55 (d, J=6.8 Hz, 6H), 1.51-1.38 (m, 2H).

Compound 81*: ¹H NMR (400 MHz, CD₃OD) δ 8.15 (d, J=8.0 Hz, 1H), 7.97 (t, J=8.0 Hz, 1H), 7.62 (d, J=7.6 Hz, 1H), 6.46 (s, 1H), 4.84-4.80 (m, 1H), 3.24-3.17 (m, 2H), 2.98-2.66 (m, 4H), 2.28-2.19 (m, 2H), 2.16-1.91 (m, 5H), 1.89-1.81 (m, 2H), 1.77-1.64 (m, 5H), 1.55 (d, J=6.8 Hz, 6H), 1.52-1.36 (m, 2H).

### Example LL—Compounds 82* and 83*: (S)-7-((1R,3r,5S,6S)-6-(1-isopropyl-3-(6-(trifluoromethyl)pyridin-2-yl)-1H-pyrazol-5-yl)bicyclo[3.1.0]hexan-3-yl)-2-thia-7-azaspiro[4.5]decane 2,2-dioxide and (R)-7-((1R,3r,5S,6S)-6-(1-isopropyl-3-(6-(trifluoromethyl)pyridin-2-yl)-1H-pyrazol-5-yl)bicyclo[3.1.0]hexan-3-yl)-2-thia-7-azaspiro[4.5]decane 2,2-dioxide

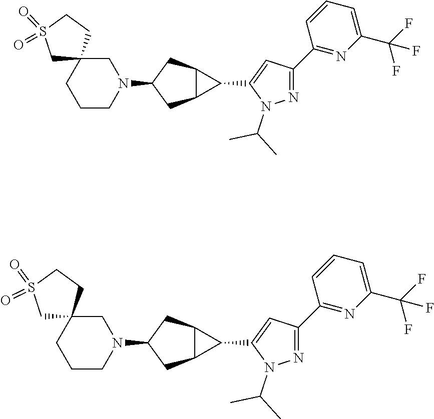 [SMILES: CC(C)n1nc(-c2cccc(C(F)(F)F)n2)cc1[C@H]1[C@@H]2C[C@H](N3CCC[C@]4(CCS(=O)(=O)C4)C3)C[C@@H]21 ; CC(C)n1nc(-c2cccc(C(F)(F)F)n2)cc1[C@H]1[C@@H]2C[C@H](N3CCC[C@@]4(CCS(=O)(=O)C4)C3)C[C@@H]21]

The title compounds were synthesized similarly to Compounds 3 and 4 using 2-thia-7-azaspiro[4.5]decane 2,2-dioxide and 2-(4,4,5,5-tetramethyl-1,3,2-dioxaborolan-2-yl)-6-(trifluoromethyl)pyridine.

This mixture of isomers was separated using chiral SFC (Daicel Chiralpak AD (250 mm*50 mm, 10 μm); 0.1% NH₃ in H₂O; EtOH; 30%; 60 mL/min) to afford the title compound 82* (first peak, 43.5 mg, 0.08 mmol, 32.1% yield), and the title compound 83* (second peak, 36.7 mg, 0.0688 mmol, 27.7% yield). LCMS (ESI) [M+H]⁺=523.2. The relative stereochemistry for both compounds was arbitrarily assigned

Compound 82*: ¹H NMR (400 MHz, CD₃OD) δ 8.15 (d, J=8.0 Hz, 1H), 7.97 (t, J=8.0 Hz, 1H), 7.62 (d, J=7.6 Hz, 1H), 6.50 (s, 1H), 4.81-4.75 (m, 1H), 3.18 (t, J=7.2 Hz, 2H), 2.95-2.92 (m, 1H), 2.71-2.62 (m, 2H), 2.57-2.45 (m, 1H), 2.26-1.97 (m, 6H), 1.87-1.77 (m, 2H), 1.72-1.56 (m, 6H), 1.53 (d, J=6.8 Hz, 6H), 1.51-1.36 (m, 2H).

Compound 83*: ¹H NMR (400 MHz, CD₃OD) δ 8.15 (d, J=8.0 Hz, 1H), 7.97 (t, J=8.0 Hz, 1H), 7.62 (d, J=7.6 Hz, 1H), 6.50 (s, 1H), 4.81-4.75 (m, 1H), 3.18 (t, J=7.6 Hz, 2H), 2.95-2.92 (m, 1H), 2.75-2.70 (m, 2H), 2.57-2.45 (m, 1H), 2.31-2.00 (m, 6H), 1.89-1.78 (m, 2H), 1.75-1.59 (m, 6H), 1.53 (d, J=6.8 Hz, 6H), 1.51-1.38 (m, 2H).

### Example MM—Compounds 86* and 87*: (S)-7-((1R,3s,5S,6S)-6-(1-(2,2-Difluoroethyl)-3-(trifluoromethyl)-1H-pyrazol-5-yl)bicyclo[3.1.0]hexan-3-yl)-2-thia-7-azaspiro[4.5]decane 2,2-dioxide, and (R)-7-((1R,3s,5S,6S)-6-(1-(2,2-difluoroethyl)-3-(trifluoromethyl)-1H-pyrazol-5-yl)bicyclo[3.1.0]hexan-3-yl)-2-thia-7-azaspiro[4.5]decane 2,2-dioxide

The title compounds were synthesized similarly to Compounds 62 and 63 using 2-thia-7-azaspiro[4.5]decane 2,2-dioxide.

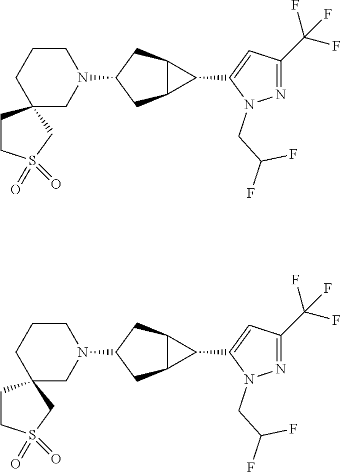

The mixture of the two cis isomers was purified by chiral SFC (Daicel Chiralpak AD (250 nm*30 mm, 10 μm); 0.1% NH₃ in H₂O; EtOH; 25%; 60 mL/min) to provide the title compound 86* (first peak on SFC, 41.4 mg, 34.5% yield) and the title compound 87* (second peak on SFC, 44.8 mg, 37.3% yield). LCMS (ESI) [M+H]⁺=468.2. The relative stereochemistry for both compounds was arbitrarily assigned

Compound 86*: ¹H NMR (400 MHz, CD₃OD) δ 6.37-6.08 (m, 2H), 4.68-4.64 (m, 2H), 3.20-3.14 (m, 2H), 2.93 (d, J=13.6 Hz, 1H), 2.71 (s, 2H), 2.54-2.46 (m, 1H), 2.26-2.21 (m, 2H), 2.19-2.11 (m, 2H), 2.10-1.97 (m, 2H), 1.83-1.76 (m, 3H), 1.75-1.72 (m, 3H), 1.64-1.44 (m, 4H).

Compound 87*: ¹H NMR (400 MHz, CD₃OD) δ 6.37-6.08 (m, 2H), 4.68-4.64 (m, 2H), 3.20-3.16 (m, 2H), 2.93 (d, J=13.6 Hz, 1H), 2.72 (s, 2H), 2.54-2.46 (m, 1H), 2.26-2.21 (m, 2H), 2.19-2.11 (m, 2H), 2.10-1.99 (m, 2H), 1.83-1.76 (m, 3H), 1.75-1.69 (m, 5H), 1.58-1.44 (m, 2H).

### Example NN—Compounds 88* and 89*: (S)-7-((1R,3r,5S,6S)-6-(1-(2,2-Difluoroethyl)-3-(trifluoromethyl)-1H-pyrazol-5-yl)bicyclo[3.1.0]hexan-3-yl)-2-thia-7-azaspiro[4.5]decane 2,2-dioxide, and (R)-7-((1R,3r,5S,6R)-6-(1-(2,2-difluoroethyl)-3-(trifluoromethyl)-1H-pyrazol-5-yl)bicyclo[3.1.0]hexan-3-yl)-2-thia-7-azaspiro[4.5]decane 2,2-dioxide

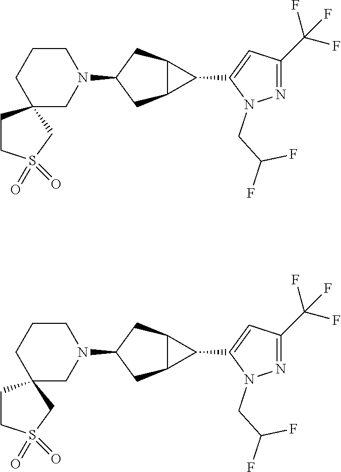 [SMILES: O=S1(=O)CC[C@]2(CCCN([C@H]3C[C@@H]4[C@H](C3)[C@H]4c3cc(C(F)(F)F)nn3CC(F)F)C2)C1 ; O=S1(=O)CC[C@@]2(CCCN([C@H]3C[C@@H]4[C@H](C3)[C@H]4c3cc(C(F)(F)F)nn3CC(F)F)C2)C1]

The title compounds were synthesized similarly to Compounds 62 and 63 using 2-thia-7-azaspiro[4.5]decane 2,2-dioxide. The mixture of trans isomers was separated using chiral SFC (Daicel Chiralpak AD (250 mm*30 mm, 10 μm); 0.1% NH₃ in H₂O; ethanol; 25%; 60 mL/min) to provide the title compound 88* (first peak on SFC, 54.7 mg, 45.1% yield) and the title compound 89* (second peak on SFC, 50.9 mg, 42% yield). LCMS (ESI) [M+H]⁺=468.2. The relative stereochemistry for both compounds was arbitrarily assigned

Compound 88*: ¹H NMR (400 MHz, CD₃OD) δ 6.39-6.10 (m, 2H), 4.70-4.65 (m, 2H), 3.21-3.17 (m, 2H), 2.93-2.87 (m, 2H), 2.85-2.78 (m, 2H), 2.23-2.09 (m, 4H), 2.07-2.01 (m, 2H), 1.98 (t, J=3.2 Hz, 1H), 1.90-1.84 (m, 2H), 1.73-1.64 (m, 5H), 1.56-1.50 (m, 1H), 1.47-1.34 (m, 1H).

Compound 89*: ¹H NMR (400 MHz, CD₃OD) δ 6.39-6.10 (m, 2H), 4.70-4.65 (m, 2H), 3.21-3.17 (m, 2H), 2.93-2.78 (m, 4H), 2.23-2.16 (m, 2H), 2.13-2.07 (m, 2H), 2.05-1.97 (m, 3H), 1.90-1.84 (m, 2H), 1.71-1.64 (m, 4H), 1.58-1.47 (m, 2H), 1.41-1.29 (m, 1H).

### Example OO—Compounds 90* and 91*: 6-((1R,3s,5S,6r)-6-(1-(2,2-Difluoroethyl)-3-(trifluoromethyl)-1H-pyrazol-5-yl)bicyclo[3.1.0]hexan-3-yl)-2-thia-6-azaspiro[3.4]octane 2,2-dioxide, and 6-((1R,3r,5S,6r)-6-(1-(2,2-difluoroethyl)-3-(trifluoromethyl)-1H-pyrazol-5-yl)bicyclo[3.1.0]hexan-3-yl)-2-thia-6-azaspiro[3.4]octane 2,2-dioxide

The title compounds were synthesized similarly to Compounds 62 and 63 using 2-thia-6-azaspiro[3.4]octane 2,2-dioxide.

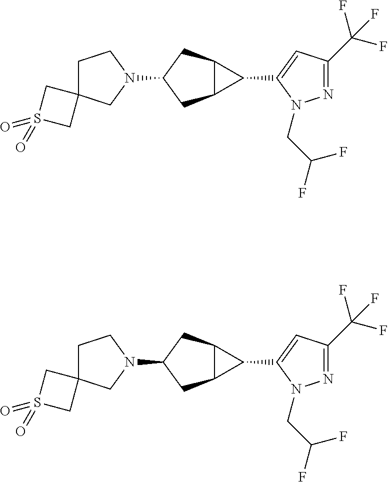 [SMILES: O=S1(=O)CC2(CCN([C@@H]3C[C@@H]4[C@H](C3)[C@H]4c3cc(C(F)(F)F)nn3CC(F)F)C2)C1 ; O=S1(=O)CC2(CCN([C@H]3C[C@@H]4[C@H](C3)[C@H]4c3cc(C(F)(F)F)nn3CC(F)F)C2)C1]

The mixture of diastereoisomers were purified by reverse phase chromatography (water (0.05% NH₃H₂O+10 mM NH₄HCO₃); ACN) to provide the title compound 90* (second peak on SFC, 48.0 mg, 45.1% yield) and the title compound 91* (first peak on SFC, 42.4 mg, 27.3% yield). LCMS (ESI) [M+H]⁺=440.2.

Compound 90*: ¹H NMR (400 MHz, CD₃OD) δ 6.38-6.08 (m, 2H), 4.69-4.64 (m, 2H), 4.15-4.08 (m, 4H), 2.85 (s, 2H), 2.72-2.66 (m, 2H), 2.58-2.50 (m, 1H), 2.28-2.18 (m, 4H), 1.86-1.81 (m, 3H), 1.75-1.74 (m, 2H).

Compound 91*: ¹H NMR (400 MHz, CD₃OD) δ 6.38-6.09 (m, 2H), 4.67-4.62 (m, 2H), 4.11-4.05 (m, 4H), 2.97-2.90 (m, 1H), 2.80 (s, 2H), 2.70 (t, J=7.2 Hz, 2H), 2.31 (t, J=3.2 Hz, 1H), 2.17-2.11 (m, 4H), 2.01-1.97 (m, 2H), 1.69 (s, 2H).

### Example PP—Compounds 92* and 93*: (R)-7-((1R,3s,5S,6R)-6-(1-Isopropyl-3-(trifluoromethyl)-1H-pyrazol-5-yl)bicyclo[3.1.0]hexan-3-yl)-2-thia-7-azaspiro[4.5]decane 2,2-dioxide, and (S)-7-((1R,3s,5S,6S)-6-(1-isopropyl-3-(trifluoromethyl)-1H-pyrazol-5-yl)bicyclo[3.1.0]hexan-3-yl)-2-thia-7-azaspiro[4.5]decane 2,2-dioxide

The title compounds were synthesized similarly to Compounds 43 and 44 using 2-thia-7-azaspiro[4.5]decane 2,2-dioxide.

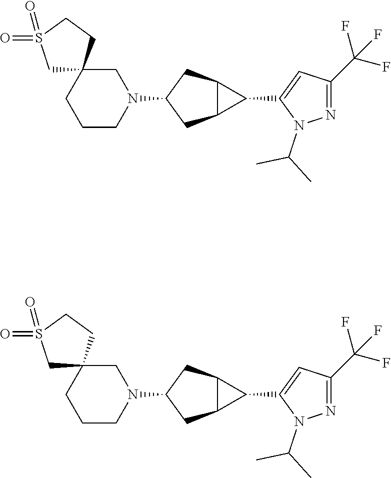 [SMILES: CC(C)n1nc(C(F)(F)F)cc1[C@H]1[C@@H]2C[C@@H](N3CCC[C@@]4(CCS(=O)(=O)C4)C3)C[C@@H]21 ; CC(C)n1nc(C(F)(F)F)cc1[C@H]1[C@@H]2C[C@@H](N3CCC[C@]4(CCS(=O)(=O)C4)C3)C[C@@H]21]

The mixture of diastereoisomers was separated using chiral SFC (Daicel Chiralpak AD (250 mm*30 mm, 10 μm); 0.1% NH₃ in H₂O, EtOH, 20%; 60 mL/min) to afford the title compound 92* (46.1 mg, 38% yield, first peak on SFC) and the title compound 93* (56.2 mg, 46% yield, second peak on SFC). LCMS (ESI) [M+H]⁺=446.1. The relative stereochemistry for both compounds was arbitrarily assigned

Compound 92*: ¹H NMR (400 MHz, CD₃OD) δ 6.15 (s, 1H), 4.80-4.75 (m, 1H), 3.19-3.14 (m, 2H), 2.93 (d, J=13.6 Hz, 1H), 2.71 (brs, 2H), 2.54-2.46 (m, 1H), 2.25-2.20 (m, 2H), 2.14-1.98 (m, 4H), 1.83-1.77 (m, 2H), 1.72-1.68 (m, 4H), 1.65-1.62 (m, 1H), 1.58-1.51 (m, 1H), 1.47 (d, J=6.8 Hz, 8H).

Compound 93*: ¹H NMR (400 MHz, CD₃OD) δ 6.15 (s, 1H), 4.82-4.75 (m, 1H), 3.19-3.15 (m, 2H), 2.93 (d, J=13.6 Hz, 1H), 2.71 (brs, 2H), 2.52-2.46 (m, 1H), 2.25-2.20 (m, 2H), 2.18-1.99 (m, 4H), 1.83-1.79 (m, 2H), 1.72-1.68 (m, 4H), 1.65-1.64 (m, 1H), 1.58-1.52 (m, 1H), 1.47 (d, J=6.8 Hz, 8H).

### Example QQ—Compounds 94* and 95*: (R)-7-((1R,3r,5S,6R)-6-(1-Isopropyl-3-(trifluoromethyl)-1H-pyrazol-5-yl)bicyclo[3.1.0]hexan-3-yl)-2-thia-7-azaspiro[4.5]decane 2,2-dioxide and (S)-7-((1R,3r,5S,6S)-6-(1-Isopropyl-3-(trifluoromethyl)-1H-pyrazol-5-yl)bicyclo[3.1.0]hexan-3-yl)-2-thia-7-azaspiro[4.5]decane 2,2-dioxide

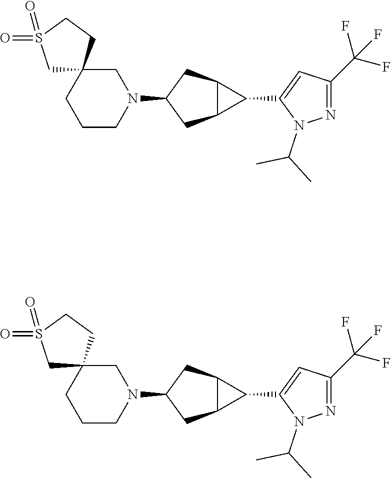 [SMILES: CC(C)n1nc(C(F)(F)F)cc1[C@H]1[C@@H]2C[C@H](N3CCC[C@@]4(CCS(=O)(=O)C4)C3)C[C@@H]21 ; CC(C)n1nc(C(F)(F)F)cc1[C@H]1[C@@H]2C[C@H](N3CCC[C@]4(CCS(=O)(=O)C4)C3)C[C@@H]21]

The title compounds were synthesized similarly to Compounds 43 and 44 using 2-thia-7-azaspiro[4.5]decane 2,2-dioxide. The mixture of trans isomers (100 mg, 0.22 mmol) was separated using chiral SFC (Daicel Chiralpak AD (250 mm*30 mm, 10 μm); 0.1% NH₃ in H₂O; EtOH; 15%; 60 mL/min) to afford the title compound 94* (46.3 mg, 45.8% yield, first peak on SFC) and the title compound 95* (40.4 mg, 39.6% yield, second peak on SFC). LCMS (ESI) [M+H]⁺=446.1. The relative stereochemistry for both compounds was arbitrarily assigned

Compound 94*: ¹H NMR (400 MHz, CD₃OD) δ 6.11 (s, 1H), 4.84-4.79 (m, 1H), 3.22-3.18 (m, 2H), 2.94-2.86 (m, 4H), 2.24-2.16 (m, 2H), 2.13-1.99 (m, 4H), 1.92 (t, J=3.2 Hz, 1H), 1.88-1.83 (m, 2H), 1.71-1.52 (m, 6H), 1.49 (d, J=6.8 Hz, 6H), 1.44 (brs, 1H).

Compound 95*: ¹H NMR (400 MHz, CD₃OD) δ 6.11 (s, 1H), 4.83-4.80 (m, 1H), 3.22-3.18 (m, 2H), 2.94-2.86 (m, 4H), 2.24-2.18 (m, 2H), 2.16-1.99 (m, 4H), 1.92 (t, J=3.2 Hz, 1H), 1.88-1.83 (m, 2H), 1.70-1.52 (m, 6H), 1.49 (d, J=6.8 Hz, 6H), 1.44 (brs, 1H).

### Example RR: 7-((1R,3s,5S,6r)-6-(1-(2,2-Difluoroethyl)-5-(trifluoromethyl)-1H-pyrazol-3-yl)bicyclo[3.1.0]hexan-3-yl)-2-thia-7-azaspiro[3.5]nonane 2,2-dioxide (Compound 96), and and 7-((1R,3r,5S,6r)-6-(1-(2,2-difluoroethyl)-5-(trifluoromethyl)-1H-pyrazol-3-yl)bicyclo[3.1.0]hexan-3-yl)-2-thia-7-azaspiro[3.5]nonane 2,2-dioxide (Compound 97)

The title compounds were synthesized similarly to Compounds 62 and 63 using 2-thia-7-azaspiro[3.5]nonane 2,2-dioxide.

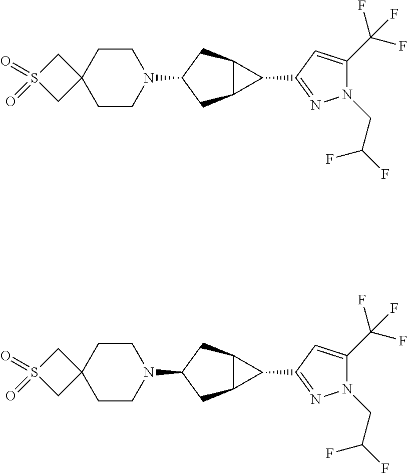 [SMILES: O=S1(=O)CC2(CCN([C@@H]3C[C@@H]4[C@H](C3)[C@H]4c3cc(C(F)(F)F)n(CC(F)F)n3)CC2)C1 ; O=S1(=O)CC2(CCN([C@H]3C[C@@H]4[C@H](C3)[C@H]4c3cc(C(F)(F)F)n(CC(F)F)n3)CC2)C1]

The mixture of the diastereoisomers were purified by reverse phase chromatography (water (0.05% NH₃H₂O+10 mM NH₄HCO₃)-ACN, 50-80%) to provide the title compound-96 (second peak, 11.2 mg, 6.8% yield) and the title compound 97 (first peak, 19.2 mg, 11.7% yield). LCMS (ESI) [M+H]⁺=454.3. The relative stereochemistry for both compounds was arbitrarily assigned

Compound 96*: ¹H NMR (400 MHz, CDCl₃) δ 6.28-5.98 (m, 2H), 4.47-4.40 (m, 2H), 3.85 (s, 4H), 2.35-2.16 (m, 7H), 1.93-1.90 (m, 4H), 1.81-1.74 (m, 3H), 1.68-1.65 (m, 2H).

Compound 97*: ¹H NMR (400 MHz, CDCl₃) δ 6.28-5.98 (m, 2H), 4.47-4.40 (m, 2H), 3.84 (s, 4H), 2.93-2.85 (m, 1H), 2.63-2.24 (m, 7H), 1.90 (t, J=5.2 Hz, 4H), 1.80-1.79 (m, 1H), 1.57-1.51 (m, 3H).

### Example SS—Compounds 98* and 99*: 6-((1R,3s,5S,6r)-6-(1-Isopropyl-3-(5-(trifluoromethyl)pyridin-3-yl)-1H-pyrazol-5-yl)bicyclo[3.1.0]hexan-3-yl)-2-thia-6-azaspiro[3.3]heptane 2,2-dioxide, and 6-((1R,3r,5S,6r)-6-(1-isopropyl-3-(5-(trifluoromethyl)pyridin-3-yl)-1H-pyrazol-5-yl)bicyclo[3.1.0]hexan-3-yl)-2-thia-6-azaspiro[3.3]heptane 2,2-dioxide

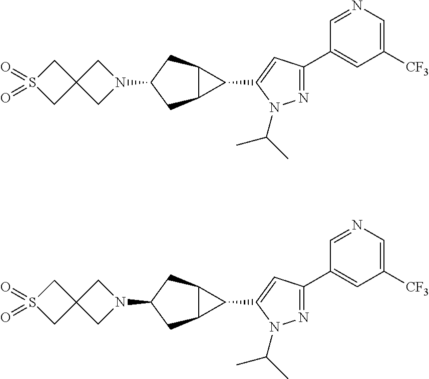 [SMILES: CC(C)n1nc(-c2cncc(C(F)(F)F)c2)cc1[C@H]1[C@@H]2C[C@@H](N3CC4(C3)CS(=O)(=O)C4)C[C@@H]21 ; CC(C)n1nc(-c2cncc(C(F)(F)F)c2)cc1[C@H]1[C@@H]2C[C@H](N3CC4(C3)CS(=O)(=O)C4)C[C@@H]21]

The title compounds were synthesized similarly to Compounds 11 and 12 using 2-thia-6-azaspiro[3.3]heptane 2,2-dioxide and 3-(4,4,5,5-tetramethyl-1,3,2-dioxaborolan-2-yl)-5-(trifluoromethyl)pyridine. The mixture of diastereoisomers was purified by reverse phase chromatography (Xtimate C18, 150*25 mm*5 μm; water (NH₃H₂O+NH₄HCO₃)-ACN, 30-60%) to provide title compounds. LCMS (ESI) [M+H]⁺=481.2.

Compound 98*: ¹H NMR (400 MHz, DMSO-d₆) δ ppm 9.21 (s, 1H), 8.84 (s, 1H), 8.36 (s, 1H), 6.63 (s, 1H), 5.75 (s, 1H), 4.74 (quin, J=6.5 Hz, 1H), 4.30 (s, 4H), 3.28 (s, 4H), 2.64-2.72 (m, 1H), 2.07 (s, 1H), 2.00 (dd, J=12.3, 6.9 Hz, 2H), 1.74-1.79 (m, 1H), 1.62 (br s, 2H), 1.49-1.59 (m, 2H), 1.44 (d, J=6.6 Hz, 6H),

Compound 99*: ¹H NMR (400 MHz, DMSO-d₆) δ ppm 9.22 (d, J=1.6 Hz, 1H), 8.85 (s, 1H), 8.37 (s, 1H), 6.60 (s, 1H), 5.76 (s, 1H), 4.66 (quin, J=6.6 Hz, 1H), 4.30 (s, 4H), 3.21 (s, 4H), 2.84 (br t, J=6.9 Hz, 1H), 2.56 (t, J=3.2 Hz, 1H), 2.08 (s, 1H), 1.82-1.93 (m, 2H), 1.72 (d, J=13.8 Hz, 2H), 1.64 (br s, 2H), 1.49 (d, J=6.6 Hz, 6H).

### Example TT: 6-((1R,3s,5S,6r)-6-(1-Isopropyl-3-(2-(trifluoromethyl)pyrimidin-5-yl)-1H-pyrazol-5-yl)bicyclo[3.1.0]hexan-3-yl)-2-thia-6-azaspiro[3.4]octane 2,2-dioxide (Compound 102), and 6-((1R,3r,5S,6r)-6-(1-Isopropyl-3-(2-(trifluoromethyl)pyrimidin-5-yl)-1H-pyrazol-5-yl)bicyclo[3.1.0]hexan-3-yl)-2-thia-6-azaspiro[3.4]octane 2,2-dioxide (Compound 103)

The title compounds were synthesized similarly to Compounds 45 and 46 using (2-(trifluoromethyl)pyrimidin-5-yl)boronic acid and 2-thia-6-azaspiro[3.4]octane 2,2-dioxide.

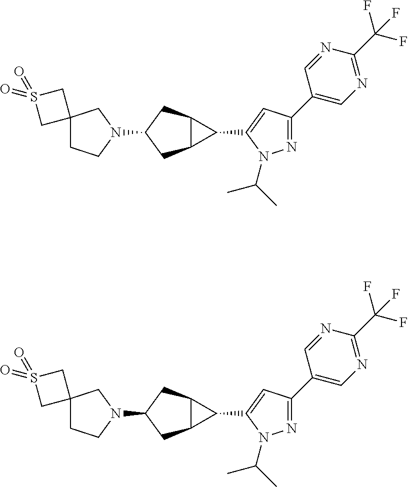 [SMILES: CC(C)n1nc(-c2cnc(C(F)(F)F)nc2)cc1[C@H]1[C@@H]2C[C@@H](N3CCC4(C3)CS(=O)(=O)C4)C[C@@H]21 ; CC(C)n1nc(-c2cnc(C(F)(F)F)nc2)cc1[C@H]1[C@@H]2C[C@H](N3CCC4(C3)CS(=O)(=O)C4)C[C@@H]21]

The mixture of the diastereoisomers was purified by reverse phase chromatography (water (0.05% NH₃H₂O+10 mM NH₄HCO₃)-ACN, 60-90%) to afford the title compound 102 (45.7 mg, 0.09 mmol, 40% yield, the first peak on basic HPLC) and the title compound 103 (58.1 mg, 0.12 mmol, 51% yield, the second peak on basic HPLC). LCMS (ESI) [M+H]⁺=496.3. The relative stereochemistry was assigned based on ¹H NMR analysis.

Compound 102: ¹H NMR (400 MHz, CD₃OD) δ 9.27 (s, 2H), 6.51 (s, 1H), 4.84-4.77 (m, 1H), 4.16-4.09 (m, 4H), 2.86 (s, 2H), 2.72 (t, J=7.2 Hz, 2H), 2.60-2.53 (m, 1H), 2.29-2.19 (m, 4H), 1.89-1.83 (m, 2H), 1.79-1.78 (m, 1H), 1.76-1.74 (m, 2H), 1.53 (d, J=6.8 Hz, 6H).

Compound 103: ¹H NMR (400 MHz, CD₃OD) δ 9.27 (s, 2H), 6.46 (s, 1H), 4.83-4.76 (m, 1H), 4.13-4.07 (m, 4H), 2.98-2.96 (m, 1H), 2.84 (s, 2H), 2.72 (t, J=7.2 Hz, 2H), 2.20-2.15 (m, 5H), 1.97-1.94 (m, 2H), 1.74-1.71 (m, 2H), 1.55 (d, J=6.8 Hz, 6H).

### Example UU—Compounds 104* and 105*: 6-((1R,3S,5S,6r)-6-(3-((1s,4S)-4-Hydroxy-4-(trifluoromethyl)cyclohexyl)-1-isopropyl-1H-pyrazol-5-yl)bicyclo[3.1.0]hexan-3-yl)-2-thia-6-azaspiro[3.4]octane 2,2-dioxide, and 6-((1R,3S,5S,6r)-6-(3-((1r,4R)-4-Hydroxy-4-(trifluoromethyl)cyclohexyl)-1-isopropyl-1H-pyrazol-5-yl)bicyclo[3.1.0]hexan-3-yl)-2-thia-6-azaspiro[3.4]octane 2,2-dioxide

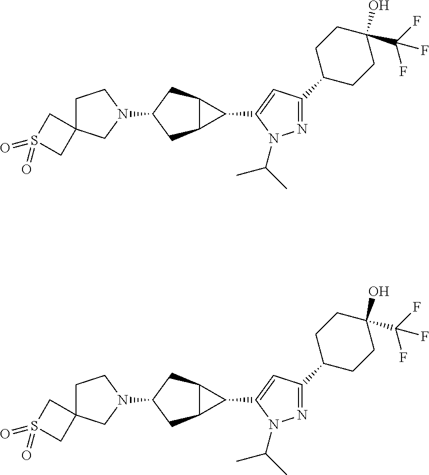 [SMILES: CC(C)n1nc([C@H]2CC[C@](O)(C(F)(F)F)CC2)cc1[C@H]1[C@@H]2C[C@@H](N3CCC4(C3)CS(=O)(=O)C4)C[C@@H]21 ; CC(C)n1nc([C@H]2CC[C@@](O)(C(F)(F)F)CC2)cc1[C@H]1[C@@H]2C[C@@H](N3CCC4(C3)CS(=O)(=O)C4)C[C@@H]21]

### Step 1: 6-((1R,3s,5S,6r)-6-(1-Isopropyl-3-(1,4-dioxaspiro[4.5]dec-7-en-8-yl)-1H-pyrazol-5-yl)bicyclo[3.1.0]hexan-3-yl)-2-thia-6-azaspiro[3.4]octane 2,2-dioxide

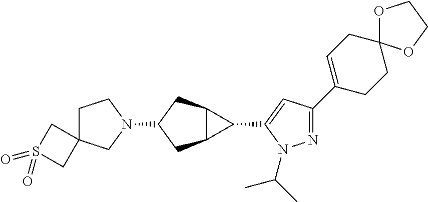

To a mixture of potassium carbonate (183 mg, 1.33 mmol) and bis(di-tert-butyl(4-dimethylaminophenyl)phosphine)dichloropalladium(II) (31 mg, 0.040 mmol) in 1,4-dioxane (20 mL) and water (3 mL) were added 6-((1R,3s,5S,6r)-6-(3-iodo-1-isopropyl-1H-pyrazol-5-yl)bicyclo[3.1.0]hexan-3-yl)-2-thia-6-azaspiro[3.4]octane 2,2-dioxide (210 mg, 0.44 mmol) and 4,4,5,5-tetramethyl-2-(1,4-dioxaspiro[4.5]dec-7-en-8-yl)-1,3,2-dioxaborolane (235 mg, 0.88 mmol). The reaction mixture was stirred at 75° C. under N₂ for 16 h. The reaction mixture was then filtered and concentrated in vacuo. The crude residue was purified by silica flash chromatography (0-50% ethyl acetate in petroleum ether) to give the title compound (220 mg, 0.36 mmol, 82% yield) as a white solid. LCMS (ESI) [M+H]⁺=488.3.

### Step 2: 6-((1R,3s,5S,6r)-6-(1-Isopropyl-3-(1,4-dioxaspiro[4.5]decan-8-yl)-1H-pyrazol-5-yl)bicyclo[3.1.0]hexan-3-yl)-2-thia-6-azaspiro[3.4]octane 2,2-dioxide

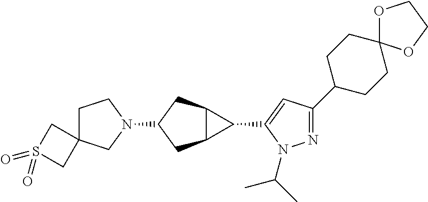

To a solution of 6-((1R,3s,5S,6r)-6-(1-isopropyl-3-(1,4-dioxaspiro[4.5]dec-7-en-8-yl)-1H-pyrazol-5-yl)bicyclo[3.1.0]hexan-3-yl)-2-thia-6-azaspiro[3.4]octane 2,2-dioxide (200 mg, 0.41 mmol) in ethanol (20 mL) was added 10% wt. Pd/C (175 mg, 0.16 mmol) at 25° C. The reaction mixture was stirred at 25° C. for 2 h under H₂ (15 psi). The reaction mixture was filtered, and the filtrate was concentrated in vacuo to afford the title compound (190 mg, 0.388 mmol, 94.6% yield) as a yellow oil. LCMS (ESI) [M+H]⁺=490.3.

### Step 3: 4-(5-((1R,3s,5S,6r)-3-(2,2-Dioxido-2-thia-6-azaspiro[3.4]octan-6-yl)bicyclo [3.1.0]hexan-6-yl)-1-isopropyl-1H-pyrazol-3-yl)cyclohexanone

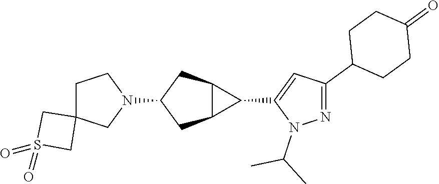 [SMILES: CC(C)n1nc(C2CCC(=O)CC2)cc1[C@H]1[C@@H]2C[C@@H](N3CCC4(C3)CS(=O)(=O)C4)C[C@@H]21]

To 6-((1R,3s,5S,6r)-6-(1-isopropyl-3-(1,4-dioxaspiro[4.5]decan-8-yl)-1H-pyrazol-5-yl)bicyclo[3.1.0]hexan-3-yl)-2-thia-6-azaspiro[3.4]octane 2,2-dioxide (190 mg, 0.388 mmol) was added HCl in water (3.0 mL, 3 mmol, 1M). The reaction mixture was stirred at 25° C. for 1 h. The pH of the reaction mixture was adjusted to 8 at 0° C. with saturated aq. NaHCO₃ solution (40 mL). The reaction mixture extracted with ethyl acetate (40 mL×3) and the combined layers were washed with brine (10 mL×3) and dried over anhydrous Na₂SO₄. The mixture was filtered and concentrated in vacuo, the residue was purified by silica flash chromatography (0-10% methanol in dichloromethane) to afford the title compound (165 mg, 0.370 mmol, 95% yield) as a colorless oil. LCMS (ESI) [M+H]⁺=446.3.

### Step 4: 6-((1R,3s,5S,6r)-6-(1-Isopropyl-3-(4-(trifluoromethyl)-4-((trimethylsilyl)oxy) cyclohexyl)-1H-pyrazol-5-yl)bicyclo[3.1.0]hexan-3-yl)-2-thia-6-azaspiro[3.4]octane 2,2-dioxide

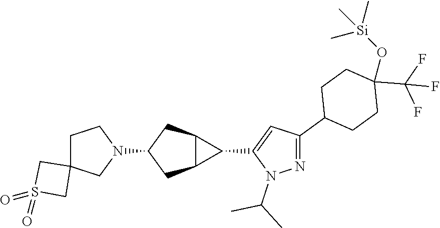 [SMILES: CC(C)n1nc(C2CCC(O[Si](C)(C)C)(C(F)(F)F)CC2)cc1[C@H]1[C@@H]2C[C@@H](N3CCC4(C3)CS(=O)(=O)C4)C[C@@H]21]

Cesium fluoride (112.0 mg, 0.74 mmol) was added to a solution of 4-(5-((1R,3s,5S,6r)-3-(2,2-dioxido-2-thia-6-azaspiro[3.4]octan-6-yl)bicyclo[3.1.0]hexan-6-yl)-1-isopropyl-1H-pyrazol-3-yl)cyclohexanone (60.0 mg, 0.13 mmol) in tetrahydrofuran (4 mL) at 0° C. Trifluoromethyltrimethylsilane (115 mg, 0.81 mmol) was slowly added, and the reaction mixture was stirred for 20 min at 0° C. The reaction mixture was diluted with water (20 mL) and extracted with ethyl acetate (50 mL×2). The combined organic layers were dried over anhydrous Na₂SO, filtered and concentrated in vacuo. The residue was purified by silica flash chromatography (0-2% dichloromethane in methanol) to afford the title compound (50 mg, 63.2% yield) as a white solid. LCMS (ESI) [M+H]⁺=588.3

### Step 5: 6-((1R,3S,5S,6r)-6-(3-((1s,4S)-4-Hydroxy-4-(trifluoromethyl)cyclohexyl)-1-isopropyl-1H-pyrazol-5-yl)bicyclo[3.1.0]hexan-3-yl)-2-thia-6-azaspiro[3.4]octane 2,2-dioxide and 6-((1R,3S,5S,6r)-6-(3-((1r,4R)-4-Hydroxy-4-(trifluoromethyl)cyclohexyl)-1-isopropyl-1H-pyrazol-5-yl)bicyclo[3.1.0]hexan-3-yl)-2-thia-6-azaspiro[3.4]octane 2,2-dioxide

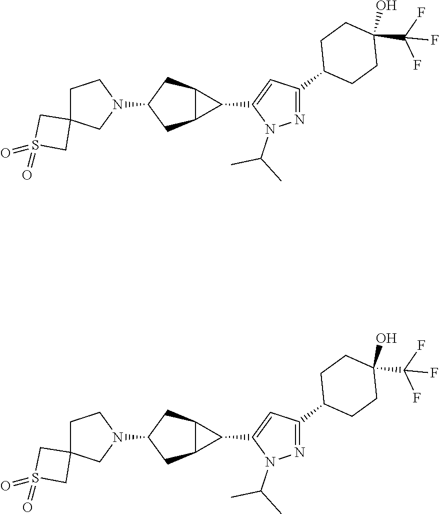 [SMILES: CC(C)n1nc([C@H]2CC[C@](O)(C(F)(F)F)CC2)cc1[C@H]1[C@@H]2C[C@@H](N3CCC4(C3)CS(=O)(=O)C4)C[C@@H]21 ; CC(C)n1nc([C@H]2CC[C@@](O)(C(F)(F)F)CC2)cc1[C@H]1[C@@H]2C[C@@H](N3CCC4(C3)CS(=O)(=O)C4)C[C@@H]21]

Triethylamine trihydrofluoride (1 mL, 6.13 mmol) was added to a solution of 6-((1R,3s,5S,6r)-6-(1-isopropyl-3-(4-(trifluoromethyl)-4-((trimethylsilyl)oxy)cyclohexyl)-1H-pyrazol-5-yl)bicyclo[3.1.0]hexan-3-yl)-2-thia-6-azaspiro[3.4]octane 2,2-dioxide (50.0 mg, 0.08 mmol) in tetrahydrofuran (3 mL). The reaction mixture was stirred at 20° C. for 1 h. The reaction mixture was quenched by addition of saturated NaHCO₃ aqueous solution (10 mL) at 0° C., and extracted with ethyl acetate (50 mL×2). The combined organic layers were dried over anhydrous Na₂SO₄, filtered and concentrated in vacuo. The residue was purified by silica flash chromatography (0-5% dichloromethane in methanol) to afford the title compound (35 mg, 79% yield) as a colorless oil. The SFC showed two peaks LCMS (ESI) [M+H]⁺=516.3.

The mixture of the diastereoisomers were separated using chiral SFC (Daicel Chiralcel AD (250 mm*30 mm, 10 μm); 0.1% NH₃ in H₂O; EtOH; 30%; 60 mL/min) to afford the title compound 104* (22 mg, 62% yield, first peak on SFC) and the title compound 105* (6.4 mg, 16.8% yield, second peak on SFC). LCMS (ESI) [M+H]⁺=516.3. The relative stereochemistry for both compounds was arbitrarily assigned.

Compound 104*: ¹H NMR (400 MHz, CD₃OD) δ 5.71 (s, 1H), 4.68-4.63 (m, 1H), 4.16-4.09 (m, 4H), 2.89-2.87 (m, 3H), 2.74-2.71 (m, 2H), 2.58-2.52 (m, 1H), 2.27-2.19 (m, 4H), 2.02-1.81 (m, 8H), 1.68-1.56 (m, 5H), 1.43 (d, J=6.8 Hz, 6H).

Compound 105*: ¹H NMR (400 MHz, CD₃OD) δ 5.69 (s, 1H), 4.69-4.62 (m, 1H), 4.15-4.08 (m, 4H), 2.86 (s, 2H), 2.73-2.71 (m, 2H), 2.58-2.52 (m, 2H), 2.25-2.18 (m, 4H), 1.89-1.64 (m, 13H), 1.43 (d, J=6.8 Hz, 6H).

### Example VV: 6-((1R,3S,5S,6r)-6-(1-Isopropyl-3-((1s,4S)-4-(trifluoromethyl)cyclohexyl)-1H-pyrazol-5-yl)bicyclo[3.1.0]hexan-3-yl)-2-thia-6-azaspiro[3.4]octane 2,2-dioxide (Compound 106), and 6-((1R,3S,5S,6r)-6-(1-isopropyl-3-((1r,4R)-4-(trifluoromethyl)cyclohexyl)-1H-pyrazol-5-yl)bicyclo[3.1.0]hexan-3-yl)-2-thia-6-azaspiro[3.4]octane 2,2-dioxide (Compound 107)

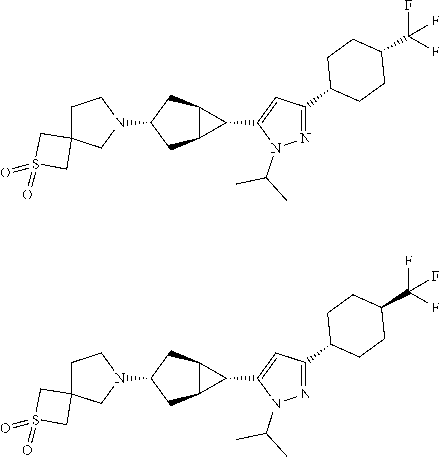 [SMILES: CC(C)n1nc([C@H]2CC[C@@H](C(F)(F)F)CC2)cc1[C@H]1[C@@H]2C[C@@H](N3CCC4(C3)CS(=O)(=O)C4)C[C@@H]21 ; CC(C)n1nc([C@H]2CC[C@H](C(F)(F)F)CC2)cc1[C@H]1[C@@H]2C[C@@H](N3CCC4(C3)CS(=O)(=O)C4)C[C@@H]21]

### Step 1: 6-((1R,3s,5S,6r)-6-(3-Iodo-1-isopropyl-1H-pyrazol-5-yl)bicyclo[3.1.0]hexan-3-yl)-2-thia-6-azaspiro[3.4]octane 2,2-dioxide and 6-((1R,3r,5S,6r)-6-(3-iodo-1-isopropyl-1H-pyrazol-5-yl)bicyclo[3.1.0]hexan-3-yl)-2-thia-6-azaspiro[3.4]octane 2,2-dioxide

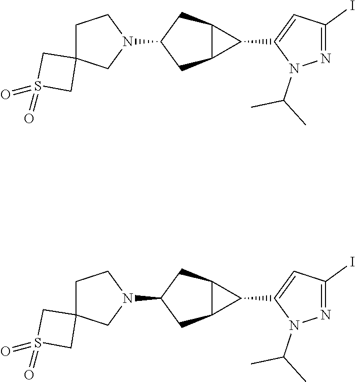 [SMILES: CC(C)n1nc(I)cc1[C@H]1[C@@H]2C[C@@H](N3CCC4(C3)CS(=O)(=O)C4)C[C@@H]21 ; CC(C)n1nc(I)cc1[C@H]1[C@@H]2C[C@H](N3CCC4(C3)CS(=O)(=O)C4)C[C@@H]21]

To a mixture of 2-thia-6-azaspiro[3.4]octane 2,2-dioxide (586 mg, 3.63 mmol) and sodium cyanoborohydride (571.0 mg, 9.09 mmol) in methanol (10 mL) was added (1R,5S)-6-(5-iodo-2-isopropyl-pyrazol-3-yl)bicyclo[3.1.0]hexan-3-one (1.0 g, 3.03 mmol) and acetic acid (909 mg, 15.14 mmol). The reaction mixture was stirred at 60° C. for 3 h. The reaction mixture was quenched with water (10 mL) and the pH was adjusted to 8 with an aqueous NaOH solution (1 M). The reaction mixture was extracted with ethyl acetate (100 mL×3). The combined organic layers were washed with brine (25 mL×3), dried over anhydrous sodium sulfate, filtered and concentrated in vacuo. The residue was purified by silica flash chromatography (0-60% ethyl acetate in petroleum ether) to afford 6-((1R,3s,5S,6r)-6-(3-iodo-1-isopropyl-1H-pyrazol-5-yl)bicyclo[3.1.0]hexan-3-yl)-2-thia-6-azaspiro[3.4]octane 2,2-dioxide (more polar on TLC, 400 mg, 0.816 mmol, 26.9% yield), and 6-((1R,3r,5S,6r)-6-(3-iodo-1-isopropyl-1H-pyrazol-5-yl)bicyclo[3.1.0]hexan-3-yl)-2-thia-6-azaspiro[3.4]octane 2,2-dioxide (less polar on TLC, 1000 mg, 2.0 mmol, 66% yield). Both isomers were obtained as white solids. LCMS (ESI) [M+H]⁺=476.2.

### Step 2: 6-((1R,3s,5S,6r)-6-(1-Isopropyl-3-(4-(trifluoromethyl)cyclohex-1-en-1-yl)-1H-pyrazol-5-yl)bicyclo[3.1.0]hexan-3-yl)-2-thia-6-azaspiro[3.4]octane 2,2-dioxide

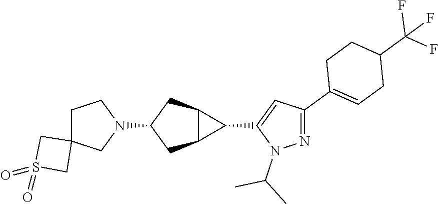 [SMILES: CC(C)n1nc(C2=CCC(C(F)(F)F)CC2)cc1[C@H]1[C@@H]2C[C@@H](N3CCC4(C3)CS(=O)(=O)C4)C[C@@H]21]

To a mixture of potassium carbonate (174 mg, 1.26 mmol) and bis(di-tert-butyl(4-dimethylaminophenyl)phosphine)dichloropalladium(II) (60 mg, 0.08 mmol) in 1,4-dioxane (10 mL) and water (2 mL) were added 6-((1R,3s,5S,6r)-6-(3-iodo-1-isopropyl-1H-pyrazol-5-yl)bicyclo[3.1.0]hexan-3-yl)-2-thia-6-azaspiro[3.4]octane 2,2-dioxide (200.0 mg, 0.420 mmol) and 4,4,5,5-tetramethyl-2-[4-(trifluoromethyl)-1-cyclohexen-1-yl]-1,3,2-dioxaborolane (232 mg, 0.84 mmol). The reaction mixture was stirred under N₂ at 75° C. for 3 h. The reaction mixture was concentrated in vacuo and the residue was purified by silica flash chromatography (0-100% ethyl acetate in petroleum ether) to afford the title compound (200 mg, 0.366 mmol, 86.9% yield) as a yellow oil. LCMS (ESI) [M+H]⁺=488.3.

### Step 3: 6-((1R,3s,5S,6r)-6-(1-Isopropyl-3-(4-(trifluoromethyl)cyclohexyl)-1H-pyrazol-5-yl)bicyclo[3.1.0]hexan-3-yl)-2-thia-6-azaspiro[3.4]octane 2,2-dioxide

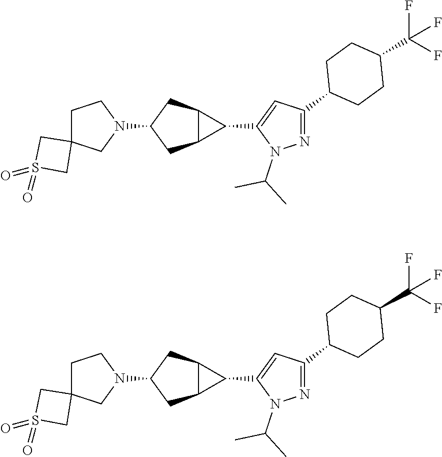 [SMILES: CC(C)n1nc([C@H]2CC[C@@H](C(F)(F)F)CC2)cc1[C@H]1[C@@H]2C[C@@H](N3CCC4(C3)CS(=O)(=O)C4)C[C@@H]21 ; CC(C)n1nc([C@H]2CC[C@H](C(F)(F)F)CC2)cc1[C@H]1[C@@H]2C[C@@H](N3CCC4(C3)CS(=O)(=O)C4)C[C@@H]21]

To a solution of 6-((1R,3s,5S,6r)-6-(1-isopropyl-3-(4-(trifluoromethyl)cyclohex-1-en-1-yl)-1H-pyrazol-5-yl)bicyclo[3.1.0]hexan-3-yl)-2-thia-6-azaspiro[3.4]octane 2,2-dioxide (200 mg, 0.40 mmol) in ethanol (10 mL) was added 10% wt. Pd/C (171 mg, 0.16 mmol) under H₂ (15 psi) for 16 hr. The reaction mixture was filtered and the filtrate was concentrated in vacuo to afford the title compound (140 mg, 0.280 mmol, 70% yield) as a white solid. LCMS (ESI) [M+H]⁺=490.3.

The mixture of the diastereoisomers were separated using chiral SFC (Daicel Chiralpak AD (250 mm*30 mm, 10 μm); 0.1% NH₃ in H₂O; EtOH; 20%; 70 mL/min) to afford the title compound 106 (62.9 mg, 0.1257 mmol, 56% yield, first peak) and the title compound 107 (38.4 mg, 0.075 mmol, 44% yield, second peak on SFC). LCMS (ESI) [M+H]⁺=500.2. The relative stereochemistry for both compounds was arbitrarily assigned.

Compound 106: ¹H NMR (400 MHz, CD₃OD) δ 5.66 (s, 1H), 4.73-4.62 (m, 1H), 4.08 (s, 4H), 3.00-2.92 (m, 1H), 2.91-2.85 (m, 1H), 2.82 (s, 2H), 2.70-2.68 (m, 2H), 2.28-2.12 (m, 5H), 2.09-1.97 (m, 3H), 1.89-1.84 (m, 2H), 1.77-1.68 (m, 6H), 1.62-1.60 (m, 2H), 1.46 (d, J=6.8 Hz, 6H).

Compound 107: ¹H NMR (400 MHz, CD₃OD) δ 5.64 (s, 1H), 4.64-4.59 (m, 1H), 4.07 (s, 4H), 3.01-2.90 (m, 1H), 2.81 (s, 2H), 2.70-2.68 (m, 2H), 2.55-2.45 (m, 1H), 2.24-2.09 (m, 5H), 2.05-1.98 (m, 5H), 1.91-1.76 (m, 2H), 1.62-1.60 (m, 2H), 1.44 (d, J=6.8 Hz, 9H), 1.29 (s, 1H).

### Example WW: 6-((1R,3s,5S,6r)-6-(3-(4,4-Difluorocyclohexyl)-1-isopropyl-1H-pyrazol-5-yl)bicyclo[3.1.0]hexan-3-yl)-2-thia-6-azaspiro[3.4]octane 2,2-dioxide (Compound 108)

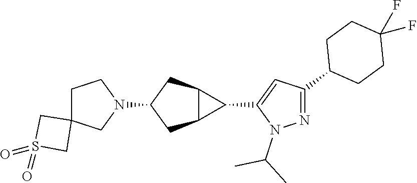

### Step 1: 6-((1R,3s,5S,6r)-6-(3-(4,4-Difluorocyclohex-1-en-1-yl)-1-isopropyl-1H-pyrazol-5-yl)bicyclo[3.1.0]hexan-3-yl)-2-thia-6-azaspiro[3.4]octane 2,2-dioxide

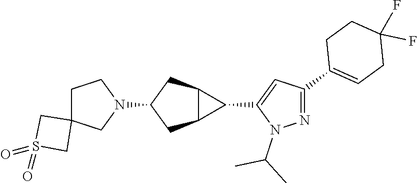

To a mixture of potassium carbonate (87 mg, 0.63 mmol) and bis(di-tert-butyl(4-dimethylaminophenyl)phosphine)dichloropalladium(II) (30 mg, 0.040 mmol) in 1,4-dioxane (5 mL) and water (1 mL) were added 6-((1R,3s,5S,6r)-6-(3-iodo-1-isopropyl-1H-pyrazol-5-yl)bicyclo[3.1.0]hexan-3-yl)-2-thia-6-azaspiro[3.4]octane 2,2-dioxide (100 mg, 0.21 mmol) and 2-(4,4-difluorocyclohexen-1-yl)-4,4,5,5-tetramethyl-1,3,2-dioxaborolane (103 mg, 0.42 mmol). The reaction mixture was then stirred at 75° C. for 3 h under N₂. The reaction mixture was concentrated in vacuo and the residue was purified by silica flash chromatography (0-10% methanol in dichloromethane) to afford the title compound (40 mg, 0.0851 mmol, 40.4% yield) as a yellow solid. LCMS (ESI) [M+H]⁺=466.3.

### Step 2: 6-((1R,3s,5S,6r)-6-(3-(4,4-Difluorocyclohexyl)-1-isopropyl-1H-pyrazol-5-yl)bicyclo[3.1.0]hexan-3-yl)-2-thia-6-azaspiro[3.4]octane 2,2-dioxide

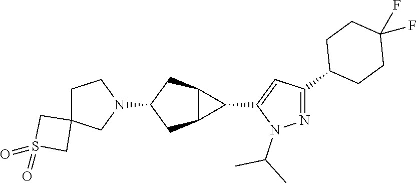

To a solution of 6-((1R,3s,5S,6r)-6-(3-(4,4-difluorocyclohex-1-en-1-yl)-1-isopropyl-1H-pyrazol-5-yl)bicyclo[3.1.0]hexan-3-yl)-2-thia-6-azaspiro[3.4]octane 2,2-dioxide (40.0 mg, 0.09 mmol) in ethanol (4 mL) was added 10% wt. Pd/C (37 mg, 0.03 mmol) under H₂ (15 psi) for 32 h. The reaction mixture was filtered and the filtrate was concentrated in vacuo. The crude product was purified by reverse phase chromatography (Xtimate C18, 150*40 mm*5 μm; water (NH₃H₂O+NH₄HCO₃)-ACN, 40-70%) to provide the title compound (23 mg, 0.05 mmol, 70% yield). LCMS (ESI) [M+H]⁺=468.2. The relative stereochemistry was assigned based on 1H NMR analysis.

Compound 108: ¹H NMR (400 MHz, CDCl₃) δ 5.56 (s, 1H), 4.72-4.53 (1H), 4.03 (s, 4H), 2.96-2.87 (m, 2H), 3.08-2.83 (m, 2H), 2.77-2.67 (m, 4H), 2.59-2.54 (m, 5H), 2.09-2.02 (m, 4H), 1.96 (brs, 3H), 1.88-1.59 (m, 3H), 1.47 (d, J=6.4 Hz, 6H).

### Biological Assay Examples

### Mouse OPC Preparation

To assess effects of treatments on OPCs, all treatments were assayed in two or more independent platings of epiblast stem cell-derived OPCs (EpiSC). EpiSC-derived OPCs were obtained using in vitro differentiation protocols and culture conditions described previously (Najm et al, 2011, Nature Methods). OPCs were expanded and frozen down in aliquots. OPCs were thawed into growth conditions for at least one passage before use in further assays.

### Determination of EC₅₀ Values of Compounds

### In Vitro Phenotypic Screening of OPCs

EpiSC-derived OPCs were grown and expanded in poly-L-ornithine (PO) and laminin-coated flasks in N2B27 media (DMEM/F12 (Gibco), N2-MAX (R&D Systems), B-27 (ThermoFisher), and GlutaMax (Gibco)) supplemented with FGF2 (10 μg/mL, R&D systems, 233-FB-025) and PDGF-AA (10 μg/mL, R&D systems, 233-AA-050) before harvesting for experiments. The cells were seeded onto poly-L-ornithine or poly-D-lysine coated CellCarrier Ultra plates (PerkinElmer) coated with laminin (Sigma, L2020) at a density of 150,000/cm² in N2B27 media without growth factors. For dose-response testing, a 1000× compound stock in dimethyl sulphoxide (DMSO) was added to assay plates, resulting in 8-point dose curves with final concentrations between 1000 nM and 0.5 nM. Positive controls and DMSO vehicle controls were included in each assay plate. Cells were incubated under standard conditions (37° C., 5% CO₂) for 3 days and fixed with 4% paraformaldehyde (PFA) in phosphate buffered saline (PBS) for 20 min. Fixed plates were washed with PBS, permeabilized with 0.1% Triton X-100, and blocked with 10% donkey serum (v/v) in PBS for 40 min. Then, cells were labelled with MBP antibodies (Abcam, ab7349; 1:200) for 2 h at room temperature, washed with PBS, and stained with Alexa Fluor conjugated secondary antibodies (1:500) for 45 min. Nuclei were visualized by DAPI staining (Sigma; 1 μg/ml), followed by further PBS washes.

### High-Content Imaging and Analysis

Cells and cell culture plates were imaged on the Operetta High Content Imaging and Analysis system (PerkinElmer). Analysis (PerkinElmer Harmony and Columbus software) began by identifying intact nuclei stained by DAPI. The peri-nuclear region of each cell was then cross-referenced with the mature myelin protein (MBP) stain to identify oligodendrocyte nuclei, and from this the percentage of oligodendrocytes was calculated. EC₅₀ values were calculated using The Levenberg-Marquardt algorithm to fit a Hill equation to dose-response data (0.5 nM to 1000 nM). The results are provided in Table 4 (OPC EC₅₀).

### Determination of Potency and Enzyme Target

### GC/MS-Based Sterol Profiling

Sterols were monitored using a modified Folch wash protocol (Hubler et al, 2018, Nature). EpiSC-derived OPCs were plated at 100,000 cells per well in PO- and laminin-coated 96-well plates in N2B27 media without growth factors. After 24 hours, cells were rinsed with saline and plates were frozen. Cholesterol-d7 standard was then added to each well before drying under nitrogen stream and derivatization with 55 μl of bis(trimethylsilyl) trifluoroacetamide. After derivatization, 2 μl were analyzed by gas chromatography/mass spectrometry using an Agilent 5973 Network Mass Selective Detector equipped with a 6890 gas chromatograph system and a HP-5MS capillary column (30 m×0.25 mm×0.25 mm). Samples were analyzed in full scan mode using electron impact ionization; ion fragment peaks were integrated to calculate sterol abundance, and quantitation was relative to cholesterol-d7. The following ion fragments were used to quantitate each metabolite: cholesterol-d7 (465), FF-Mas (482), cholesterol (368), zymostenol (458), zymosterol (456), Desmosterol (456, 343), 7-dehydrocholesterol (456, 325), lanosterol (393), lathosterol (458), 14-dehydrozymostenol (456, 351). For reference, Table 2 shows sterol GC-MS analytes and their relationship with inhibitors of cholesterol biosynthesis. All standards were obtained from Avanti Polar Lipids unless otherwise indicated. Calibration curves were generated by injecting varying concentrations of sterol standards and maintaining a fixed amount of cholesterol-D7. For normalized zymostenol accumulation results, the total amount of zymostenol measured after drug treatment was divided by the total amount of zymostenol accumulated after 24 hr treatment with 100 nM positive control reference. EC₅₀ values were calculated using The Levenberg-Marquardt algorithm to fit a Hill equation to dose-response data (8 doses from 0.15 nM to 333 nM). EC₅₀ values for zymostenol (Zymo GCMS EC₅₀) are provided in Table 4.

TABLE 2

| Analyte increased | Cholesterol enzyme inhibited |
|---|---|
| Lanosterol | CYP51 |
| FF-MAS | TM7SF2 (Sterol 14 Reductase) |
| 14-dehydrozymostenol | TM7SF2 (Sterol 14 Reductase) |
| Zymosterol | EBP |
| Zymostenol | EBP |
| 7-dehydrocholesterol | DHCR7 |

### Determination of Binding Affinity

Membrane preparation: To examine compound binding affinity to EBP, human EBP was overexpressed in human embryonic kidney 293 cells. Cell pellet was lysed in 10 times weight binding buffer (50 mM Tris, 5 mM MgCl₂, 0.1 mM EDTA, lx protease inhibitor cocktail, pH 7.5) on ice by using a dounce homogenizer. The solution was centrifuged at 25,000 g for 50 min at 4° C. The membrane pellet was re-suspended in binding buffer and run through a 25⅝ gauge needle. After checking the concentration by Bradford assay, the whole cell membrane solution was adjusted to 20 mg/mL and stored at −80° C.

Determination of equilibrium dissociation constant Kd of radioligand: Membrane prepared as described above was pre-incubated with PVT-WGA SPA beads (Perkinelmer Cat #RPNQ0003) at a ratio of 0.3 mg beads with 5 μg membrane per 25 μL binding buffer at 20° C. for 2 hours with gentle shaking. This binding solution was centrifuged at 400 g for 5 minutes to collect the bead/membrane mixture. After re-suspending the pellet in binding buffer at the same calculated volume with 0.01% BSA (Sigma A1933), the bead/membrane mixture was added in 384-well low-binding surface plate (PerkinElmer Cat #6057480) at 25 l/well. Radioligand at different concentrations with and without the non-radio-labeled same ligand 5 uM (for nonspecific and total signal, respectively) was added to bring final volume to 50 l/well with DMSO concentration at 0.1%. At equilibrium (3 hours after ligand addition), radiometric signal CPM was counted by using a Microbeta2 microplate counter (Perkinelmer). The Kd was determined by nonlinear regression fitting of specific signal plot against the concentration of radioligand [3H]-Ifenprodil (Table 3).

TABLE 3

| Target | Radioligand | Kd | [L] |
|---|---|---|---|
| EBP | [3H]-Ifenprodil, (Perkinelmer | 15.86 nM | 10 nM |
| | Cat# NET1089250UC) | | |

[L], concentration of radioligand used in assay

Competition binding assay to determine compound affinity: The same conditions of the radioligand Kd study were used for compound single dose percentage inhibition and equilibrium dissociation constant Ki examinations, except 50 nL compound DMSO stock was pre-added in 384-well low-binding surface plate (PerkinElmer Cat #6057480) by Echo 550 (Labcyte) to reach the final concentration for single dose test at 1 uM, and dose response test from 0.06 nM to 5 uM (8 dose, 5 times dilution). A pre-incubated bead/membrane mixture was added in compound plate at 0.3 mg beads and 5 g membrane per well. Radioligand [3H]-Ifenprodil was added to reach optimized concentration [L] and bring assay volume to 50 μl. At equilibrium (3 hours after ligand addition), radiometric signal was counted as described above. The percentage inhibition of compound at each testing concentration was calculated by normalizing each condition's CPM readout to full block (5 uM non-radiolabeled ligand) and non-block (DMSO) control conditions. Compound binding inhibition IC₅₀ was determined by nonlinear regression fitting of percentage inhibition plot against compound concentration. Compound Ki was calculated from the equation Ki=IC₅₀/(1+[L]/Kd), which [L] was radioligand concentration used in assay. All tests had N bigger or equal to 2. The data from this experiment is shown in Table 4 (hEBP SPA Ki).

### Determination of Binding Affinity to EBP-7-Dehydrocholesterol Reductase

### Materials and Instruments

| Materials | Vendor | Cat# |
|---|---|---|
| (S)-6-(2-Methyl-3-(6-(trifluoromethyl) | Moravek | MT-1003106 |
| pyridin-3-yl)propyl)-2-thia-6-azaspiro | | |
| [3.4]octane 2,2-dioxide or | | |
| (R)-6-(2-Methyl-3-(6-(trifluoromethyl) | | |
| pyridin-3-yl)propyl)-2-thia-6- | | |
| azaspiro[3.4]octane 2,2-dioxide, [3H] | | |
| Ifenprodil (+)-tartrate salt | Sigma | I2892 |
| Bovine Serum Albumin (BSA) | Sigma | A1933 |
| Ethylenediaminetetraacetic acid | Sigma | E5391 |
| tetrasodium salt hydrate (EDTA) | | |
| Magnesium Chloride Hexahydrate | Sigma | M2670 |
| (MgCl2·6H2O) | | |
| Tris(hydroxymethyl)aminomethane (Tris) | Alfa Aesar | A18494 |
| Polyethylenimine, branched (PEI) | Sigma | 408727 |

### Key Instruments and Consumables

| Item | Supplier | Cat# |
|---|---|---|
| MicroBeta2 Microplate Counter | PerkinElmer | 2450-0060 |
| UniFilter-96 GF/B | PerkinElmer | 6005177 |
| TopSeal | Biotss | SF-800 |
| MicroBeta Filtermate-96 | PerkinElmer | D961962 |
| Seven Compact pH meter | Mettler Toledo | S220 |
| Ultrapure Water Meter | Sichuan Ulupure | UPH-III-20T |
| Benchtop Centrifuge | Hunan Xiangyi | L550 |
| Microplate Shaker | Allsheng | MX100-4A |
| 384-Well Polypropylene Microplate | Labcyte | PP-0200 |
| 96-Well U bottom Plate | Corning | 7007 |
| Echo | LABCYTE | 550 |

### Assay Buffer Preparation

| Reagent | Concentration | |
|---|---|---|
| Tris | 50 | mM |
| MgCl₂ | 5 | mM |
| EDTA | 0.1 | mM |
| BSA | 0.01% | (w/v) |

Adjust pH to 7.4 followed by 0.2 μM sterile filtration

Membrane Preparation: human emopamil binding protein and human 7-dehydrocholesterol reductase co-expressing cells were generated by transient transfecting host human embryonic kidney (HEK) 293 cells with 2 DNA constructs containing each protein's coding sequence. Cells were suspension cultured at 37° C. with 5% CO₂ in FREESTYLE 293 Expression Medium (Thermofisher). Whole cell membrane was prepared by harvesting the cell pellet, adding cold membrane buffer (50 mM Tris, pH7.5, lx Roche COMPLETE EDTA-free protease inhibitor cocktail) 10 times volume of the cell pellets weight, lysing cell pellet on ice by using Dounce homogenizer, spinning at 200 g 4° C. for 15 min, collecting supernatant and spinning again at 25000 g 4° C. for 50 min, transferring pellet to Dounce homogenizer, re-suspending pellet by homogenizing in membrane buffer on ice to reach ˜25 mg/mL, then keeping whole cell membrane aliquots at −80° C.

Compounds were prepared in a 96-well U bottom plate using an Echo550 machine and 10 mM compound DMSO stock solution, followed by an 8-dose 5-fold serial dilutions protocol with final testing compound concentration ranging from 0.06 to 5000 nM, with DMSO back fill to 100 nL/well and n=2. DMSO and Ifenprodil 5 uM wells were added in each plate as 0 and 100% inhibition reference controls with n=8 for each condition. The UniFilter-96 GF/B plates were pre-treated by adding 50 l/well of 0.3% (v/v) PEI to UniFilter-96 GF/B plates. The plates were sealed and incubated at 4° C. for 3 hrs. Then, the plates were washed with ice-cold assay buffer 3 times. The radioligand binding assay was prepared by adding assay buffer diluted hEBP-DHCR7 membrane at 66.7 μg/ml×150 l/well into the 96-well compound plate to reach 10 μg membrane per well. Then, the assay buffer diluted [3H]—((S)-6-(2-Methyl-3-(6-(trifluoromethyl)pyridin-3-yl)propyl)-2-thia-6-azaspiro[3.4]octane 2,2-dioxide or (R)-6-(2-Methyl-3-(6-(trifluoromethyl)pyridin-3-yl)propyl)-2-thia-6-azaspiro[3.4]octane 2,2-dioxide) was added at 25 nM×50 l/well. Following this, the plate was centrifuged at 1000 rpm for 30 secs. The plate was then sealed and agitated at 600 rpm at 22° C. for 5 min, and then incubated at 22° C. for 3 hrs. The incubation was stopped by transferring the binding solution to the pre-treated UniFilter-96 GF/B plate, vacuum filtrated, and then washed four times with ice-cold assay buffer. Following this, the plates were dried at 37° C. for 45 min. The plates were then sealed at the bottom. 40 l/well of scintillation cocktail was added to the plates. A MicroBeta2 microplate counter was then used to read the plate and analyze the data. For reference and test compounds, the results are expressed as % Inhibition, using the normalization equation: N=100−100×(U−C2)/(C1−C2), where U is the unknown value, C1 is the average of high controls, and C2 is the average value of low controls. The IC50 was determined by fitting percentage of inhibition as a function of compound concentrations with Hill equation using XLfit. Results are expressed as hEBP-DHCR7 Ki (uM) in Table 4. Ki was calculated as described above; * indicates isolated isomer or isomers, but that the stereochemistry has not been assigned.

TABLE 4

| Cmpd. | hEBP SPA | hEBP-DHCR7 | Zymostenol GCMS | OPC EC₅₀ |
|---|---|---|---|---|
| No. | Ki (μM) | Ki (uM) | EC₅₀ (μM) | (μM) |
| 1 | ND | 0.0131 | 0.0012 | 0.0013 |
| 2 | 0.038 | 0.00591 | 0.00181 | 0.0009 |
| 3 | ND | 0.0162 | 0.0039 | 0.034 |
| 4 | 0.031 | 0.0145 | 0.0030 | 0.014 |
| 5 | 0.022 | 0.043 | 0.0036 | 0.008 |
| 6 | 0.019 | 0.0181 | 0.029 | 0.20 |
| 7* | ND | 0.0173 | 0.0089 | 0.037 |
| 8* | ND | 0.0181 | 0.0036 | 0.0025 |
| 9* | 0.026 | 0.0201 | 0.0027 | 0.0038 |
| 10* | ND | 0.0199 | 0.0048 | 0.013 |
| 11 | 0.036 | 0.0132 | 0.0072 | 0.014 |
| 12 | 0.017 | 0.012 | 0.0037 | 0.0097 |
| 13 | ND | 0.0371 | 0.0065 | 0.017 |
| 14 | ND | 0.013 | 0.0020 | 0.0029 |
| 15 | 0.019 | 0.020 | 0.0032 | 0.0066 |
| 16 | 0.020 | 0.021 | 0.0067 | 0.024 |
| 17 | 0.067 | 0.116 | 0.042 | 0.42 |
| 18 | 0.030 | 0.017 | 0.0023 | 0.015 |
| 19 | 0.016 | 0.0096 | 0.0025 | 0.013 |
| 20 | 0.062 | 0.0489 | 0.0012 | 0.039 |
| 21 | 0.043 | 0.0171 | 0.0031 | 0.0066 |
| 22 | 0.030 | 0.0124 | 0.012 | 0.053 |
| 23 | 0.013 | 0.0124 | 0.012 | 0.053 |
| 24 | 0.030 | 0.0199 | 0.0018 | 0.0093 |
| 25 | 0.026 | 0.0146 | 0.00036 | 0.0042 |
| 26 | ND | 0.0198 | 0.0044 | 0.0065 |
| 27 | ND | 0.0116 | 0.0023 | 0.0016 |
| 28 | 0.022 | 0.0246 | 0.006 | 0.039 |
| 31 | 0.037 | 0.0192 | 0.0071 | 0.030 |
| 32 | 0.023 | 0.0135 | 0.0053 | 0.029 |
| 38 | 0.026 | 0.041 | 0.0029 | 0.0049 |
| 40 | 0.026 | 0.0184 | 0.0059 | 0.015 |
| 43* | ND | 0.0503 | 0.011 | 0.048 |
| 44* | ND | 0.027 | 0.0068 | 0.014 |
| 45* | ND | 0.131 | 0.0081 | 0.014 |
| 46* | ND | 0.0346 | 0.024 | 0.044 |
| 47 | 0.019 | 0.175 | 0.0019 | 0.0017 |
| 48 | 0.015 | 0.0125 | 0.010 | 0.015 |
| 49 | 0.024 | 0.0649 | 0.0027 | 0.0046 |
| 50 | 0.018 | 0.049 | 0.020 | 0.069 |
| 51 | ND | 0.0117 | 0.0064 | 0.037 |
| 52 | ND | 0.0103 | 0.0024 | ND |
| 53* | ND | 0.0973 | 0.0068 | 0.033 |
| 54* | ND | 0.0328 | 0.0091 | 0.031 |
| 55 | ND | 0.0131 | 0.003 | 0.0033 |
| 56 | ND | 0.00919 | 0.0057 | 0.019 |
| 57 | ND | 0.026 | 0.0035 | 0.018 |
| 58 | ND | 0.012 | 0.0033 | 0.019 |
| 62* | ND | 0.134 | 0.012 | 0.060 |
| 63* | ND | 0.0652 | 0.019 | 0.13 |
| 64* | ND | 0.203 | 0.044 | ND |
| 65* | ND | 0.0519 | 0.018 | 0.065 |
| 66* | ND | 0.0264 | 0.035 | ND |
| 67* | ND | 0.171 | 0.037 | ND |
| 68* | ND | 0.0862 | 0.014 | 0.06 |
| 69* | ND | 0.0343 | 0.0044 | 0.027 |
| 70* | ND | 0.0553 | 0.0094 | 0.024 |
| 71* | ND | 0.0178 | 0.017 | 0.076 |
| 72 | ND | 0.303 | 0.047 | ND |
| 73 | ND | 0.0591 | 0.032 | 0.092 |
| 74 | ND | ND | 0.0039 | 0.013 |
| 75 | ND | 0.0149 | 0.0022 | 0.0062 |
| 76 | ND | 0.0118 | 0.0022 | ND |
| 77* | ND | 0.093 | 0.330 | ND |
| 78* | ND | 0.0336 | 0.053 | ND |
| 79 | ND | ND | 0.003 | ND |
| 80* | ND | ND | 0.0043 | ND |
| 81* | ND | ND | 0.027 | ND |
| 82* | ND | ND | 0.0036 | ND |
| 83* | ND | ND | 0.041 | ND |
| 86* | ND | 0.0212 | 0.013 | ND |
| 87* | ND | 0.0946 | >0.33 | ND |
| 88* | ND | 0.0189 | 0.0077 | ND |
| 89* | ND | 0.128 | 0.27 | ND |
| 90* | ND | 0.0705 | 0.021 | 0.19 |
| 91* | ND | 0.0163 | 0.024 | ND |
| 92* | ND | ND | 0.0451 | 0.0584 |
| 93* | ND | ND | 0.24 | ND |
| 94* | ND | ND | 0.00347 | ND |
| 95* | ND | ND | 0.0583 | ND |
| 96* | ND | 0.438 | 0.0379 | ND |
| 97 | ND | 0.00685 | 0.0221 | ND |
| 98* | 0.051 | 0.054 | 0.055 | 0.30 |
| 99* | 0.058 | 0.048 | 0.086 | 0.30 |
| 102 | ND | 0.0425 | 0.014 | 0.0646 |
| 103 | ND | ND | ND | ND |
| 104* | ND | 0.164 | 0.0258 | 0.175 |
| 105* | ND | 0.554 | 0.11 | ND |
| 106 | ND | 0.0131 | 0.0146 | ND |
| 107 | ND | 0.016 | 0.00759 | 0.0525 |
| 108 | ND | 0.0216 | 0.0106 | 0.102 |

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, NY; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

1. A compound of Formula I: 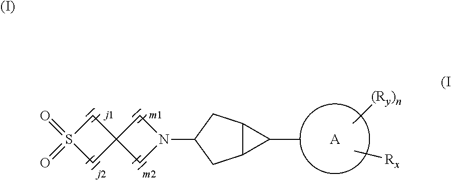
or a pharmaceutically acceptable salt thereof, wherein,
j₁ and m₁ are each independently 1, 2, or 3;
j₂ and m₂ are each independently 0, 1, 2, or 3;
wherein the sum of j₁ and j₂ and the sum of m₁ and m₂ are each no more than 5, and the total sum of j₁, j₂, m₁, and m₂ is no more than 9; and, when one of m₂ and j₂ is 0, the other is 1, 2 or 3;
Ring A is a 5-membered heteroaryl comprising one, two, or three heteroatoms independently selected from the group consisting of O, N, and S;
R_{y}, if present, in each instance is independently selected from the group consisting of halogen, C₁-C₆ alkyl, halo-C₁-C₆ alkyl, C₁-C₆ alkoxy, halo-C₁-C₆ alkoxy, C₃-C₇ cycloalkyl, C₃-C₇ halocycloalkyl, and —CN;
n is 0, 1, 2, or 3;
Rₓ is selected from the group consisting of halogen, C₁-C₁₀ alkyl, halo-C₁-C₆ alkyl, C₁-C₁₀ alkenyl, halo-C₁-C₆ alkenyl, C₁-C₆ alkoxy, halo-C₁-C₆ alkoxy, 5- to 7-membered heterocyclyl, C₃-C₇ cycloalkyl, C₆-C₁₀ aryl, 5- to 6-membered heteroaryl, and —CN;
wherein said heterocyclyl, cycloalkyl, aryl, or heteroaryl is optionally substituted with (R_{xA});
wherein q is 0, 1, 2, 3, 4, or 5; and
each R_{xA} is independently selected from the group consisting of halogen, C₁-C₆ alkyl, hydroxy, C₁-C₆ alkoxy, halo-C₁-C₆ alkoxy, halo-C₁-C₆ alkyl, —SO₂(C₁-C₆ alkyl), and —CN.

2.-3. (canceled)

4. The compound of claim 1, wherein the compound is of Formula Ta: 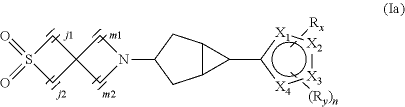
or a pharmaceutically acceptable salt thereof, wherein X₁, X₂, X₃, and X₄ are each individually N, NH, N substituted with Rₓ or R_{y}, C substituted with Rₓ or R_{y}, or CH, wherein one, two, or three of X₁, X₂, X₃, and X₄ are N, NH, or substituted N.

5.-7. (canceled)

8. The compound of claim 4, wherein the compound is of Formula Ib: 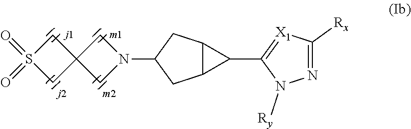
or a pharmaceutically acceptable salt thereof, wherein X₁ is CH or N and R_{y} is C₁-C₆ alkyl or C₃-C₅ cycloalkyl.

9. The compound of claim 1, or a pharmaceutically acceptable salt thereof, wherein R_{y}, in each instance, is selected from the group consisting of methyl, ethyl, propyl, butyl, and cyclobutyl.

10. (canceled)

11. The compound of claim 9, or a pharmaceutically acceptable salt thereof, wherein R_{y} is isopropyl.

12. The compound of claim 1, or a pharmaceutically acceptable salt thereof, wherein Rₓ is selected from the group consisting of C₁-C₆ alkyl, halo-C₁-C₆ alkyl, C₁-C₁₀ alkenyl, halo-C₁-C₆ alkenyl, halo-C₁-C₆ alkoxy, C₃-C₇ cycloalkyl, C₆-C₁₀ aryl, and 5- to 6-membered heteroaryl; wherein said cycloalkyl, aryl, or heteroaryl is substituted with (R_{xA})_{q}, wherein q is 0, 1, 2, or 3, and, if present, each R_{xA} is independently selected from the group consisting of halogen, C₁-C₆ alkyl, hydroxy, C₁-C₆ alkoxy, halo-C₁-C₆ alkoxy, and halo-C₁-C₆ alkyl.

13.-14. (canceled)

15. The compound of claim 12, or a pharmaceutically acceptable salt thereof, wherein Rₓ is trifluoromethyl.

16.-17. (canceled)

18. The compound of claim 8, wherein the compound is of Formula Ic: 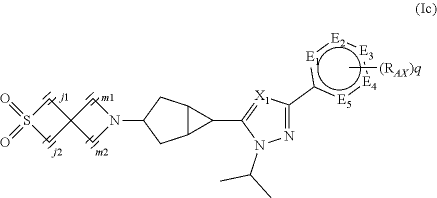
or a pharmaceutically acceptable salt thereof, wherein E₁, E₂, E₃, E₄, and E₅ are each independently N, C when bound to R_{XA}, or CH, wherein up to three of E₁, E₂, E₃, E₄, and E₅ are N; and q is 1 or 2.

19. (canceled)

20. The compound of claim 18, or a pharmaceutically acceptable salt thereof, wherein;
(a) E₁, E₂, E₃, and E₅ are CH and E₄ is C—R_{XA};
(b) E₁ is CH, E₂ is N, E₃ is CH, E₄ is C—R_{XA}, and E₅ is CH;
(c) E₁, E₂, E₄, and E₅ are CH, and E₃ is C—R_{XA};
(d) E₁ is CH, E₂ is N, E₃ is C—R_{XA}, E₄ is N, and E₅ is CH;
(e) E₁, E₂, and E₃ are CH, E₄ is C—R_{XA}, and E₅ is N;
(f) E₁ is CH, E₂ is N, E₃ is C—R_{XA}, and E₄ and E₅ are CH;
(g) E₁ is CH, E₂ is CH, E₃ is N, E₄ is C—R_{XA}, and E₅ is N;
(h) E₁ is CH, E₂ is CH, E₃ is N, E₄ is C—R_{XA}, and E₅ is CH; or
(i) E₁ is N, E₂ is CH, E₃ is CH, E₄ is C—R_{XA}, and E₅ is CH.

21.-28. (canceled)

29. The compound of claim 18, or a pharmaceutically acceptable salt thereof, wherein R_{XA} is halo-C₁-C₆ alkyl.

30. (canceled)

31. The compound of claim 29, or a pharmaceutically acceptable salt thereof, wherein R_{XA} is trifluoromethyl.

32. The compound of claim 4, or a pharmaceutically acceptable salt thereof, wherein X₁ is CH.

33.-34. (canceled)

35. The compound of claim 1, or a pharmaceutically acceptable salt thereof, wherein one of m₁ and m₂ is 1 and the other is 2; or wherein m₁ and m₂ are each 2.

36.-39. (canceled)

40. The compound of claim 35, or a pharmaceutically acceptable salt thereof, wherein one of j₁ and j₂ is 1 and the other is 2.

41. (canceled)

42. The compound of claim 35, or a pharmaceutically acceptable salt thereof, wherein j₁ and j₂ are each 1.

43.-46. (canceled)

47. The compound of claim 1, or a pharmaceutically acceptable salt thereof, wherein the center bicyclic 3.1.0 ring is:

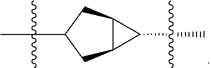

48. The compound of claim 1, wherein the compound is selected from the group consisting of 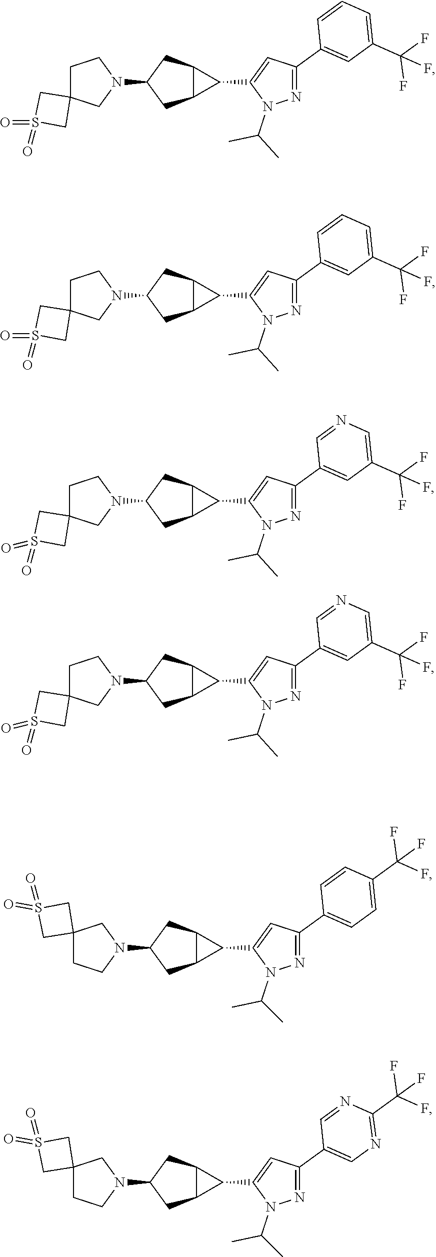 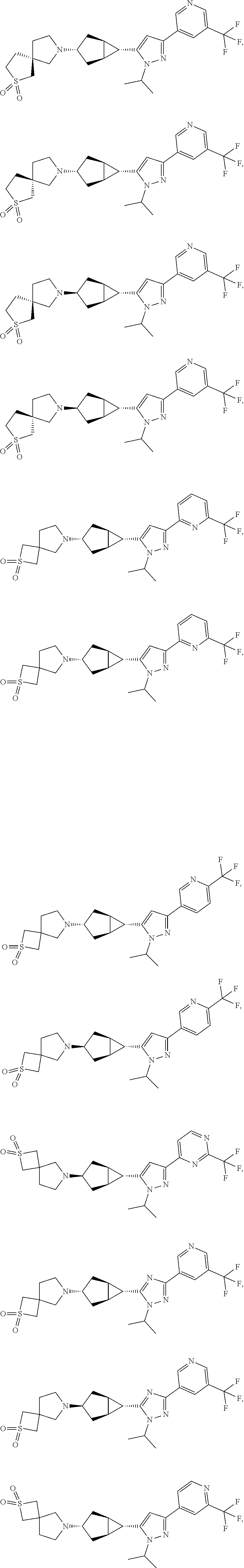 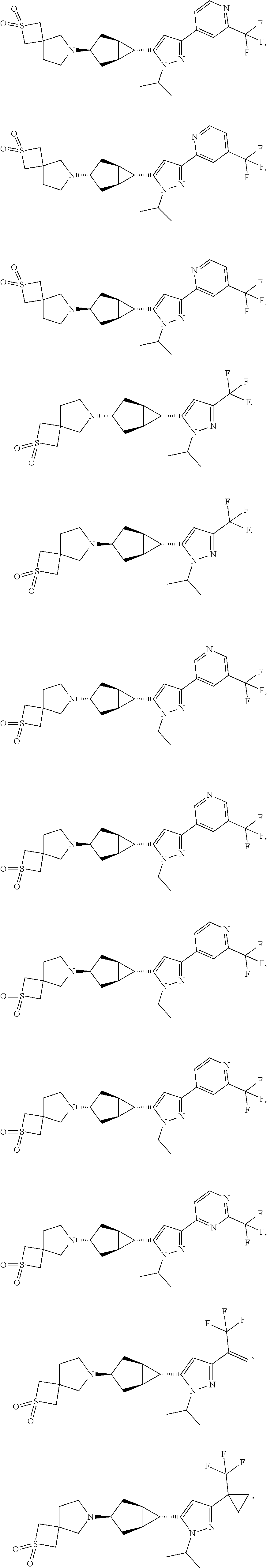 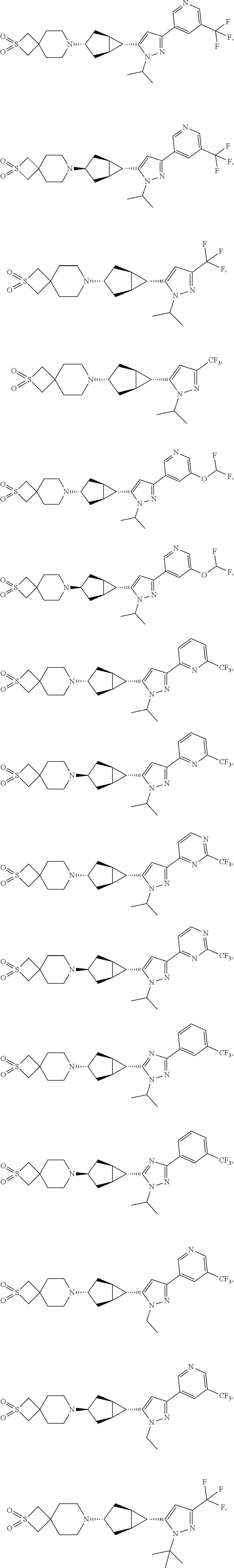 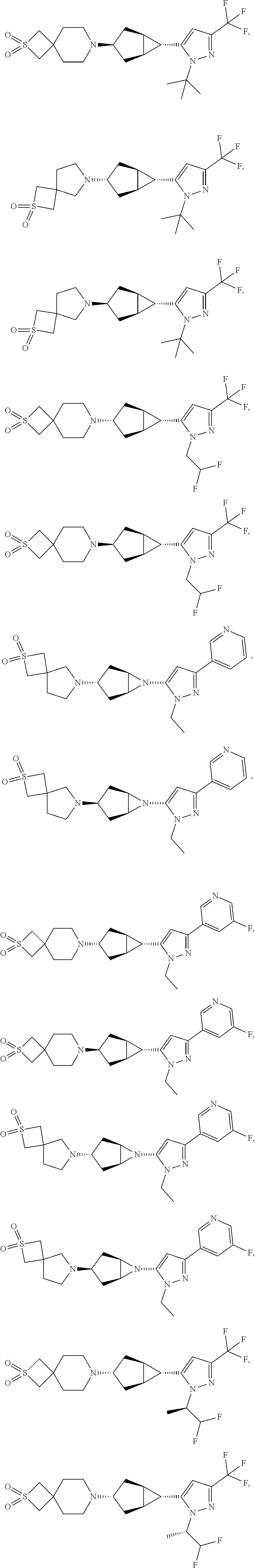 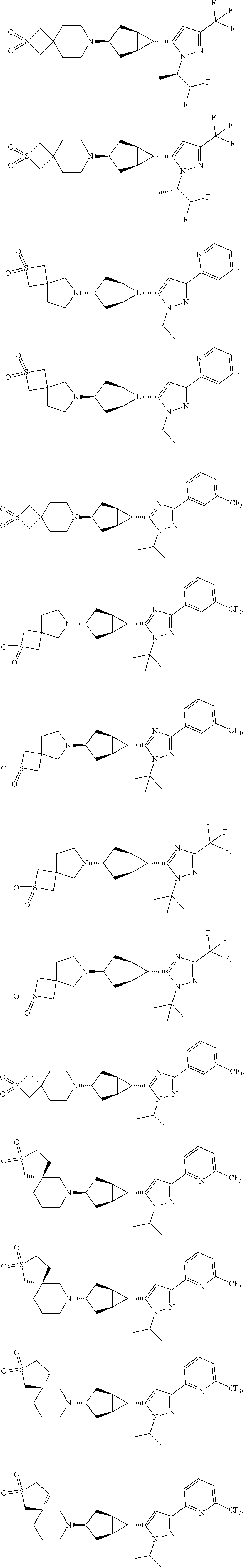 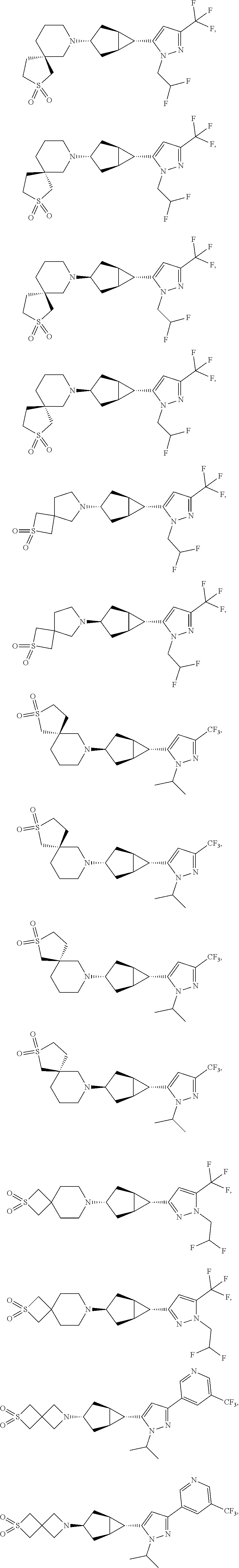 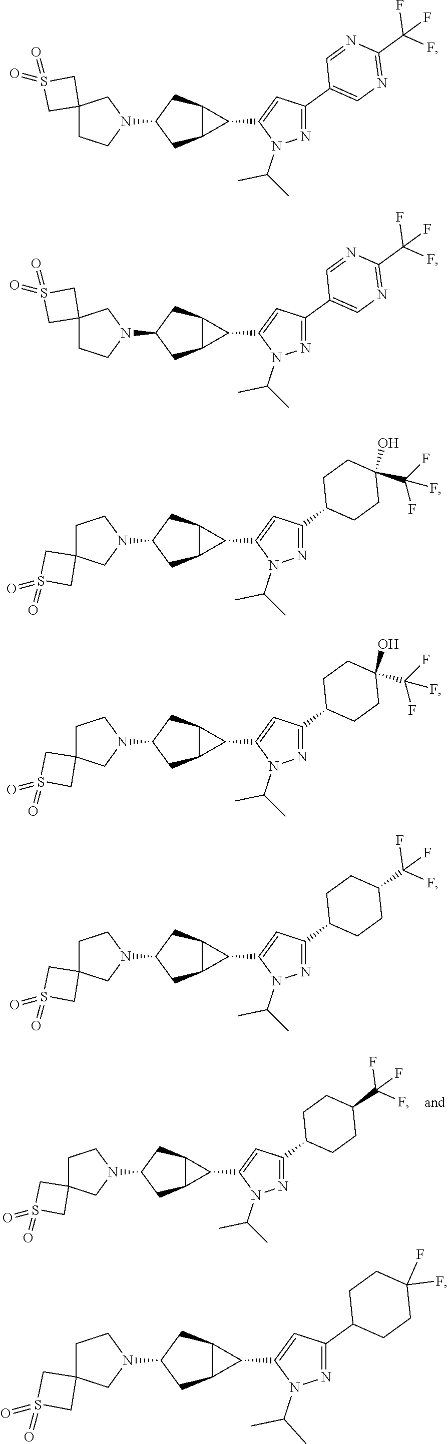
or a pharmaceutically acceptable salt thereof.

49. A pharmaceutical composition comprising a compound of claim 1 or a pharmaceutically acceptable salt thereof, and a pharmaceutically acceptable excipient.

50. A method of treating a disorder in a subject in need thereof, the method comprising administering to the subject in need thereof a therapeutically effective amount of a compound of claim 1, or a pharmaceutically acceptable salt thereof.

51.-52. (canceled)

53. A method of promoting myelination in a subject in need thereof, comprising administering to the subject a therapeutically effective amount of a compound claim 1, or a pharmaceutically acceptable salt thereof.

54.-57. (canceled)
